(19) United States
(12) Patent Application Publication
(10) Pub. No.: US 2024/0067729 A1
(43) Pub. Date: Feb. 29, 2024

(54) ANTI-PD-L1 ANTIBODIES AND FUSION PROTEINS THEREOF

(71) Applicant: Palleon Pharmaceuticals Inc., Waltham, MA (US)

(72) Inventors: Li Peng, Lexington, MA (US); Jenny Che, Ashland, MA (US); Lihui Xu, Chestnut Hill, MA (US)

(21) Appl. No.: 18/260,364

(22) PCT Filed: Jan. 6, 2022
(86) PCT No.: PCT/US2022/011504

§ 371 Date: Jul. 5, 2023

Related U.S. Application Data
(60) Provisional application No. 63/134,412, filed on Jan. 6, 2021.

(51) Int. Cl. C07K 16/28; A61P 35/00; C12N 9/24

(52) U.S. Cl. CPC C07K 16/2827; A61P 35/00; C12N 9/2402; C12Y 302/01018; A61K 2039/505; C07K 2317/24; C07K 2317/622; C07K 2317/76; C07K 2317/77; C07K 2317/92; C07K 2319/33

(57) ABSTRACT

The invention relates generally to anti-PD-L1 antibodies, and recombinant sialidase and anti-PD-L1 immunoglobulin antigen-binding domain fusion proteins. The invention also provides antibody conjugates including a sialidase and an anti-PD-L1 antibody or a portion thereof. The invention further relates to methods of using the antibodies, sialidase fusion proteins, or antibody conjugates for treating cancer.

### CROSS-REFERENCE TO RELATED APPLICATIONS

This application claims the benefit of and priority to U.S. Provisional Patent Application No. 63/134,412, filed Jan. 6, 2021, the disclosure of which is incorporated herein by reference in its entirety for all purposes.

### FIELD OF THE INVENTION

The invention relates generally to antibodies, recombinant sialidase fusion proteins, and antibody conjugates, and their use in the treatment of cancer.

### BACKGROUND

Programmed death-ligand 1 (PD-L1) also known as cluster of differentiation 274 (CD274) or B7 homolog 1 (B7-H1) is a protein that in humans is encoded by the CD274 gene. Upregulation of PD-L1 may allow certain cancers to evade the host immune system. An analysis of 196 tumor specimens from patients with renal cell carcinoma found that high tumor expression of PD-L1 was associated with increased tumor aggressiveness and a 4.5-fold increased risk of death (Thompson et al. (2004) PROC. NATL. ACAD. SCI. USA 101(49) 17174-17179). PD-L1 expression is detected in many human cancers, including bladder, breast, cervical, esophageal, gastric, kidney, lung, ovary and pancreatic cancer (Wang et al. (2016) ONCO. TARGETS THER. 9:5023-5039). For certain cancers, expression of PD-L1 is associated with reduced numbers of tumor infiltrating lymphocytes and poor prognosis (Ohaegbulam et al. (2015) TRENDS MOL. MED. 21(1): 24-33). A number of anti-PD-L1 antibodies have already been approved in the United States for treating a variety of cancers. For example, atezolizumab has been approved for use in, for example, urothelial carcinomas, non-small cell lung cancers (NSCLC), triple-negative breast cancers, and small cell lung cancers, durvalumab has been approved for use in, for example, urothelial carcinomas, and NSCLCs, and avelumab has been approved for use in Merkel cell carcinomas, urothelial carcinomas, and renal cell carcinomas. Other PD-L1 antibodies are in still in development as immuno-oncology therapies and are showing good results in clinical trials including for treating NSCLC and melanoma (Akinleye et al. (2019) J. HEMATOL. ONCOL. 12(1):92).

A growing body of evidence supports roles for glycans, and sialoglycans in particular, at various pathophysiological steps of tumor progression. Glycans regulate tumor proliferation, invasion, hematogenous metastasis and angiogenesis (Fuster et al. (2005) NAT. REV. CANCER 5(7): 526-42). The sialylation of cell surface glycoconjugates is frequently altered in cancers, resulting in the expression of sialylated tumor-associated carbohydrate antigens. The expression of sialylated glycans by tumor cells is often associated with increased aggressiveness and metastatic potential of a tumor (Julien S., Delannoy P. (2015) “Sialic Acid and Cancer”, In: Taniguchi N., Endo T., Hart G., Seeberger P., Wong C H. (eds) Glycoscience: Biology and Medicine. Springer, Tokyo. https://doi.org/10.1007/978-4-431-54841-6_193).

It has recently become apparent that Siglecs (sialic acid-binding immunoglobulin-like lectins), a family of sialic acid binding lectins, play a role in cancer immune suppression by binding to hypersialylated cancer cells and mediating the suppression of signals from activating NK cell receptors, thereby inhibiting NK cell-mediated killing of tumor cells (Jandus et al. (2014) J. CLIN. INVEST. 124: 1810-1820; Laubli et al. (2014) PROC. NATL. ACAD. SCI. USA 111: 14211-14216; Hudak et al. (2014) NAT. CHEM. BIOL. 10: 69-75). Likewise, enzymatic removal of sialic acids by treatment with sialidase can enhance NK cell-mediated killing of tumor cells (Jandus, supra; Hudak, supra; Xiao et al. (2016) PROC. NATL. ACAD. SCI. USA 113(37): 10304-9).

Cancer immunotherapy with immune checkpoint inhibitors, including antibodies that block the PD-1/PD-L1 pathway, has improved the outcome of many cancer patients. However, despite advances that have been made to date, many patients do not respond to currently available immune checkpoint inhibitors. Accordingly, there is still a need for effective interventions that overcome the immune suppressive tumor microenvironment and for treating cancers associated with hypersialylated cancer cells.

### SUMMARY OF THE INVENTION

The invention is based, in part, upon the discovery of anti-PD-L1 antibodies that impact or otherwise down regulate signaling mediated by PD-1 or PD-L1. In the appropriate circumstances, the antibodies can remove the PD-1 or PD-L1-mediated repression of a subject's immune system to mediate the removal of non-natural cells, for example, cancerous cells.

The invention is also based, in part, upon the discovery that it is possible to produce fusion proteins containing a sialidase enzyme and an anti-PD-L1 immunoglobulin or a portion thereof, e.g., an antigen-binding domain and/or an immunoglobulin Fc domain, and/or antibody conjugates including a sialidase enzyme and an anti-PD-L1 antibody or a portion thereof, e.g., an antigen-binding domain and/or an immunoglobulin Fc domain. The sialidase enzyme portion of the fusion protein and/or antibody conjugate may comprise at least one mutation relative to a wild-type sialidase. The mutations, or combination of mutations, can improve the expression, activity or both the expression and activity of the sialidase to improve its use in cancer diagnosis and/or treatment.

The fusion proteins and/or antibody conjugates have suitable substrate specificities and activities to be useful in removing sialic acid and/or sialic acid containing molecules from the surface of cancer cells, e.g., PD-L1-expressing cancer cells, and/or removing sialic acid and/or sialic acid containing molecules from the tumor microenvironment, and/or reducing the concentration of sialic acid and/or sialic acid containing molecules in the tumor microenvironment.

Accordingly, in one aspect, the invention provides an isolated antibody that binds human PD-L1.

In certain embodiments, the antibody comprises an immunoglobulin heavy chain variable region comprising a CDRH1 comprising the amino acid sequence of SEQ ID NO: 161, a CDRH2 comprising the amino acid sequence of SEQ ID NO: 162, and a CDRH3 comprising the amino acid sequence of SEQ ID NO: 163 (PAL769-VH, h769-VH); and/or an immunoglobulin light chain variable region comprising a CDRL1 comprising the amino acid sequence of SEQ ID NO: 165, a CDRL2 comprising the amino acid sequence of SEQ ID NO: 142, and a CDRL3 comprising the amino acid sequence of SEQ ID NO: 166 (PAL769-VL, h769-IF3-VL, h769-tm2-VL, h769-tm3-VL).

In certain embodiments, the antibody comprises an immunoglobulin heavy chain variable region comprising a CDRH1 comprising the amino acid sequence of SEQ ID NO: 250, a CDRH2 comprising the amino acid sequence of SEQ ID NO: 251, and a CDRH3 comprising the amino acid sequence of SEQ ID NO: 163 (PAL769-VH); and/or an immunoglobulin light chain variable region comprising a CDRL1 comprising the amino acid sequence of SEQ ID NO: 253, a CDRL2 comprising the amino acid sequence of SEQ ID NO: 254, and a CDRL3 comprising the amino acid sequence of SEQ ID NO: 166 (PAL769-VL).

In certain embodiments, the antibody comprises an immunoglobulin heavy chain variable region comprising a CDRH1 comprising the amino acid sequence of SEQ ID NO: 250, a CDRH2 comprising the amino acid sequence of SEQ ID NO: 252, and a CDRH3 comprising the amino acid sequence of SEQ ID NO: 163 (h769-VH); and/or an immunoglobulin light chain variable region comprising a CDRL1 comprising the amino acid sequence of SEQ ID NO: 255, a CDRL2 comprising the amino acid sequence of SEQ ID NO: 254, and a CDRL3 comprising the amino acid sequence of SEQ ID NO: 166 (h769-IF3-VL, h769-tm2-VL, h769-tm3-VL).

In certain embodiments, the antibody comprises an immunoglobulin heavy chain variable region comprising a CDR_{H1} comprising the amino acid sequence of SEQ ID NO: 161, a CDRH2 comprising the amino acid sequence of SEQ ID NO: 162, and a CDRH3 comprising the amino acid sequence of SEQ ID NO: 163 (PAL769-VH, h769-VH); and/or an immunoglobulin light chain variable region comprising a CDR_{L1} comprising the amino acid sequence of SEQ ID NO: 165, a CDR_{L2} comprising the amino acid sequence of SEQ ID NO: 142, and a CDR_{L3} comprising the amino acid sequence of SEQ ID NO: 203 (h769.T-VL).

In certain embodiments, the antibody comprises an immunoglobulin heavy chain variable region comprising a CDRH1 comprising the amino acid sequence of SEQ ID NO: 250, a CDRH2 comprising the amino acid sequence of SEQ ID NO: 252, and a CDRH3 comprising the amino acid sequence of SEQ ID NO: 163 (h769-VH); and/or an immunoglobulin light chain variable region comprising a CDRL1 comprising the amino acid sequence of SEQ ID NO: 255, a CDRL2 comprising the amino acid sequence of SEQ ID NO: 254, and a CDRL3 comprising the amino acid sequence of SEQ ID NO: 203 (h769.T-VL).

In certain embodiments, the antibody comprises an immunoglobulin heavy chain variable region comprising a CDRH1 comprising the amino acid sequence of SEQ ID NO: 129, a CDRH2 comprising the amino acid sequence of SEQ ID NO: 130, and a CDRH3 comprising the amino acid sequence of SEQ ID NO: 131 (PAL752-VH); and/or an immunoglobulin light chain variable region comprising a CDRL1 comprising the amino acid sequence of SEQ ID NO: 133, a CDRL2 comprising the amino acid sequence of SEQ ID NO: 134, and a CDRL3 comprising the amino acid sequence of SEQ ID NO: 135 (PAL752-VL).

In certain embodiments, the antibody comprises an immunoglobulin heavy chain variable region comprising a CDRH1 comprising the amino acid sequence of SEQ ID NO: 137, a CDRH2 comprising the amino acid sequence of SEQ ID NO: 138, and a CDRH3 comprising the amino acid sequence of SEQ ID NO: 139 (PAL759-VH); and/or an immunoglobulin light chain variable region comprising a CDRL1 comprising the amino acid sequence of SEQ ID NO: 141, a CDRL2 comprising the amino acid sequence of SEQ ID NO: 142, and a CDRL3 comprising the amino acid sequence of SEQ ID NO: 143 (PAL759-VL).

In certain embodiments, the antibody comprises an immunoglobulin heavy chain variable region comprising a CDRH1 comprising the amino acid sequence of SEQ ID NO: 145, a CDRH2 comprising the amino acid sequence of SEQ ID NO: 146, and a CDRH3 comprising the amino acid sequence of SEQ ID NO: 147 (PAL760-VH); and/or an immunoglobulin light chain variable region comprising a CDRL1 comprising the amino acid sequence of SEQ ID NO: 149, a CDRL2 comprising the amino acid sequence of SEQ ID NO: 150, and a CDRL3 comprising the amino acid sequence of SEQ ID NO: 151 (PAL760-VL).

In certain embodiments, the antibody comprises an immunoglobulin heavy chain variable region comprising a CDRH1 comprising the amino acid sequence of SEQ ID NO: 153, a CDRH2 comprising the amino acid sequence of SEQ ID NO: 154, and a CDRH3 comprising the amino acid sequence of SEQ ID NO: 155 (PAL767-VH); and/or an immunoglobulin light chain variable region comprising a CDRL1 comprising the amino acid sequence of SEQ ID NO: 157, a CDRL2 comprising the amino acid sequence of SEQ ID NO: 158, and a CDRL3 comprising the amino acid sequence of SEQ ID NO: 159 (PAL767-VL).

In certain embodiments, the antibody comprises an immunoglobulin heavy chain variable region comprising a CDRH1 comprising the amino acid sequence of SEQ ID NO: 161, a CDRH2 comprising the amino acid sequence of SEQ ID NO: 168, and a CDRH3 comprising the amino acid sequence of SEQ ID NO: 169 (PAL771-VH); and/or an immunoglobulin light chain variable region comprising a CDRL1 comprising the amino acid sequence of SEQ ID NO: 171, a CDRL2 comprising the amino acid sequence of SEQ ID NO: 172, and a CDRL3 comprising the amino acid sequence of SEQ ID NO: 173 (PAL771-VL).

In certain embodiments, the antibody comprises an immunoglobulin heavy chain variable region comprising a CDRH1 comprising the amino acid sequence of SEQ ID NO: 175, a CDRH2 comprising the amino acid sequence of SEQ ID NO: 176, and a CDRH3 comprising the amino acid sequence of SEQ ID NO: 177 (PAL785-VH); and/or an immunoglobulin light chain variable region comprising a CDRL1 comprising the amino acid sequence of SEQ ID NO: 179, a CDRL2 comprising the amino acid sequence of SEQ ID NO: 180, and a CDRL3 comprising the amino acid sequence of SEQ ID NO: 181 (PAL785-VL).

In certain embodiments, the antibody comprises an immunoglobulin heavy chain variable region comprising a CDRH1 comprising the amino acid sequence of SEQ ID NO: 183, a CDRH2 comprising the amino acid sequence of SEQ ID NO: 184, and a CDRH3 comprising the amino acid sequence of SEQ ID NO: 185 (PAL787-VH); and/or an immunoglobulin light chain variable region comprising a CDRL1 comprising the amino acid sequence of SEQ ID NO: 187, a CDRL2 comprising the amino acid sequence of SEQ ID NO: 188, and a CDRL3 comprising the amino acid sequence of SEQ ID NO: 189 (PAL787-VL).

In certain embodiments, the antibody comprises an immunoglobulin heavy chain variable region comprising a CDRH1 comprising the amino acid sequence of SEQ ID NO: 191, a CDRH2 comprising the amino acid sequence of SEQ ID NO: 192, and a CDRH3 comprising the amino acid sequence of SEQ ID NO: 193 (PAL788-VH); and/or an immunoglobulin light chain variable region comprising a CDRL1 comprising the amino acid sequence of SEQ ID NO: 195, a CDRL2 comprising the amino acid sequence of SEQ ID NO: 196, and a CDRL3 comprising the amino acid sequence of SEQ ID NO: 197 (PAL788-VL).

In certain embodiments of any of the foregoing antibodies, the CDRs are interposed between human or humanized immunoglobulin framework regions.

In certain embodiments, the antibody comprises an immunoglobulin heavy chain variable region comprising the amino acid sequence of SEQ ID NO: 164 (PAL769-VH), and an immunoglobulin light chain variable region comprising the amino acid sequence of SEQ ID NO: 167 (PAL769-VL).

In certain embodiments, the antibody comprises an immunoglobulin heavy chain variable region comprising the amino acid sequence of SEQ ID NO: 199 (h769-VH), and/or an immunoglobulin light chain variable region comprising the amino acid sequence of SEQ ID NO: 200 (h769-IF3-VL).

In certain embodiments, the antibody comprises an immunoglobulin heavy chain variable region comprising the amino acid sequence of SEQ ID NO: 199 (h769-VH), and/or an immunoglobulin light chain variable region comprising the amino acid sequence of SEQ ID NO: 201 (h769-tm2-VL).

In certain embodiments, the antibody comprises an immunoglobulin heavy chain variable region comprising the amino acid sequence of SEQ ID NO: 199 (h769-VH), and/or an immunoglobulin light chain variable region comprising the amino acid sequence of SEQ ID NO: 202 (h769-tm3-VL).

In certain embodiments, the antibody comprises an immunoglobulin heavy chain variable region comprising the amino acid sequence of SEQ ID NO: 199 (h769-VH), and/or an immunoglobulin light chain variable region comprising the amino acid sequence of SEQ ID NO: 204 (h769.T-VL).

In certain embodiments, the antibody comprises an immunoglobulin heavy chain variable region comprising the amino acid sequence of SEQ ID NO: 132 (PAL752-VH), and/or an immunoglobulin light chain variable region comprising the amino acid sequence of SEQ ID NO: 136 (PAL752-VL).

In certain embodiments, the antibody comprises an immunoglobulin heavy chain variable region comprising the amino acid sequence of SEQ ID NO: 140 (PAL759-VH), and/or an immunoglobulin light chain variable region comprising the amino acid sequence of SEQ ID NO: 144 (PAL759-VL).

In certain embodiments, the antibody comprises an immunoglobulin heavy chain variable region comprising the amino acid sequence of SEQ ID NO: 148 (PAL760-VH), and/or an immunoglobulin light chain variable region comprising the amino acid sequence of SEQ ID NO: 152 (PAL760-VL).

In certain embodiments, the antibody comprises an immunoglobulin heavy chain variable region comprising the amino acid sequence of SEQ ID NO: 156 (PAL767-VH), and/or an immunoglobulin light chain variable region comprising the amino acid sequence of SEQ ID NO: 160 (PAL767-VL).

In certain embodiments, the antibody comprises an immunoglobulin heavy chain variable region comprising the amino acid sequence of SEQ ID NO: 170 (PAL771-VH), and/or an immunoglobulin light chain variable region comprising the amino acid sequence of SEQ ID NO: 174 (PAL771-VL).

In certain embodiments, the antibody comprises an immunoglobulin heavy chain variable region comprising the amino acid sequence of SEQ ID NO: 178 (PAL785-VH), and/or an immunoglobulin light chain variable region comprising the amino acid sequence of SEQ ID NO: 182 (PAL785-VL).

In certain embodiments, the antibody comprises an immunoglobulin heavy chain variable region comprising the amino acid sequence of SEQ ID NO: 186 (PAL787-VH), and/or an immunoglobulin light chain variable region comprising the amino acid sequence of SEQ ID NO: 190 (PAL787-VL).

In certain embodiments, the antibody comprises an immunoglobulin heavy chain variable region comprising the amino acid sequence of SEQ ID NO: 194 (PAL788-VH), and/or an immunoglobulin light chain variable region comprising the amino acid sequence of SEQ ID NO: 198 (PAL788-VL).

In certain embodiments of any of the foregoing antibodies, the antibody further comprises a heavy chain constant region (e.g., an IgG1, IgG2, IgG3, and IgG4 heavy chain constant region) and/or light chain constant region.

In certain embodiments of any of the foregoing antibodies, the antibody binds to human PD-L1 with a KD of 5 nM or lower, 3 nM or lower, 2.5 nM or lower, 2 nM or lower, 1 nM or lower, 0.75 nM or lower, 0.5 nM or lower, 0.1 nM, 0.075 nM, or 0.05 nM or lower, as measured by surface plasmon resonance or bio-layer interferometry. In certain embodiments, the antibody also binds to Macaca fascicularis (cynomolgus) PD-L1.

In another aspect, the invention provides an isolated antibody that competes with any of the foregoing antibodies for binding to human PD-L1 and/or binds to the same epitope on human PD-L1 as any of the foregoing antibodies.

In another aspect, the invention provides an isolated nucleic acid comprising a nucleotide sequence encoding an immunoglobulin heavy chain variable region of any of the foregoing antibodies and/or an immunoglobulin light chain variable region of any of the foregoing antibodies.

In another aspect, the invention provides an expression vector comprising: (i) a nucleic acid comprising a nucleotide sequence encoding an immunoglobulin heavy chain variable region of any of the foregoing antibodies; and/or (ii) a nucleic acid comprising a nucleotide sequence encoding an immunoglobulin light chain variable region of any of the foregoing antibodies.

In another aspect, the invention provides a host cell comprising any of the foregoing nucleic acids or expression vectors.

In another aspect, the invention provides a fusion protein comprising (or consisting essentially of): (a) sialidase enzyme; and (b) an anti-PD-L1 immunoglobulin antigen-binding domain derived from any of the foregoing antibodies.

In certain embodiments, the sialidase is a human sialidase, e.g., a recombinant mutant human sialidase. In certain embodiments, the sialidase comprises: (a) a substitution or deletion of a methionine residue at a position corresponding to position 1 of wild-type human Neu2 (M1); (b) a substitution of a valine residue at a position corresponding to position 6 of wild-type human Neu2 (V6); (c) a substitution of a lysine residue at a position corresponding to position 9 of wild-type human Neu2 (K9); (d) a substitution of an alanine residue at a position corresponding to position 42 of wild-type human Neu2 (A42); (e) a substitution of a proline residue at a position corresponding to position 62 of wild-type human Neu2 (P62); (f) a substitution of an alanine residue at a position corresponding to position 93 of wild-type human Neu2 (A93); (g) a substitution of a glutamine residue at a position corresponding to position 126 of wild-type human Neu2 (Q126); (h) a substitution of an isoleucine residue at a position corresponding to position 187 of wild-type human Neu2 (I187); (i) a substitution of an alanine residue at a position corresponding to position 242 of wild-type human Neu2 (A242); (j) a substitution of a glutamine residue at a position corresponding to position 270 of wild-type human Neu2 (Q270); (k) a substitution of a serine residue at a position corresponding to position 301 of wild-type human Neu2 (S301); (l) a substitution of a tryptophan residue at a position corresponding to position 302 of wild-type human Neu2 (W302); (m) a substitution of a cysteine residue at a position corresponding to position 332 of wild-type human Neu2 (C332); (n) a substitution of a valine residue at a position corresponding to position 363 of wild-type human Neu2 (V363); or (o) a substitution of a leucine residue at a position corresponding to position 365 of wild-type human Neu2 (L365); or a combination of any of the foregoing substitutions.

In certain embodiments, in the sialidase: (a) the methionine residue at a position corresponding to position 1 of wild-type human Neu2 is deleted (AMI), is substituted by alanine (MIA), or is substituted by aspartic acid (MID); (b) the valine residue at a position corresponding to position 6 of wild-type human Neu2 is substituted by tyrosine (V6Y); (c) the lysine residue at a position corresponding to position 9 of wild-type human Neu2 is substituted by aspartic acid (K9D); (d) the alanine residue at a position corresponding to position 42 of wild-type human Neu2 is substituted by arginine (A42R) or aspartic acid (A42D); (e) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (f) the alanine residue at a position corresponding to position 93 of wild-type human Neu2 is substituted by glutamic acid (A93E) or lysine (A93K); (g) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (h) the isoleucine residue at a position corresponding to position 187 of wild-type human Neu2 is substituted by lysine (I187K); (i) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (j) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (k) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y); (l) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (m) the cysteine residue at a position corresponding to position 332 of wild-type human Neu2 is substituted by alanine (C332A); (n) the valine residue at a position corresponding to position 363 of wild-type human Neu2 is substituted by arginine (V363R); or (o) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from ΔM1, M1A, M1D, V6Y, K9D, A42R, P62G, P62N, P62S, P62T, A93E, Q126Y, I187K, A242F, A242W, A242Y, Q270A, Q270T, S301A, S301R, W302K, W302R, C332A, V363R, and L365I, or a combination of any of the foregoing substitutions.

In certain embodiments, the sialidase comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; (k) the M1D, V6Y, P62G, A93E, Q126Y, I187K, A242F, Q270T, and C332A substitutions; or (l) the M1D, V6Y, A42R, P62G, A93E, Q126Y, I187K, A242F, Q270T, and C332A mutations.

In certain embodiments, the sialidase is selected from Neu1, Neu2, Neu3, and Neu4, e.g., the sialidase is Neu2.

In certain embodiments, the sialidase has a different substrate specificity than the corresponding wild-type sialidase. For example, in certain embodiments the sialidase can cleave α2,3, α2,6, and/or α2,8 linkages. In certain embodiments the sialidase can cleave α2,3 and α2,8 linkages.

In certain embodiments, the sialidase comprises any one of SEQ ID NOs: 48-62, 94, 97, 100, 126, or 234, or an amino acid sequence that has at least 85%, 90%, 95%, 96%, 97%, 98%, or 99% sequence identity to any one of SEQ ID NOs: 48-62, 94, 97, 100, 126, or 234.

In certain embodiments, the sialidase comprises mutation or combination of mutations set forth in any one of Tables 1-9.

In certain embodiments, the fusion protein further comprises an immunoglobulin Fc domain. In certain embodiments, the immunoglobulin Fc domain is derived from a human IgG1, IgG2, IgG3, IgG4, IgA1, IgA2, IgD, IgE, or IgM Fc domain, e.g., the immunoglobulin Fc domain is derived from a human IgG1, IgG2, IgG3, or IgG4 Fc domain, e.g., the immunoglobulin Fc domain is derived from a human IgG1 Fc domain.

In certain embodiments, the anti-PD-L1 immunoglobulin antigen-binding domain is associated (for example, covalently or non-covalently associated) with a second anti-PD-L1 immunoglobulin antigen-binding domain to produce an anti-PD-L1 antigen-binding site. For example, in certain embodiments the anti-PD-L1 immunoglobulin antigen-binding domain is an immunoglobulin heavy chain fragment that is associated with an immunoglobulin light chain fragment to produce an anti-PD-L1 antigen-binding site. In other embodiments the anti-PD-L1 immunoglobulin antigen-binding domain is an immunoglobulin light chain fragment that is associated with an immunoglobulin heavy chain fragment to produce an anti-PD-L1 antigen-binding site.

In certain embodiments, the sialidase and the immunoglobulin Fc domain and/or the anti-PD-L1 immunoglobulin antigen-binding domain are linked by a peptide bond or an amino acid linker.

In certain embodiments, the fusion protein comprises any one of SEQ ID NOs: 205-207, 211, 213, 214, and 219.

In another aspect, the invention provides an antibody conjugate comprising any of the foregoing fusion proteins. In certain embodiments, the antibody conjugate comprises a single sialidase. In other embodiments, the antibody conjugate comprises two sialidases, which can be the same or different. In certain embodiments the antibody conjugate comprises two identical sialidases. In certain embodiments, the antibody conjugate comprises a single anti-PD-L1 antigen-binding site. In other embodiments, the antibody conjugate comprises two anti-PD-L1 antigen-binding sites, which can be the same or different. In certain embodiments, the antibody conjugate comprises two identical anti-PD-L1 antigen-binding sites.

In certain embodiments, the antibody conjugate has a molecular weight from about 135 kDa to about 165 kDa, or the antibody conjugate has a molecular weight from about 215 kDa to about 245 kDa.

In certain embodiments, the antibody conjugate comprises: (a) a first polypeptide comprising an immunoglobulin light chain; (b) a second polypeptide comprising an immunoglobulin heavy chain; and (c) a third polypeptide comprising an immunoglobulin Fc domain and a sialidase; wherein the first and second polypeptides are covalently linked together and the second and third polypeptides are covalently linked together, and wherein the first polypeptide and the second polypeptide together define an anti-PD-L1 antigen-binding site. The third polypeptide may, for example, comprise the sialidase and the immunoglobulin Fc domain in an N- to C-terminal orientation. The first polypeptide may, for example, comprise SEQ ID NO: 205, the second polypeptide may, for example, comprise any one of SEQ ID NOs: 206 or 213, and/or the third polypeptide may, for example, comprise any one of SEQ ID NOs: 207, 211, 214, or 219.

In certain embodiments, the antibody conjugate comprises: (a) a first polypeptide comprising a first immunoglobulin light chain; (b) a second polypeptide comprising a first immunoglobulin heavy chain and a first sialidase; (c) a third polypeptide comprising a second immunoglobulin heavy chain and a second sialidase; and (d) a fourth polypeptide comprising a second immunoglobulin light chain; wherein the first and second polypeptides are covalently linked together, the third and fourth polypeptides are covalently linked together, and the second and third polypeptides are covalently linked together, and wherein the first polypeptide and the second polypeptide together define a first anti-PD-L1 antigen-binding site, and the third polypeptide and the fourth polypeptide together define a second anti-PD-L1 antigen-binding site. The second and third polypeptides may, for example, comprise the first and second immunoglobulin heavy chain and the first and second sialidase, respectively, in an N- to C-terminal orientation.

In certain embodiments, the antibody conjugate comprises: (a) a first polypeptide comprising a first sialidase, a first immunoglobulin Fc domain, and a first single chain variable fragment (scFv); and (b) a second polypeptide comprising a second sialidase, a second immunoglobulin Fc domain, and an optional second single chain variable fragment (scFv); wherein the first and second polypeptides are covalently linked together, and wherein the first scFv defines a first anti-PD-L1 antigen-binding site, and the second scFv, when present, defines a second anti-PD-L1 antigen-binding site. The first polypeptide may, for example comprise the first sialidase, the first immunoglobulin Fc domain, and the first scFv in an N- to C-terminal orientation. The second polypeptide may, for example, comprise the second sialidase, the second immunoglobulin Fc domain, and the optional second scFv in an N- to C-terminal orientation.

In certain embodiments, the antibody conjugate comprises: (a) a first polypeptide comprising an immunoglobulin light chain; (b) a second polypeptide comprising an immunoglobulin heavy chain and a single chain variable fragment (scFv); and (c) a third polypeptide comprising an immunoglobulin Fc domain and a sialidase, wherein the first and second polypeptides are covalently linked together and the second and third polypeptides are covalently linked together, and wherein the immunoglobulin light chain and immunoglobulin heavy chain together define a first anti-PD-L1 antigen-binding site and the scFv defines a second anti-PD-L1 antigen-binding site. The second polypeptide may, for example comprise the immunoglobulin heavy chain and the scFv in an N- to C-terminal orientation. The third polypeptide may, for example, comprise the sialidase and the immunoglobulin Fc domain in an N- to C-terminal orientation.

In another aspect, the invention provides an isolated nucleic acid comprising a nucleotide sequence encoding at least a portion of any of the foregoing antibodies, any of the foregoing fusion proteins, or at least a portion of any of the foregoing antibody conjugates. In another aspect, the invention provides an expression vector comprising any of the foregoing nucleic acids. In another aspect, the invention provides a host cell comprising any of the foregoing expression vectors.

In another aspect, the invention provides a pharmaceutical composition comprising any of the foregoing antibodies, any of the foregoing fusion proteins, or any of the foregoing antibody conjugates.

In another aspect, the invention provides a method of treating cancer in a subject in need thereof. The method comprises administering to the subject an effective amount of any of the foregoing antibodies, any of the foregoing fusion proteins, any of the foregoing antibody conjugates, or any of the foregoing pharmaceutical compositions.

In certain embodiments, the cancer is selected from non-small cell lung cancer (NSCLC), melanoma, bladder, breast, cervical, esophageal, gastric, kidney, lung, ovary, metastatic Merkel cell carcinoma (MCC), metastatic urothelial carcinoma (UC), and pancreatic cancer.

These and other aspects and features of the invention are described in the following detailed description and claims.

### DESCRIPTION OF THE DRAWINGS

The invention can be more completely understood with reference to the following drawings.

FIG. 1 depicts an SDS-PAGE gel showing recombinant human Neu1, Neu2, Neu3, and Salmonella typhimurium (St-sialidase) under non-reducing and reducing conditions. Monomer and dimer species are indicated.

FIG. 2 is a bar graph showing the enzymatic activity of recombinant human Neu1, Neu2, and Neu3.

FIG. 3 is a line graph showing enzymatic activity as a function of substrate concentration for recombinant human Neu2 and Neu3 at the indicated pH.

FIGS. 4A-4I depict schematic representations of certain antibody conjugate constructs containing a sialidase enzyme, e.g., a human sialidase enzyme, and an anti-PD-L1 antigen binding site. For each antibody conjugate construct that contains more than one (e.g., two) sialidase, each sialidase may be the same or different. For each antibody conjugate construct that contains more than one (e.g., two) anti-PD-L1 antigen binding site, each anti-PD-L1 antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIG. 5 depicts schematic representations of certain antibody conjugate constructs containing a sialidase enzyme, e.g., a human sialidase enzyme, and an antigen binding site. For each antibody conjugate construct that contains more than one (e.g., two) antigen binding site, each antigen binding site may be the same or different. For each antibody conjugate construct that contains an Fc domain, it is understood that the Fc domain can be a wild type Fc domain or can be an engineered Fc domain. For example, the Fc domain may be engineered to contain either a “knob” mutation, e.g., T366Y, or a “hole” mutation, e.g., Y407T, or both, to promote heterodimerization, or the Fc domain may be engineered to contain one or more modifications, e.g., point mutations, to provide any other modified Fc domain functionality.

FIGS. 6A-6D are schematic representations of exemplary fusion protein conjugates referred to as a Raptor antibody sialidase conjugate (FIG. 6A), a Janus antibody sialidase conjugate (FIG. 6B), a Lobster antibody sialidase conjugate (FIG. 6C), a Bunk antibody sialidase conjugate (FIG. 6D), and a Lobster-Fab antibody sialidase conjugate (FIG. 6E).

FIG. 7 provides a graph showing the fold induction of a PD-1/PD-L1 linked NFAT driven luciferase reporter by the indicated hybridoma supernatant comprising anti-PD-L1 antibodies, showing the ability of the antibodies to functionally block the interaction between PD-L1 and PD-1. FIG. 7A, FIG. 7B, and FIG. 7C represent different hybridomas where candidate antibodies with fold inductions greater than 4 are identified.

FIGS. 8A, 8B, 8C, and 8D provide graphs showing ForteBio octet binding kinetics for human PD-L1 binding to purified hybridoma antibodies.

FIGS. 9A, 9B, 9C, and 9D provide graphs showing ForteBio octet binding kinetics for cynomolgus PD-L1 binding to purified hybridoma antibodies.

FIG. 10 provides a graph showing ELISA results indicative of the ability of purified hybridoma antibodies to block human PD-1-Fc binding to human PD-L1. The IC₅₀ (nM) for each antibody is shown.

FIG. 11 provides a graph showing ForteBio octet binding kinetics for human PD-L1 (FIG. 11A and FIG. 11B) and cynomolgus PD-L1 (FIG. 11C and FIG. 11D) binding to chimeric IgG antibodies.

FIG. 12 provides a graph showing ELISA results indicative of the ability of anti-hPD-L1 chimeric IgGs to block PD-1/PD-L1 interaction. The IC₅₀ (nM) for each antibody is shown.

FIG. 13 provides size exclusion chromatography (SEC) profiles of selected chimeric IgGs. Monomeric percentage of each chimeric antibody is shown.

FIG. 14 is a graph showing binding of chimeric PD-L1 antibodies to HCC827 (FIG. 14A) and NCI-H292 (FIG. 14B) lung epithelial cell lines. Binding was measured using a FACS analysis with a fluorescein-labeled secondary antibody. MFI=mean fluorescence intensity. The apparent Kd (nM) for each antibody is shown.

FIG. 15 is a graph depicting the level of binding and internalization of chimeric PD-L1 antibodies on human Monocyte-derived Dendritic Cells (moDC). Cells were incubated with the indicated antibodies at 1 nM, 10 nM and 10 nM antibody in the presence or absence (no-stim) of PAM3K.

FIG. 16 depicts the ability of chimeric PD-L1 antibodies to functionally block the interaction between PD-L1 and PD-1 using the PD-L1/PD-1 bioassay. Fold induction was calculated by dividing the RLU of induced cells minus background by the RLU of the no antibody control minus background. Apparent Kd (nM) for each antibody is indicated.

FIG. 17 depicts the specificity of binding of chimeric PD-L1 antibodies to CHO cells expressing PD-L1 (CHO-PD-L1, staining at 10 nM) vs parental CHO cells (staining at 100 nM).

FIGS. 18A-D are graphs depicting the enhancement of T cell proliferation and cytokine response to allogeneic moDC in the presence of the indicated PD-L1 antibodies compared to isotype control (001-1). Levels of CD4 T cell proliferation (FIG. 18A), CD8 T cell proliferation (FIG. 18B), TNFα (FIG. 18C) and IFN-γ levels (FIG. 18D) are depicted.

FIG. 19 is a graph depicting the enhancement of cytokine response in T cells to allogeneic moDC in the presence of indicated PD-L1 antibodies compared to isotype control (001-1). Levels of IL-2 (FIG. 19A), IL-4 (FIG. 19B), IL-6 (FIG. 19C) and IL-10 (FIG. 19D) are depicted.

FIG. 20 is a graph depicting the enhancement of degranulation in moDC-T cell mixed lymphocyte reactions (MLR) in the presence of the indicated PD-L1 antibodies compared to isotype control (001-1). Levels of soluble Fas Ligand (FIG. 20A), Granzyme A (FIG. 20B), perforin (FIG. 20C) and granulysin (FIG. 20D) are depicted.

FIG. 21A depicts an alignment between murine 769VH-wt (SEQ ID NO: 164) and humanized h769VH-mF0 (SEQ ID NO: 199). FIG. 21B depicts an alignment between murine 769Vk-wt (SEQ ID NO: 167) and humanized h769Vk-mF0 (SEQ ID NO: 242), h769Vk-T53I (SEQ ID NO: 243), h769Vk-A55F (SEQ ID NO: 244), h769Vk-S67Y (SEQ ID NO: 245), and h769Vk-Y87F (SEQ ID NO: 246). FIG. 21C depicts an alignment between humanized h769Vk-IY (SEQ ID NO: 247), h769Vk-IF2 (SEQ ID NO: 248), h769Vk-tm1 (SEQ ID NO: 249), h769Vk-IF3 (SEQ ID NO: 200), h769Vk-tm2 (SEQ ID NO: 201), and h769Vk-tm3 (SEQ ID NO: 202). Framework regions are shown in grey. Vkappa back mutations that were made as part of the humanization are shown in boxes.

FIG. 22 depicts SEC profiles of selected 769-hIgG1 humanized variants.

FIG. 23 provides graphs showing ForteBio octet binding kinetics of human PD-L1 (FIG. 23A) and cynomolgus PD-L1 (FIG. 23B) to selected 769-hIgG1 humanized variants.

FIG. 24 provides a graph showing the ability of humanized PD-L1 antibodies to block PD-1/PD-L1 interaction. The IC₅₀ (nM) for each antibody is shown.

FIG. 25 provides a graph showing ForteBio octet binding kinetics of selected 769-hIgG1 humanized variants to human PD-L1.

FIG. 26 depicts SEC profiles of selected 769-hIgG1 humanized variants.

FIG. 27 provides graphs depicting the ability of selected 769-hIgG1 humanized variants to enhance T cell response to allogeneic moDC. Levels of CD4 T cell proliferation (FIG. 27A), Granzyme B (FIG. 27B), and IFN-γ (FIG. 27C) as well as CD8 T cell proliferation (FIG. 27D), Granzyme A (FIG. 27E) and TNFα levels (FIG. 27F) are shown.

FIG. 28 provides graphs depicting the levels of the indicated cytokines released in response to selected 769-hIgG1 humanized variants, suggesting that the 769-hIgG1 humanized variants are capable of enhancing T cell response to allogeneic moDC. Perforin (FIG. 28A), soluble Fas (FIG. 28B), IL-6 (FIG. 28C), Granulysin (FIG. 28D), soluble Fas Ligand (FIG. 28E) and IL-10 levels (FIG. 28F) are shown.

FIG. 29 provides graphs depicting the levels of the indicated cytokines released from peripheral blood mononuclear cells (PBMCs) in response to the cytomegalovirus pp65 peptide mix (“CMV pp65”) and in the presence of selected 769-hIgG1 humanized variants. “No stim”=unstimulated control (i.e., cells not exposed to CMV pp65). Levels of IL-2 (FIG. 29A) and TNFα (FIG. 29B) in pg/ml are shown.

FIG. 30 provides graphs depicting the levels of the indicated cytokines released from peripheral blood mononuclear cells (PBMCs) in response to the cytomegalovirus pp65 peptide mix (“CMV pp65”) and in the presence of selected 769-hIgG1 humanized variants. “No stim”=unstimulated control (i.e., cells not exposed to CMV pp65). Levels of IL-6 (FIG. 30A) and IL-17A (FIG. 30B) in pg/ml are shown.

FIG. 31 provides graphs depicting the levels of the indicated cytokines released from peripheral blood mononuclear cells (PBMCs) in response to the cytomegalovirus pp65 peptide mix (“CMV pp65”) and in the presence of selected 769-hIgG1 humanized variants. “No stim”=unstimulated control (i.e., cells not exposed to CMV pp65). Levels of Granzyme A (FIG. 31A) and Granzyme B (FIG. 31B) in pg/ml are shown.

FIG. 32 provides graphs depicting the levels of the indicated cytokines released from peripheral blood mononuclear cells (PBMCs) in response to the cytomegalovirus pp65 peptide mix (“CMV pp65”) and in the presence of selected 769-hIgG1 humanized variants. “No stim”=unstimulated control (i.e., cells not exposed to CMV pp65). Levels of Perforin (FIG. 32A) and Granulysin (FIG. 32B) in pg/ml are shown.

FIG. 33 provides graphs depicting the levels of IFN-γ released from peripheral blood mononuclear cells (PBMCs) in response to the cytomegalovirus pp65 peptide mix (“CMV pp65”) and in the presence of selected 769-hIgG1 humanized variants. “No stim”=unstimulated control (i.e., cells not exposed to CMV pp65).

FIG. 34 depicts an epitope binning sandwich assay for the antibodies indicated.

FIG. 35 depicts the biochemical characterization of a sialidase-anti-PD-L1 conjugate. FIG. 35A provides a photograph of the non-reduced and reduced PAGE of the purified molecule. FIG. 35B shows the SEC profile of a sialidase-anti-PD-L1 conjugate (ASC1) with a demonstrated purity of 89%.

FIG. 36 provides graphs showing ForteBio octet binding kinetics of selected 769-hIgG1 humanized variants, a PD-L1 antibody sialidase conjugate (ASC1), and atezolizumab to human PD-L1.

FIGS. 37A and B depicts the biochemical characterization of a second sialidase-anti-PD-L1 conjugate (ASC3). FIG. 37A depicts the SEC profile of the second sialidase-anti-PD-L1 conjugate (ASC3). FIG. 37B is a graph showing the relative fluorescence units (RFU), indicative of sialidase activity, over increasing substrate concentration. Three batches of the purified second sialidase-anti-PD-L1 conjugate, WG7, WG8, and WG9, were tested and had similar activity.

FIG. 38 depicts the SEC profile of a third sialidase-anti-PD-L1 conjugate with an inactivate sialidase (ASC4 loss of function or LOF).

FIGS. 39A and B depict the biochemical characterization of a fourth sialidase-anti-PD-L1 conjugate. FIG. 39A depicts the SEC profile of the fourth sialidase-anti-PD-L1 conjugate (ASC5). FIG. 39B is a graph showing the relative fluorescence units (RFU), indicative of sialidase activity of the fourth sialidase-anti-PD-L1 conjugate (ASC5), over increasing substrate concentration.

FIG. 40A depicts the SEC profile of a fifth sialidase-anti-PD-L1 conjugate (ASC2). FIG. 40B is a graph showing the relative fluorescence units (RFU), indicative of the sialidase activity of the fifth sialidase-anti-PD-L1 conjugate (ASC2), over increasing substrate concentration. ASC is also depicted.

FIG. 41 provides graphs showing ForteBio octet binding kinetics of human PD-L1 (FIG. 41A) and cynomolgus PD-L1 (FIG. 41B) binding to selected sialidase-anti-PD-L1 conjugates as compared to h769.T-1A, where the second sialidase PD-L1 heterodimer is ASC2, the third sialidase PD-L1 heterodimer is ASC3, and the fourth sialidase PD-L1 heterodimer is ASC4 LOF.

FIG. 42 provides the results of an experiment in which a PD-L1 antibody sialidase conjugate or a control molecule was bound to PD-L1-expressing cell lines (HCC827 and NC-H292), and then exposed to a secondary antibody conjugated to a fluorescent moiety. The graphs show mean fluorescence units (MFI) over increasing concentrations of sialidase-anti-PD-L1 conjugate or control molecule, depicting binding of the indicated ASC or antibody to HCC827 (FIG. 42A) and NCI-H292 (FIG. 42B) lung epithelial cell lines.

FIG. 43 depicts MFI, which is indicative of de-sialylation, over increasing concentrations of sialidase-anti-PD-L1 conjugates on K562 cells (FIG. 43A) and HT-29 cells (FIG. 43B).

FIG. 44 depicts the in vivo efficacy of the indicated anti-PD-L1 antibody sialidase conjugates in a mouse syngeneic subcutaneous tumor model. Mean tumor volumes over 21 days for the indicated treatments are indicated in FIG. 44A. Triangles indicate dosing. Individual tumor volumes on day 21 are depicted in FIG. 44B. One-way ANOVA (*p<0.05; **p<0.005; ns Non-significant).

FIG. 45 depicts blocking of the interaction between human PD-L1 and human PD-1-Fc by ASC5, as measured by ELISA. Two independent preparations of ASC5 (Lot 1 and Lot 2) were tested. Results for h769.T-1A and atezolizumab are also shown. Human PD-1-Fc only (no Ab; full PD-L1/PD-1 binding) and buffer only (no antibody and no human PD-1-Fc; no PD-L1/PD-1 binding) were used as controls.

FIG. 46 depicts blocking of the PD-L1 and PD-1 interaction by ASC5, as measured by fold induction of a PD-1/PD-L1 linked NFAT driven luciferase reporter. Results are shown for three independent preparations of ASC5 (First Lot, Second Lot, and Third Lot). Results are also shown for bivalent anti-PD-L1 antibodies h769.T-1A and atezolizumab.

FIG. 47 depicts the effect of ASC5 on cytokine release in a DC-T co-culture experiment. ASC5 was tested at 700 nM (100 mg/ml), h769.T-1A and atezolizumab at 70 nM (10 mg/ml), and isotype control (001-1G) at 100 mg/ml. Each data point represents a separate DC-T donor pair. FIG. 47A depicts the fold change of IL-2 for ASC5, h769.T-1A, atezolizumab, and isotype control. FIG. 47B, FIG. 47C, and FIG. 47D show similar data for IFN-γ, IL-8, and MCP1, respectively.

FIG. 48 depicts in vivo efficacy of ASC5 and h769.T-1A, each at the indicated dose, in a MC38 mouse syngeneic subcutaneous tumor model. Isotype antibody (001-1G) and atezolizumab were used as controls. Mean tumor volumes±SEM over 18 days are depicted in FIG. 48A. Triangles indicate drug administration. Individual tumor volumes on day 18 are depicted in FIG. 48B. One-way ANOVA (**p<0.005).

FIG. 49 depicts in vivo efficacy of ASC5 and h769.T-1A in a CT26 mouse syngeneic subcutaneous tumor model. Isotype antibody (001-1G) was used as a control. Tumor growth inhibition over 18 days is depicted in FIG. 49A. Individual tumor volumes on day 18 are depicted in FIG. 49B. One-way ANOVA (****p<0.05; ns Non-significant).

FIG. 50 depicts in vivo efficacy of ASC5, ASC4 LOF, and h769.T-1A, each at the indicated dose, in a CT26 mouse syngeneic subcutaneous tumor model. Isotype antibody (001-1G) and atezolizumab were used as controls. Mean tumor volumes±SEM over 16 days are depicted in FIG. 50A. Individual tumor volumes on day 16 are depicted in FIG. 50B. One-way ANOVA (***p<0.05; ns Non-significant).

FIG. 51A depicts CDR and framework sequences for heavy chain variable region sequences SEQ ID NO: 164 and SEQ ID NO: 199. FIG. 51B depicts CDR and framework sequences for light chain variable region sequences SEQ ID NO: 167, SEQ ID NO: 200, SEQ ID NO: 201, SEQ ID NO: 202, SEQ ID NO: 204, SEQ ID NO: 242, SEQ ID NO: 243, SEQ ID NO: 244, SEQ ID NO: 245, SEQ ID NO: 246, SEQ ID NO: 247, SEQ ID NO: 248, and SEQ ID NO: 249. Framework sequences are shown in grey.

### DETAILED DESCRIPTION

The invention is based, in part, upon the discovery of anti-PD-L1 antibodies that impact or otherwise down regulate signaling mediated by PD-1 or PD-L1.

Furthermore, the invention is also based, in part, upon the discovery that it is possible to produce fusion proteins containing a sialidase enzyme and an anti-PD-L1 immunoglobulin or a portion thereof, e.g., an antigen-binding domain and/or an immunoglobulin Fc domain, and/or antibody conjugates including a sialidase enzyme and an anti-PD-L1 antibody or a portion thereof, e.g., an antigen-binding domain and/or an immunoglobulin Fc domain. The sialidase enzyme portion of the fusion protein and/or antibody conjugate may comprise at least one mutation relative to a wild-type sialidase. The mutations, or combination of mutations, can improve the expression, activity or both the expression and activity of the sialidase to improve its use in cancer diagnosis and/or treatment. The fusion proteins and/or antibody conjugates have suitable substrate specificities and activities to be useful in removing sialic acid and/or sialic acid containing molecules from the surface of cancer cells, e.g., PD-L1-expressing cancer cells, and/or removing sialic acid and/or sialic acid containing molecules from the tumor microenvironment, and/or reducing the concentration of sialic acid and/or sialic acid containing molecules in the tumor microenvironment.

The invention further relates to pharmaceutical compositions and methods of using antibodies, fusion proteins, and/or antibody conjugates to treat cancer.

### I. Anti-PD-L1 Antibodies

Among other things, the invention provides antibodies that bind PD-L1 and have the ability to inhibit PD-L1 and/or PD-L1 mediated downstream activities and, therefore, are useful in treating disorders associated with elevated levels of PD-L1, for example, cancer, for example, a cancer that evades a subject's immune system via PD-L1 mediated suppression of a subject's immune system. It is believed that, in certain embodiments, the anti-PD-L1 antibodies described herein disrupt the interaction between PD-L1 and PD-1.

In general, antibodies are multimeric proteins that contain four polypeptide chains. Two of the polypeptide chains are called immunoglobulin heavy chains (H chains), and two of the polypeptide chains are called immunoglobulin light chains (L chains). The immunoglobulin heavy and light chains are connected by an interchain disulfide bond. The immunoglobulin heavy chains are connected by interchain disulfide bonds. A light chain consists of one variable region (V_{L}) and one constant region (C_{L}). The heavy chain consists of one variable region (V_{H}) and at least three constant regions (CH₁, CH₂ and CH₃). The variable regions determine the binding specificity of the antibody.

Each variable region contains three hypervariable regions known as complementarity determining regions (CDRs) flanked by four relatively conserved regions known as framework regions (FRs). The extent of the FRs and CDRs has been defined (Kabat, E. A., et al. (1991) SEQUENCES OF PROTEINS OF IMMUNOLOGICAL INTEREST, FIFTH EDITION, U.S. Department of Health and Human Services, NIH Publication No. 91-3242; and Chothia, C. et al. (1987) J. MOL. BIOL. 196:901-917). The three CDRs, referred to as CDR₁, CDR₂, and CDR₃, contribute to the antibody binding specificity. Naturally occurring antibodies have been used as starting material for engineered antibodies, such as chimeric antibodies and humanized antibodies.

As used herein, unless otherwise indicated, the term “antibody” is understood to mean an intact antibody (e.g., an intact monoclonal antibody) or a fragment thereof, such as an antigen-binding fragment of an antibody (e.g., an antigen-binding fragment of a monoclonal antibody) or a Fc fragment of an antibody (e.g., an Fc fragment of a monoclonal antibody), including an intact antibody, antigen-binding fragment, or Fc fragment that has been modified, engineered, or chemically conjugated. Examples of antigen-binding fragments include Fab, Fab′, (Fab′)₂, Fv, single chain antibodies (e.g., scFv), minibodies, and diabodies. Examples of antibodies that have been modified or engineered include chimeric antibodies, humanized antibodies, and multispecific antibodies (e.g., bispecific antibodies). An example of a chemically conjugated antibody is an antibody conjugated to a toxin moiety.

As disclosed herein, antibodies of the invention may comprise: (a) an immunoglobulin heavy chain variable region comprising the structure CDR_{H1}-CDR_{H2}-CDR_{H3} and (b) an immunoglobulin light chain variable region comprising the structure CDR_{L1}-CDR_{L2}-CDR_{L3}, wherein the heavy chain variable region and the light chain variable region together define a single binding site for binding PD-L1.

In certain embodiments, an antibody can comprise: an immunoglobulin heavy chain variable region comprising a CDRH1 comprising the amino acid sequence of SEQ ID NO: 161, a CDRH2 comprising the amino acid sequence of SEQ ID NO: 162, and a CDRH3 comprising the amino acid sequence of SEQ ID NO: 163, wherein CDR_{H1}, CDR_{H2}, and CDR_{H3} sequences are interposed between immunoglobulin FR sequences (PAL769-VH, h769-VH); and/or an immunoglobulin light chain variable region comprising a CDRL1 comprising the amino acid sequence of SEQ ID NO: 165, a CDRL2 comprising the amino acid sequence of SEQ ID NO: 142, and a CDRL3 comprising the amino acid sequence of SEQ ID NO: 166, wherein the CDR_{L1}, CDR_{L2}, and CDR_{L3} sequences are interposed between immunoglobulin FR sequences (PAL769-VL, h769-IF3-VL, h769-tm2-VL, h769-tm3-VL).

In certain embodiments, an antibody can comprise: an immunoglobulin heavy chain variable region comprising a CDR_{H1} comprising the amino acid sequence of SEQ ID NO: 250, a CDRH2 comprising the amino acid sequence of SEQ ID NO: 251, and a CDRH3 comprising the amino acid sequence of SEQ ID NO: 163, wherein CDR_{H1}, CDR_{H2}, and CDR_{H3} sequences are interposed between immunoglobulin FR sequences (PAL769-VH); and/or an immunoglobulin light chain variable region comprising a CDR_{L1} comprising the amino acid sequence of SEQ ID NO: 253, a CDR_{L2} comprising the amino acid sequence of SEQ ID NO: 254, and a CDR_{L3} comprising the amino acid sequence of SEQ ID NO: 166, wherein the CDR_{L1}, CDR_{L2}, and CDR_{L3} sequences are interposed between immunoglobulin FR sequences (PAL769-VL).

In certain embodiments, an antibody can comprise: an immunoglobulin heavy chain variable region comprising a CDR_{H1} comprising the amino acid sequence of SEQ ID NO: 250, a CDRH2 comprising the amino acid sequence of SEQ ID NO: 252, and a CDRH3 comprising the amino acid sequence of SEQ ID NO: 163, wherein CDR_{H1}, CDR_{H2}, and CDR_{H3} sequences are interposed between immunoglobulin FR sequences (h769-VH); and/or an immunoglobulin light chain variable region comprising a CDRL1 comprising the amino acid sequence of SEQ ID NO: 255, a CDR_{L2} comprising the amino acid sequence of SEQ ID NO: 254, and a CDR_{L3} comprising the amino acid sequence of SEQ ID NO: 166, wherein the CDR_{L1}, CDR_{L2}, and CDR_{L3} sequences are interposed between immunoglobulin FR sequences (h769-IF3-VL, h769-tm2-VL, h769-tm3-VL).

In certain embodiments, an antibody can comprise: an immunoglobulin heavy chain variable region comprising a CDRH1 comprising the amino acid sequence of SEQ ID NO: 161, a CDRH2 comprising the amino acid sequence of SEQ ID NO: 162, and a CDRH3 comprising the amino acid sequence of SEQ ID NO: 163, wherein CDR_{H1}, CDR_{H2}, and CDR_{H3} sequences are interposed between immunoglobulin FR sequences (PAL769-VH, h769-VH); and/or an immunoglobulin light chain variable region comprising a CDRL1 comprising the amino acid sequence of SEQ ID NO: 165, a CDRL2 comprising the amino acid sequence of SEQ ID NO: 142, and a CDRL3 comprising the amino acid sequence of SEQ ID NO: 203, wherein the CDR_{L1}, CDR_{L2}, and CDR_{L3} sequences are interposed between immunoglobulin FR sequences (h769.T-VL).

In certain embodiments, an antibody can comprise: an immunoglobulin heavy chain variable region comprising a CDR_{H1} comprising the amino acid sequence of SEQ ID NO: 250, a CDR_{H2} comprising the amino acid sequence of SEQ ID NO: 252, and a CDRH3 comprising the amino acid sequence of SEQ ID NO: 163, wherein CDR_{H1}, CDR_{H2}, and CDR_{H3} sequences are interposed between immunoglobulin FR sequences (h769-VH); and/or an immunoglobulin light chain variable region comprising a CDRL1 comprising the amino acid sequence of SEQ ID NO: 255, a CDR_{L2} comprising the amino acid sequence of SEQ ID NO: 254, and a CDR_{L3} comprising the amino acid sequence of SEQ ID NO: 203, wherein the CDR_{L1}, CDR_{L2}, and CDR_{L3} sequences are interposed between immunoglobulin FR sequences (h769.T-VL).

In certain embodiments, an antibody can comprise: an immunoglobulin heavy chain variable region comprising a CDRH1 comprising the amino acid sequence of SEQ ID NO: 129, a CDRH2 comprising the amino acid sequence of SEQ ID NO: 130, and a CDRH3 comprising the amino acid sequence of SEQ ID NO: 131, wherein CDR_{H1}, CDR_{H2}, and CDR_{H3} sequences are interposed between immunoglobulin FR sequences (PAL752-VH); and/or an immunoglobulin light chain variable region comprising a CDRL1 comprising the amino acid sequence of SEQ ID NO: 133, a CDRL2 comprising the amino acid sequence of SEQ ID NO: 134, and a CDRL3 comprising the amino acid sequence of SEQ ID NO: 135, wherein the CDR_{L1}, CDR_{L2}, and CDR_{L3} sequences are interposed between immunoglobulin FR sequences (PAL752-VL).

In certain embodiments, an antibody can comprise: an immunoglobulin heavy chain variable region comprising a CDRH1 comprising the amino acid sequence of SEQ ID NO: 137, a CDRH2 comprising the amino acid sequence of SEQ ID NO: 138, and a CDRH3 comprising the amino acid sequence of SEQ ID NO: 139, wherein CDR_{H1}, CDR_{H2}, and CDR_{H3} sequences are interposed between immunoglobulin FR sequences (PAL759-VH); and/or an immunoglobulin light chain variable region comprising a CDRL1 comprising the amino acid sequence of SEQ ID NO: 141, a CDRL2 comprising the amino acid sequence of SEQ ID NO: 142, and a CDRL3 comprising the amino acid sequence of SEQ ID NO: 143, wherein the CDR_{L1}, CDR_{L2}, and CDR_{L3} sequences are interposed between immunoglobulin FR sequences (PAL759-VL).

In certain embodiments, an antibody can comprise: an immunoglobulin heavy chain variable region comprising a CDRH1 comprising the amino acid sequence of SEQ ID NO: 145, a CDRH2 comprising the amino acid sequence of SEQ ID NO: 146, and a CDRH3 comprising the amino acid sequence of SEQ ID NO: 147, wherein CDR_{H1}, CDR_{H2}, and CDR_{H3} sequences are interposed between immunoglobulin FR sequences (PAL760-VH); and/or an immunoglobulin light chain variable region comprising a CDRL1 comprising the amino acid sequence of SEQ ID NO: 149, a CDRL2 comprising the amino acid sequence of SEQ ID NO: 150, and a CDRL3 comprising the amino acid sequence of SEQ ID NO: 151, wherein the CDR_{L1}, CDR_{L2}, and CDR_{L3} sequences are interposed between immunoglobulin FR sequences (PAL760-VL).

In certain embodiments, an antibody can comprise: an immunoglobulin heavy chain variable region comprising a CDRH1 comprising the amino acid sequence of SEQ ID NO: 153, a CDRH2 comprising the amino acid sequence of SEQ ID NO: 154, and a CDRH3 comprising the amino acid sequence of SEQ ID NO: 155, wherein CDR_{H1}, CDR_{H2}, and CDR_{H3} sequences are interposed between immunoglobulin FR sequences (PAL767-VH); and/or an immunoglobulin light chain variable region comprising a CDRL1 comprising the amino acid sequence of SEQ ID NO: 157, a CDRL2 comprising the amino acid sequence of SEQ ID NO: 158, and a CDRL3 comprising the amino acid sequence of SEQ ID NO: 159, wherein the CDR_{L1}, CDR_{L2}, and CDR_{L3} sequences are interposed between immunoglobulin FR sequences (PAL767-VL).

In certain embodiments, an antibody can comprise: an immunoglobulin heavy chain variable region comprising a CDRH1 comprising the amino acid sequence of SEQ ID NO: 161, a CDRH2 comprising the amino acid sequence of SEQ ID NO: 168, and a CDRH3 comprising the amino acid sequence of SEQ ID NO: 169, wherein CDR_{H1}, CDR_{H2}, and CDR_{H3} sequences are interposed between immunoglobulin FR sequences (PAL771-VH); and/or an immunoglobulin light chain variable region comprising a CDRL1 comprising the amino acid sequence of SEQ ID NO: 171, a CDRL2 comprising the amino acid sequence of SEQ ID NO: 172, and a CDRL3 comprising the amino acid sequence of SEQ ID NO: 173, wherein the CDR_{L1}, CDR_{L2}, and CDR_{L3} sequences are interposed between immunoglobulin FR sequences (PAL771-VL).

In certain embodiments, an antibody can comprise: an immunoglobulin heavy chain variable region comprising a CDRH1 comprising the amino acid sequence of SEQ ID NO: 175, a CDRH2 comprising the amino acid sequence of SEQ ID NO: 176, and a CDRH3 comprising the amino acid sequence of SEQ ID NO: 177, wherein CDR_{H1}, CDR_{H2}, and CDR_{H3} sequences are interposed between immunoglobulin FR sequences (PAL785-VH); and/or an immunoglobulin light chain variable region comprising a CDRL1 comprising the amino acid sequence of SEQ ID NO: 179, a CDRL2 comprising the amino acid sequence of SEQ ID NO: 180, and a CDRL3 comprising the amino acid sequence of SEQ ID NO: 181, wherein the CDR_{L1}, CDR_{L2}, and CDR_{L3} sequences are interposed between immunoglobulin FR sequences (PAL785-VL).

In certain embodiments, an antibody can comprise: an immunoglobulin heavy chain variable region comprising a CDRH1 comprising the amino acid sequence of SEQ ID NO: 183, a CDRH2 comprising the amino acid sequence of SEQ ID NO: 184, and a CDRH3 comprising the amino acid sequence of SEQ ID NO: 185, wherein CDR_{H1}, CDR_{H2}, and CDR_{H3} sequences are interposed between immunoglobulin FR sequences (PAL787-VH); and/or an immunoglobulin light chain variable region comprising a CDRL1 comprising the amino acid sequence of SEQ ID NO: 187, a CDRL2 comprising the amino acid sequence of SEQ ID NO: 188, and a CDRL3 comprising the amino acid sequence of SEQ ID NO: 189, wherein the CDR_{L1}, CDR_{L2}, and CDR_{L3} sequences are interposed between immunoglobulin FR sequences (PAL787-VL).

In certain embodiments, an antibody can comprise: an immunoglobulin heavy chain variable region comprising a CDRH1 comprising the amino acid sequence of SEQ ID NO: 191, a CDRH2 comprising the amino acid sequence of SEQ ID NO: 192, and a CDRH3 comprising the amino acid sequence of SEQ ID NO: 193, wherein CDR_{H1}, CDR_{H2}, and CDR_{H3} sequences are interposed between immunoglobulin FR sequences (PAL788-VH); and/or an immunoglobulin light chain variable region comprising a CDRL1 comprising the amino acid sequence of SEQ ID NO: 195, a CDRL2 comprising the amino acid sequence of SEQ ID NO: 196, and a CDRL3 comprising the amino acid sequence of SEQ ID NO: 197, wherein the CDR_{L1}, CDR_{L2}, and CDR_{L3} sequences are interposed between immunoglobulin FR sequences (PAL788-VL).

Similarly, the antibodies disclosed herein can comprise an immunoglobulin heavy chain variable region and an immunoglobulin light chain variable region.

In certain embodiments, the antibody comprises an immunoglobulin heavy chain variable region comprising an amino acid sequence selected from SEQ ID NO: 164, SEQ ID NO: 199, SEQ ID NO: 132, SEQ ID NO: 140, SEQ ID NO: 148, SEQ ID NO: 156, SEQ ID NO: 170, SEQ ID NO: 178, SEQ ID NO: 186, and SEQ ID NO: 194; and/or an immunoglobulin light chain variable region comprising an amino acid sequence selected from SEQ ID NO: 167, SEQ ID NO: 200, SEQ ID NO: 201, SEQ ID NO: 202, SEQ ID NO: 204, SEQ ID NO: 136, SEQ ID NO: 144, SEQ ID NO: 152, SEQ ID NO: 160, SEQ ID NO: 174, SEQ ID NO: 182, SEQ ID NO: 190, SEQ ID NO: 198, SEQ ID NO: 242, SEQ ID NO: 243, SEQ ID NO: 244, SEQ ID NO: 245, SEQ ID NO: 246, SEQ ID NO: 247, SEQ ID NO: 248 and SEQ ID NO: 249.

In certain embodiments, the antibody comprises an immunoglobulin heavy chain variable region comprising the amino acid sequence of SEQ ID NO: 164 (PAL769-VH), and an immunoglobulin light chain variable region comprising the amino acid sequence of SEQ ID NO: 167 (PAL769-VL).

In certain embodiments, the antibody comprises an immunoglobulin heavy chain variable region comprising the amino acid sequence of SEQ ID NO: 199 (h769-VH), and an immunoglobulin light chain variable region comprising the amino acid sequence of SEQ ID NO: 200 (h769-IF3-VL).

In certain embodiments, the antibody comprises an immunoglobulin heavy chain variable region comprising the amino acid sequence of SEQ ID NO: 199 (h769-VH), and an immunoglobulin light chain variable region comprising the amino acid sequence of SEQ ID NO: 201 (h769-tm2-VL).

In certain embodiments, the antibody comprises an immunoglobulin heavy chain variable region comprising the amino acid sequence of SEQ ID NO: 199 (h769-VH), and an immunoglobulin light chain variable region comprising the amino acid sequence of SEQ ID NO: 202 (h769-tm3-VL).

In certain embodiments, the antibody comprises an immunoglobulin heavy chain variable region comprising the amino acid sequence of SEQ ID NO: 199 (h769-VH), and an immunoglobulin light chain variable region comprising the amino acid sequence of SEQ ID NO: 204 (h769.T-VL).

In certain embodiments, the antibody comprises an immunoglobulin heavy chain variable region comprising the amino acid sequence of SEQ ID NO: 132 (PAL752-VH), and an immunoglobulin light chain variable region comprising the amino acid sequence of SEQ ID NO: 136 (PAL752-VL).

In certain embodiments, the antibody comprises an immunoglobulin heavy chain variable region comprising the amino acid sequence of SEQ ID NO: 140 (PAL759-VH), and an immunoglobulin light chain variable region comprising the amino acid sequence of SEQ ID NO: 144 (PAL759-VL).

In certain embodiments, the antibody comprises an immunoglobulin heavy chain variable region comprising the amino acid sequence of SEQ ID NO: 148 (PAL760-VH), and an immunoglobulin light chain variable region comprising the amino acid sequence of SEQ ID NO: 152 (PAL760-VL).

In certain embodiments, the antibody comprises an immunoglobulin heavy chain variable region comprising the amino acid sequence of SEQ ID NO: 156 (PAL767-VH), and an immunoglobulin light chain variable region comprising the amino acid sequence of SEQ ID NO: 160 (PAL767-VL).

In certain embodiments, the antibody comprises an immunoglobulin heavy chain variable region comprising the amino acid sequence of SEQ ID NO: 170 (PAL771-VH), and an immunoglobulin light chain variable region comprising the amino acid sequence of SEQ ID NO: 174 (PAL771-VL).

In certain embodiments, the antibody comprises an immunoglobulin heavy chain variable region comprising the amino acid sequence of SEQ ID NO: 178 (PAL785-VH), and an immunoglobulin light chain variable region comprising the amino acid sequence of SEQ ID NO: 182 (PAL785-VL).

In certain embodiments, the antibody comprises an immunoglobulin heavy chain variable region comprising the amino acid sequence of SEQ ID NO: 186 (PAL787-VH), and an immunoglobulin light chain variable region comprising the amino acid sequence of SEQ ID NO: 190 (PAL787-VL).

In certain embodiments, the antibody comprises an immunoglobulin heavy chain variable region comprising the amino acid sequence of SEQ ID NO: 194 (PAL788-VH), and an immunoglobulin light chain variable region comprising the amino acid sequence of SEQ ID NO: 198 (PAL788-VL).

In certain embodiments the antibody comprises an immunoglobulin heavy chain variable region comprising an amino acid sequence of

(SEQ ID NO: 236)
EVQLX₁X₂SGAEX₃X₄KPGAX₅VX₆X₇SCX₈X₉SGFNIKDTYMHWVX₁₀Q
X₁₁PX₁₂X₁₃GLEWX₁₄GX₁₅IDPANDNTX₁₆YX₁₇X₁₈KFQX₁₉X₂₀
X₂₁TITADTSX₂₂DTAYX₂₃X₂₄LSSLX₂₅SEDTAVYYCAREGYGGSYGE
GYWGQGTX₂₆X₂₇TVSS,

wherein X₁ is Gln or Val, X₂ is Glu or Gln, X₃ is Leu or Val, X₄ is Val or Lys, X₅ is Ser or Thr, X₆ is Thr or Lys, X₇ is Leu or Ile, X₈ is Thr or Lys, X₉ is Ala or Val, X₁₀ is Lys or Gln, X₁₁ is Arg or Ala, X₁₂ is Glu or Gly, X₁₃ is Gln or Lys, X₁₄ is Ile or Met, X₁₅ is Arg or Leu, X₁₆ is Lys or Ile, X₁₇ is Asp or Ala, X₁₈ is Pro or Glu, X₁₉ is Asp or Gly, X₂₀ is Lys or Arg, X₂₁ is Ala or Val, X₂₂ is Ser or Thr, X₂₃ is Leu or Met, X₂₄ is Arg or Glu, X₂₅ is Thr or Arg, X₂₆ is Thr or Leu, and X₂₇ is Leu or Val; and/or an immunoglobulin light chain variable region comprising an amino acid sequence of

(SEQ ID NO: 237)
X₁IVMTQX₂PX₃X₄LX₅X₆SX₇GX₈RVTX₉X₁₀CX₁₁ASQSVSNDX₁₂
X₁₃WYQQKPGQX₁₄PX₁₅LLIYYASIRFTGX₁₆PX₁₇RFX₁₈GSGX₁₉GT
DFTX₂₀TIX₂₁X₂₂X₂₃QX₂₄EDX₂₅AVYX₂₆CQQDYX₂₇SPWTFGX₂₈G
TKX₂₉EIK,

wherein X₁ is Ser or Glu, X₂ is Thr or Ser, X₃ is Lys or Pro, X₄ is Phe or Thr, X₅ is Leu or Ser, X₆ is Val or Leu, X₇ is Ala or Pro, X₈ is Asp or Glu, X₉ is Ile or Leu, X₁₀ is Thr or Ser, X₁₁ is Lys or Arg, X₁₂ is Val or Leu, X₁₃ is Ile or Ser, X₁₄ is Ser or Ala, X₁₅ is Lys or Arg, X₁₆ is Val or Ile, X₁₇ is Asp or Ala, X₁₈ is Ala or Ser, X₁₉ is Tyr or Ser, X₂₀ is Phe or Leu, X₂₁ is Asn or Ser, X₂₂ is Thr or Ser, X₂₃ is Val or Leu, X₂₄ is Ala or Pro, X₂₅ is Leu or Phe, X₂₆ is Phe or Tyr, X₂₇ is Tyr or Thr, X₂₈ is Gly or Gln, and X₂₉ is Leu or Val.

In certain embodiments, an isolated antibody that binds PD-L1 comprises an immunoglobulin heavy chain variable region comprising an amino acid sequence that is at least 70%, 75%, 80%, 85%, 86%, 87%, 88%, 89%, 89.5%, 90%, 90.5%, 91%, 92%, 93%, 94%, 95%, 96%, 97%, 98%, or 99% identical to the entire variable region and/or the framework region sequences of an amino acid sequence selected from SEQ ID NO: 164, SEQ ID NO: 199, SEQ ID NO: 132, SEQ ID NO: 140, SEQ ID NO: 148, SEQ ID NO: 156, SEQ ID NO: 170, SEQ ID NO: 178, SEQ ID NO: 186, and SEQ ID NO: 194. Alternatively or in addition, an isolated antibody that binds PD-L1 comprises an immunoglobulin light chain variable region comprising an amino acid sequence that is at least 70%, 75%, 80%, 85%, 86%, 87%, 88%, 89%, 90%, 91%, 92%, 93%, 94%, 94.5%, 95%, 95.5%, 96%, 97%, 98%, or 99% identical to the entire variable region and/or the framework region sequences of an amino acid sequence selected from SEQ ID NO: 167, SEQ ID NO: 200, SEQ ID NO: 201, SEQ ID NO: 202, SEQ ID NO: 204, SEQ ID NO: 136, SEQ ID NO: 144, SEQ ID NO: 152, SEQ ID NO: 160, SEQ ID NO: 174, SEQ ID NO: 182, SEQ ID NO: 190, SEQ ID NO: 198, SEQ ID NO: 242, SEQ ID NO: 243, SEQ ID NO: 244, SEQ ID NO: 245, SEQ ID NO: 246, SEQ ID NO: 247, SEQ ID NO: 248 and SEQ ID NO: 249. Identification of CDR and framework sequences is within the level of ordinary skill in the art, and it is understood that the boundaries between CDR and framework sequences may depend upon the definition or convention that is used (e.g., Kabat, Chothia, IMGT, etc.). Exemplary CDR and framework sequences for heavy chain variable region sequences SEQ ID NO: 164 and SEQ ID NO: 199 are depicted in FIG. 51A, and exemplary CDR and framework sequences for light chain variable region sequences SEQ ID NO: 167, SEQ ID NO: 200, SEQ ID NO: 201, SEQ ID NO: 202, SEQ ID NO: 204, SEQ ID NO: 242, SEQ ID NO: 243, SEQ ID NO: 244, SEQ ID NO: 245, SEQ ID NO: 246, SEQ ID NO: 247, SEQ ID NO: 248, and SEQ ID NO: 249 are depicted in FIG. 51B.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases, see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; −W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=for all programs, not applicable to blastn; and −Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In each of the foregoing embodiments, it is contemplated herein that immunoglobulin heavy chain variable region sequences and/or light chain variable region sequences that together bind PD-L1 may each contain amino acid alterations (e.g., at least 1, 2, 3, 4, 5, or 10 amino acid substitutions, deletions, or additions) in the framework regions of the heavy and/or light chain variable regions.

In certain embodiments, it is contemplated that a heavy chain variable region sequence, for example, the V H sequence of SEQ ID NO: 164, SEQ ID NO: 199, SEQ ID NO: 132, SEQ ID NO: 140, SEQ ID NO: 148, SEQ ID NO: 156, SEQ ID NO: 170, SEQ ID NO: 178, SEQ ID NO: 186, or SEQ ID NO: 194, or the amino acid variants thereof, may be covalently linked to a variety of heavy chain constant region sequences known in the art. Similarly, it is contemplated that a light chain variable region sequence, for example, the VL of SEQ ID NO: 167, SEQ ID NO: 200, SEQ ID NO: 201, SEQ ID NO: 202, SEQ ID NO: 204, SEQ ID NO: 136, SEQ ID NO: 144, SEQ ID NO: 152, SEQ ID NO: 160, SEQ ID NO: 174, SEQ ID NO: 182, SEQ ID NO: 190, SEQ ID NO: 198, SEQ ID NO: 242, SEQ ID NO: 243, SEQ ID NO: 244, SEQ ID NO: 245, SEQ ID NO: 246, SEQ ID NO: 247, SEQ ID NO: 248 or SEQ ID NO: 249, or the amino acid variants thereof, may be covalently linked to a variety of light chain constant region sequences known in the art.

For example, the antibody molecule may have a heavy chain constant region chosen from, e.g., the heavy chain constant regions of IgG1, IgG2, IgG3, IgG4, IgM, IgA1, IgA2, IgD, and IgE; particularly, chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. The constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment the antibody has effector function and can fix complement. In other embodiments the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the constant region of the heavy chain of the antibody is a human IgG1 isotype, having an amino acid sequence:

(SEQ ID NO: 227)
ASTKGPSVFPLAPSSKSTSGGTAALGCLVKDYFPEPVTVSWNSGALTSGVHTFPAVLOSS

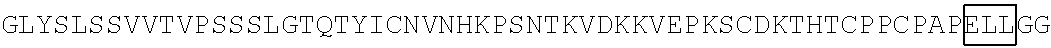

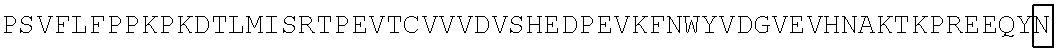

STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK.

In certain embodiments, the human IgG1 constant region is modified at amino acid Asn297 (boxed in SEQ ID NO: 227 in the preceding paragraph) to prevent to glycosylation of the antibody, for example Asn297Ala (N297A) or Asn297Gly (N297G). In certain embodiments, the constant region of the antibody is modified at amino acid Leu235 (boxed in SEQ ID NO: 227 in the preceding paragraph) to alter Fc receptor interactions, for example Leu235Glu (L235E) or Leu235Ala (L235A). In certain embodiments, the constant region of the antibody is modified at amino acid Leu234 (boxed in SEQ ID NO: 227 in the preceding paragraph) to alter Fc receptor interactions, e.g., Leu234Ala (L234A). In certain embodiments, the constant region of the antibody is modified at amino acid Glu233 (boxed in SEQ ID NO: 227 in the preceding paragraph), e.g., Glu233Pro (E233P). In certain embodiments, the constant region of the antibody is altered at both amino acid 234 and 235, for example Leu234Ala and Leu235Ala (L234A/L235A). In certain embodiments, the constant region of the antibody is altered at amino acids 233, 234, and 235, for example, Glu233Pro, Leu234Ala, and Leu235Ala (E233P L234A/L235A) (Armour K L. et al. (1999) EUR. J. IMMUNOL. 29(8):2613-24). In certain embodiments, the constant region of the antibody is altered at amino acids 234, 235 and 329, for example, Leu234Ala, Leu235Ala and Pro329Gly. (see, e.g., U.S. Pat. No. 8,969,526). All residue numbers are according to EU numbering (Kabat, E. A., et al., supra).

In certain embodiments, the constant region of the heavy chain of the antibody is a human IgG1 isotype, having an amino acid sequence:

(SEQ ID NO: 221)
ASTKGPSVFPLAPSSKSTSGGTAALGCLVKDYFPEPVTVSWNSGALTSGVHTFPAVLOSS

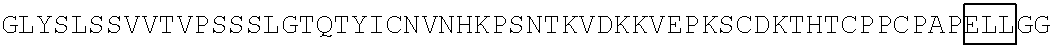

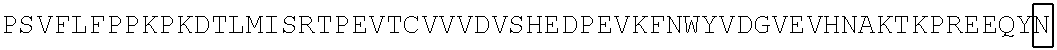

STYRVVSVLTVLHQDWLNGKEYKCKVSNKALPAPIEKTISKAKGQPREPQVYTLPPSREE
MTKNQVSLTCLVKGFYPSDIAVEWESNGOPENNYKTTPPVLDSDGSFFLYSKLTVDKSRW
QQGNVFSCSVMHEALHNHYTQKSLSLSPGK.

In certain embodiments, the human IgG1 constant region is modified at amino acid Asn297 (boxed in SEQ ID NO: 221 the preceding paragraph) to prevent to glycosylation of the antibody, for example Asn297Ala (N297A) or Asn297Gly (N297G). For example, in certain embodiments, the human IgG1 constant region comprises SEQ ID NO: 222, SEQ ID NO: 225, or SEQ ID NO: 226. In certain embodiments, the constant region of the antibody is modified at amino acid Leu235 (boxed in SEQ ID NO: 221 the preceding paragraph) to alter Fc receptor interactions, for example Leu235Glu (L235E) or Leu235Ala (L235A). In certain embodiments, the constant region of the antibody is modified at amino acid Leu234 (boxed in SEQ ID NO: 221 the preceding paragraph) to alter Fc receptor interactions, e.g., Leu234Ala (L234A). In certain embodiments, the constant region of the antibody is modified at amino acid Glu233 (boxed in SEQ ID NO: 221 the preceding paragraph), e.g., Glu233Pro (E233P). In certain embodiments, the constant region of the antibody is altered at both amino acid 234 and 235, for example Leu234Ala and Leu235Ala (L234A/L235A). In certain embodiments, the constant region of the antibody is altered at amino acids 233, 234, and 234, for example, Glu233Pro, Leu234Ala, and Leu235Ala (E233P L234A/L235A) (Armour K L. et al. (1999) EUR. J. IMMUNOL. 29(8):2613-24). In certain embodiments, the constant region of the antibody is altered at amino acids 234, 235 and 329, for example, Leu234Ala, Leu235Ala and Pro329Gly. (see, e.g., U.S. Pat. No. 8,969,526). All residue numbers are according to EU numbering (Kabat, E. A., et al., supra).

In certain embodiments, the human IgG1 constant region is modified to comprise either a “knob” mutation, e.g., T366Y, or a “hole” mutation, e.g., Y407T, for heterodimerization with a second constant region (residue numbers according to EU numbering (Kabat, E. A., et al., supra)). For example, in certain embodiments, the human IgG1 constant region comprises a Y407T mutation (e.g., the human IgG1 constant region comprises SEQ ID NO: 223 or SEQ ID NO: 225). In certain embodiments, the human IgG1 constant region comprises a T366Y mutation (e.g., the human IgG1 constant region comprises SEQ ID NO: 224 or SEQ ID NO: 226).

In certain embodiments, the constant region of the heavy chain of the antibody is a human IgG1 isotype, e.g., an allotype of the human IgG1 isotype, e.g., the IgG1 Glm3 allotype. Exemplary human IgG1 allotypes are described in Magdelaine-Beuzelin et al. (2009) PHARMACOGENET. GENOMICS 19(5):383-7.

In certain embodiments, the constant region of the heavy chain of the antibody is a human IgG2 isotype, having an amino acid sequence:

(SEQ ID NO: 228)
ASTKGPSVFPLAPCSRSTSESTAALGCLVKDYFPEPVTVSWNSGALTSGVHTFPAVLQSS
GLYSLSSVVTVPSSNFGTQTYTCNVDHKPSNTKVDKTVERKCCVECPPCPAPPVAGPSVF

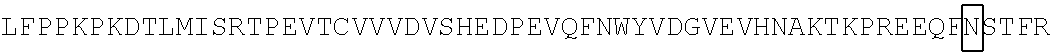

VVSVLTVVHQDWLNGKEYKCKVSNKGLPAPIEKTISKTKGQPREPQVYTLPPSREEMTKN
QVSLTCLVKGFYPSDISVEWESNGQPENNYKTTPPMLDSDGSFFLYSKLTVDKSRWQQGN
VFSCSVMHEALHNHYTQKSLSLSPGK.

In certain embodiments, the human IgG2 constant region is modified at amino acid Asn297 (boxed in SEQ ID NO: 228 in the preceding paragraph) to prevent to glycosylation of the antibody, e.g., Asn297A1a (N297A) or Asn297Gly (N297G), where the residue numbers are according to EU numbering (Kabat, E. A., et al., supra).

In certain embodiments, the constant region of the heavy chain of the antibody is an human IgG3 isotype, having an amino acid sequence:

(SEQ ID NO: 229)
ASTKGPSVFPLAPCSRSTSGGTAALGCLVKDYFPEPVTVSWNSGALTSGVHTFPAVLQSS
GLYSLSSVVTVPSSSLGTQTYTCNVNHKPSNTKVDKRVELKTPLGDTTHTCPRCPEPKSC
DTPPPCPRCPEPKSCDTPPPCPRCPEPKSCDTPPPCPRCPAPELLGGPSVFLFPPKPKDT

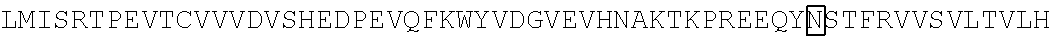

QDWLNGKEYKCKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQGNIFSCSVMHE

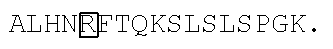

In certain embodiments, the human IgG3 constant region is modified at amino acid Asn297 (boxed in SEQ ID NO: 229 in the preceding paragraph) to prevent to glycosylation of the antibody, e.g., Asn297Ala (N297A) or Asn297Gly (N297G). In certain embodiments, the human IgG3 constant region is modified at amino acid Arg435 (boxed in SEQ ID NO: 229 in the preceding paragraph) to extend the half-life, e.g., Arg435H (R435H). All residue numbers are according to EU numbering (Kabat, E. A., et al., supra).

In certain embodiments, the constant region of the heavy chain of the antibody is an human IgG4 isotype, having an amino acid sequence:

(SEQ ID NO: 230)
ASTKGPSVFPLAPCSRSTSESTAALGCLVKDYFPEPVTVSWNSGALTSGVHTFPAVLQSS

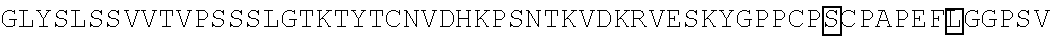

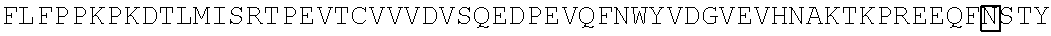

RVVSVLTVLHQDWLNGKEYKCKVSNKGLPSSIEKTISKAKGQPREPQVYTLPPSQEEMTK
NQVSLTCLVKGFYPSDIAVEWESNGOPENNYKTTPPVLDSDGSFFLYSRLTVDKSRWQEG
NVFSCSVMHEALHNHYTQKSLSLSLGK.

In certain embodiments, the human IgG4 constant region is modified within the hinge region to prevent or reduce strand exchange, e.g., in certain embodiments human IgG4 constant region is modified at Ser228 (boxed in SEQ ID NO: 230 in the preceding paragraph), e. Ser228Pro (S228P). In other embodiments, the human IgG4 constant region is modified at amino acid Leu235 (boxed in SEQ ID NO: 230 in the preceding paragraph) to alter Fc receptor interactions, e.g., Leu235Glu (1,235E). In certain embodiments, the human IgG4 constant region is modified at both Ser228 and Leu335, e.g., Ser228Pro and. Leu235Glu. (S228P/L235E). In certain embodiments, the human IgG4 constant region is modified at amino acid Asn297 (boxed in SEQ ID NO: 230 in the preceding paragraph) to prevent to glycosylation of the antibody, e.g., Asn297Ala (N297A) or Asn297Gly (N297G). All residue numbers are according to EU numbering (Kabat, E. A., et al., supra).

In certain embodiments, the human IgG constant region is modified to enhance FcRn binding. Examples of Fc mutations that enhance binding to FcRn are Met252Tyr, Ser254Thr, Thr256Glu (M252Y, S2541, T256E, respectively) (Dall'Acqua et al (2006) J. BIOL. CHEM. 281(33): 23514-23524), or Met428Leu and Asn434Ser (M428L, N434S) (Zalevsky et al (2010) NATURE BIOTECH. 28(2): 157-159). All residue numbers are according to EU numbering (Kabat, E. A., et al., supra).

In certain embodiments, the human IgG constant region is modified to alter antibody-dependent cellular cytotoxicity (ADCC) and/or complement-dependent cytotoxicity (CDC), e.g., the amino acid modifications described in Natsume et al. (2008) CANCER RES. 68(10): 3863-72; Idusogie et al. (2001) J. IMMUNOL. 166(4): 2571-5; Moore et al, (2010) MABS 2(2): 181-189; Lazar et al. (2006) PROC. NATL. ACAD. SCI. USA 103(11): 4005-4010, Shields et al. (2001) J. BIOL. CHEM. 276(9): 6591-6604; Stavenhagen et al. (2007) CANCER RES. 67(18): 8882-8890; Stavenhagen et al. (2008) ADVAN. ENZYME REGUL. 48: 152-164; Alegre et al. (1992) J. IMMUNOL. 148: 3461-3468.

In certain embodiments, the human IgG constant region is modified to induce heterodimerization. For example, a heavy chain having an amino acid modification within the CH3 domain at Thr366, e.g., a substitution with a more bulky amino acid, e.g., Tyr (T366W), is able to preferentially pair with a second heavy chain having a CH3 domain having amino acid modifications to less bulky amino acids at positions Thr366, Leu368, and Tyr407, e.g., Ser, Ala and Val, respectively (T366S/L368A/Y407V). Heterodimerization via CH3 modifications can be further stabilized by the introduction of a disulfide bond, for example by changing Ser354 to Cys (S354C) and Y349 to Cys (Y349C) on opposite CH3 domains (see, Carter (2001) J. IMMUNOL. METHODS 248: 7-15).

In certain embodiments, the constant region of the light chain of the antibody is a human kappa constant region, e.g., a human kappa constant region having the amino acid sequence:

(SEQ ID NO: 231)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC,

In certain embodiments, the constant region of the light chain of the antibody is a human kappa constant region, e.g., a human kappa constant region having the amino acid sequence:

(SEQ ID NO: 232)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In certain embodiments, the constant region of the light chain of the antibody is a human lambda constant region, e.g., a human lambda constant region having the amino acid sequence:

(SEQ ID NO: 233)
GQPKANPTVTLFPPSSEELQANKATLVCLISDFYPGAVTVAWKADGSPVK
AGVETTKPSKQSNNKYAASSYLSLTPEQWKSHRSYSCQVTHEGSTVEKTV
APTEC

In certain embodiments, the antibody comprises an immunoglobulin heavy chain comprising the amino acid sequence of SEQ ID NO: 235, or an amino acid sequence that has at least 85%, 90%, 95%, 95.5%, 96%, 96.5%, 97%, 98%, or 99% sequence identity to SEQ ID NO: 235; and/or an immunoglobulin light chain comprising the amino acid sequence of SEQ ID NO: 205, or an amino acid sequence that has at least 85%, 90%, 94.5% 95%, 95.5%, 96%, 97%, 98%, or 99% sequence identity to SEQ ID NO: 205.

In certain embodiments, the antibody binds human PD-L1 with a K_{D} of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured using standard binding assays, for example, surface plasmon resonance or bio-layer interferometry. In certain embodiments, the antibody binds human PD-L1 with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, or from about 0.05 nM to about 0.035 nM, as measured using standard binding assays, for example, surface plasmon resonance or bio-layer interferometry.

In certain embodiments, in addition to binding human PD-L1, a disclosed antibody also binds to Macaca fascicularis (cynomolgus) PD-L1. For example, the antibody binds cynomolgus PD-L1 with a K_{D} of 20 nM, 15 nM, 10 nM, 9 nM, 8 nM, 7 nM, 6 nM, 5 nM, 4 nM, 3 nM, 2 nM, 1 nM, 0.75 nM, 0.5 nM, 0.1 nM, 0.075 nM, or 0.05 nM or lower, as measured using standard binding assays, for example, surface plasmon resonance or bio-layer interferometry. In certain embodiments, the antibody binds cynomolgus PD-L1 with a K_{D} of from about 20 nM to about 0.05 nM, from about 20 nM to about 0.075 nM, from about 20 nM to about 0.1 nM, from about 20 nM to about 0.5 nM, from about 20 nM to about 1 nM, from about 10 nM to about 0.05 nM, from about 10 nM to about 0.075 nM, from about 10 nM to about 0.1 nM, from about 10 nM to about 0.5 nM, from about 10 nM to about 1 nM, from about 5 nM to about 0.05 nM, from about 5 nM to about 0.075 nM, from about 5 nM to about 0.1 nM, from about 5 nM to about 0.5 nM, from about 5 nM to about 1 nM, from about 3 nM to about 0.05 nM, from about 3 nM to about 0.075 nM, from about 3 nM to about 0.1 nM, from about 3 nM to about 0.5 nM, from about 3 nM to about 1 nM, from about 3 nM to about 2 nM, from about 2 nM to about 0.05 nM, from about 2 nM to about 0.075 nM, from about 2 nM to about 0.1 nM, from about 2 nM to about 0.5 nM, from about 2 nM to about 1 nM, from about 1 nM to about 0.05 nM, from about 1 nM to about 0.075 nM, from about 1 nM to about 0.1 nM, from about 1 nM to about 0.5 nM, from about 0.5 nM to about 0.05 nM, from about 0.5 nM to about 0.075 nM, from about 0.5 nM to about 0.1 nM, from about 0.1 nM to about 0.05 nM, from about 0.1 nM to about 0.075 nM, or from about 0.075 nM to about 0.05 nM, as measured using standard binding assays, for example, surface plasmon resonance or bio-layer interferometry.

In certain embodiments, the antibody interferes with the binding of PD-L1 to PD-1.

In certain embodiments, the invention provides antibodies that bind to the same epitope present in PD-L1 as that bound by an antibody disclosed herein. In certain embodiments, the invention provides antibodies that compete for binding to PD-L1 with an antibody disclosed herein.

Competition assays for determining whether an antibody binds to the same epitope as, or competes for binding with a disclosed antibody are known in the art. Exemplary competition assays include immunoassays (e.g., ELISA assays, RIA assays), surface plasmon resonance, (e.g., BIAcore analysis), bio-layer interferometry, and flow cytometry.

Typically, a competition assay involves the use of an antigen (e.g., a human PD-L1 protein or fragment thereof) bound to a solid surface or expressed on a cell surface, a test PD-L1-binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (i.e., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

In certain embodiments, the antibody (i) comprises an immunoglobulin heavy chain variable region comprising an amino acid sequence that is at least 70%, 75%, 80%, 85%, 86%, 87%, 88%, 89%, 89.5%, 90%, 90.5%, 91%, 92%, 93%, 94%, 95%, 96%, 97%, 98%, or 99% identical to SEQ ID NO: 164, and an immunoglobulin light chain variable region comprising an amino acid sequence that is at least 70%, 75%, 80%, 85%, 86%, 87%, 88%, 89%, 90%, 91%, 92%, 93%, 94%, 94.5%, 95%, 95.5%, 96%, 97%, 98%, or 99% identical to SEQ ID NO: 167, and (ii) competes for binding to human PD-L1 with and/or binds to same epitope on human PD-L1 as an antibody comprising an immunoglobulin heavy chain variable region comprising the amino acid sequence of SEQ ID NO: 164, and an immunoglobulin light chain variable region comprising the amino acid sequence of SEQ ID NO: 167.

In certain embodiments, the antibody (i) comprises an immunoglobulin heavy chain variable region comprising an amino acid sequence that is at least 70%, 75%, 80%, 85%, 86%, 87%, 88%, 89%, 89.5%, 90%, 90.5%, 91%, 92%, 93%, 94%, 95%, 96%, 97%, 98%, or 99% identical to SEQ ID NO: 199, and an immunoglobulin light chain variable region comprising an amino acid sequence that is at least 70%, 75%, 80%, 85%, 86%, 87%, 88%, 89%, 90%, 91%, 92%, 93%, 94%, 94.5%, 95%, 95.5%, 96%, 97%, 98%, or 99% identical to SEQ ID NO: 200, and (ii) competes for binding to human PD-L1 with and/or binds to same epitope on human PD-L1 as an antibody comprising an immunoglobulin heavy chain variable region comprising the amino acid sequence of SEQ ID NO: 199, and an immunoglobulin light chain variable region comprising the amino acid sequence of SEQ ID NO: 200.

In certain embodiments, the antibody (i) comprises an immunoglobulin heavy chain variable region comprising an amino acid sequence that is at least 70%, 75%, 80%, 85%, 86%, 87%, 88%, 89%, 89.5%, 90%, 90.5%, 91%, 92%, 93%, 94%, 95%, 96%, 97%, 98%, or 99% identical to SEQ ID NO: 199, and an immunoglobulin light chain variable region comprising an amino acid sequence that is at least 70%, 75%, 80%, 85%, 86%, 87%, 88%, 89%, 90%, 91%, 92%, 93%, 94%, 94.5%, 95%, 95.5%, 96%, 97%, 98%, or 99%, and (ii) competes for binding to human PD-L1 with and/or binds to same epitope on human PD-L1 as an antibody comprising an immunoglobulin heavy chain variable region comprising the amino acid sequence of SEQ ID NO: 199, and an immunoglobulin light chain variable region comprising the amino acid sequence of SEQ ID NO: 201.

In certain embodiments, the antibody (i) comprises an immunoglobulin heavy chain variable region comprising an amino acid sequence that is at least 70%, 75%, 80%, 85%, 86%, 87%, 88%, 89%, 89.5%, 90%, 90.5%, 91%, 92%, 93%, 94%, 95%, 96%, 97%, 98%, or 99% identical to SEQ ID NO: 199, and an immunoglobulin light chain variable region comprising an amino acid sequence that is at least 70%, 75%, 80%, 85%, 86%, 87%, 88%, 89%, 90%, 91%, 92%, 93%, 94%, 94.5%, 95%, 95.5%, 96%, 97%, 98%, or 99% identical to SEQ ID NO: 202, and (ii) competes for binding to human PD-L1 with and/or binds to same epitope on human PD-L1 as an antibody comprising an immunoglobulin heavy chain variable region comprising the amino acid sequence of SEQ ID NO: 199, and an immunoglobulin light chain variable region comprising the amino acid sequence of SEQ ID NO: 202.

In certain embodiments, the antibody (i) comprises an immunoglobulin heavy chain variable region comprising an amino acid sequence that is at least 70%, 75%, 80%, 85%, 86%, 87%, 88%, 89%, 89.5%, 90%, 90.5%, 91%, 92%, 93%, 94%, 95%, 96%, 97%, 98%, or 99% identical to SEQ ID NO: 199, and an immunoglobulin light chain variable region comprising an amino acid sequence that is at least 70%, 75%, 80%, 85%, 86%, 87%, 88%, 89%, 90%, 91%, 92%, 93%, 94%, 94.5%, 95%, 95.5%, 96%, 97%, 98%, or 99% identical to SEQ ID NO: 204, and (ii) competes for binding to human PD-L1 with and/or binds to same epitope on human PD-L1 as an antibody comprising an immunoglobulin heavy chain variable region comprising the amino acid sequence of SEQ ID NO: 199, and an immunoglobulin light chain variable region comprising the amino acid sequence of SEQ ID NO: 204.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 132, and an immunoglobulin light chain variable region comprising an amino acid sequence that is at least 70%, 75%, 80%, 85%, 90%, 95%, 96%, 97%, 98%, or 99% identical to SEQ ID NO: 136, and (ii) competes for binding to human PD-L1 with and/or binds to same epitope on human PD-L1 as an antibody comprising an immunoglobulin heavy chain variable region comprising the amino acid sequence of SEQ ID NO: 132, and an immunoglobulin light chain variable region comprising the amino acid sequence of SEQ ID NO: 136.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 140, and an immunoglobulin light chain variable region comprising an amino acid sequence that is at least 70%, 75%, 80%, 85%, 90%, 95%, 96%, 97%, 98%, or 99% identical to SEQ ID NO: 144, and (ii) competes for binding to human PD-L1 with and/or binds to same epitope on human PD-L1 as an antibody comprising an immunoglobulin heavy chain variable region comprising the amino acid sequence of SEQ ID NO: 140, and an immunoglobulin light chain variable region comprising the amino acid sequence of SEQ ID NO: 144.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 148, and an immunoglobulin light chain variable region comprising an amino acid sequence that is at least 70%, 75%, 80%, 85%, 90%, 95%, 96%, 97%, 98%, or 99% identical to SEQ ID NO: 152, and (ii) competes for binding to human PD-L1 with and/or binds to same epitope on human PD-L1 as an antibody comprising an immunoglobulin heavy chain variable region comprising the amino acid sequence of SEQ ID NO: 148, and an immunoglobulin light chain variable region comprising the amino acid sequence of SEQ ID NO: 152.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 156, and an immunoglobulin light chain variable region comprising an amino acid sequence that is at least 70%, 75%, 80%, 85%, 90%, 95%, 96%, 97%, 98%, or 99% identical to SEQ ID NO: 160, and (ii) competes for binding to human PD-L1 with and/or binds to same epitope on human PD-L1 as an antibody comprising an immunoglobulin heavy chain variable region comprising the amino acid sequence of SEQ ID NO: 156, and an immunoglobulin light chain variable region comprising the amino acid sequence of SEQ ID NO: 160.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 170, and an immunoglobulin light chain variable region comprising an amino acid sequence that is at least 70%, 75%, 80%, 85%, 90%, 95%, 96%, 97%, 98%, or 99% identical to SEQ ID NO: 174, and (ii) competes for binding to human PD-L1 with and/or binds to same epitope on human PD-L1 as an antibody comprising an immunoglobulin heavy chain variable region comprising the amino acid sequence of SEQ ID NO: 170, and an immunoglobulin light chain variable region comprising the amino acid sequence of SEQ ID NO: 174.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 178, and an immunoglobulin light chain variable region comprising an amino acid sequence that is at least 70%, 75%, 80%, 85%, 90%, 95%, 96%, 97%, 98%, or 99% identical to SEQ ID NO: 182, and (ii) competes for binding to human PD-L1 with and/or binds to same epitope on human PD-L1 as an antibody comprising an immunoglobulin heavy chain variable region comprising the amino acid sequence of SEQ ID NO: 178, and an immunoglobulin light chain variable region comprising the amino acid sequence of SEQ ID NO: 182.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 186, and an immunoglobulin light chain variable region comprising an amino acid sequence that is at least 70%, 75%, 80%, 85%, 90%, 95%, 96%, 97%, 98%, or 99% identical to SEQ ID NO: 190, and (ii) competes for binding to human PD-L1 with and/or binds to same epitope on human PD-L1 as an antibody comprising an immunoglobulin heavy chain variable region comprising the amino acid sequence of SEQ ID NO: 186, and an immunoglobulin light chain variable region comprising the amino acid sequence of SEQ ID NO: 190.

In certain embodiments, the antibody (i) comprises an immunoglobulin heavy chain variable region comprising an amino acid sequence that is at least 70%, 75%, 80%, 85%, 90%, 95%, 96%, 97%, 98%, or 99% identical to SEQ ID NO: 194, and an immunoglobulin light chain variable region comprising an amino acid sequence that is at least 70%, 75%, 80%, 85%, 90%, 95%, 96%, 97%, 98%, or 99% identical to SEQ ID NO: 198, and (ii) competes for binding to human PD-L1 with and/or binds to same epitope on human PD-L1 as an antibody comprising an immunoglobulin heavy chain variable region comprising the amino acid sequence of SEQ ID NO: 194, and an immunoglobulin light chain variable region comprising the amino acid sequence of SEQ ID NO: 198.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. Affinity-maturation procedures are within ordinary skill in the art. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (i.e., a process referred to as germlining).

Generally, an optimized antibody has at least the same, or substantially the same, affinity for the antigen as the non-optimized (or parental) antibody from which it was derived. Preferably, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

### II. Sialidase Anti-PD-L1 Fusion Proteins

To promote the selective removal of sialic acids on cells, e.g., hypersialylated cancer cells such as PD-L1 expressing cancer cells, and/or in the tumor microenvironment, it may be helpful to target a sialidase as described herein to such a cell or to such a tumor microenvironment. Additionally, in order to promote the removal of sialic acid by a sialidase in a subject, it may be helpful to extend the plasma half-life of the sialidase in the subject. These can be achieved by including the sialidase in a fusion protein and/or antibody conjugate (e.g., a chemically conjugated conjugate).

Accordingly, the invention further provides fusion proteins comprising a sialidase enzyme, or a functional fragment thereof, and a portion or fragment of an anti-PD-L1 antibody, such as an immunoglobulin Fc domain (also referred to herein as an Fc domain), or an immunoglobulin antigen-binding domain (also referred to herein as an antigen-binding domain). In certain embodiments, the sialidase and anti-PD-L1 antibody or portion thereof (e.g., immunoglobulin Fc domain or antigen-binding domain) are linked by a peptide bond or an amino acid linker.

As used herein, unless otherwise indicated, the term “fusion protein” is understood to refer to a single polypeptide chain comprising amino acid sequences based upon two or more separate proteins or polypeptide chains, where the two amino acid sequences may be fused together directly or via an intervening linker sequence, e.g., via an intervening amino acid linker. A nucleotide sequence encoding such a fusion protein can, for example, be created using conventional recombinant DNA technologies.

In certain embodiments, a fusion protein comprises a tag, such as a Strep tag (e.g., a Strep II tag), a His tag (e.g., a 10× His tag), a myc tag, or a FLAG tag. The tag can be located on the C-terminus or the N-terminus of the fusion protein.

a. Sialidase Portion

As used herein, the term “sialidase” refers to any enzyme, or a functional fragment thereof, that cleaves a terminal sialic acid residue from a substrate, for example, a glycoprotein or a glycolipid. The term sialidase includes variants having one or more amino acid substitutions, deletions, or insertions relative to a wild-type sialidase sequence, and/or fusion proteins or conjugates including a sialidase. Sialidases are also called neuraminidases, and, unless indicated otherwise, the two terms are used interchangeably herein. As used herein, the term “functional fragment” of a sialidase refers to fragment of a full-length sialidase that retains, for example, at least 10%, at least 20%, at least 30%, at least 40%, at least 50%, at least 60%, at least 70%, at least 80%, at least 90%, at least 95%, or 100% of the enzymatic activity of the corresponding full-length, naturally occurring sialidase. Sialidase enzymatic activity may be assayed by any method known in the art, including, for example, by measuring the release of sialic acid from the fluorogenic substrate 4-methylumbelliferyl-N-acetylneuraminic acid (4MU-NeuAc). In certain embodiments, the functional fragment comprises at least 100, 150, 200, 250, 300, 310, 320, 330, 340, 350, 360, or 370 consecutive amino acids present in a full-length, naturally occurring sialidase.

In certain embodiments, a sialidase portion of a sialidase-anti-PD-L1 fusion protein is derived from a eukaryotic sialidase, e.g., a mammalian sialidase, e.g., a human or mouse sialidase.

Four sialidases are encoded in the human genome: Neu1, Neu2, Neu3 and Neu4. Human Neu1 is a lysosomal neuraminidase enzyme which functions in a complex with beta-galactosidase and cathepsin A. The amino acid sequence of human Neu1 is depicted in SEQ ID NO: 7, and a nucleotide sequence encoding human Neu1 is depicted in SEQ ID NO: 23.

Human Neu2 is a cytosolic sialidase enzyme. The amino acid sequence of human Neu2 is depicted in SEQ ID NO: 1, and a nucleotide sequence encoding human Neu2 is depicted in SEQ ID NO: 24. Unless stated otherwise, as used herein, wild-type human Neu2 refers to human Neu2 having the amino acid sequence of SEQ ID NO: 1.

Human Neu3 is a plasma membrane sialidase with an activity specific for gangliosides. Human Neu3 has two isoforms: isoform 1 and isoform 2. The amino acid sequence of human Neu3, isoform 1 is depicted in SEQ ID NO: 8, and a nucleotide sequence encoding human Neu3, isoform 1 is depicted in SEQ ID NO: 25. The amino acid sequence of human Neu3, isoform 2 is depicted in SEQ ID NO: 9, and a nucleotide sequence encoding human Neu3, isoform 2 is depicted in SEQ ID NO: 34.

Human Neu4 has two isoforms: isoform 1 is a peripheral membrane protein and isoform 2 localizes to the lysosome lumen. The amino acid sequence of human Neu4, isoform 1 is depicted in SEQ ID NO: 10, and a nucleotide sequence encoding human Neu4, isoform 1 is depicted in SEQ ID NO: 26. The amino acid sequence of human Neu4, isoform 2 is depicted in SEQ ID NO: 11, and a nucleotide sequence encoding human Neu4, isoform 2 is depicted in SEQ ID NO: 35.

Four sialidases have also been found in the mouse genome and are referred to as Neu1, Neu2, Neu3 and Neu4. The amino acid sequence of mouse Neu1 is depicted in SEQ ID NO: 38, and a nucleotide sequence encoding mouse Neu1 is depicted in SEQ ID NO: 42. The amino acid sequence of mouse Neu2 is depicted in SEQ ID NO: 39 and a nucleotide sequence encoding mouse Neu2 is depicted in SEQ ID NO: 43. The amino acid sequence of mouse Neu3 is depicted in SEQ ID NO: 40, and a nucleotide sequence encoding mouse Neu3 is depicted in SEQ ID NO: 44. The amino acid sequence of mouse Neu4 is depicted in SEQ ID NO: 41, and a nucleotide sequence encoding mouse Neu4 is depicted in SEQ ID NO: 45.

In certain embodiments, a sialidase portion of a sialidase-anti-PD-L1 fusion protein is derived from a prokaryotic sialidase. Exemplary prokaryotic sialidases include sialidases from Salmonella typhimurium and Vibrio cholera. The amino acid sequence of Salmonella typhimurium sialidase (St-sialidase) is depicted in SEQ ID NO: 30, and a nucleotide sequence encoding Salmonella typhimurium sialidase is depicted in SEQ ID NO: 6. The amino acid sequence of Vibrio cholera sialidase is depicted in SEQ ID NO: 36, and a nucleotide sequence encoding Vibrio cholera sialidase is depicted in SEQ ID NO: 37.

In certain embodiments, the sialidase portion of a sialidase-anti-PD-L1 fusion protein is a mutant sialidase, e.g., a recombinant mutant human sialidase. In certain embodiments, the recombinant mutant human sialidase has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding (or template) wild-type human sialidase.

In certain embodiments, the recombinant mutant human sialidase has the same substrate specificity as the corresponding wild-type human sialidase. In other embodiments, the recombinant mutant human sialidase has a different substrate specificity than the corresponding wild-type human sialidase. For example, in certain embodiments the recombinant mutant human sialidase can cleave α2,3, α2,6, and/or α2,8 linkages. In certain embodiments the sialidase can cleave α2,3 and α2,8 linkages.

In certain embodiments, the expression yield of the recombinant mutant human sialidase in mammalian cells, e.g., HEK293 cells, CHO cells, murine myeloma cells (NS0, Sp2/0), or human fibrosarcoma cells (HT-1080), e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of the corresponding wild-type human sialidase.

In certain embodiments, the recombinant mutant human sialidase has about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more than 100% of the enzymatic activity of a corresponding wild-type human sialidase, and the expression yield of the recombinant mutant human sialidase in mammalian cells, e.g., HEK293 cells, is greater than about 10%, about 20%, about 50%, about 75%, about 100%, about 150%, about 200%, about 250%, about 300%, about 400%, about 500%, about 600%, about 700%, about 800%, about 900%, or about 1,000% of the expression yield of a corresponding wild-type human sialidase.

In certain embodiments, the amino acid sequence of the recombinant mutant human sialidase has at least 50%, 55%, 60%, 65%, 70%, 75%, 80%, 85%, 90%, 95%, 96%, 97%, 98%, or 99% sequence identity to the amino acid sequence of a corresponding wild-type human sialidase.

1. Substitution of Cysteine Residues

In certain embodiments, the recombinant mutant human sialidase comprises a substitution of at least one cysteine (cys, C) residue. It has been discovered that certain cysteine residues in sialidases may inhibit expression of functional protein as a result of protein aggregation. Accordingly, in certain embodiments, the recombinant mutant human sialidase contains at least one mutation to remove a free cysteine (e.g., for Neu1 (SEQ ID NO: 7), a mutation of, for example, one or more of C111, C117, C171, C183, C218, C240, C242, and C252; for Neu2 (SEQ ID NO: 1), a mutation of, for example, one or more of C125, C196, C219, C272, C332, and C352; for Neu3 (SEQ ID NO: 8), a mutation of, for example, one or more of C7, C90, C99, C106, C127, C136, C189, C194, C226, C242, C250, C273, C279, C295, C356, C365, C368, C384, C383, C394, and C415; and for Neu4 (SEQ ID NO: 10), a mutation of, for example, one or more of C88, C125, C126, C186, C191, C211, C223, C239, C276, C437, C453, C480, and C481). Free cysteines can be substituted with any amino acid. In certain embodiments, the free cysteine is substituted with serine (ser, S), isoleucine (iso, I), valine (val, V), phenylalanine (phe, F), leucine (leu, L), or alanine (ala, A). Exemplary cysteine substitutions in Neu2 include C125A, C125I, C125S, C125V, C196A, C196L, C196V, C272S, C272V, C332A, C332S, C332V, C352L, and C352V.

In certain embodiments, the recombinant mutant human sialidase comprises two or more cysteine substitutions. Exemplary double or triple cysteine substitutions in Neu2 include: C125S and C332S; C272V and C332A; C272V and C332S; C332A and C352L; C125S and C196L; C196L and C352L; C196L and C332A; C332A and C352L; and C196L, C332A and C352L.

In certain embodiments, the recombinant mutant human sialidase is a Neu2 sialidase and comprises the substitutions C322A and C352L.

In certain embodiments, the sialidase contains an amino acid substitution at 2, 3, 4, 5, or 6 cysteines typically present in a human sialidase, e.g., Neu2 or Neu3.

In certain embodiments, the recombinant mutant human sialidase comprises a substitution or combination of substitutions corresponding to a substitution or combination of substitutions listed in TABLE 1 (amino acid positions corresponding to wild-type human Neu2 (SEQ ID NO: 1)).

TABLE 1
Substitution(s)
C125A
C125I
C125S
C125V
C196A
C196L
C196V
C272S
C272V
C332A
C332S
C332V
C352L
C352V
C125S + C332S
C272V + C332A
C272V + C332S
C332A + C352L
C125S + C196L
C196L + C352L
C196L + C332A
C196L + C332A + C352L

2. Substitutions of Residues to Increase pI and/or Decrease Hydrophobicity

The isoelectric point (pI) of a protein is the pH at which the net charge is zero. The pI also generally indicates the pH at which the protein is least soluble, which may affect the ability to express and purify the protein. Generally, a protein has good solubility if its pI is greater than 2 units above the pH of the solution. Human Neu2 has a predicted pI of 7.5. Thus, human Neu2 is least soluble around neutral pH, which is undesirable because expression and physiological systems are at neutral pH. In contrast, the sialidase from Salmonella typhimurium (St-sialidase), which exhibits good solubility and recombinant expression, has a pI of 9.6. Accordingly, to increase expression of human Neu2 or the other human sialidases, a recombinant mutant human sialidase may be designed to contain one or more amino acid substitution(s) wherein the substitution(s) increase(s) the pI of the sialidase relative to a sialidase without the substitution. Additionally, decreasing the number of hydrophobic amino acids on the surface of a sialidase may improve expression of sialidase by, for example, reducing aggregation. Accordingly, to increase expression of human Neu2 or the other human sialidases, a recombinant mutant human sialidase may be designed to contain one or more amino acid substitution(s) wherein the substitution(s) decrease(s) the hydrophobicity of a surface of the sialidase relative to a sialidase without the substitution(s).

Accordingly, in certain embodiments, the recombinant mutant human sialidase comprises at least one amino acid substitution, wherein the substitution increases the isoelectric point (pI) of the sialidase and/or decreases the hydrophobicity of the sialidase relative to a sialidase without the substitution. This may be achieved by introducing one or more charged amino acids, for example, positively or negatively charged amino acids, into the recombinant sialidase. In certain embodiments, the amino acid substitution is to a charged amino acid, for example, a positively charged amino acid such as lysine (lys, K), histidine (his, H), or arginine (arg, R), or a negatively charged amino acid such as aspartic acid (asp, D) or glutamic acid (glu, E). In certain embodiments, the amino acid substitution is to a lysine residue. In certain embodiments, the substitution increases the pI of the sialidase to about 7.75, about 8, about 8.25, about 8.5, about 8.75, about 9, about 9.25, about 9.5, or about 9.75.

In certain embodiments, the amino acid substitution occurs at a surface exposed D or E amino acid, in a helix or loop, or in a position that has a K or R in the corresponding position of St-sialidase. In certain embodiments, the amino acid substitution occurs at an amino acid that is remote from the catalytic site or otherwise not involved in catalysis, an amino acid that is not conserved with the other human Neu proteins or with St-Sialidase or Clostridium NanH, or an amino acid that is not located in a domain important for function (e.g., an Asp-box or beta strand).

Exemplary amino acid substitutions in Neu2 that increase the isoelectric point (pI) of the sialidase and/or decrease the hydrophobicity of the sialidase relative to a sialidase without the substitution include A2E, A2K, D215K, V325E, V325K, E257K, and E319K. In certain embodiments, the recombinant mutant human sialidase comprises two or more amino acid substitutions, including, for example, A2K and V325E, A2K and V325K, E257K and V325K, A2K and E257K, and E257K and A2K and V325K.

In certain embodiments, the recombinant mutant human sialidase comprises a substitution or combination of substitutions corresponding to a substitution or combination of substitutions listed in TABLE 2 (amino acid positions corresponding to wild-type human Neu2 (SEQ ID NO: 1)).

TABLE 2
Substitution(s)
A2K
E72K
D215K
E257K
V325K
A2K + E257K
A2K + V325E
A2K + V325K
E257K + V325K

3. Addition of N-Terminal Peptides and N- or C-Terminal Substitutions

It has been discovered that the addition of a peptide sequence of two or more amino acids to the N-terminus of a human sialidase can improve expression and/or activity of the sialidase. In certain embodiments, the peptide is at least 2 amino acids in length, for example, from 2 to 20, from 2 to 10, from 2 to 5, or 2, 3, 4, 5, 6, 7, 8, 9, 10, 11, 12, 13, 14, 15, 16, 17, 18, 19, or 20 amino acids in length. In certain embodiments, the peptide may form, or have a propensity to form, an α-helix.

In mice, a Neu2 isoform (type B) found in thymus contains six amino acids not present in the canonical isoform of Neu2 found in skeletal muscle. In certain embodiments herein, the N-terminal six amino acids of the mouse thymus Neu2 isoform, MEDLRP (SEQ ID NO: 4), or variations thereof, can be added onto a human Neu, e.g., human Neu2. In certain embodiments, the recombinant mutant human sialidase comprises a peptide at least two amino acid residues in length covalently associated with an N-terminal amino acid of the sialidase. In certain embodiments the recombinant mutant human sialidase comprises the peptide MEDLRP (SEQ ID NO: 4) or EDLRP (SEQ ID NO: 3) covalently associated with an N-terminal amino acid of the sialidase. In certain embodiments, the sialidase may further comprise a cleavage site, e.g., a proteolytic cleavage site, located between the peptide, e.g., MEDLRP (SEQ ID NO: 4) or EDLRP (SEQ ID NO: 3), and the remainder of the sialidase. In certain embodiments, the peptide, e.g., MEDLRP (SEQ ID NO: 4) or EDLRP (SEQ ID NO: 3), may be post-translationally cleaved from the remainder of the sialidase.

Alternatively to, or in combination with, the N-terminal addition, 1-5 amino acids of the 12 amino acid N-terminal region of the recombinant mutant human sialidase may be removed, e.g., the N-terminal methionine can be removed. In certain embodiments, if the recombinant mutant human sialidase is Neu2, the N-terminal methionine can be removed, the first five amino acids (MASLP; SEQ ID NO: 12) can be removed, or the second through fourth amino acids (ASLP; SEQ ID NO: 13) can be removed.

In certain embodiments, 1-5 amino acids of the 12 amino acid N-terminal region of the recombinant mutant human sialidase are substituted with MEDLRP (SEQ ID NO: 4), EDLRP (SEQ ID NO: 3), or TVEKSVVF (SEQ ID NO: 14). For example, in certain embodiments, if the recombinant mutant human sialidase is Neu2, the amino acids MASLP (SEQ ID NO: 12), ASLP (SEQ ID NO: 13) or M are substituted with MEDLRP (SEQ ID NO: 4), EDLRP (SEQ ID NO: 3) or TVEKSVVF (SEQ ID NO: 14).

Human sialidases have a β-propeller structure, characterized by 6 blade-shaped β-sheets arranged toroidally around a central axis. Generally, hydrophobic interactions between the blades of a β-propeller, including between the N- and C-terminal blades, enhance stability. Accordingly, in order to increase expression of human Neu2 or the other human sialidases, a recombinant mutant human sialidase can be designed comprising an amino acid substitution that increases hydrophobic interactions and/or hydrogen bonding between the N- and C-terminal β-propeller blades of the sialidase.

Accordingly, in certain embodiments, the recombinant mutant human sialidase comprises a substitution of at least one wild-type amino acid residue, wherein the substitution increases hydrophobic interactions and/or hydrogen bonding between the N- and C-termini of the sialidase relative to a sialidase without the substitution. In certain embodiments, the wild-type amino acid is substituted with asparagine (asn, N), lysine (lys, K), tyrosine (tyr, Y), phenylalanine (phe, F), or tryptophan (trp, W). Exemplary substitutions in Neu2 that increase hydrophobic interactions and/or hydrogen bonding between the N- and C-termini include L4N, L4K, V6Y, L7N, L4N and L7N, L4N and V6Y and L7N, V12N, V12Y, V12L, V6Y, V6F, or V6W. In certain embodiments, the sialidase comprises the V6Y substitution.

In certain embodiments, the recombinant mutant human sialidase comprises a combination of the above substitutions. For example, a recombinant mutant human Neu2 sialidase can comprise the additional amino acids MEDLRP (SEQ ID NO: 4), EDLRP (SEQ ID NO: 3), or TVEKSVVF (SEQ ID NO: 14) at the N-terminus and, in combination, can comprise at least one L4N, L4K, V6Y, L7N, L4N and L7N, L4N and V6Y and L7N, V12N, V12Y, V12L, V6Y, V6F, or V6W substitution. In certain embodiments, the amino acids MASLP (SEQ ID NO: 12), ASLP (SEQ ID NO: 13) or M of a recombinant mutant human Neu2 sialidase are replaced with MEDLRP (SEQ ID NO: 4), EDLRP (SEQ ID NO: 3) or TVEKSVVF (SEQ ID NO: 14) and the recombinant mutant human Neu2 sialidase also comprises at least one L4N, L4K, V6Y, L7N, L4N and L7N, L4N and V6Y and L7N, V12N, V12Y, V12L, V6Y, V6F, or V6W substitution.

In certain embodiments, the recombinant mutant human sialidase comprises a mutation or combination of mutations corresponding to a mutation or combination of mutations listed in TABLE 3 (amino acid positions corresponding to wild-type human Neu2 (SEQ ID NO: 1)).

TABLE 3
Mutation(s)
Substitute M at the N-terminus with EDLRP (SEQ ID NO: 3)
Substitute M at the N-terminus with MEDLRP (SEQ ID NO: 4)
Insert MEDLRP (SEQ ID NO: 4) at the N-terminus
Substitute MASLP (SEQ ID NO: 12) at the N-terminus with
MEDLRP (SEQ ID NO: 4)
L4N
V6Y
L7N
V6F
V6W

Additionally, in certain embodiments, the sialidase comprises a substitution or deletion of an N-terminal methionine at the N-terminus of the sialidase. For example, in certain embodiments, the sialidase comprises a substitution of a methionine residue at a position corresponding to position 1 of wild-type human Neu2 (SEQ ID NO: 1), e.g., the methionine at a position corresponding to position 1 of wild-type human Neu2 is substituted by alanine (M1A) or aspartic acid (M1D). In other embodiments, the sialidase comprises a deletion of a methionine residue at a position corresponding to position 1 (ΔM1) of wild-type human Neu2 (SEQ ID NO: 1).

In certain embodiments, the recombinant mutant human sialidase comprises a substitution or combination of substitutions corresponding to a substitution or combination of substitutions listed in TABLE 4 (amino acid positions corresponding to wild-type human Neu2 (SEQ ID NO: 1)).

TABLE 4
Mutation(s)
Deletion of M1, V6Y, I187K
M1R, V6Y, I187K
M1H, V6Y, I187K
M1K, V6Y, I187K
M1D, V6Y, I187K
M1T, V6Y, I187K
M1N, V6Y, I187K
M1Q, V6Y, I187K
M1G, V6Y, I187K
M1A, V6Y, I187K
M1V, V6Y, I187K
M1L, V6Y, I187K
M1F, V6Y, I187K
M1Y, V6Y, I187K

4. Substitutions of Residues to Decrease Proteolytic Cleavage

It has been discovered that certain sialidases (e.g., human Neu2) are susceptible to cleavage by a protease (e.g., trypsin). As a result, proteolytic cleavage of the sialidase may occur during recombinant protein production, harvesting, purification, or formulation, during administration to a subject, or after administration to a subject. Accordingly, in certain embodiments, the recombinant mutant human sialidase comprises a substitution of at least one wild-type amino acid residue, wherein the substitution decreases cleavage of the sialidase by a protease (e.g., trypsin) relative to a sialidase without the substitution.

In certain embodiments, incubation of the recombinant mutant human sialidase with a protease (e.g., trypsin) results in from about 1% to about 50%, from about 1% to about 40%, from about 1%, to about 30%, from about 1% to about 20%, from about 1% to about 10%, from about 1% to about 5%, from about 5% to about 50%, from about 5% to about 40%, from about 5% to about 30%, from about 5% to about 20%, from about 5% to about 10%, from about 10% to about 50%, from about 10% to about 40%, from about 10% to about 30%, from about 10% to about 20%, from about 20% to about 50%, from about 20% to about 40%, from about 20% to about 30%, from about 30% to about 50%, from about 30% to about 40%, or from about 40% to about 50% of the proteolytic cleavage of a corresponding wild-type sialidase when incubated with the protease under the same conditions. In certain embodiments, incubation of the recombinant mutant human sialidase with a protease (e.g., trypsin) results in less than 50%, less than 40%, less than 30%, less than 10%, less than 5%, less than 3%, less than 1%, or less than 0.5% of the proteolytic cleavage of a corresponding wild-type sialidase when incubated with the protease under the same conditions. Proteolytic cleavage can be assayed by any method known in the art, including for example, by SDS-PAGE as described in Example 4 herein.

Exemplary substitutions that increase resistance to proteolytic cleavage include: (i) a substitution of an alanine residue at a position corresponding to position 242 of wild-type human Neu2 (SEQ ID NO: 1), e.g., a substitution by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ii) a substitution of an arginine residue at a position corresponding to position 243 of wild-type human Neu2 (SEQ ID NO: 1), e.g., a substitution by glutamic acid (R243E), histidine (R243H), asparagine (R243N), glutamine (R243Q), or lysine (R243K); (iii) a substitution of a valine residue at a position corresponding to position 244 of wild-type human Neu2 (SEQ ID NO: 1), e.g., a substitution by isoleucine (V244I), lysine (V244K), or proline (V244P); or (iv) a combination of any of the foregoing. In certain embodiments, the recombinant mutant human sialidase comprises a substitution selected from A242C, A242F, A242Y, and A242W. In certain embodiments, the recombinant mutant human sialidase comprises a substitution or a combination of substitutions corresponding to a substitution or combination of substitutions listed in TABLE 5 (amino acid positions corresponding to wild-type human Neu2 (SEQ ID NO: 1)).

TABLE 5

| Wild Type | |
|---|---|
| Human Neu2 | |
| (SEQ ID NO: 1) | |
| Amino Acid | Exemplary Substitution(s) at Specified Position(s) |
| A242 | C, F, G, H, I, K, L, M, N, P, Q, R, S, V, W, Y |
| R243 | E, H, N, Q, K |
| V244 | I, K, P |

Additional exemplary substitutions that increase resistance to proteolytic cleavage (and/or increase expression yield and/or enzymatic activity) include: (i) a substitution of a leucine residue at a position corresponding to position 240 of wild-type human Neu2 (SEQ ID NO: 1), e.g., a substitution by aspartic acid (L240D), asparagine (L240N), or tyrosine (L240Y); (ii) a substitution of an alanine residue at a position corresponding to position 213 of wild-type human Neu2 (SEQ ID NO: 1), e.g., a substitution by cysteine (A213C), asparagine (A213N), serine (A213S), or threonine (A213T); (iii) a substitution of an arginine residue at a position corresponding to position 241 of wild-type human Neu2 (SEQ ID NO: 1), e.g., a substitution by alanine (R241A), aspartic acid (R241D), leucine (R241L), glutamine (R241Q), or tyrosine (R241Y); (iv) a substitution of a serine residue at a position corresponding to position 258 of wild-type human Neu2 (SEQ ID NO: 1), e.g., a substitution by cysteine (S258C); (v) a substitution of a leucine residue at a position corresponding to position 260 of wild-type human Neu2 (SEQ ID NO: 1), e.g., a substitution by aspartic acid (L260D), phenylalanine (L260F), glutamine (L260Q), or threonine (L260T); (vi) a substitution of a valine residue at a position corresponding to position 265 of wild-type human Neu2 (SEQ ID NO: 1), e.g., a substitution by phenylalanine (V265F); or (vii) a combination of any of the foregoing. It is contemplated that, in certain embodiments, a substitution or a combination of substitutions at these positions may improve hydrophobic and/or aromatic interaction between secondary structure elements in the sialidase (e.g., between an α-helix and the nearest (3-sheet) thereby stabilizing the structure and improving resistance to proteolytic cleavage.

In certain embodiments, the recombinant mutant sialidase comprises a mutation at position L240. In certain embodiments, the recombinant mutant sialidase comprises a combination of mutations at positions (i) A213 and A242, (ii) A213, A242, and S258, (iii) L240 and L260, (iv) R241 and A242, (v) A242 and L260, (vi) A242 and V265, or (vii) L240 and A242. In certain embodiments, the recombinant mutant human sialidase comprises a combination of substitutions selected from (i) A213C, A242F, and S258C, (ii) A213C and A242F, (iii) A213T and A242F, (iv) R241Y and A242F, and (v) L240Y and A242F. In certain embodiments, the recombinant mutant human sialidase comprises a substitution or combination of substitutions corresponding to a substitution or combination of substitutions listed in TABLE 6 (amino acid positions corresponding to wild-type human Neu2 (SEQ ID NO: 1)).

TABLE 6
Substitution(s)
A242C, V244P
A242R, V244R
A242R, V244H
A242Y, V244P
A242T, V244P
A242N, V244P
A213C, A242F
A213S, A242F
A213T, A242F
A213N, A242F
A213C, A242F, S258C
A242F, L260F
A242F, V265F
L240Y
L240Y, L260F
L240D, L260T
L240N, L260T
L240N, L260D
L240N, L260Q
L240Y, A242F
R241A, A242F
R241Y, A242F

5. Other Substitutions

In certain embodiments, the recombinant mutant human sialidase comprises at least one of the following substitutions: I187K, A328E, K370N, or H210N. In certain embodiments, a recombinant mutant human Neu2 comprises the substitution of the amino acids GDYDAPTHQVQW (SEQ ID NO: 15) with the amino acids SMDQGSTW (SEQ ID NO: 16) or STDGGKTW (SEQ ID NO: 17). In certain embodiments, a recombinant mutant human Neu2 comprises the substitution of the amino acids PRPPAPEA (SEQ ID NO: 18) with the amino acids QTPLEAAC (SEQ ID NO: 19). In certain embodiments, a recombinant mutant human Neu2 comprises the substitution of the amino acids NPRPPAPEA (SEQ ID NO: 20) with the amino acids SQNDGES (SEQ ID NO: 21).

In certain embodiments, the recombinant mutant human sialidase comprises at least one substitution at a position corresponding to V212, A213, Q214, D215, T216, L217, E218, C219, Q220, V221, A222, E223, V224, E225, or T225.

In certain embodiments, the recombinant mutant human sialidase comprises an amino acid substitution at a position identified in TABLE 7 (amino acid positions corresponding to wild-type human Neu2 (SEQ ID NO: 1). In certain embodiments, the sialidase comprises an amino acid substitution identified in TABLE 7. In certain embodiments, the sialidase comprises a combination of any amino acid substitutions identified in TABLE 7.

TABLE 7

| Wild Type | |
|---|---|
| Human Neu2 | |
| (SEQ ID NO: 1) | |
| Amino Acid | Exemplary Substitution(s) at Specified Position(s) |
| M1 | D |
| L4 | S, T, Y, L, F, A, P, V, I, N, D, or H |
| P5 | G |
| V6 | Y |
| L7 | F, Y, S, I, T, or N |
| K9 | D |
| V12 | L, A, P, V, N, D, or H |
| F13 | S, N, R, K, T, G, D, E, or A |
| I22 | S, N, R, K, T, G, D, E, A, Y, L, F, P, V, I, or H |
| A24 | S, N, R, K, T, G, D, E, A, Y, L, F, P, V, I, or H |
| L34 | S, T, Y, L, F, A, P, V, I, N, D, or H |
| A36 | S, T, Y, L, F, A, P, V, I, N, D, or H |
| A42 | R or D |
| K44 | R or E |
| K45 | A, E, or R |
| L54 | M |
| P62 | H, G, N, T, S, F, I, D, or E |
| H64 | F, Y, S, I, T, or N |
| Q69 | H |
| R78 | K |
| D80 | P |
| P89 | S, T, Y, L, F, A, P, V, I, N, D, H, or M |
| A93 | E or K |
| G107 | D |
| Q108 | H |
| Q112 | R or K |
| C125 | Y, F, or L |
| Q126 | E, F, H, I, L, or Y |
| A150 | V |
| T156 | R, N, D, C, G, H, I, L, F, S, Y, V, A, P, or T |
| F157 | R, N, D, C, G, H, I, L, F, S, Y, V, A, or P |
| A158 | R, N, D, C, G, H, I, L, F, S, Y, V, A, P, or T |
| V159 | R, N, D, C, G, H, I, L, F, S, Y, V, A, or P |
| G160 | R, N, D, C, G, H, I, L, F, S, Y, V, A, P, or T |
| P161 | R, N, D, C, G, H, I, L, F, S, Y, V, A, or P |
| G162 | R, N, D, C, G, H, I, L, F, S, Y, V, A, P, or T |
| H163 | R, N, D, C, G, H, I, L, F, S, Y, V, A, or P |
| C164 | R, N, D, C, G, H, I, L, F, S, Y, V, A, P, or T |
| L165 | R, N, D, C, G, H, I, L, F, S, Y, V, A, or P |
| R170 | P |
| A171 | G |
| V176 | R, N, D, C, G, H, I, L, F, S, Y, V, P, or A |
| P177 | S, T, Y, L, F, A, P, V, I, N, D, or H |
| A178 | S, T, Y, L, F, A, P, V, I, N, D, or H |
| L184 | S, N, R, K, T, G, D, E, A, F, H, I, L, P, V, or Y |
| H185 | S, N, R, K, T, G, D, E, or A |
| P186 | S, N, R, K, T, G, D, E, A, F, H, I, L, P, V, or Y |
| I187 | S, N, R, K, T, G, D, E, or A |
| Q188 | P, S, N, R, K, T, G, D, E, or A |
| R189 | P |
| P190 | F, M, A, D, G, H, N, P, R, S, or T |
| I191 | M, A, D, F, H, I, L, N, P, S, T, V, Y, E, G, K, or R |
| A194 | S, T, Y, L, F, A, P, V, I, N, D, or H |
| A213 | C, N, S, or T |
| L217 | R, N, D, C, G, H, I, L, F, S, Y, or V |
| C219 | R, N, D, C, G, H, I, L, F, S, Y, or V |
| A222 | D |
| E225 | P |
| H239 | P |
| L240 | D, N, or Y |
| R241 | A, D, L, Q, or Y |
| A242 | C, F, G, H, I, K, L, M, N, Q, R, S, V, W, or Y |
| V244 | I or P |
| T249 | A |
| D251 | G |
| E257 | P |
| S258 | C |
| L260 | D, F, Q, or T |
| V265 | F |
| Q270 | S, T, A, H, P, or F |
| G271 | S, N, R, K, T, G, D, E, or A |
| C272 | S, N, R, K, T, G, D, E, A, C, H, Y, F, H, L, P, or V |
| W292 | R |
| S301 | A, D, E, F, G, H, I, K, L, M, N, P, Q, T, V, W, Y, C, or R |
| W302 | A, D, E, F, G, H, I, L, M, N, P, Q, R, S, T, V, Y, or K |
| E319 | D |
| V325 | F, Y, S, I, T, N, A, D, H, L, P, or V |
| L326 | F, Y, S, I, T, N, A, D, H, L, P, or V |
| L327 | F, Y, S, I, T, N, A, D, H, L, P, or V |
| C332 | A, D, G, H, N, P, R, S, or T |
| Y359 | A or S |
| V363 | R, S, T, Y, L, F, A, P, V, I, N, D, or H |
| L365 | K, Q, F, Y, S, I, T, N, A, D, H, L, P, or V |

For example, in certain embodiments, the recombinant mutant human sialidase comprises: (a) a substitution of a proline residue at a position corresponding to position 5 of wild-type human Neu2 (P5); (b) a substitution of a lysine residue at a position corresponding to position 9 of wild-type human Neu2 (K9); (c) a substitution of an alanine residue at a position corresponding to position 42 of wild-type human Neu2 (A42); (d) a substitution of a lysine residue at a position corresponding to position 44 of wild-type human Neu2 (K44); (e) a substitution of a lysine residue at a position corresponding to position 45 of wild-type human Neu2 (K45); (f) a substitution of a leucine residue at a position corresponding to position 54 of wild-type human Neu2 (L54); (g) a substitution of a proline residue at a position corresponding to position 62 of wild-type human Neu2 (P62); (h) a substitution of a glutamine residue at a position corresponding to position 69 of wild-type human Neu2 (Q69); (i) a substitution of an arginine residue at a position corresponding to position 78 of wild-type human Neu2 (R78); (j) a substitution of an aspartic acid residue at a position corresponding to position 80 of wild-type human Neu2 (D80); (k) a substitution of an alanine residue at a position corresponding to position 93 of wild-type human Neu2 (A93); (1) a substitution of a glycine residue at a position corresponding to position 107 of wild-type human Neu2 (G107); (m) a substitution of a glutamine residue at a position corresponding to position 108 of wild-type human Neu2 (Q108); (n) a substitution of a glutamine residue at a position corresponding to position 112 of wild-type human Neu2 (Q112); (o) a substitution of a cysteine residue at a position corresponding to position 125 of wild-type human Neu2 (C125); (p) a substitution of a glutamine residue at a position corresponding to position 126 of wild-type human Neu2 (Q126); (q) a substitution of an alanine residue at a position corresponding to position 150 of wild-type human Neu2 (A150); (r) a substitution of a cysteine residue at a position corresponding to position 164 of wild-type human Neu2 (C164); (s) a substitution of an arginine residue at a position corresponding to position 170 of wild-type human Neu2 (R170); (t) a substitution of an alanine residue at a position corresponding to position 171 of wild-type human Neu2 (A171); (u) a substitution of a glutamine residue at a position corresponding to position 188 of wild-type human Neu2 (Q188); (v) a substitution of an arginine residue at a position corresponding to position 189 of wild-type human Neu2 (R189); (w) a substitution of an alanine residue at a position corresponding to position 213 of wild-type human Neu2 (A213); (x) a substitution of a leucine residue at a position corresponding to position 217 of wild-type human Neu2 (L217); (y) a substitution of a glutamic acid residue at a position corresponding to position 225 of wild-type human Neu2 (E225); (z) a substitution of a histidine residue at a position corresponding to position 239 of wild-type human Neu2 (H239); (aa) a substitution of a leucine residue at a position corresponding to position 240 of wild-type human Neu2 (L240); (bb) a substitution of an arginine residue at a position corresponding to position 241 of wild-type human Neu2 (R241); (cc) a substitution of an alanine residue at a position corresponding to position 242 of wild-type human Neu2 (A242); (dd) a substitution of a valine residue at a position corresponding to position 244 of wild-type human Neu2 (V244); (ee) a substitution of a threonine residue at a position corresponding to position 249 of wild-type human Neu2 (T249); (ff) a substitution of an aspartic acid residue at a position corresponding to position 251 of wild-type human Neu2 (D251); (gg) a substitution of a glutamic acid residue at a position corresponding to position 257 of wild-type human Neu2 (E257); (hh) a substitution of a serine residue at a position corresponding to position 258 of wild-type human Neu2 (S258); (ii) a substitution of a leucine residue at a position corresponding to position 260 of wild-type human Neu2 (L260); (jj) a substitution of a valine residue at a position corresponding to position 265 of wild-type human Neu2 (V265); (kk) a substitution of a glutamine residue at a position corresponding to position 270 of wild-type human Neu2 (Q270); (ll) a substitution of a tryptophan residue at a position corresponding to position 292 of wild-type human Neu2 (W292); (mm) a substitution of a serine residue at a position corresponding to position 301 of wild-type human Neu2 (S301); (nn) a substitution of a tryptophan residue at a position corresponding to position 302 of wild-type human Neu2 (W302); (oo) a substitution of a valine residue at a position corresponding to position 363 of wild-type human Neu2 (V363); or (pp) a substitution of a leucine residue at a position corresponding to position 365 of wild-type human Neu2 (L365); or a combination of any of the foregoing substitutions. For example, the sialidase may comprise a substitution of K9, A42, P62, A93, Q216, A242, Q270, 5301, W302, V363, or L365, or a combination of any of the foregoing substitutions.

In certain embodiments, in the sialidase: (a) the proline residue at a position corresponding to position 5 of wild-type human Neu2 is substituted by histidine (P5H); (b) the lysine residue at a position corresponding to position 9 of wild-type human Neu2 is substituted by aspartic acid (K9D); (c) the alanine residue at a position corresponding to position 42 of wild-type human Neu2 is substituted by arginine (A42R) or aspartic acid (A42D); (d) the lysine residue at a position corresponding to position 44 of wild-type human Neu2 is substituted by arginine (K44R) or glutamic acid (K44E); (e) the lysine residue at a position corresponding to position 45 of wild-type human Neu2 is substituted by alanine (K45A), arginine (K45R), or glutamic acid (K45E); (f) the leucine residue at a position corresponding to position 54 of wild-type human Neu2 is substituted by methionine (L54M); (g) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T); (h) the glutamine residue at a position corresponding to position 69 of wild-type human Neu2 is substituted by histidine (Q69H); (i) the arginine residue at a position corresponding to position 78 of wild-type human Neu2 is substituted by lysine (R78K); (j) the aspartic acid residue at a position corresponding to position 80 of wild-type human Neu2 is substituted by proline (D80P); (k) the alanine residue at a position corresponding to position 93 of wild-type human Neu2 is substituted by glutamic acid (A93E) or lysine (A93K); (l) the glycine residue at a position corresponding to position 107 of wild-type human Neu2 is substituted by aspartic acid (G107D); (m) the glutamine residue at a position corresponding to position 108 of wild-type human Neu2 is substituted by histidine (Q108H); (n) the glutamine residue at a position corresponding to position 112 of wild-type human Neu2 is substituted by arginine (Q112R) or lysine (Q112K); (o) the cysteine residue at a position corresponding to position 125 of wild-type human Neu2 is substituted by leucine (C125L); (p) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y); (q) the alanine residue at a position corresponding to position 150 of wild-type human Neu2 is substituted by valine (A150V); (r) the cysteine residue at a position corresponding to position 164 of wild-type human Neu2 is substituted by glycine (C164G); (s) the arginine residue at a position corresponding to position 170 of wild-type human Neu2 is substituted by proline (R170P); (t) the alanine residue at a position corresponding to position 171 of wild-type human Neu2 is substituted by glycine (A171G); (u) the glutamine residue at a position corresponding to position 188 of wild-type human Neu2 is substituted by proline (Q188P); (v) the arginine residue at a position corresponding to position 189 of wild-type human Neu2 is substituted by proline (R189P); (w) the alanine residue at a position corresponding to position 213 of wild-type human Neu2 is substituted by cysteine (A213C), asparagine (A213N), serine (A213S), or threonine (A213T); (x) the leucine residue at a position corresponding to position 217 of wild-type human Neu2 is substituted by alanine (L217A) or valine (L217V); (y) the threonine residue at a position corresponding to position 249 of wild-type human Neu2 is substituted by alanine (T249A); (z) the aspartic acid residue at a position corresponding to position 251 of wild-type human Neu2 is substituted by glycine (D251G); (aa) the glutamic acid residue at a position corresponding to position 225 of wild-type human Neu2 is substituted by proline (E225P); (bb) the histidine residue at a position corresponding to position 239 of wild-type human Neu2 is substituted by proline (H239P); (cc) the leucine residue at a position corresponding to position 240 of wild-type human Neu2 is substituted by aspartic acid (L240D), asparagine (L240N), or tyrosine (L240Y); (dd) the arginine residue at a position corresponding to position 241 of wild-type human Neu2 is substituted by alanine (R241A), aspartic acid (R241D), leucine (R241L), glutamine (R241Q), or tyrosine (R241Y); (ee) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y); (ff) the valine residue at a position corresponding to position 244 of wild-type human Neu2 is substituted by isoleucine (V244I), lysine (V244K), or proline (V244P); (gg) the glutamic acid residue at a position corresponding to position 257 of wild-type human Neu2 is substituted by proline (E257P); (hh) the serine residue at a position corresponding to position 258 is substituted by cysteine (S258C); (ii) the leucine residue at a position corresponding to position 260 of wild-type human Neu2 is substituted by aspartic acid (L260D), phenylalanine (L260F), glutamine (L260Q), or threonine (L260T); (jj) the valine residue at a position corresponding to position 265 of wild-type human Neu2 is substituted by phenylalanine (V265F); (kk) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T); (ll) the tryptophan residue at a position corresponding to position 292 of wild-type human Neu2 is substituted by arginine (W292R); (mm) the serine residue at a position corresponding to position 301 of wild-type human Neu2 is substituted by alanine (S301A), aspartic acid (S301D), glutamic acid (S301E), phenylalanine (S301F), glycine (S301G), histidine (S301H), isoleucine (S301I), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y); (nn) the tryptophan residue at a position corresponding to position 302 of wild-type human Neu2 is substituted by alanine (W302A), aspartic acid (W302D), glutamic acid (W302E), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y); (oo) the valine residue at a position corresponding to position 363 of wild-type human Neu2 is substituted by arginine (V363R); or (pp) the leucine residue at a position corresponding to position 365 of wild-type human Neu2 is substituted by glutamine (L365Q), histidine (L365H), isoleucine (L365I), lysine (L365K) or serine (L365S); or the sialidase comprises a combination of any of the foregoing substitutions. For example, the sialidase may comprise a substitution selected from K9D, A42R, P62G, P62N, P62S, P62T, D80P, A93E, Q126H, Q126Y, R189P, H239P, A242T, Q270A, Q270S, Q270T, S301A, S301R, W302K, W302R, V363R, and L365I, or a combination of any of the foregoing substitutions.

In certain embodiments, the recombinant mutant human sialidase comprises a deletion of a leucine residue at a position corresponding to position 184 of wild-type human Neu2 (ΔL184), a deletion of a histidine residue at a position corresponding to position 185 of wild-type human Neu2 (ΔH185), a deletion of a proline residue at a position corresponding to position 186 of wild-type human Neu2 (ΔP186), a deletion of an isoleucine residue at a position corresponding to position 187 of wild-type human Neu2 (ΔI187), and a deletion of a glutamine residue at a position corresponding to position 184 of wild-type human Neu2 (ΔQ188), or a combination of any of the foregoing deletions.

In certain embodiments, the recombinant mutant human sialidase comprises an insertion between a threonine residue at a position corresponding to position 216 of wild-type human Neu2 and a leucine residue at a position corresponding to position 217 of wild-type human Neu2, for example, an insertion of an amino acid selected from S, T, Y, L, F, A, P, V, I, N, D, and H.

Additional exemplary sialidase mutations, and combinations of sialidase mutations, are described in International (PCT) Patent Application Publication No. WO 2019/136167, including in the Detailed Description in the section entitled “I. Recombinant Human Sialidases,” and in the Examples in Examples 1, 2, 3, 4, 5, and 6, and International (PCT) Patent Application Publication No. WO 2021/003469, including in the Detailed Description in the section entitled “I. Recombinant Human Sialidases,” and in the Examples in Examples 2, 3, 4, and 5, and in International (PCT) Patent Application No. PCT/US2021/040240, filed Jul. 2, 2021, including in the Detailed Description in the section entitled “I. Recombinant Human Sialidases,” and in the Examples in Examples 2, 3, 4, and 5.

6. Combinations of Substitutions

In certain embodiments, the recombinant mutant human sialidase comprises a combination of any of the mutations contemplated herein. For example, the recombinant mutant sialidase enzyme may comprise a combination of 2, 3, 4, 5, 6, 7, 8, 9, 10, 11, 12, 13, 14, 15 or more of the mutations contemplated herein. It is contemplated that the recombinant mutant sialidase enzyme may comprise 1-15, 1-10, 1-7, 1-6, 1-5, 1-4, 1-3, 1-2, 2-15, 2-10, 2-7, 2-6, 2-5, 2-4, 2-3, 3-15, 3-10, 3-7, 3-6, 3-5, or 3-4 of the mutations contemplated herein.

For example, the recombinant mutant sialidase enzyme may comprise a M1 deletion (ΔM1), M1A substitution, M1D substitution, V6Y substitution, K9D substitution, P62G substitution, P62N substitution, P62S substitution, P62T substitution, A93E substitution, I187K substitution, Q270A substitution, S301R substitution, W302K substitution, C332A substitution, V363R substitution, L365I substitution, or a combination of any of the foregoing.

In certain embodiments, the recombinant mutant sialidase enzyme comprises a M1 deletion (ΔM1), M1A substitution, M1D substitution, V6Y substitution, I187K substitution, C332A substitution, or a combination of any of the foregoing. For example, the recombinant mutant sialidase enzyme may comprise a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises (i) an amino acid substitution identified in TABLE 7, or a combination of any amino acid substitutions identified in TABLE 7, and (ii) an M1 deletion (ΔM1), M1A substitution, M1D substitution, V6Y substitution, I187K substitution, C332A substitution, or a combination of any of the foregoing. For example, the recombinant mutant sialidase enzyme may comprise (i) an amino acid substitution identified in TABLE 7, or a combination of any amino acid substitutions identified in TABLE 7, and (ii) a combination of mutations selected from: M1A and V6Y; M1A and I187K; M1A and C332A; M1D and V6Y; M1D and I187K; M1D and C332A; ΔM1 and V6Y; ΔM1 and I187K; ΔM1 and C332A; V6Y and I187K; V6Y and C332A; I187K and C332A; M1A, V6Y, and I187K; M1A, V6Y, and C332A; M1A, I187K, and C332A; M1D, V6Y, and I187K; M1D, V6Y, and C332A; M1D, I187K, and C332A; ΔM1, V6Y, and I187K; ΔM1, V6Y, and C332A; ΔM1, I187K, and C332A; V6Y, I187K, and C332A; M1A, V6Y, I187K, and C332A; M1D, V6Y, I187K, and C332A; and ΔM1, V6Y, I187K, and C332A.

In certain embodiments, the recombinant mutant sialidase enzyme comprises: (a) the M1D, V6Y, P62G, A93E, I187K, and C332A substitutions; (b) the M1D, V6Y, K9D, A93E, I187K, C332A, V363R, and L365I substitutions; (c) the M1D, V6Y, P62N, I187K, and C332A substitutions; (d) the M1D, V6Y, I187K, Q270A, S301R, W302K, and C332A substitutions; (e) the M1D, V6Y, P62S, I187K, Q270A, S301R, W302K, and C332A substitutions; (f) the M1D, V6Y, P62T, I187K, Q270A, S301R, W302K, and C332A substitutions; (g) the M1D, V6Y, P62N, I187K, Q270A, S301R, W302K, and C332A substitutions; (h) the M1D, V6Y, P62G, A93E, I187K, S301A, W302R, and C332A substitutions; (i) the M1D, V6Y, P62G, A93E, Q126Y, I187K, Q270T, and C332A substitutions; (j) the M1D, V6Y, P62G, A93E, Q126Y, I187K, and C332A substitutions; (k) the M1D, V6Y, P62G, A93E, Q126Y, I187K, A242F, Q270T, and C332A substitutions; or (1) the M1D, V6Y, A42R, P62G, A93E, Q126Y, I187K, A242F, Q270T, and C332A mutations.

In certain embodiments, the recombinant mutant human sialidase comprises a substitution of a serine residue at a position corresponding to position 301 of wild-type human Neu2 (S301) in combination with a substitution of a tryptophan residue at a position corresponding to position 302 of wild-type human Neu2 (W302). For example, the recombinant mutant human sialidase may comprise a combination of substitutions corresponding to a combination of substitutions listed in a row of TABLE 8 (amino acid positions corresponding to wild-type human Neu2 (SEQ ID NO: 1)). For example, the recombinant mutant human sialidase may comprise: the S301K and W302R substitutions; the S301K and W302K substitutions; or the S301A and W302S substitutions.

TABLE 8
Substitutions
S301A, W302R
S301A, W302S
S301A, W302T
S301K, W302S
S301N, W302S
S301T, W302S
S301T, W302T
S301T, W302R
S301A, W302A
S301K, W302R
S301K, W302T
S301N, W302T
S301K, W302K
S301P, W302R
S301P, W302S
S301P, W302T

In certain embodiments, the recombinant mutant human sialidase comprises a combination of substitutions corresponding to a combination of substitutions listed in a row of TABLE 9 (amino acid positions corresponding to wild-type human Neu2 (SEQ ID NO: 1)).

TABLE 9
Substitutions
M1D, V6Y, P62G, I187K, C332A
M1D, V6Y, K9D, I187K, C332A, V363R, L365I
M1D, V6Y, P62G, A93E, I187K, C332A
M1D, V6Y, K9D, I187K, C332A, V363R, L365K
M1D, V6Y, K9D, I187K, C332A, V363R, L365S
M1D, V6Y, K9D, I187K, C332A, V363R, L365Q
M1D, V6Y, K9D, I187K, C332A, V363R, L365H
M1D, V6Y, A93K, I187K, C332A
M1D, V6Y, A93E, I187K, C332A
V6Y, I187K, W292R
V6Y, G107D, I187K
V6Y, C125L
C125L, I187K
V6Y, C125L, I187K
M1D, V6Y, K45A, I187K, C332A
M1D, V6Y, Q270A, I187K, C332A
M1D, V6Y, K44R, K45R, I187K, C332A
M1D, V6Y, Q112R, I187K, C332A
M1D, V6Y, Q270F, I187K, C332A
M1D, V6Y, I187K, S301R, W302K, C332A
M1D, V6Y, K44E, K45E, I187K, C332A
M1D, V6Y, I187K, L217V, C332A
M1D, V6Y, I187K, L217A, C332A
M1D, V6Y, K44E, K45E, I187K, S301R, W302K, C332A
M1D, V6Y, Q112R, I187K, S301R, W302K, C332A
M1D, V6Y, I187K, Q270A, S301R, W302K, C332A
M1D, V6Y, K44E, K45E, Q112R, I187K, C332A
M1D, V6Y, K44E, K45E, I187K, Q270A, C332A
M1D, V6Y, K45A, I187K, Q270A, C332A
M1D, V6Y, 1187K, Q270H, C332A
M1D, V6Y, I187K, Q270P, C332A
M1D, V6Y, Q112K, I187K, C332A
M1D, V6Y, P62S, I187K, Q270A, S301R, W302K, C332A
M1D, V6Y, P62T, I187K, Q270A, S301R, W302K, C332A
M1D, V6Y, P62N, I187K, Q270A, S301R, W302K, C332A
V6Y, P62H, I187K
V6Y, Q108H, I187K
M1D, V6Y, P62H, I187K, C332A
M1D, V6Y, P62G, I187K, C332A
V6Y, P62G, I187K
M1D, V6Y, P62H, I187K
M1D, V6Y, Q108H, I187K
M1D, V6Y, P62N, I187K, C332A
M1D, V6Y, P62D, I187K, C332A
M1D, V6Y, P62E, I187K, C332A
V6Y, C164G, I187K, T249A
V6Y, C164G, I187K
V6Y, Q126L, I187K D251G
V6Y, L54M, Q69H, R78K, A171G, I187K
V6Y, P62T, I187K
V6Y, A150V, I187K
P5H, V6Y, P62S, I187K
V6Y, C164G, I187K
Q126Y, Q170T
Q126Y, A242F, Q270T
M1D, V6Y, P62G, A93E, Q126E, I187K, C332A
M1D, V6Y, P62G, A93E, Q126I, I187K, C332A
M1D, V6Y, P62G, A93E, Q126L, I187K, C332A
M1D, V6Y, P62G, A93E, Q126Y, I187K, C332A
M1D, V6Y, P62G, A93E, Q126F, I187K, C332A
M1D, V6Y, P62G, A93E, Q126H, I187K, C332A
M1D, V6Y, P62G, A93E, I187K, Q270S, C332A
M1D, V6Y, P62G, A93E, I187K, Q270T, C332A
M1D, V6Y, P62G, A93E, Q126Y, I187K, Q270T, C332A
M1D, V6Y, P62G, A93E, Q126Y, I187K, A242F, Q270T, C332A
M1D, V6Y, P62G, D80P, A93E, I187K, C332A
M1D, V6Y, P62G, A93E, R170P, I187K, C332A
M1D, V6Y, P62G, A93E, I187K, Q188P, C332A
M1D, V6Y, P62G, A93E, I187K, R189P, C332A
M1D, V6Y, P62G, A93E, I187K, E225P, C332A
M1D, V6Y, P62G, A93E, I187K, H239P, C332A
M1D, V6Y, P62G, A93E, I187K, E257P, C332A
M1D, V6Y, P62G, A93E, I187K, S301A, C332A
M1D, V6Y, P62G, A93E, I187K, S301D, C332A
M1D, V6Y, P62G, A93E, I187K, S301E, C332A
M1D, V6Y, P62G, A93E, I187K, S301F, C332A
M1D, V6Y, P62G, A93E, 1187K, S301H, C332A
M1D, V6Y, P62G, A93E, I187K, S301K, C332A
M1D, V6Y, P62G, A93E, I187K, S301L, C332A
M1D, V6Y, P62G, A93E, I187K, S301M, C332A
M1D, V6Y, P62G, A93E, I187K, S301N, C332A
M1D, V6Y, P62G, A93E, I187K, S301P, C332A
M1D, V6Y, P62G, A93E, I187K, S301Q, C332A
M1D, V6Y, P62G, A93E, I187K, S301R, C332A
M1D, V6Y, P62G, A93E, I187K, S301T, C332A
M1D, V6Y, P62G, A93E, I187K, S301V, C332A
M1D, V6Y, P62G, A93E, I187K, S301W, C332A
M1D, V6Y, P62G, A93E, I187K, S301Y, C332A
M1D, V6Y, P62G, A93E, I187K, W302A, C332A
M1D, V6Y, P62G, A93E, I187K, W302D, C332A
M1D, V6Y, P62G, A93E, I187K, W302F, C332A
M1D, V6Y, P62G, A93E, I187K, W302G, C332A
M1D, V6Y, P62G, A93E, I187K, W302H, C332A
M1D, V6Y, P62G, A93E, I187K, W302I, C332A
M1D, V6Y, P62G, A93E, I187K, W302L, C332A
M1D, V6Y, P62G, A93E, I187K, W302M, C332A
M1D, V6Y, P62G, A93E, I187K, W302N, C332A
M1D, V6Y, P62G, A93E, I187K, W302P, C332A
M1D, V6Y, P62G, A93E, I187K, W302Q, C332A
M1D, V6Y, P62G, A93E, I187K, W302R, C332A
M1D, V6Y, P62G, A93E, I187K, W302S, C332A
M1D, V6Y, P62G, A93E, I187K, W302T, C332A
M1D, V6Y, P62G, A93E, I187K, W302V, C332A
M1D, V6Y, P62G, A93E, I187K, W302Y, C332A
M1D, V6Y, P62G, A93E, I187K, S301A, W302A, C332A
M1D, V6Y, P62G, A93E, I187K, S301A, W302R, C332A
M1D, V6Y, P62G, A93E, I187K, S301A, W302S, C332A
M1D, V6Y, P62G, A93E, I187K, S301A, W302T, C332A
M1D, V6Y, P62G, A93E, I187K, S301K, W302S, C332A
M1D, V6Y, P62G, A93E, I187K, S301K, W302R, C332A
M1D, V6Y, P62G, A93E, I187K, S301K, W302T, C332A
M1D, V6Y, P62G, A93E, I187K, S301N, W302S, C332A
M1D, V6Y, P62G, A93E, I187K, S301N, W302T, C332A
M1D, V6Y, P62G, A93E, I187K, S301T, W302R, C332A
Q126Y, Q270T
Q126Y, A242F, Q270T
M1D, V6Y, A42R, P62G, A93E, Q126Y, I187K, A242F, Q270T, and C332A

In certain embodiments, the recombinant mutant human sialidase comprises the amino acid sequence of any one of SEQ ID NOs: 48-62, 94, 97, 100, 126, or 234, or an amino acid sequence that has at least 85%, 90%, 95%, 96%, 97%, 98%, or 99% sequence identity to any one of SEQ ID NOs: 48-62, 94, 97, 100, 126, or 234.

In certain embodiments, the recombinant mutant human sialidase comprises the amino acid sequence of

(SEQ ID NO: 238)
X₁X₂SX₃X₄X₅LQX₆SVFQSGAHAYRIPALLYLPGQQSLLAFAEQRX₇S
X₈X₉DEHAELIVX₁₀RRGDYDAX₁₁THQVQWX₁₂AQEVVAQAX₁₃LX₁₄G
HRSMNPCPLYDX₁₅QTGTLFLFFIAIPX₁₆X₁₇VTEX₁₈QQLQTRANVTR
LX₁₉X₂₀VTSTDHGRTWSSPRDLTDAAIGPX₂₁YREWSTFAVGPGHX₂₂L
QLHDX₂₃X₂₄RSLVVPAYAYRKLHPX₂₅X₂₆X₂₇PIPSAFX₂₈FLSHDHG
RTWARGHFVX₂₉QDTX₃₀ECQVAEVX₃₁TGEQRVVTLNARSX₃₂X₃₃X₃₄
X₃₅RX₃₆QAQSX₃₇NX₃₈GLDFQX₃₉X₄₀QX₄₁VKKLX₄₂EPPPX₄₃G
X₄₄QGSVISFPSPRSGPGSPAQX₄₅LLYTHPTHX₄₆X₄₇QRADLGAYLNP
RPPAPEAWSEPX₄₈LLAKGSX₄₉AYSDLQSMGTGPDGSPLFGX₅₀LYEAN
DYEEIX₅₁FX₅₂MFTLKQAFPAEYLPQ,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂₅ is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃₁ is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄₁ is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, and X₅₂ is Leu, Gln, His, Ile, Lys, or Ser, and the sialidase comprises at least one mutation relative to wild-type human Neu2 (SEQ ID NO: 1).

In certain embodiments, the recombinant mutant human sialidase comprises the amino acid sequence of

(SEQ ID NO: 239)
X₁ASLPX₂LQX₃ESVFQSGAHAYRIPALLYLPGQQSLLAFAEQRX₄SKKD
EHAELIVLRRGDYDAX₅THQVQWQAQEVVAQARLDGHRSMNPCPLYDX₆Q
TGTLFLFFIAIPGQVTEQQQLQTRANVTRLCX₇VTSTDHGRTWSSPRDLT
DAAIGPAYREWSTFAVGPGHCLQLHDRARSLVVPAYAYRKLHPX₈QRPIP
SAFCFLSHDHGRTWARGHEVAQDTLECQVAEVETGEQRVVTLNARSHLR
X₉RVQAQSTNDGLDFQESQLVKKLVEPPPX₁₀GCQGSVISFPSPRSGPGS
PAQWLLYTHPTHX₁₁X₁₂QRADLGAYLNPRPPAPEAWSEPVLLAKGSX₁₃
AYSDLQSMGTGPDGSPLFGCLYEANDYEEIX₁₄FX₁₅MFTLKQAFPAEYL
PQ,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, and X₁₅ is Leu, Gln, His, Ile, Lys, or Ser, and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile.

In certain embodiments, the recombinant mutant human sialidase comprises a conservative substitution relative to a recombinant mutant human sialidase sequence disclosed herein. As used herein, the term “conservative substitution” refers to a substitution with a structurally similar amino acid. For example, conservative substitutions may include those within the following groups: Ser and Cys; Leu, Ile, and Val; Glu and Asp; Lys and Arg; Phe, Tyr, and Trp; and Gln, Asn, Glu, Asp, and His. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix).

b. Antibody Portion

In certain embodiments, the fusion protein comprises an immunoglobulin Fc domain. As used herein, unless otherwise indicated, the term “immunoglobulin Fc domain” refers to a fragment of an immunoglobulin heavy chain constant region which, either alone or in combination with a second immunoglobulin Fc domain, is capable of binding to an Fc receptor. An immunoglobulin Fc domain may include, e.g., immunoglobulin CH2 and CH3 domains. An immunoglobulin Fc domain may include, e.g., immunoglobulin CH2 and CH3 domains and an immunoglobulin hinge region. Boundaries between immunoglobulin hinge regions, CH2, and CH3 domains are well known in the art, and can be found, e.g., in the PROSITE database (available on the world wide web at prosite.expasy.org).

In certain embodiments, the immunoglobulin Fc domain is derived from a human IgG1, IgG2, IgG3, IgG4, IgA1, IgA2, IgD, IgE, and IgM Fc domain. A single amino acid substitution (S228P according to Kabat numbering; designated IgG4Pro) may be introduced to abolish the heterogeneity observed in recombinant IgG4 antibody. See Angal, S. et al. (1993) MOL. IMMUNOL. 30:105-108.

In certain embodiments, the immunoglobulin Fc domain is derived from a human IgG1 isotype or another isotype that elicits antibody-dependent cell-mediated cytotoxicity (ADCC) and/or complement mediated cytotoxicity (CDC). In certain embodiments, the immunoglobulin Fc domain is derived from a human IgG1 isotype (e.g., SEQ ID NO: 31 or SEQ ID NO: 5).

In certain embodiments, the immunoglobulin Fc domain is derived from a human IgG4 isotype or another isotype that elicits little or no antibody-dependent cell-mediated cytotoxicity (ADCC) and/or complement mediated cytotoxicity (CDC). In certain embodiments, the immunoglobulin Fc domain is derived from a human IgG4 isotype.

In certain embodiments, the immunoglobulin Fc domain comprises either a “knob” mutation, e.g., T366Y, or a “hole” mutation, e.g., Y407T, for heterodimerization with a second polypeptide (residue numbers according to EU numbering, Kabat, E. A., et al. (1991) SEQUENCES OF PROTIENS OF IMMUNOLOGICAL IN INTEREST, FIFTH EDITION, U.S. Department of Health and Human Services, NIH Publication No. 91-3242). For example, in certain embodiments, the immunoglobulin Fc domain is derived from a human IgG1 Fc domain and comprises a Y407T mutation (e.g., the immunoglobulin Fc domain comprises SEQ ID NO: 32 or SEQ ID NO: 92). In certain embodiments, the immunoglobulin Fc domain is derived from a human IgG1 Fc domain and comprises a T366Y mutation (e.g., the second polypeptide comprises SEQ ID NO: 33 or SEQ ID NO: 93).

In certain embodiments, the immunoglobulin Fc domain is modified to prevent to glycosylation of the Fc domain. For example, in certain embodiments, the immunoglobulin Fc domain is derived from a human IgG1 Fc domain and comprises a mutation at position N297, for example, an N297A or N297G mutation (residue numbers according to EU numbering, Kabat, E. A., et al., supra). For example, in certain embodiments, the fusion protein comprises SEQ ID NO: 222, SEQ ID NO: 225, or SEQ ID NO: 226.

In certain embodiments, the fusion protein comprises an immunoglobulin antigen-binding domain. The inclusion of such a domain may improve targeting of a fusion protein to a sialylated cancer cell, e.g., a PD-L1 expressing cancer cell, and/or to the tumor microenvironment. As used herein, unless otherwise indicated, the term “immunoglobulin antigen-binding domain” refers to a polypeptide that, alone or in combination with another immunoglobulin antigen-binding domain, defines an antigen-binding site. Exemplary immunoglobulin antigen-binding domains include, for example, immunoglobulin heavy chain variable region and an immunoglobulin light chain variable region, where the variable regions together define an antigen binding site, e.g., an anti-PD-L1 antigen binding site.

In certain embodiments, the immunoglobulin antigen-binding domain is derived from an anti-PD-L1 antibody. Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, atezolizumab (Tecentriq®, Genentech), durvalumab (AstraZeneca), MEDI4736, avelumab, CS1001 (CStone Therapeutics), KL-A167, CK-301 (Checkpoint Therapeutics), TQB2450, KN035, SHR-1316, STI-A1014, BGB-A333, MSB2311, HLX-20 and BMS-936559 by Bristol-Myers Squibb.

In certain embodiments, the immunoglobulin antigen-binding domain is derived from avelumab. The avelumab heavy chain amino acid sequence is depicted in SEQ ID NO: 63, and the avelumab light chain amino acid sequence is depicted in SEQ ID NO: 64. The amino acid sequence of an exemplary scFv derived from avelumab is depicted in SEQ ID NO: 125.

In certain embodiments, the immunoglobulin antigen-binding domain is derived from an anti-PD-L1 antibody disclosed herein, for example, an antibody comprising: (i) an immunoglobulin heavy chain variable region comprising the amino acid sequence of SEQ ID NO: 164 (PAL769-VH), and an immunoglobulin light chain variable region comprising the amino acid sequence of SEQ ID NO: 167 (PAL769-VL); (ii) an immunoglobulin heavy chain variable region comprising the amino acid sequence of SEQ ID NO: 199 (h769 VH), and an immunoglobulin light chain variable region comprising the amino acid sequence of SEQ ID NO: 200 (h769-IF3-VL); (iii) an immunoglobulin heavy chain variable region comprising the amino acid sequence of SEQ ID NO: 199 (h769-VH), and an immunoglobulin light chain variable region comprising the amino acid sequence of SEQ ID NO: 201 (h769-tm2-VL); (iv) an immunoglobulin heavy chain variable region comprising the amino acid sequence of SEQ ID NO: 199 (h769-VH), and an immunoglobulin light chain variable region comprising the amino acid sequence of SEQ ID NO: 202 (h769-tm3-VL); (v) an immunoglobulin heavy chain variable region comprising the amino acid sequence of SEQ ID NO: 199 (h769-VH), and an immunoglobulin light chain variable region comprising the amino acid sequence of SEQ ID NO: 204 (h769.T-VL); (vi) an immunoglobulin heavy chain variable region comprising the amino acid sequence of SEQ ID NO: 132 (PAL752-VH), and an immunoglobulin light chain variable region comprising the amino acid sequence of SEQ ID NO: 136 (PAL752-VL); (vii) an immunoglobulin heavy chain variable region comprising the amino acid sequence of SEQ ID NO: 140 (PAL759-VH), and an immunoglobulin light chain variable region comprising the amino acid sequence of SEQ ID NO: 144 (PAL759-VL); (viii) an immunoglobulin heavy chain variable region comprising the amino acid sequence of SEQ ID NO: 148 (PAL760-VH), and an immunoglobulin light chain variable region comprising the amino acid sequence of SEQ ID NO: 152 (PAL760-VL); (ix) an immunoglobulin heavy chain variable region comprising the amino acid sequence of SEQ ID NO: 156 (PAL767-VH), and an immunoglobulin light chain variable region comprising the amino acid sequence of SEQ ID NO: 160 (PAL767-VL); (x) an immunoglobulin heavy chain variable region comprising the amino acid sequence of SEQ ID NO: 170 (PAL771-VH), and an immunoglobulin light chain variable region comprising the amino acid sequence of SEQ ID NO: 174 (PAL771-VL); (xi) an immunoglobulin heavy chain variable region comprising the amino acid sequence of SEQ ID NO: 178 (PAL785-VH), and an immunoglobulin light chain variable region comprising the amino acid sequence of SEQ ID NO: 182 (PAL785-VL); (xii) an immunoglobulin heavy chain variable region comprising the amino acid sequence of SEQ ID NO: 186 (PAL787-VH), and an immunoglobulin light chain variable region comprising the amino acid sequence of SEQ ID NO: 190 (PAL787-VL); or (xiii) an immunoglobulin heavy chain variable region comprising the amino acid sequence of SEQ ID NO: 194 (PAL788-VH), and an immunoglobulin light chain variable region comprising the amino acid sequence of SEQ ID NO: 198 (PAL788-VL).

c. Linker

In certain embodiments, the sialidase portion of the fusion protein can be linked or fused directly to the anti-PD-L1 antibody portion (e.g., immunoglobulin Fc domain and/or immunoglobulin antigen-binding domain) of the fusion protein. In other embodiments, the sialidase portion can be covalently bound to the anti-PD-L1 antibody portion by a linker.

The linker may couple, with one or more natural amino acids, the sialidase, or functional fragment thereof, and the antibody portions or fragments, where the amino acid (for example, a cysteine amino acid) may be introduced by site-directed mutagenesis. The linker may include one or more unnatural amino acids. It is contemplated that, in certain circumstances, a linker containing for example, one or more sulfhydryl reactive groups (e.g., a maleimide) may covalently link a cysteine in the sialidase portion or the antibody portion that is a naturally occurring cysteine residue or is the product of site-specific mutagenesis.

The linker may be a cleavable linker or a non-cleavable linker. Optionally or in addition, the linker may be a flexible linker or an inflexible linker.

The linker should be a length sufficiently long to allow the sialidase and the antibody portions to be linked without steric hindrance from one another and sufficiently short to retain the intended activity of the fusion protein. The linker preferably is sufficiently hydrophilic to avoid or minimize instability of the fusion protein. The linker preferably is sufficiently hydrophilic to avoid or minimize insolubility of the fusion protein. The linker should be sufficiently stable in vivo (e.g., it is not cleaved by serum, enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer A in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker that connects or fuses the sialidase portion of the fusion protein to the anti-PD-L1 antibody portion (e.g., immunoglobulin Fc domain and/or immunoglobulin antigen-binding domain) of the fusion protein. For example, it is contemplated that a gene encoding a sialidase portion linked directly or indirectly (for example, via an amino acid containing linker) to an antibody portion can be created and expressed using conventional recombinant DNA technologies. For example, the amino terminus of a sialidase portion can be linked to the carboxy terminus of either the light or the heavy chain of an antibody portion. For example, for a Fab fragment, the amino terminus or carboxy terminus of the sialidase can be linked to the first constant domain of the heavy antibody chain (CH1). When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, or 5-10 amino acid residues. Exemplary linkers include glycine and serine-rich linkers, e.g., (GlyGlyPro)ₙ, or (GlyGlyGlyGlySer)ₙ, where n is 1-5. In certain embodiments, the linker comprises, consists, or consists essentially of GGGGS (SEQ ID NO: 121). In certain embodiments, the linker comprises, consists, or consists essentially of GGGGSGGGGS (SEQ ID NO: 90). In certain embodiments, the linker comprises, consists, or consists essentially of EPKSS (SEQ ID NO: 91). Additional exemplary linker sequences are disclosed, e.g., in George et al. (2003) PROTEIN ENGINEERING 15:871-879, and U.S. Pat. Nos. 5,482,858 and 5,525,491.

In certain embodiments, the fusion protein comprises the amino acid sequence of any one of SEQ ID NOs: 65-75, 78, 81-89, 95, 96, 98, 99, 101, 102, 104, 106, 108, 110, 112, 114, 122-124, 127, 128, 205-207, 211, 213, 214, or 219, or an amino acid sequence that has at least 85%, 90%, 95%, 96%, 97%, 98%, or 99% sequence identity to any one of SEQ ID NOs: 65-75, 78, 81-89, 95, 96, 98, 99, 101, 102, 104, 106, 108, 110, 112, 114, 122-124, 127, 128, 205-207, 211, 213, 214, or 219.

d. Antibody Conjugates

The invention further provides antibody conjugates containing one or more of the fusion proteins disclosed herein. As used herein, unless otherwise indicated, the term “antibody conjugate” is understood to refer to an antibody, or a functional fragment thereof, that comprises antigen-binding activity (e.g., anti-PD-L1 antigen-binding activity) and/or Fc receptor-binding activity, conjugated (e.g., covalently coupled) to an additional functional moiety. In certain embodiments, the antibody or functional antibody fragment is conjugated to a sialidase enzyme, e.g., a recombinant mutant human sialidase enzyme disclosed herein. In certain embodiments, an antibody conjugate comprises a single polypeptide chain. In certain embodiments, an antibody conjugate comprises two, three, four, or more polypeptide chains that are covalently or non-covalently associated together to produce a multimeric complex, e.g., a dimeric, trimeric or tetrameric complex. For example, an antibody conjugate may comprise a first polypeptide (fusion protein) comprising a recombinant mutant human sialidase enzyme and an immunoglobulin heavy chain, and a second polypeptide comprising an immunoglobulin light chain, where, for example, the immunoglobulin heavy and light chains together define a single antigen-binding site, e.g., an anti-PD-L1 antigen-binding site.

In certain embodiments, the antibody conjugate can include a single sialidase. In other embodiments, the antibody conjugate can include more than one (e.g., two) sialidases. If more than one sialidase is included, the sialidases can be the same or different. In certain embodiments, the antibody conjugate can include a single anti-PD-L1 antigen-binding site. In other embodiments, the antibody conjugate can include more than one (e.g., two) anti-PD-L1 antigen-binding sites. If two antigen-binding sites are used, they can be the same or different. In certain embodiments, the antibody conjugate comprises an immunoglobulin Fc fragment.

In certain embodiments, the antibody conjugate comprises one or two immunoglobulin heavy chains, or a functional fragment thereof. In certain embodiments, the antibody conjugate comprises one or two immunoglobulin light chains, or a functional fragment thereof. In certain embodiments, the antibody conjugate comprises a sialidase fused to the N- or C-terminus of an immunoglobulin heavy chain or an immunoglobulin light chain.

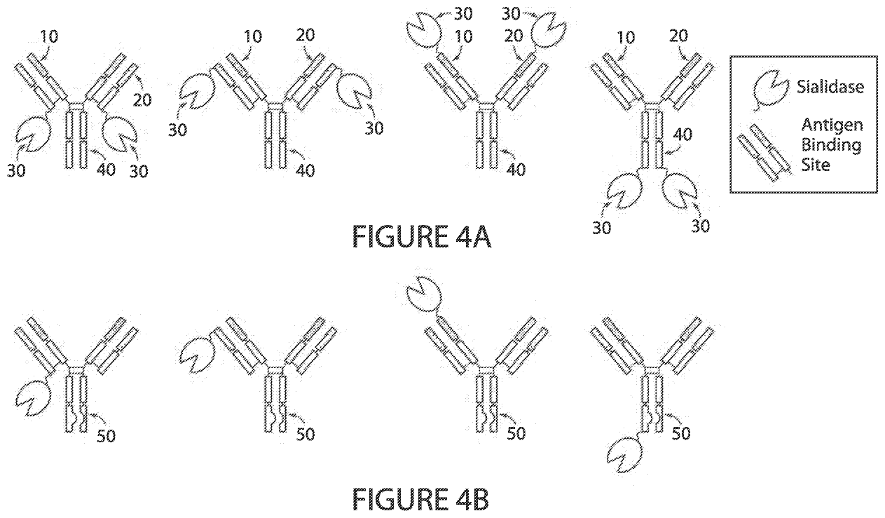

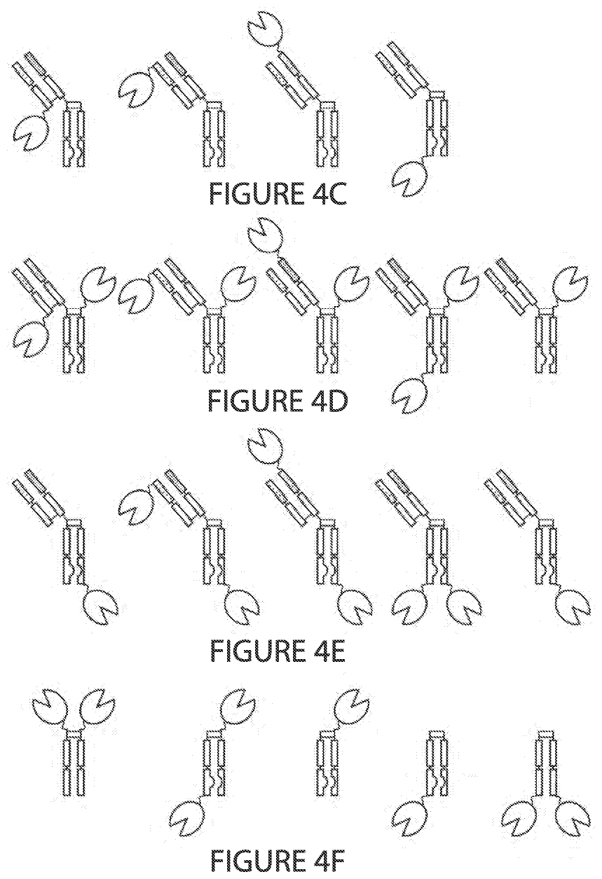

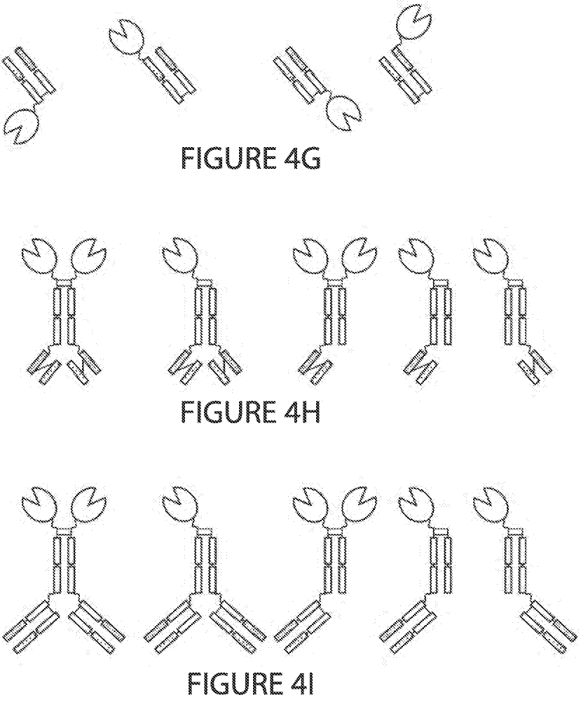

FIG. 4 depicts exemplary antibody conjugate constructs containing one or more sialidase enzymes. For example, in FIG. 4A, a first anti-PD-L1 antigen-binding site (e.g., defined by a VH and VL domains) is depicted as 10, a second anti-PD-L1 antigen-binding site is depicted as 20, a sialidase is depicted as 30, and a Fc is depicted as 40. In each of the constructs depicted in FIGS. 4A-4I it is understood that the Fc may optionally be modified in some manner, e.g., using Knobs-into-Holes type technology, e.g., as depicted by 50 in FIG. 4B. Throughout FIG. 4 similar structures are depicted by similar schematic representations.

FIG. 4A depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-PD-L1 antigen-binding site as depicted as 10, and the third polypeptide and the fourth polypeptide together define a second anti-PD-L1 antigen-binding site as depicted as 20. A sialidase enzyme as depicted as 30 can be conjugated to the N- or C-terminus of the first and second immunoglobulin light chain or the first and second immunoglobulin heavy chain.

FIG. 4B depicts antibody conjugate constructs comprising a first polypeptide comprising a first immunoglobulin light chain; a second polypeptide comprising a first immunoglobulin heavy chain; a third polypeptide comprising a second immunoglobulin heavy chain; and a fourth polypeptide comprising a second immunoglobulin light chain. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-PD-L1 antigen-binding site, and the third polypeptide and the fourth polypeptide together define a second anti-PD-L1 antigen-binding site. A sialidase enzyme can be conjugated to the N- or C-terminus of the first immunoglobulin light chain or the first immunoglobulin heavy chain.

FIG. 4C depicts antibody conjugate constructs comprising a first polypeptide comprising an immunoglobulin light chain; a second polypeptide comprising an immunoglobulin heavy chain; and a third polypeptide comprising an immunoglobulin Fc domain. The first and second polypeptides can be covalently linked together and the second and third polypeptides can be covalently linked together. The covalent linkages can be disulfide bonds. In certain embodiments, the first polypeptide and the second polypeptide together define an anti-PD-L1 antigen-binding site. A sialidase enzyme can be conjugated to the N- or C-terminus of the first immunoglobulin light chain or the first immunoglobulin heavy chain.

FIG. 4D depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define an anti-PD-L1 antigen-binding site. An optional second sialidase enzyme can be conjugated to the N- or C-terminus of the first immunoglobulin light chain or the first immunoglobulin heavy chain.

FIG. 4E depicts antibody conjugate constructs comprising a first polypeptide comprising an immunoglobulin light chain; a second polypeptide comprising an immunoglobulin heavy chain; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The third polypeptide comprises the immunoglobulin Fc domain and the sialidase in an N- to C-terminal orientation. In certain embodiments, the first polypeptide and the second polypeptide together define an anti-PD-L1 antigen-binding site. An optional second sialidase enzyme can be conjugated to the N- or C-terminus of the first immunoglobulin light chain or the first immunoglobulin heavy chain.

FIG. 4F depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N- or C-terminus of the first immunoglobulin Fc domain or to the N- or C-terminus of the second immunoglobulin Fc domain. An optional second sialidase enzyme can be conjugated to the N- or C-terminus of the first immunoglobulin Fc domain or to the N- or C-terminus of the second immunoglobulin Fc domain.

FIG. 4G depicts antibody conjugate constructs comprising a first polypeptide comprising an immunoglobulin light chain; and a second polypeptide comprising an immunoglobulin heavy chain variable region. The first and second polypeptides can be covalently linked together. The covalent linkages can be disulfide bonds. In certain embodiments, the first polypeptide and the second polypeptide together define an anti-PD-L1 antigen-binding site. The sialidase enzyme can be conjugated to the N- or C-terminus of the immunoglobulin light chain or the immunoglobulin heavy chain variable region.

FIG. 4H depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. A single chain variable fragment (scFv) can be conjugated to the C-terminus of the first immunoglobulin Fc domain or the second immunoglobulin Fc domain. An optional second single chain variable fragment (scFv) can be conjugated to the C-terminus of the first immunoglobulin Fc domain or the second immunoglobulin Fc domain, respectively.

FIG. 4I depicts antibody conjugate constructs similar to those depicted in FIG. 4H except that each scFv is replaced with an immunoglobulin antigen binding fragment, e.g., a Fab. For example, FIG. 4I depicts antibody conjugate constructs comprising a first polypeptide comprising a first immunoglobulin Fc domain, and a second polypeptide comprising a second immunoglobulin Fc domain. The first and second polypeptides can be covalently linked together. The covalent linkages can be disulfide bonds. A sialidase enzyme can be conjugated to the N-terminus of the first immunoglobulin Fc domain or the second immunoglobulin Fc domain. An optional second sialidase enzyme can be conjugated to the N-terminus of the second immunoglobulin Fc domain or the first immunoglobulin Fc domain, respectively. An antibody fragment (Fab) can be conjugated or fused to the C-terminus of the first immunoglobulin Fc domain or the second immunoglobulin Fc domain. An optional second antibody fragment (Fab) can be conjugated or fused to the C-terminus of the second immunoglobulin Fc domain or the first immunoglobulin Fc domain, respectively. In the case of a fusion, the C terminus of the Fc domain is linked (either by a bond or an amino acid linker) to a first polypeptide chain defining an anti-PD-L1 immunoglobulin antigen binding fragment. In the case of antibodies that have an antigen binding site defined by a single variable region, then this may be sufficient to impart binding affinity to a target antigen, e.g., PD-L1. In other instances, e.g., in the case of a human antibody, the first polypeptide chain defining an immunoglobulin antigen binding fragment can be conjugated (e.g., covalently conjugated, e.g., via a disulfide bond) to a second polypeptide chain defining an immunoglobulin antigen binding fragment, there the two antigen binding fragments together define an antigen binding site for binding the target antigen, e.g., PD-L1.

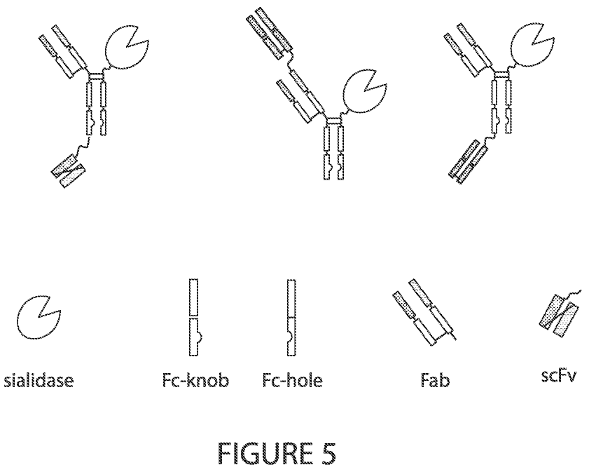

FIG. 5 depicts additional antibody conjugate constructs. For example, FIG. 5 depicts an antibody conjugate construct comprising a first polypeptide comprising an immunoglobulin light chain; a second polypeptide comprising an immunoglobulin heavy chain and an scFv; and a third polypeptide comprising an immunoglobulin Fc domain and a first sialidase enzyme. The first and second polypeptides can be covalently linked together and the second and third polypeptides can be covalently linked together. The covalent linkages can be disulfide bonds. The second polypeptide comprises the heavy chain and the scFv in an N- to C-terminal orientation. The third polypeptide comprises the sialidase and the immunoglobulin Fc domain in an N- to C-terminal orientation. In certain embodiments, the first polypeptide and the second polypeptide together define a first antigen-binding site. In certain embodiments, the scFv defines a second antigen-binding site. FIG. 5 depicts an additional antibody construct comprising a first polypeptide comprising an immunoglobulin light chain; a second polypeptide comprising an immunoglobulin heavy chain; and a third polypeptide comprising an immunoglobulin Fc domain and a first sialidase enzyme, wherein a Fab fragment is conjugated to the N-terminus of the immunoglobulin heavy chain. The first and second polypeptides can be covalently linked together and the second and third polypeptides can be covalently linked together. The covalent linkages can be disulfide bonds. The third polypeptide comprises the sialidase and the immunoglobulin Fc domain in an N- to C-terminal orientation. In certain embodiments, the first polypeptide and the second polypeptide together define a first antigen-binding site. In certain embodiments, the Fab fragment defines a second antigen-binding site. In each of the constructs depicted in FIG. 5 it is understood that an scFv, when present, may be replaced with a Fab fragment, or a Fab fragment, when present, may be replaced with an scFv. In each of the constructs depicted in FIG. 5, it is understood that the Fc may optionally be modified in some manner.

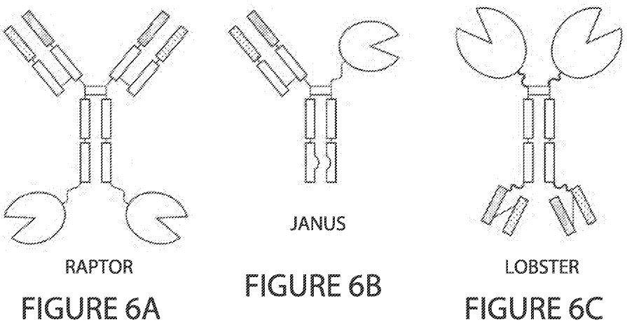

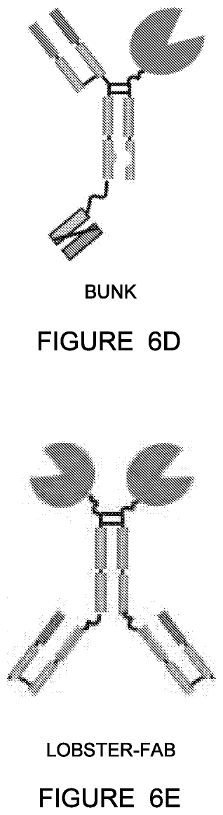

In certain embodiments, the antibody conjugate comprises a first polypeptide comprising a first immunoglobulin light chain; a second polypeptide comprising a first immunoglobulin heavy chain and a first sialidase; a third polypeptide comprising a second immunoglobulin heavy chain and a second sialidase; and a fourth polypeptide comprising a second immunoglobulin light chain. An example of this embodiment is shown in FIG. 6A. The first and second polypeptides can be covalently linked together, the third and fourth polypeptides can be covalently linked together, and the second and third polypeptides can be covalently linked together. The covalent linkages can be disulfide bonds. In certain embodiments, the first polypeptide and the second polypeptide together define a first anti-PD-L1 antigen-binding site, and the third polypeptide and the fourth polypeptide together define a second anti-PD-L1 antigen-binding site. In certain embodiments, the second and third polypeptides comprise the first and second immunoglobulin heavy chain and the first and second sialidase, respectively, in an N- to C-terminal orientation. In certain embodiments, the second and third polypeptides comprise the first and second sialidase and the first and second immunoglobulin heavy chain, respectively, in an N- to C-terminal orientation.

In certain embodiments, the antibody conjugate comprises a first polypeptide comprising an immunoglobulin light chain; a second polypeptide comprising an immunoglobulin heavy chain; and a third polypeptide comprising an immunoglobulin Fc domain and a sialidase. An example of this embodiment is shown in FIG. 6B. The first and second polypeptides can be covalently linked together and the second and third polypeptides can be covalently linked together. The covalent linkages can be disulfide bonds. In certain embodiments, the first polypeptide and the second polypeptide together define an anti-PD-L1 antigen-binding site. In certain embodiments, the third polypeptide comprises the sialidase and the immunoglobulin Fc domain in an N- to C-terminal orientation, or the immunoglobulin Fc domain and the sialidase in an N- to C-terminal orientation.

In certain embodiments, the first polypeptide comprises the amino acid sequence of any one of SEQ ID NOs: 65 or 205, or an amino acid sequence that has at least 85%, 90%, 95%, 96%, 97%, 98%, or 99% sequence identity to any one of SEQ ID NOs: 65 or 205. In certain embodiments, the second polypeptide comprises the amino acid sequence of any one of SEQ ID NOs: 66, 104, 124, 206, or 213, or an amino acid sequence that has at least 85%, 90%, 95%, 96%, 97%, 98%, or 99% sequence identity to any one of SEQ ID NOs: 66, 104, 124, 206, or 213. In certain embodiments, the third polypeptide comprises the amino acid sequence of any one of SEQ ID NOs: 67-73, 78, 81-87, 95, 96, 98, 99, 101, 102, 106, 108, 112, 122, 123, 127, 128, 207, 211, 214, or 219, or an amino acid sequence that has at least 85%, 90%, 95%, 96%, 97%, 98%, or 99% sequence identity to any one of SEQ ID NOs: 67-73, 78, 81-87, 95, 96, 98, 99, 101, 102, 106, 108, 112, 122, 123, 127, 128, 207, 211, 214, or 219.

In certain embodiments, the third polypeptide comprises the amino acid sequence of

(SEQ ID NO: 240)
X₁X₂SX₃X₄X₅LQX₆SVFQSGAHAYRIPALLYLPGQQSLLAFAEQRX₇S
X₈X₉DEHAELIVX₁₀RRGDYDAX₁₁THQVQWX₁₂AQEVVAQAX₁₃LX₁₄G
HRSMNPCPLYDX₁₅QTGTLFLFFIAIPX₁₆X₁₇VTEX₁₈QQLQTRANVTR
LX₁₉X₂₀VTSTDHGRTWSSPRDLTDAAIGPX₂₁YREWSTFAVGPGHX₂₂L
QLHDX₂₃X₂₄RSLVVPAYAYRKLHPX₂₅X₂₆X₂₇PIPSAFX₂₈FLSHDHG
RTWARGHFVX₂₉QDTX₃₀ECQVAEVX₃₁TGEQRVVTLNARSX₃₂X₃₃X₃₄
X₃₅RX₃₆QAQSX₃₇NX₃₈GLDFQX₃₉X₄₀QX₄₁VKKLX₄₂EPPPX₄₃G
X₄₄QGSVISFPSPRSGPGSPAQX₄₅LLYTHPTHX₄₆X₄₇QRADLGAYLNP
RPPAPEAWSEPX₄₈LLAKGSX₄₉AYSDLQSMGTGPDGSPLFGX₅₀LYEAN
DYEEIX₅₁FX₅₂MFTLKQAFPAEYLPQX₅₃DKTHTCPPCPAPELLGGPSV
FLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENN
YKTTPPVLDSDGSFFLTSKLTVDKSRWQQGNVFSCSVMHEALHNHYTQKS
LSLSPGK,

wherein X₁ is Ala, Arg, Asn, Asp, Gln, Glu, Gly, His, Leu, Lys, Met, Phe, Thr, Val, or not present, X₂ is Ala or Lys, X₃ is Asn or Leu, X₄ is Pro or His, X₅ is Phe, Trp, Tyr or Val, X₆ is Lys or Asp, X₇ is Ala or Arg, X₈ is Lys, Arg, or Glu, X₉ is Lys, Ala, Arg, or Glu, X₁₀ is Leu or Met, X₁₁ is Pro, Asn, Asp, His, Glu, Gly, Ser or Thr, X₁₂ is Gln or His, X₁₃ is Arg or Lys, X₁₄ is Asp or Pro, X₁₅ is Ala, Glu or Lys, X₁₆ is Gly or Asp, X₁₇ is Gln or His, X₁₈ is Gln, Arg, or Lys, X₁₉ is Ala, Cys, Ile, Ser, Val, or Leu, X₂₀ is Gln, Leu, Glu, Phe, His, Ile, Leu, or Tyr, X₂₁ is Ala or Val, X₂₂ is Cys or Gly, X₂₃ is Arg or Pro, X₂₄ is Ala or Gly, X₂₅ is Arg, Ile, or Lys, X₂₆ is Gln or Pro, X₂₇ is Arg or Pro, X₂₈ is Ala, Cys, Leu, or Val, X₂₉ is Ala, Cys, Asn, Ser, or Thr, X₃₀ is Leu, Ala, or Val, X₃₁ is Glu or Pro, X₃₂ is His or Pro, X₃₃ is Leu, Asp, Asn, or Tyr, X₃₄ is Arg, Ala, Asp, Leu, Gln, or Tyr, X₃₅ is Ala, Cys, Phe, Gly, His, Ile, Lys, Leu, Met, Asn, Gln, Arg, Ser, Val, Trp, or Tyr, X₃₆ is Val, Ile, or Lys, X₃₇ is Thr or Ala, X₃₈ is Asp or Gly, X₃₉ is Glu, Lys, or Pro, X₄₀ is Ser or Cys, X₄₁ is Leu, Asp, Phe, Gln, or Thr, X₄₂ is Val or Phe, X₄₃ is Gln, Ala, His, Phe, Pro, Ser, or Thr, X₄₄ is Cys or Val, X₄₅ is Trp or Arg, X₄₆ is Ser, Arg, Ala, Asp, Glu, Phe, Gly, His, Ile, Lys, Leu, Met, Asn, Pro, Gln, Thr, Val, Trp, or Tyr, X₄₇ is Trp, Lys, Ala, Asp, Glu, Phe, Gly, His, Ile, Lys, Leu, Met, Asn, Pro, Gln, Arg, Ser, Thr, Val, or Tyr, X₄₈ is Lys or Val, X₄₉ is Ala, Cys, Ser, or Val, X₅₀ is Cys, Leu, or Val, X₅₁ is Val or Arg, X₅₂ is Leu, Gln, His, Ile, Lys, or Ser, and X₅₃ is GGGGS (SEQ ID NO: 121), GGGGSGGGGS (SEQ ID NO: 90), or EPKSS (SEQ ID NO: 91), and the sialidase comprises at least one mutation relative to wild-type human Neu2 (SEQ ID NO: 1).

In certain embodiments, the third polypeptide comprises the amino acid sequence of

(SEQ ID NO: 241)
X₁ASLPX₂LQX₃ESVFQSGAHAYRIPALLYLPGQQSLLAFAEQRX₄SKKD
EHAELIVLRRGDYDAX₅THQVQWQAQEVVAQARLDGHRSMNPCPLYDX₆Q
TGTLFLFFIAIPGQVTEQQQLQTRANVTRLCX₇VTSTDHGRTWSSPRDLT
DAAIGPAYREWSTFAVGPGHCLQLHDRARSLVVPAYAYRKLHPX₈QRPIP
SAFCFLSHDHGRTWARGHFVAQDTLECQVAEVETGEQRVVTLNARSHLR
X₉RVQAQSTNDGLDFQESQLVKKLVEPPPX₁₀GCQGSVISFPSPRSGPGS
PAQWLLYTHPTHX₁₁X₁₂QRADLGAYLNPRPPAPEAWSEPVLLAKGSX₁₃
AYSDLQSMGTGPDGSPLFGCLYEANDYEEIX₁₄FX₁₅MFTLKQAFPAEYL
PQX₁₆DKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSREEMTKNQVSL
TCLVKGFYPSDIAVEWESNGQPENNYKTTPPVLDSDGSFFLTSKLTVDKS
RWQQGNVFSCSVMHEALHNHYTQKSLSLSPGK,

wherein X₁ is Ala, Arg, Asn, Asp, Gln, Glu, Gly, His, Leu, Lys, Met, Phe, Thr, Val, or not present, X₂ is Phe, Trp, Tyr or Val, X₃ is Lys or Asp, X₄ is Arg or Ala, X₅ is Pro, Asn, Asp, His, Glu, Gly, Ser or Thr, X₆ is Ala, Glu, or Lys, X₇ is Gln, Leu, Glu, Phe, His, Ile, Leu, or Tyr, X₈ is Arg, Ile, or Lys, X₉ is Ala, Cys, Phe, Gly, His, Ile, Lys, Leu, Met, Asn, Gln, Arg, Ser, Val, Trp, or Tyr, X₁₀ is Gln, Ala, His, Phe, Pro, Ser, or Thr, X₁₁ is Ser, Arg, Ala, Asp, Glu, Phe, Gly, His, Ile, Lys, Leu, Met, Asn, Pro, Gln, Thr, Val, Trp, or Tyr, X₁₂ is Trp, Lys, Ala, Asp, Glu, Phe, Gly, His, Ile, Lys, Leu, Met, Asn, Pro, Gln, Arg, Ser, Thr, Val, or Tyr, X₁₃ is Ala, Cys, Ser, or Val, X₁₄ is Val or Arg, X₁₅ is Leu, Gln, His, Ile, Lys, or Ser, and X₁₆ is GGGGS (SEQ ID NO: 121), GGGGSGGGGS (SEQ ID NO: 90), or EPKSS (SEQ ID NO: 91), and the sialidase comprises at least one mutation relative to wild-type human Neu2 (SEQ ID NO: 1). In certain embodiments, X₁ is Ala, Asp, Met, or not present, X₂ is Tyr or Val, X₃ is Lys or Asp, X₄ is Arg or Ala, X₅ is Pro, Asn, Gly, Ser or Thr, X₆ is Ala or Glu, X₇ is Gln or Tyr, X₈ is Ile or Lys, X₉ is Ala or Thr, X₁₀ is Gln, Ala, or Thr, X₁₁ is Ser, Arg, or Ala, X₁₂ is Trp, Lys, or Arg, X₁₃ is Ala or Cys, X₁₄ is Val or Arg, and X₁₅ is Leu or Ile.

In certain embodiments, the first polypeptide comprises SEQ ID NO: 65, the second polypeptide comprises SEQ ID NO: 66, and the third polypeptide comprises SEQ ID NO: 67. In certain embodiments, the first polypeptide comprises SEQ ID NO: 65, the second polypeptide comprises SEQ ID NO: 66, and the third polypeptide comprises SEQ ID NO: 68. In certain embodiments, the first polypeptide comprises SEQ ID NO: 65, the second polypeptide comprises SEQ ID NO: 66, and the third polypeptide comprises SEQ ID NO: 69. In certain embodiments, the first polypeptide comprises SEQ ID NO: 65, the second polypeptide comprises SEQ ID NO: 66, and the third polypeptide comprises SEQ ID NO: 70. In certain embodiments, the first polypeptide comprises SEQ ID NO: 65, the second polypeptide comprises SEQ ID NO: 66, and the third polypeptide comprises SEQ ID NO: 71. In certain embodiments, the first polypeptide comprises SEQ ID NO: 65, the second polypeptide comprises SEQ ID NO: 66, and the third polypeptide comprises SEQ ID NO: 72. In certain embodiments, the first polypeptide comprises SEQ ID NO: 65, the second polypeptide comprises SEQ ID NO: 66, and the third polypeptide comprises SEQ ID NO: 73. In certain embodiments, the first polypeptide comprises SEQ ID NO: 65, the second polypeptide comprises SEQ ID NO: 66, and the third polypeptide comprises SEQ ID NO: 78. In certain embodiments, the first polypeptide comprises SEQ ID NO: 65, the second polypeptide comprises SEQ ID NO: 66, and the third polypeptide comprises SEQ ID NO: 81. In certain embodiments, the first polypeptide comprises SEQ ID NO: 65, the second polypeptide comprises SEQ ID NO: 66, and the third polypeptide comprises SEQ ID NO: 82. In certain embodiments, the first polypeptide comprises SEQ ID NO: 65, the second polypeptide comprises SEQ ID NO: 66, and the third polypeptide comprises SEQ ID NO: 83. In certain embodiments, the first polypeptide comprises SEQ ID NO: 65, the second polypeptide comprises SEQ ID NO: 66, and the third polypeptide comprises SEQ ID NO: 84. In certain embodiments, the first polypeptide comprises SEQ ID NO: 65, the second polypeptide comprises SEQ ID NO: 66, and the third polypeptide comprises SEQ ID NO: 85. In certain embodiments, the first polypeptide comprises SEQ ID NO: 65, the second polypeptide comprises SEQ ID NO: 66, and the third polypeptide comprises SEQ ID NO: 86. In certain embodiments, the first polypeptide comprises SEQ ID NO: 65, the second polypeptide comprises SEQ ID NO: 66, and the third polypeptide comprises SEQ ID NO: 87. In certain embodiments, the first polypeptide comprises SEQ ID NO: 65, the second polypeptide comprises SEQ ID NO: 66, and the third polypeptide comprises SEQ ID NO: 95. In certain embodiments, the first polypeptide comprises SEQ ID NO: 65, the second polypeptide comprises SEQ ID NO: 66, and the third polypeptide comprises SEQ ID NO: 96. In certain embodiments, the first polypeptide comprises SEQ ID NO: 65, the second polypeptide comprises SEQ ID NO: 66, and the third polypeptide comprises SEQ ID NO: 98. In certain embodiments, the first polypeptide comprises SEQ ID NO: 65, the second polypeptide comprises SEQ ID NO: 66, and the third polypeptide comprises SEQ ID NO: 99. In certain embodiments, the first polypeptide comprises SEQ ID NO: 65, the second polypeptide comprises SEQ ID NO: 66, and the third polypeptide comprises SEQ ID NO: 101. In certain embodiments, the first polypeptide comprises SEQ ID NO: 65, the second polypeptide comprises SEQ ID NO: 66, and the third polypeptide comprises SEQ ID NO: 102. In certain embodiments, the first polypeptide comprises SEQ ID NO: 65, the second polypeptide comprises SEQ ID NO: 66, and the third polypeptide comprises SEQ ID NO: 106. In certain embodiments, the first polypeptide comprises SEQ ID NO: 65, the second polypeptide comprises SEQ ID NO: 66, and the third polypeptide comprises SEQ ID NO: 112. In certain embodiments, the first polypeptide comprises SEQ ID NO: 65, the second polypeptide comprises SEQ ID NO: 66, and the third polypeptide comprises SEQ ID NO: 127. In certain embodiments, the first polypeptide comprises SEQ ID NO: 65, the second polypeptide comprises SEQ ID NO: 66, and the third polypeptide comprises SEQ ID NO: 128. In certain embodiments, the first polypeptide comprises SEQ ID NO: 65, the second polypeptide comprises SEQ ID NO: 104, and the third polypeptide comprises SEQ ID NO: 108. In certain embodiments, the first polypeptide comprises SEQ ID NO: 65, the second polypeptide comprises SEQ ID NO: 124, and the third polypeptide comprises SEQ ID NO: 122. In certain embodiments, the first polypeptide comprises SEQ ID NO: 65, the second polypeptide comprises SEQ ID NO: 124, and the third polypeptide comprises SEQ ID NO: 123.

In certain embodiments, the first polypeptide comprises SEQ ID NO: 205, the second polypeptide comprises SEQ ID NO: 206, and the third polypeptide comprises SEQ ID NO: 207. In certain embodiments, the first polypeptide comprises SEQ ID NO: 205, the second polypeptide comprises SEQ ID NO: 206, and the third polypeptide comprises SEQ ID NO: 211. In certain embodiments, the first polypeptide comprises SEQ ID NO: 205, the second polypeptide comprises SEQ ID NO: 213, and the third polypeptide comprises SEQ ID NO: 214. In certain embodiments, the first polypeptide comprises SEQ ID NO: 205, the second polypeptide comprises SEQ ID NO: 213, and the third polypeptide comprises SEQ ID NO: 219.

In certain embodiments, the antibody conjugate comprises a first polypeptide comprising a first sialidase, a first immunoglobulin Fc domain, and a first single chain variable fragment (scFv) (it is also understood that the scFv may be replaced by a first polypeptide chain of an immunoglobulin antigen binding fragment, e.g., Fab fragment); and a second polypeptide comprising a second sialidase, a second immunoglobulin Fc domain, and a second single chain variable fragment (scFv) (it is also understood that the scFv may be replaced by a second polypeptide chain of an immunoglobulin antigen binding fragment, e.g., Fab fragment). An example of this embodiment is shown in FIG. 6C (in the construct depicted in FIG. 6C it is understood that an scFv, when present, may be replaced with a Fab fragment, or a Fab fragment, when present, may be replaced with an scFv). The first and second polypeptides can be covalently linked together. The covalent linkages can be disulfide bonds. In certain embodiments, the first scFv defines a first anti-PD-L1 antigen-binding site, and the second scFv defines a second anti-PD-L1 antigen-binding site. In certain embodiments, the first polypeptide comprises the first sialidase, the first immunoglobulin Fc domain, and the first scFv in an N- to C-terminal orientation. In certain embodiments, the first polypeptide comprises the first scFv, the first immunoglobulin Fc domain, and the first sialidase in an N- to C-terminal orientation. In certain embodiments, the second polypeptide comprises the second sialidase, the second immunoglobulin Fc domain, and the second scFv in an N- to C-terminal orientation. In certain embodiments, the second polypeptide comprises the second scFv, the second immunoglobulin Fc domain, and the second sialidase in an N- to C-terminal orientation.

In certain embodiments, the antibody conjugate comprises: a first polypeptide comprising an immunoglobulin light chain; a second polypeptide comprising an immunoglobulin heavy chain and a single chain variable fragment (scFv) (it is also understood that the scFv may be replaced by a first polypeptide chain of an immunoglobulin antigen binding fragment, e.g., Fab fragment); and a third polypeptide comprising an immunoglobulin Fc domain and a sialidase. It is also understood that the immunoglobulin light chain and the immunoglobulin heavy chain variable region may be swapped. An example of this embodiment is shown in FIG. 6D. The first and second polypeptides can be covalently linked together and the second and third polypeptides can be covalently linked together. The covalent linkages can be disulfide bonds. In certain embodiments, the first polypeptide and the second polypeptide together define a first anti-PD-L1 antigen-binding site (i.e., the immunoglobulin light chain and immunoglobulin heavy chain together define a first anti-PD-L1 antigen-binding site). In certain embodiments, the scFv defines a second anti-PD-L1 antigen-binding site. In certain embodiments, the second polypeptide comprises the immunoglobulin heavy chain and the scFv in an N- to C-terminal orientation, or the scFv and the immunoglobulin heavy chain in an N- to C-terminal orientation. In certain embodiments, the third polypeptide comprises the sialidase and the immunoglobulin Fc domain in an N- to C-terminal orientation, or the sialidase and the immunoglobulin Fc domain in an N- to C-terminal orientation.

In certain embodiments, the antibody conjugate comprises a first polypeptide comprising an immunoglobulin light chain; a second polypeptide comprising a first sialidase, a first immunoglobulin Fc domain, and a first immunoglobulin heavy chain variable region; a third polypeptide comprising a second sialidase, a second immunoglobulin Fc domain, and a second immunoglobulin heavy chain variable region; and a fourth polypeptide comprising a second immunoglobulin light chain. It is also understood that an immunoglobulin light chain may be replaced by an immunoglobulin heavy chain variable region and an immunoglobulin heavy chain variable region may be replaced by an immunoglobulin light chain (e.g., the antibody conjugate may comprise a first polypeptide comprising an immunoglobulin heavy chain variable region; a second polypeptide comprising a first sialidase, a first immunoglobulin Fc domain, and a first immunoglobulin light chain; a third polypeptide comprising a second sialidase, a second immunoglobulin Fc domain, and a second immunoglobulin light chain; and a fourth polypeptide comprising a second immunoglobulin heavy chain variable region). An example of this embodiment is shown in FIG. 6E. The second and third polypeptides can be covalently linked together. The covalent linkages can be disulfide bonds. In certain embodiments, the first and second polypeptides defines a first anti-PD-1 antigen-binding site, and the third and fourth polypeptides defines a second anti-PD-1 antigen-binding site. In certain embodiments, the second polypeptide comprises the first sialidase, the first immunoglobulin Fc domain, and the first immunoglobulin heavy chain variable region in an N- to C-terminal orientation. In certain embodiments, the third polypeptide comprises the second sialidase, the second immunoglobulin Fc domain, and the second immunoglobulin heavy chain variable region in an N- to C-terminal orientation.

In certain embodiments, the antibody conjugate has a molecular weight from about 135 kDa to about 165 kDa, e.g., about 140 kDa. In other embodiments, the antibody conjugate has a molecular weight from about 215 kDa to about 245 kDa, e.g., about 230 kDa.

In certain embodiments, the antibody conjugate comprises two polypeptides that each comprise an immunoglobulin Fc domain, and the first polypeptide has either a “knob” mutation, e.g., T366Y, or a “hole” mutation, e.g., Y407T, for heterodimerization with the second polypeptide, and the second polypeptide has either a respective “knob” mutation, e.g., T366Y, or a “hole” mutation, e.g., Y407T, for heterodimerization with the first polypeptide (residue numbers according to EU numbering, Kabat, E. A., et al. (1991) supra). For example, in certain embodiments, the antibody comprises two polypeptides that each comprise an immunoglobulin Fc domain derived from human IgG1 Fc domain, and the first polypeptide comprises a Y407T mutation (e.g., the first polypeptide comprises SEQ ID NO: 32 or SEQ ID NO: 92), and the second polypeptide comprises a T366Y mutation (e.g., the second polypeptide comprises SEQ ID NO: 33 or SEQ ID NO: 93).

In certain embodiments, the antibody conjugate comprises an immunoglobulin Fc domain that is modified to prevent to glycosylation of the Fc domain. For example, in certain embodiments, the immunoglobulin Fc domain is derived from a human IgG1 Fc domain and comprises a mutation at position N297, for example, an N297A or N297G mutation (residue numbers according to EU numbering, Kabat, E. A., et al., supra). For example, in certain embodiments, the antibody conjugate comprises SEQ ID NO: 222, SEQ ID NO: 225, or SEQ ID NO: 226.

As used herein, the term “multispecific antibody” is understood to mean an antibody that specifically binds to at least two different antigens, i.e., an antibody that comprises at least two antigen-binding sites that bind to at least two different antigens. As used herein, the term “bispecific antibody” is understood to mean an antibody that specifically binds to two different antigens, i.e., an antibody that comprises two antigen-binding sites each of which bind to separate and distinct antigens. In other words, a first binding site binds a first antigen and a second binding site binds a second, different antigen. A multispecific or bispecific antibody may, for example, be a human or humanized antibody, and/or be a full length antibody or an antibody fragment (e.g., a F(ab′)2 bispecific antibody).

The present invention encompasses antibody conjugates comprising antibody fragments, which may be generated by traditional means, such as enzymatic digestion, or by recombinant techniques. For a review of certain antibody fragments, see Hudson et al. (2003) supra.

In certain embodiments, the antibody conjugate or fusion protein can be covalently or non-covalently associated with a biological modifier, wherein the biological modifier can be used to enhance the solubility of the antibody, increase binding specificity, decrease immunogenicity or toxicity or modify the pharmacokinetic profile of the antibody. For example, the biological modifier can be used to increase the molecular weight of the antibody to increase its circulating half-life.

It is contemplated that the antibody conjugate or fusion protein may be covalently bound to one or more (for example, 2, 3, 4, 5, 6, 8, 9, 10 or more) biological modifiers that may comprise linear or branched polymers. Exemplary biological modifiers may include, for example, a variety of polymers, such as those described in U.S. Pat. No. 7,842,789. Particularly useful are polyalkylene ethers such as polyethylene glycol (PEG) and derivatives thereof (for example, alkoxy polyethylene glycol, for example, methoxypolyethylene glycol, ethoxypolyethylene glycol and the like); block copolymers of polyoxyethylene and polyoxypropylene (Pluronics); polymethacrylates; carbomers; and branched or unbranched polysaccharides which comprise the saccharide monomers such as D-mannose, D- and L-galactose, fucose, fructose, D-xylose, L-arabinose, and D-glucuronic acid.

In other embodiments, the biological modifier can be a hydrophilic polyvinyl polymer such as polyvinyl alcohol and polyvinylpyrrolidone (PVP)-type polymers. The biological modifier can be a functionalized polyvinylpyrrolidone, for example, carboxy or amine functionalized on one (or both) ends of the polymer (as available from PolymerSource). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). Alternatively, the biological modifier can include Poly N-(2-hydroxypropyl)methacrylamide (HPMA), or functionalized HPMA (amine, carboxy, etc.), Poly(N-isopropylacrylamide) or functionalized poly(N-isopropylacrylamide). The modifier prior to conjugation need not be, but preferably is, water soluble, but the final conjugate should be water soluble.

In general, the biological modifier may have a molecular weight from about 2 kDa to about 5 kDa, from about 2 kDa to about 10 kDa, from about 2 kDa to about 20 kDa, from about 2 kDa to about 30 kDa, from about 2 kDa to about 40 kDa, from about 2 kDa to about 50 kDa, from about 2 kDa to about 60 kDa, from about 2 kDa to about 70 kDa, from about 2 kDa to about 80 kDa, from about 2 kDa to about 90 kDa, from about 2 kDa to about 100 kDa, from about 2 kDa to about 150 kDa, from about 5 kDa to about 10 kDa, from about 5 kDa to about 20 kDa, from about 5 kDa to about 30 kDa, from about 5 kDa to about 40 kDa, from about 5 kDa to about 50 kDa, from about 5 kDa to about 60 kDa, from about 5 kDa to about 70 kDa, from about 5 kDa to about 80 kDa, from about 5 kDa to about 90 kDa, from about 5 kDa to about 100 kDa, from about 5 kDa to about 150 kDa, from about 10 kDa to about 20 kDa, from about 10 kDa to about 30 kDa, from about 10 kDa to about 40 kDa, from about 10 kDa to about 50 kDa, from about 10 kDa to about 60 kDa, from about 10 kDa to about 70 kDa, from about 10 kDa to about 80 kDa, from about 10 kDa to about 90 kDa, from about 10 kDa to about 100 kDa, from about 10 kDa to about 150 kDa, from about 20 kDa to about 30 kDa, from about 20 kDa to about 40 kDa, from about 20 kDa to about 50 kDa, from about 20 kDa to about 60 kDa, from about 20 kDa to about 70 kDa, from about 20 kDa to about 80 kDa, from about 20 kDa to about 90 kDa, from about 20 kDa to about 100 kDa, from about 20 kDa to about 150 kDa, from about 30 kDa to about 40 kDa, from about 30 kDa to about 50 kDa, from about 30 kDa to about 60 kDa, from about 30 kDa to about 70 kDa, from about 30 kDa to about 80 kDa, from about 30 kDa to about 90 kDa, from about 30 kDa to about 100 kDa, from about 30 kDa to about 150 kDa, from about 40 kDa to about 50 kDa, from about 40 kDa to about 60 kDa, from about 40 kDa to about 70 kDa, from about 40 kDa to about 80 kDa, from about 40 kDa to about 90 kDa, from about 40 kDa to about 100 kDa, from about 40 kDa to about 150 kDa, from about 50 kDa to about 60 kDa, from about 50 kDa to about 70 kDa, from about 50 kDa to about 80 kDa, from about 50 kDa to about 90 kDa, from about 50 kDa to about 100 kDa, from about 50 kDa to about 150 kDa, from about 60 kDa to about 70 kDa, from about 60 kDa to about 80 kDa, from about 60 kDa to about 90 kDa, from about 60 kDa to about 100 kDa, from about 60 kDa to about 150 kDa, from about 70 kDa to about 80 kDa, from about 70 kDa to about 90 kDa, from about 70 kDa to about 100 kDa, from about 70 kDa to about 150 kDa, from about 80 kDa to about 90 kDa, from about 80 kDa to about 100 kDa, from about 80 kDa to about 150 kDa, from about 90 kDa to about 100 kDa, from about 90 kDa to about 150 kDa, or from about 100 kDa to about 150 kDa.

It is contemplated that the antibody conjugate or fusion protein is attached to about 10 or fewer polymer molecules (e.g., 9, 8, 7, 6, 5, 4, 3, 2, or 1), each polymer molecule having a molecular weight of at least about 20,000 D, or at least about 30,000 D, or at least about 40,000 D.

Although a variety of polymers can be used as biological modifiers, it is contemplated that the antibody conjugates or fusion proteins described herein may be attached to polyethylene glycol (PEG) polymers. In one embodiment, the antibody conjugate or fusion protein described herein is covalently attached to at least one PEG having an actual MW of at least about 20,000 D. In another embodiment, the antibody conjugate or fusion protein described herein is covalently attached to at least one PEG having an actual MW of at least about 30,000 D. In another embodiment, the antibody conjugate or fusion protein described herein is covalently attached to at least one PEG having an actual MW of at least about 40,000 D. In certain embodiments, the PEG is methoxyPEG (5000)-succinimidylpropionate (mPEG-SPA), methoxyPEG (5000)-succinimidylsuccinate (mPEG-SS). Such PEGS are commercially available from Nektar Therapeutics or SunBiowest.

Attachment sites on an antibody conjugate or fusion protein for a biological modifier include the N-terminal amino group and epsilon amino groups found on lysine residues, as well as other amino, imino, carboxyl, sulfhydryl, hydroxyl or other hydrophilic groups. The polymer may be covalently bonded directly to the antibody conjugate or fusion protein with or without the known use of a multifunctional (ordinarily bifunctional) crosslinking agent using chemistries and used in the art. For example, sulfhydryl groups can be derivatized by coupling to maleimido-substituted PEG (e.g., alkoxy-PEG amine plus sulfosuccinimidyl 4-(N-maleimidomethyl)cyclohexane-1-carboxylate), or PEG-maleimide commercially available from Shearwater Polymers, Inc., Huntsville, Ala.).

### II. Methods of Making an Antibody, Fusion Protein, or Antibody Conjugate

Methods for producing antibodies, fusion proteins, or antibody conjugates, e.g., those disclosed herein, are known in the art. For example, DNA molecules encoding light chain variable regions and/or heavy chain variable regions can be synthesized chemically or by recombinant DNA methodologies. For example, the sequences of the antibodies can be cloned from hybridomas by conventional hybridization techniques or polymerase chain reaction (PCR) techniques, using the appropriate synthetic nucleic acid primers. The resulting DNA molecules encoding the variable regions of interest can be ligated to other appropriate nucleotide sequences, including, for example, constant region coding sequences, and expression control sequences, to produce conventional gene expression constructs (i.e., expression vectors) encoding the desired antibodies. Production of defined gene constructs is within routine skill in the art.

Nucleic acids encoding desired antibodies, fusion proteins, and/or antibody conjugates can be incorporated (ligated) into expression vectors, which can be introduced into host cells through conventional transfection or transformation techniques. Exemplary host cells are E. coli cells, Chinese hamster ovary (CHO) cells, human embryonic kidney 293 (HEK 293) cells, HeLa cells, baby hamster kidney (BHK) cells, monkey kidney cells (COS), human hepatocellular carcinoma cells (e.g., Hep G2), and myeloma cells that do not otherwise produce IgG protein. Transformed host cells can be grown under conditions that permit the host cells to express the genes that encode the immunoglobulin light and/or heavy chain variable regions.

Specific expression and purification conditions will vary depending upon the expression system employed. For example, if a gene is to be expressed in E. coli, it is first cloned into an expression vector by positioning the engineered gene downstream from a suitable bacterial promoter, e.g., Trp or Tac, and a prokaryotic signal sequence. The expressed protein may be secreted. The expressed protein may accumulate in refractile or inclusion bodies, which can be harvested after disruption of the cells by French press or sonication. The refractile bodies then are solubilized, and the protein may be refolded and/or cleaved by methods known in the art.

If the engineered gene is to be expressed in eukaryotic host cells, e.g., CHO cells, it is first inserted into an expression vector containing a suitable eukaryotic promoter, a secretion signal, a poly A sequence, and a stop codon. Optionally, the vector or gene construct may contain enhancers and introns. In embodiments involving antibodies or fusion proteins comprising an antibody or portion thereof, the expression vector optionally contains sequences encoding all or part of a constant region, enabling an entire, or a part of, a heavy or light chain to be expressed. The gene construct can be introduced into eukaryotic host cells using conventional techniques.

In certain embodiments, the host cells express an antibody, fusion protein and/or antibody conjugate comprising a sialidase and V_{L} or V_{H} fragments, V_{L}-V_{H} heterodimers, V_{H}-V_{L} or V_{L}-V_{H} single chain polypeptides, complete heavy or light immunoglobulin chains, or portions thereof, each of which may be attached to a moiety having another function (e.g., cytotoxicity). In some embodiments involving antibodies, fusion proteins and/or antibody conjugates, a host cell is transfected with a single vector expressing a polypeptide expressing a sialidase and an entire, or part of, a heavy chain (e.g., a heavy chain variable region) or a sialidase and a light chain (e.g., a light chain variable region), or a polypeptide expressing an entire, or part of, a heavy chain (e.g., a heavy chain variable region) or a light chain (e.g., a light chain variable region). In some embodiments, a host cell is transfected with a single vector encoding (a) a polypeptide comprising a heavy chain variable region and a polypeptide comprising a light chain variable region, or (b) an entire immunoglobulin heavy chain and an entire immunoglobulin light chain, wherein in (a) or in (b), the polypeptide may also comprise a sialidase. In some embodiments, a host cell is co-transfected with more than one expression vector (e.g., one expression vector expressing a polypeptide comprising an entire, or part of, a heavy chain or heavy chain variable region, optionally comprising a sialidase fused thereto, and another expression vector expressing a polypeptide comprising an entire, or part of, a light chain or light chain variable region, optionally comprising a sialidase fused thereto).

A polypeptide comprising an antibody or a fusion protein, e.g., an antibody or a fusion protein comprising an immunoglobulin heavy chain variable region and/or light chain variable region, can be produced by growing (culturing) a host cell transfected with an expression vector encoding such a variable region, under conditions that permit expression of the polypeptide. Following expression, the polypeptide can be harvested and purified or isolated using techniques known in the art, e.g., affinity tags such as glutathione-S-transferase (GST) or histidine tags.

In certain embodiments, an antibody, fusion protein, and/or antibody conjugate can be produced by growing (culturing) a host cell transfected with: (a) an expression vector that encodes a complete or partial immunoglobulin heavy chain, and a separate expression vector that encodes a complete or partial immunoglobulin light chain; or (b) a single expression vector that encodes both chains (e.g., complete or partial heavy and light chains), under conditions that permit expression of both chains. In embodiments in which a fusion protein and/or antibody conjugate is produced, the sialidase is fused to one or more of the chains. The intact antibody, fusion protein, and/or antibody conjugate can be harvested and purified or isolated using techniques known in the art, e.g., Protein A, Protein G, affinity tags such as glutathione-S-transferase (GST) or histidine tags. It is within ordinary skill in the art to express the heavy chain and the light chain from a single expression vector or from two separate expression vectors.

In certain embodiments, in order to express a protein, e.g., an antibody and/or a fusion protein, as a secreted protein, a native N-terminal signal sequence of the protein is replaced, e.g., with MDMRVPAQLLGLLLLWLPGARC (SEQ ID NO: 28). In certain embodiments, to express a protein, e.g., an antibody and/or a fusion protein, as a secreted protein, an N-terminal signal sequence, e.g., MDMRVPAQLLGLLLLWLPGARC (SEQ ID NO: 28), is added. Additional exemplary N-terminal signal sequences include signal sequences from interleukin-2, CD-5, IgG kappa light chain, trypsinogen, serum albumin, and prolactin. In certain embodiments, in order to express a protein, e.g., an antibody and/or a fusion protein, as a secreted protein, a C terminal lysosomal signal motif, e.g., YGTL (SEQ ID NO: 29) is removed.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321: 522-525; Riechmann et al. (1988) NATURE 332: 323-327; Verhoeyen et al. (1988) SCIENCE 239: 1534-1536; and Winter (1998) FEBS LETT 430: 92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

In addition, it is possible to create fully human antibodies in mice. Fully human mAbs lacking any non-human sequences can be prepared from human immunoglobulin transgenic mice by techniques referenced in, e.g., Lonberg et al., NATURE 368:856-859, 1994; Fishwild et al., NATURE BIOTECHNOLOGY 14:845-851, 1996; and Mendez et al., NATURE GENETICS 15:146-156, 1997. Fully human monoclonal antibodies can also be prepared and optimized from phage display libraries by techniques referenced in, e.g., Knappik et al., J. MOL. BIOL. 296:57-86, 2000; and Krebs et al., J. IMMUNOL. METH. 254:67-84 2001).

The present invention encompasses antibody fragments, or fusion proteins comprising antibody fragments, which may be generated by traditional means, such as enzymatic digestion, or by recombinant techniques. For a review of certain antibody fragments, see Hudson et al. (2003) NAT. MED. 9:129-134.

Various techniques have been developed for the production of antibody fragments. Traditionally, these fragments were derived via proteolytic digestion of intact antibodies (see, e.g., Morimoto et al. (1992) JOURNAL OF BIOCHEMICAL AND BIOPHYSICAL METHODS 24:107-117; and Brennan et al. (1985) SCIENCE 229:81). However, these fragments can now be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab′)2 fragments (Carter et al. (1992) BIO/TECHNOLOGY 10:163-167). According to another approach, F(ab′)₂ fragments can be isolated directly from recombinant host cell culture. Fab and F(ab′)₂ fragments with increased in vivo half-life comprising salvage receptor binding epitope residues are described in U.S. Pat. No. 5,869,046. Other techniques for the production of antibody fragments will be apparent to the skilled practitioner. In certain embodiments, an antibody is a single chain Fv fragment (scFv). See U.S. Pat. Nos. 5,571,894 and 5,587,458.

Methods for making bispecific antibodies are known in the art. See Milstein and Cuello (1983) NATURE 305:537, International (PCT) Publication No. WO93/08829, and Traunecker et al. (1991) EMBO J., 10:3655. For further details of generating bispecific antibodies see, for example, Suresh et al. (1986) METHODS ENZYMOL. 121:210. Bispecific antibodies include cross-linked or “heteroconjugate” or “heterodimer” antibodies. For example, one of the antibodies in the heterodimer can be coupled to avidin, the other to biotin. Heterodimer antibodies may be made using any convenient cross-linking method. Suitable cross-linking agents are well known in the art, and are disclosed in U.S. Pat. No. 4,676,980, along with a number of cross-linking techniques.

Examples of heterodimeric or asymmetric IgG-like molecules include but are not limited to those obtained with the following technologies or using the following formats: Triomab/Quadroma, Knobs-into-Holes, CrossMabs, electrostatically-matched antibodies, LUZ-Y, Strand Exchange Engineered Domain body, Biclonic and DuoBody.

Advantages of using antibody fragments (e.g., F(ab) and F(ab′)2 fragments) include the elimination of non-specific binding between Fc portions of antibodies and Fc receptors on cells (such as macrophages, dendritic cells, neutrophils, NK cells and B cells). In addition, they may be able to penetrate tissues more efficiently due to their smaller size.

Heterodimeric antibodies, or asymmetric antibodies, allow for greater flexibility and new formats for attaching a variety of drugs to the antibody arms. One of the general formats for creating a heterodimeric antibody is the “knobs-into-holes” format. This format is specific to the heavy chain part of the constant region in antibodies. The “knobs” part is engineered by replacing a small amino acid with a larger one, which fits into a “hole”, which is engineered by replacing a large amino acid with a smaller one. What connects the “knobs” to the “holes” are the disulfide bonds between each chain. The “knobs-into-holes” shape facilitates antibody dependent cell mediated cytotoxicity. Single chain variable fragments (scFv) are connected to the variable domain of the heavy and light chain via a short linker peptide. The linker is rich in glycine, which gives it more flexibility, and serine/threonine, which gives it specificity. Two different scFv fragments can be connected together, via a hinge region, to the constant domain of the heavy chain or the constant domain of the light chain. This gives the antibody bispecificity, allowing for the binding specificities of two different antigens. The “knobs-into-holes” format enhances heterodimer formation but doesn't suppress homodimer formation.

Several approaches to support heterodimerization have been described, for example in International (PCT) Publication Nos. WO96/27011, WO98/050431, WO2007/110205, WO2007/147901, WO2009/089004, WO2010/129304, WO2011/90754, WO2011/143545, WO2012/058768, WO2013/157954, and WO2013/096291, and European Patent Publication No. EP1870459. Typically, in the approaches known in the art, the CH₃ domain of the first heavy chain and the CH₃ domain of the second heavy chain are both engineered in a complementary manner so that the heavy chain comprising one engineered CH₃ domain can no longer homodimerize with another heavy chain of the same structure (e.g., a CH₃-engineered first heavy chain can no longer homodimerize with another CH₃-engineered first heavy chain; and a CH₃-engineered second heavy chain can no longer homodimerize with another CH₃-engineered second heavy chain). Thereby the heavy chain comprising one engineered CH₃ domain is forced to heterodimerize with another heavy chain comprising the CH₃ domain, which is engineered in a complementary manner. As a result, the CH₃ domain of the first heavy chain and the CH₃ domain of the second heavy chain are engineered in a complementary manner by amino acid substitutions, such that the first heavy chain and the second heavy chain are forced to heterodimerize, whereas the first heavy chain and the second heavy chain can no longer homodimerize (e.g., for steric reasons).

### III. Pharmaceutical Compositions

For therapeutic use, an antibody, fusion protein, and/or antibody conjugate preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (see, Remington's Pharmaceutical Sciences, 18th ed. (Mack Publishing Company, 1990).

In certain embodiments, a pharmaceutical composition may contain nanoparticles, e.g., polymeric nanoparticles, liposomes, or micelles (See Anselmo et al. (2016) BIOENG. TRANSL. MED. 1: 10-29).

In certain embodiments, a pharmaceutical composition may contain a sustained- or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(−)-3-hydroxybutyric acid. Sustained release compositions may also include liposomes that can be prepared by any of several methods known in the art.

Pharmaceutical compositions containing an antibody, sialidase fusion protein, or an antibody conjugate disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, intrathecal and rectal administration. In certain embodiments, an antibody, sialidase fusion protein, or an antibody conjugate disclosed herein is administered by IV infusion. In certain embodiments, an antibody, sialidase fusion protein, or an antibody conjugate disclosed herein is administered by intratumoral injection. Useful formulations can be prepared by methods known in the pharmaceutical art. For example, see Remington's Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, NJ) or phosphate buffered saline (PBS). The carrier should be stable under the conditions of manufacture and storage, and should be preserved against microorganisms. The carrier can be a solvent or dispersion medium containing, for example, water, ethanol, polyol (for example, glycerol, propylene glycol, and liquid polyethylene glycol), and suitable mixtures thereof.

In certain embodiments, a pharmaceutical composition may contain a stabilizing agent. In certain embodiments, the stabilizing agent is a cation, such as a divalent cation. In certain embodiments, the cation is calcium or magnesium. The cation can be in the form of a salt, such as calcium chloride (CaCl₂) or magnesium chloride (MgCl₂).

In certain embodiments, the stabilizing agent is present in an amount from about 0.05 mM to about 5 mM. For example, the stabilizing agent may be present in an amount of from about 0.05 mM to about 4 mM, from about 0.05 mM to about 3 mM, from about 0.05 mM to about 2 mM, from about 0.05 mM to about 1 mM, from about 0.05 mM to about 0.5 mM, from about 0.5 mM to about 4 mM, from about 0.5 mM to about 3 mM, from about 0.5 mM to about 2 mM, from about 0.5 mM to about 1 mM, from about 1 mM to about 4 mM, from about 1 mM to about 3 mM, of from about 1 mM to about 2 mM.

Pharmaceutical formulations preferably are sterile. Sterilization can be accomplished by any suitable method, e.g., filtration through sterile filtration membranes. Where the composition is lyophilized, filter sterilization can be conducted prior to or following lyophilization and reconstitution.

The compositions described herein may be administered locally or systemically. Administration will generally be parenteral administration. In a preferred embodiment, the pharmaceutical composition is administered subcutaneously and in an even more preferred embodiment intravenously. Preparations for parenteral administration include sterile aqueous or non-aqueous solutions, suspensions, and emulsions.

Generally, a therapeutically effective amount of active component, for example, an antibody, fusion protein, and/or antibody conjugate, is in the range of 0.1 mg/kg to 100 mg/kg, e.g., 1 mg/kg to 100 mg/kg, 1 mg/kg to 10 mg/kg. The amount administered will depend on variables such as the type and extent of disease or indication to be treated, the overall health of the patient, the in vivo potency of the antibody, the pharmaceutical formulation, and the route of administration. The initial dosage can be increased beyond the upper level in order to rapidly achieve the desired blood-level or tissue-level. Alternatively, the initial dosage can be smaller than the optimum, and the daily dosage may be progressively increased during the course of treatment. Human dosage can be optimized, e.g., in a conventional Phase I dose escalation study designed to run from 0.5 mg/kg to 20 mg/kg. Dosing frequency can vary, depending on factors such as route of administration, dosage amount, serum half-life of the antibody, fusion protein, and/or antibody conjugate, and the disease being treated. Exemplary dosing frequencies are once per day, once per week and once every two weeks. A preferred route of administration is parenteral, e.g., intravenous infusion. In certain embodiments, an antibody, fusion protein, and/or antibody conjugate is lyophilized, and then reconstituted in buffered saline, at the time of administration.

### IV. Therapeutic Uses

The compositions and methods disclosed herein can be used to treat various forms of cancer in a subject or inhibit cancer growth in a subject. The invention provides a method of treating a cancer in a subject. The method comprises administering to the subject an effective amount of an anti-PD-L1 antibody, a sialidase anti-PD-L1 fusion protein, and/or antibody conjugate, e.g., an antibody, fusion protein, or antibody conjugate disclosed herein, either alone or in a combination with another therapeutic agent to treat the cancer in the subject. The term “effective amount” as used herein refers to the amount of an active agent (e.g., an antibody, fusion protein, or antibody conjugate according to the present invention) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of cancers include solid tumors, soft tissue tumors, hematopoietic tumors and metastatic lesions. Examples of hematopoietic tumors include, leukemia, acute leukemia, acute lymphoblastic leukemia (ALL), B-cell, T-cell or FAB ALL, acute myeloid leukemia (AML), chronic myelocytic leukemia (CIVIL), chronic lymphocytic leukemia (CLL), e.g., transformed CLL, diffuse large B-cell lymphomas (DLBCL), follicular lymphoma, hairy cell leukemia, myelodyplastic syndrome (MDS), a lymphoma, Hodgkin's disease, a malignant lymphoma, non-Hodgkin's lymphoma, Burkitt's lymphoma, multiple myeloma, or Richter's Syndrome (Richter's Transformation). Examples of solid tumors include malignancies, e.g., sarcomas, adenocarcinomas, and carcinomas, of the various organ systems, such as those affecting head and neck (including pharynx), thyroid, lung (small cell or non-small cell lung carcinoma (NSCLC)), breast, lymphoid, gastrointestinal (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), CNS (e.g., neural or glial cells, e.g., neuroblastoma or glioma), or skin (e.g., melanoma and metastatic Merkel cell carcinoma (MCC)).

In certain embodiments the cancer is an epithelial cancer, e.g., an epithelial cancer that upregulates the expression of sialylated glycans. Exemplary epithelial cancers include, but are not limited to, endometrial cancer, colon cancer, ovarian cancer, cervical cancer, vulvar cancer, uterine cancer or fallopian tube cancer, breast cancer, prostate cancer, lung cancer, pancreatic cancer, urinary cancer, bladder cancer, head and neck cancer, oral cancer and liver cancer. Epithelial cancers also include carcinomas, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, baso squamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

In certain embodiments the cancer is selected from lung bronchioloalveolar carcinoma (BAC), bladder cancer, a female genital tract malignancy (e.g., uterine serous carcinoma, endometrial carcinoma, vulvar squamous cell carcinoma, and uterine sarcoma), an ovarian surface epithelial carcinoma (e.g., clear cell carcinoma of the ovary, epithelial ovarian cancer, fallopian tube cancer, and primary peritoneal cancer), breast carcinoma, non-small cell lung cancer (NSCLC), a male genital tract malignancy (e.g., testicular cancer), retroperitoneal or peritoneal carcinoma, gastroesophageal adenocarcinoma, esophagogastric junction carcinoma, liver hepatocellular carcinoma, esophageal and esophagogastric junction carcinoma, cervical cancer, cholangiocarcinoma, pancreatic adenocarcinoma, extrahepatic bile duct adenocarcinoma, a small intestinal malignancy, gastric adenocarcinoma, cancer of unknown primary (CUP), colorectal adenocarcinoma, esophageal carcinoma, prostatic adenocarcinoma, kidney cancer, head and neck squamous carcinoma, thymic carcinoma, non-melanoma skin cancer, thyroid carcinoma (e.g., papillary carcinoma), a head and neck cancer, anal carcinoma, non-epithelial ovarian cancer (non-EOC), metastatic urothelial carcinoma (UC), uveal melanoma, malignant pleural mesothelioma, small cell lung cancer (SCLC), a central nervous system cancer, a neuroendocrine tumor, and a soft tissue tumor.

In certain embodiments, the cancer is melanoma, non-small cell lung cancer, colon cancer, breast cancer, bladder cancer, or kidney cancer.

In certain embodiments, the cancer is an adenocarcinoma. In certain embodiments, the cancer is a metastatic cancer. In certain embodiments, the cancer is a refractory cancer.

In certain embodiments, the cancer is resistant to or non-responsive to treatment with an antibody, e.g., an antibody with ADCC activity, e.g., avelumab.

In certain embodiments, the cancer is a PD-L1-expressing cancer, e.g., the cancer comprises cells that express PD-L1. An analysis of 196 tumor specimens from patients with renal cell carcinoma found that high tumor expression of PD-L1 was associated with increased tumor aggressiveness and a 4.5-fold increased risk of death. High expression of PD-L1 is associated with reduced numbers of tumor infiltrating lymphocytes and poor prognosis. In certain embodiments, the PD-L1 status of a cancer can be determined using immunohistochemistry staining protocols, such as DAKO 22C3 and VENTANA SP142 FDA approved protocols, which are used as companion diagnostics for anti-PD-L1 antibodies pembrolizumab, durvalumab, atezolizumab, and avelumab.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more additional therapies, e.g., surgery, radiation therapy, or administration of another therapeutic preparation. In certain embodiments, the additional therapy may include chemotherapy, e.g., a cytotoxic agent. In certain embodiments the additional therapy may include a targeted therapy, e.g., a tyrosine kinase inhibitor, a proteasome inhibitor, or a protease inhibitor. In certain embodiments, the additional therapy may include an anti-inflammatory, anti-angiogenic, anti-fibrotic, or anti-proliferative compound, e.g., a steroid, a biologic immunomodulator, a monoclonal antibody, an antibody fragment, an aptamer, an siRNA, an antisense molecule, a fusion protein, a cytokine, a cytokine receptor, a bronchodilator, a statin, an anti-inflammatory agent (e.g., methotrexate), or an NSAID. In certain embodiments, the additional therapy may include a combination of therapeutics of different classes.

In certain embodiments, a method or composition described herein is administered in combination with a second checkpoint inhibitor. The checkpoint inhibitor may, for example, be selected from a PD-1 antagonist, a second PD-L1 antagonist, CTLA-4 antagonist, adenosine A2A receptor antagonist, B7-H3 antagonist, B7-H4 antagonist, BTLA antagonist, KIR antagonist, LAG3 antagonist, TIM-3 antagonist, VISTA antagonist or TIGIT antagonist.

In certain embodiments, the checkpoint inhibitor is a PD-1 or a second PD-L1 inhibitor. PD-1 is a receptor present on the surface of T-cells that serves as an immune system checkpoint that inhibits or otherwise modulates T-cell activity at the appropriate time to prevent an overactive immune response. Cancer cells, however, can take advantage of this checkpoint by expressing ligands, for example, PD-L1, that interact with PD-1 on the surface of T-cells to shut down or modulate T-cell activity. Exemplary PD-1/PD-L1 based immune checkpoint inhibitors include antibody based therapeutics. Exemplary treatment methods that employ PD-1/PD-L1 based immune checkpoint inhibition are described in U.S. Pat. Nos. 8,728,474 and 9,073,994, and EP Patent No. 1537878B1, and, for example, include the use of anti-PD-1 antibodies. Exemplary anti-PD-1 antibodies are described, for example, in U.S. Pat. Nos. 8,952,136, 8,779,105, 8,008,449, 8,741,295, 9,205,148, 9,181,342, 9,102,728, 9,102,727, 8,952,136, 8,927,697, 8,900,587, 8,735,553, and 7,488,802. Exemplary anti-PD-1 antibodies include, for example, nivolumab (Opdivo®, Bristol-Myers Squibb Co.), pembrolizumab (Keytruda®, Merck Sharp & Dohme Corp.), PDR001 (Novartis Pharmaceuticals), and pidilizumab (CT-011, Cure Tech). Exemplary anti-PD-L1 antibodies are described, for example, in U.S. Pat. Nos. 9,273,135, 7,943,743, 9,175,082, 8,741,295, 8,552,154, and 8,217,149. Exemplary anti-PD-L1 antibodies include, atezolizumab (Tecentriq®, Genentech), durvalumab (AstraZeneca), MEDI4736, avelumab, and BMS 936559 (Bristol Myers Squibb Co.).

In certain embodiments, a method or composition described herein is administered in combination with a CTLA-4 inhibitor. In the CTLA-4 pathway, the interaction of CTLA-4 on a T-cell with its ligands (e.g., CD80, also known as B7-1, and CD86) on the surface of an antigen presenting cells (rather than cancer cells) leads to T-cell inhibition. Exemplary CTLA-4 based immune checkpoint inhibition methods are described in U.S. Pat. Nos. 5,811,097, 5,855,887, 6,051,227. Exemplary anti-CTLA-4 antibodies are described in U.S. Pat. Nos. 6,984,720, 6,682,736, 7,311,910; 7,307,064, 7,109,003, 7,132,281, 6,207,156, 7,807,797, 7,824,679, 8,143,379, 8,263,073, 8,318,916, 8,017,114, 8,784,815, and 8,883,984, International (PCT) Publication Nos. WO98/42752, WO00/37504, and WO01/14424, and European Patent No. EP 1212422 B1. Exemplary CTLA-4 antibodies include ipilimumab or tremelimumab.

In certain embodiments, a method or composition described herein is administered in combination with a CTLA-4 inhibitor, e.g., a CTLA-4 inhibitor disclosed herein.

In certain embodiments, a method or composition described herein is administered in combination with an IDO inhibitor. Exemplary IDO inhibitors include 1-methyl-D-tryptophan (known as indoximod), epacadostat (INCB24360), navoximod (GDC-0919), and BMS-986205.

Exemplary cytotoxic agents that can be administered in combination with a method or composition described herein include, for example, antimicrotubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with a method or composition described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

The invention also provides a method of increasing the expression of HLA-DR, CD86, CD83, IFN-γ, IL-1b, IL-6, TNFα, IL-17A, IL-2, or IL-6 in a cell, tissue, or subject. The method comprises contacting the cell, tissue, or subject with an effective amount of an antibody, fusion protein, and/or antibody conjugate, e.g., an antibody, fusion protein, or antibody conjugate disclosed herein. In certain embodiments, the cell is selected from a dendritic cell and a peripheral blood mononuclear cell (PBMC).

In certain embodiments, expression of HLA-DR, CD86, CD83, IFN-γ, IL-1b, IL-6, TNFα, IL-17A, IL-2, or IL-6 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the antibody, fusion protein, or antibody conjugate. Gene expression may be measured by any suitable method known in the art, for example, by ELISA, or by Luminex multiplex assays.

The invention also provides a method of promoting infiltration of immune cells into a tumor in a subject in need thereof. The method comprises administering to the subject an effective amount of an antibody, fusion protein, and/or antibody conjugate, e.g., an antibody, fusion protein, or antibody conjugate disclosed herein. In certain embodiments, the immune cells are T-cells, e.g., CD4+ and/or CD8+ T-cells, e.g., CD69⁺CD8⁺ and/or GzmB⁺CD8⁺ T-cells. In certain embodiments, the immune cells are natural killer (NK) cells.

In certain embodiments, the infiltration of immune cells into the tumor in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor and/or subject that has not been administered the antibody, fusion protein, or antibody conjugate. Infiltration of immune cells into a tumor may be measured by any suitable method known in the art, for example, antibody staining.

The invention also provides a method of increasing the number of circulating natural killer (NK) cells in a subject in need thereof. The method comprises administering to the subject an effective amount of an antibody, fusion protein, and/or antibody conjugate, e.g., an antibody, fusion protein, or antibody conjugate disclosed herein, so as to increase the number of circulating NK cells relative to prior to administration of the antibody, fusion protein, antibody conjugate or pharmaceutical composition.

In certain embodiments, the number of circulating NK cells in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the antibody, fusion protein, or antibody conjugate. Circulating NK cells in a subject may be measured by any suitable method known in the art, for example, antibody staining.

The invention also provides a method of increasing the number of T-cells in the draining lymph node in a subject in need thereof. The method comprises administering to the subject an effective amount of an antibody, fusion protein, and/or antibody conjugate, e.g., an antibody, fusion protein, or antibody conjugate disclosed herein, so as to increase the number of T-cells in the draining lymph node relative to prior to administration of the antibody, fusion protein, antibody conjugate, or pharmaceutical composition. In certain embodiments, the immune cells are T-cells, e.g., CD4+ and/or CD8+ T-cells.

In certain embodiments, the number of T-cells in the draining lymph node in the subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical subject that has not been administered the antibody, fusion protein, or antibody conjugate, or pharmaceutical composition. T-cells in the draining lymph node in a subject may be measured by any suitable method known in the art, for example, antibody staining.

The invention also provides a method of increasing expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in a cell, tissue, or subject. The method comprises contacting the cell, tissue, or subject with an effective amount of an antibody, fusion protein, and/or antibody conjugate, e.g., an antibody, fusion protein, or antibody conjugate disclosed herein, so as to increase the expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 relative to the cell, tissue or subject prior to contact with the antibody, fusion protein, or antibody conjugate.

In certain embodiments, expression of Cd3, Cd4, Cd8, Cd274, Ctla4, Icos, Pdcd1, Lag3, Il6, Il1b, Il2, Ifng, Ifna1, Mx1, Gzmb, Cxcl9, Cxcl12, and/or Ccl5 in the cell, tissue, or subject is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell, tissue, or subject that has not been contacted with the antibody, fusion protein, or antibody conjugate. Gene expression may be measured by any suitable method known in the art, for example, by ELISA, Luminex multiplex assays, or Nanostring technology.

The invention also provides a method of removing sialic acid from a cell or tissue. The method comprises contacting the cell or tissue with an effective amount of a fusion protein and/or antibody conjugate, e.g., a fusion protein or antibody conjugate disclosed herein. The invention also provides a method of removing sialic acid from a cell in a subject, the method comprising administering to the subject an effective amount of a pharmaceutical composition comprising a fusion protein and/or antibody conjugate, e.g., a fusion protein or antibody conjugate disclosed herein, thereby to remove sialic acid from the cell.

In certain embodiments, the cell is tumor cell, dendritic cell (DC) or monocyte. In certain embodiments, the cell is a monocyte, and the method results in increased expression of an MHC-II molecule (e.g., HLA-DR) on the monocyte. In certain embodiments, expression of an MHC-II molecule in the cell or tissue is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical cell or tissue that has not been contacted with the fusion protein and/or antibody conjugate. Gene expression may be measured by any suitable method known in the art, for example, by ELISA, by Luminex multiplex assays, or by flow cytometry.

The invention also provides a method of enhancing phagocytosis of a tumor cell. The method comprises contacting the tumor cell with a fusion protein and/or antibody conjugate, e.g., a fusion protein or antibody conjugate disclosed herein, in an amount effective to remove sialic acid from the tumor cell, thereby enhancing phagocytosis of the tumor cell. In certain embodiments, the disclosure relates to a method of increasing phagocytosis of a tumor cell in a subject, the method comprising administering to the subject an effective amount of a pharmaceutical composition a fusion protein and/or antibody conjugate, e.g., a fusion protein or antibody conjugate disclosed herein, in an amount effective to remove sialic acid from the tumor cell, thereby to increase phagocytosis of the tumor cell.

In certain embodiments, phagocytosis is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical tumor cell or population of tumor cells that has not or have not been contacted with the fusion protein and/or antibody conjugate. Phagocytosis may be measured by any suitable method known in the art.

The invention also provides a method of activating a dendritic cell (DC). The method comprises contacting the DC with a tumor cell that has been treated with a fusion protein and/or antibody conjugate, e.g., a fusion protein or antibody conjugate disclosed herein. In certain embodiments, the disclosure relates to a method of activating a dendritic cell (DC) or a population of DCs in a subject, the method comprising administering to the subject an amount of a pharmaceutical composition comprising a fusion protein and/or antibody conjugate, e.g., a fusion protein or antibody conjugate disclosed herein, effective to remove sialic acid from a tumor cell in the subject, thereby to activate the DC or the population of DCs in the subject.

In certain embodiments, activation of the DC or a population of DCs is increased by at least about 10%, at least about 20%, at least about 50%, at least about 75%, at least about 100%, at least about 150%, at least about 200%, at least about 250%, at least about 300%, at least about 400%, at least about 500%, at least about 600%, at least about 700%, at least about 800%, at least about 900%, or at least about 1,000%, relative to a similar or otherwise identical DC or population of DCs that has not or have not been contacted with a tumor cell that has been treated with the fusion protein and/or antibody conjugate. Activation may be measured by any suitable method known in the art.

The invention also provides a method of reducing Siglec-15 binding activity, thereby to increase anti-tumor activity in a tumor microenvironment, the method comprising contacting a T cell with an antibody, fusion protein, and/or antibody conjugate, e.g., an antibody, fusion protein, or antibody conjugate disclosed herein. In certain embodiments, the disclosure relates to a method of reducing Siglec-15 binding activity, thereby to increase anti-tumor activity in a tumor microenvironment of a patient, the method comprising administering to the subject an effective amount of a pharmaceutical composition comprising an antibody, fusion protein, and/or antibody conjugate, e.g., an antibody, fusion protein, or antibody conjugate disclosed herein, thereby to increase anti-tumor activity (e.g., T cell activity) in the subject.

In certain embodiments, Siglec-15 binding activity is reduced by at least about 10%, at least about 20%, at least about 50%, at least about 75%, or about 100%, relative to Siglec-15 that has not or have not been contacted with the antibody, fusion protein, antibody conjugate, and/or pharmaceutical composition. Binding may be measured by any suitable method known in the art.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1

This example describes the construction of recombinant human sialidases (Neu1, Neu2, and Neu3).

The human sialidases Neu1, Neu2, Neu3 (isoform 1), and Neu4 (isoform 1) were expressed as secreted proteins with a 10×His tag. To express Neu1 as a secreted protein, the native N terminal signal peptide (MTGERPSTALPDRRWGPRILGFWGGCRVWVFAAIFLLLSLAASWSKA; SEQ ID NO: 27) was replaced by MDMRVPAQLLGLLLLWLPGARC (SEQ ID NO: 28), and the C terminal lysosomal signal motif (YGTL; SEQ ID NO: 29) was removed. To express Neu2, Neu3, and Neu4 as secreted proteins, the N terminal signal peptide MDMRVPAQLLGLLLLWLPGARC (SEQ ID NO: 28) was added to each.

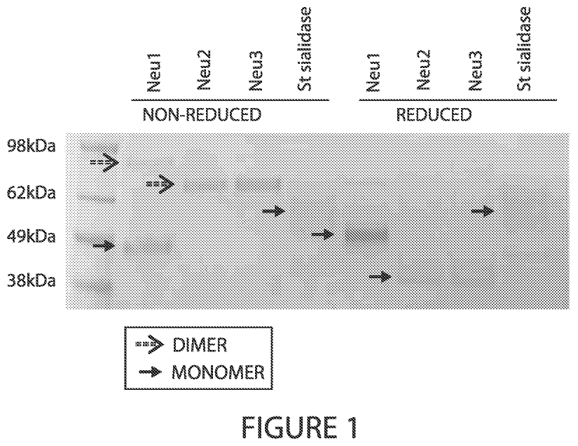

Sialidases were expressed in a 200 mL transfection of HEK293F human cells in 24-well plates using the pCEP4 mammalian expression vector with an N-terminal 6×His tag. Sialidases were purified using Ni-NTA columns, quantified with a UV-Vis spectrophotometer (NanoDrop), and examined by SDS-PAGE as shown in FIG. 1. Neu1 expressed well, with a yield of ˜3 μg/ml, and was present primarily in a monomeric form. Neu2 and Neu3 expression each gave yields of ˜0.15 μg/mL and each were present primarily in a dimeric form. Neu4 had no detectable expression yield as measured by NanoDrop. Bacterial sialidase from Salmonella typhimurium (St-sialidase; SEQ ID NO: 30), which was used as a positive control for expression, gave a comparable yield to Neu1, and was present primarily in a monomeric form.

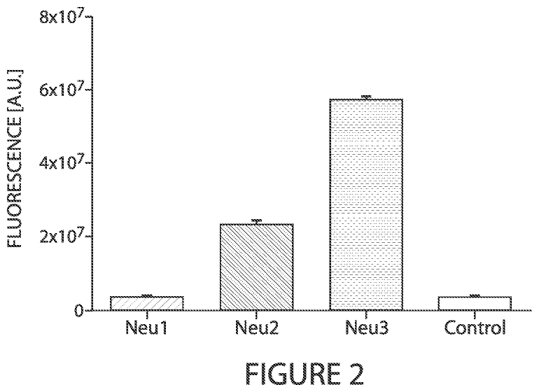

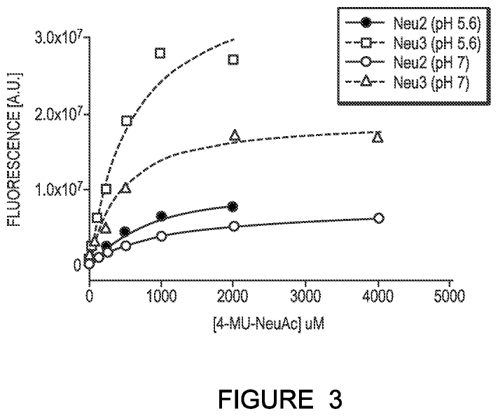

The activity of the recombinantly expressed sialidases was assayed by measuring the release of sialic acid from the fluorogenic substrate 4-methylumbelliferyl-N-acetylneuraminic acid (4MU-NeuAc). As shown in FIG. 2, Neu1 has no detectable activity above a no-enzyme control, which is consistent with previous reports indicating that Neu1 is inactive unless it is in complex with beta-galactosidase and protective protein/cathepsin A (PPCA). Neu2 and Neu3 were active. An enzyme kinetics assay was performed with Neu2 and Neu3. A fixed concentration of enzyme at 1 nM was incubated with fluorogenic substrate 4MU-NeuAc at concentrations ranging from 4000 μM to 7.8 μM. Assays were conducted at both acidic (pH 5.6) and neutral (pH 7) conditions. As shown in FIG. 3, both Neu2 and Neu3 were active at acidic and neutral conditions and showed enzyme kinetics that were comparable to those previously reported.

Most of the recombinantly expressed sialidases ran as aggregates or dimers on a non-reducing SDS-PAGE gel. Subsequent treatment with the reducing agent dithiothreitol (DTT) resulted in a monomeric form of the enzyme that ran at 42 kDa on a reducing SDS-PAGE gel (FIG. 1).

### Example 2

This example describes PD-L1 antibody discovery and hybridoma screening.

Antibodies were generated using two different methods. In the first method (Green Mountain Antibody, Vermont), 3 SJL/J mice and BALB/cJ mice were immunized using hPD-L1-hFc following the 28-day RIMMS protocol. PEG fusion of splenocytes and lymphocytes from the high titer mice with NS1 myeloma cells was performed to generate hybridomas.

In the second method (Aldevron, WI), human PD-L1 extracellular domain (ECD) was cloned into a vector plasmid also containing a detection tag for immunization and control. The plasmid constructs were transfected into mammalian cells. hPD-L1 expression was validated using flow cytometry with an hPD-L1 antibody and anti-tag antibody, the vector control using an irrelevant anti-tag antibody. Five mice were immunized with validated PD-L1 ECD plasmid DNA. The immune response was checked with mice sera using flow cytometry on cells transfected with hPD-L1. PEG fusion of splenocytes and lymphocytes from the high titer mice with NS1 myeloma cells was performed to generate hybridomas.

Hybridoma supernatants were screened using an ELISA to determine binding to human PD-L1 (hPD-L1) as well as cynomolgus PD-L1 (cPD-L1). Hybridoma supernatants were diluted 10× in ELISA binding buffer prior to loading on a hPD-L1-his tagged or cPD-L1-his tagged coated ELISA wells. The binding of mouse IgGs were detected using HRP-conjugated Goat-anti-mouse polyclonal antibody. The plate was developed with TMB and Stop buffer, the absorbance at 450 nm was read using SpectraMax plate reader.

A second assay was also utilized wherein hybridoma supernatants were screened for the ability to block biotinylated hPD-1-Fc from binding to hPD-L1. Hybridoma supernatants were diluted 3× in ELISA binding buffer and mixed with biotin-hPD-1-Fc at a final concentration of 1 μg/mL. The mixtures were loaded to hPD-L1-Fc coated ELISA wells for binding. The antibodies that recognized the hPD-1/hPD-L1 epitope bin competed for binding and reduced the hPD-1-Fc binding signal. The residual binding of biotin-hPD-1-Fc to hPD-L1-Fc was detected with HRP conjugated Streptavidin. The plate was developed with TMB and Stop buffer, and the absorbance at 450 nm was read using SpectraMax plate reader. The A450 absorbance was normalized to the hybridoma-conditioned medium control.

TABLE 10 is a summary of representative hybridoma supernatant screening results. Selected clones with good binding to hPD-L1 and cPD-L1 and with low residual binding were further characterized.

TABLE 10
Summary of Hybridoma Supernatant Screening

| | | | Block |
|---|---|---|---|
| Clone | huPD-L1-His | cyPD-L1-His | Residual % |
| 01A11 | 0.04325 | 0.0444 | 68% |
| 01E09 | 2.36185 | 1.5722 | 35% |
| 01G01 | 0.0496 | 0.0484 | 85% |
| 01G07 | 1.9901 | 1.3883 | 11% |
| 02C01 | 1.8316 | 1.3126 | 11% |
| 02C03 | 0.05075 | 0.04525 | 94% |
| 02C04 | 2.06255 | 1.441 | 47% |
| 02D06 | 0.03525 | 0.0367 | 87% |
| 02D10 | 1.83325 | 1.36395 | 9% |
| 02E09 | 1.96075 | 1.27385 | 82% |
| 02F06 | 0.0491 | 0.0451 | 92% |
| 02G09 | 0.04745 | 0.0418 | 89% |
| 02H01 | 0.1207 | 0.08695 | 91% |
| 02H02 | 0.0385 | 0.0375 | 99% |
| 02H07 | 2.3958 | 1.46115 | 30% |
| 03D03 | 0.0425 | 0.0402 | 84% |
| 03G01 | 0.04025 | 0.04055 | 82% |
| 03G08 | 0.04285 | 0.04215 | 93% |
| 03H06 | 0.04915 | 0.04295 | 92% |
| 04C01 | 0.0441 | 0.041 | 92% |
| 04C11 | 1.996 | 1.07865 | 33% |
| 04D05 | 2.1616 | 1.3421 | 39% |
| 04G02 | 0.0483 | 0.0447 | 89% |
| 04H04 | 0.04255 | 0.0387 | 82% |
| 05A04 | 0.03905 | 0.03935 | 82% |
| 05C02 | 0.03905 | 0.0385 | 88% |
| 05D01 | 0.04775 | 0.0431 | 79% |
| 05F02 | 0.0395 | 0.0398 | 81% |
| 05G01 | 0.06305 | 0.04815 | 96% |
| 05H01 | 0.04225 | 0.0419 | 83% |
| 05H03 | 0.0427 | 0.04095 | 83% |
| 05H07 | 0.05695 | 0.0472 | 81% |
| 06A10 | 0.0427 | 0.04205 | 102% |
| 06H03 | 0.0386 | 0.0392 | 99% |
| 07C04 | 2.5075 | 1.385 | 43% |
| 07D11 | 0.037 | 0.03705 | 99% |
| 07G03 | 0.23005 | 0.17695 | 112% |
| 07G09 | 2.57165 | 1.49485 | 119% |
| 08F01 | 0.03855 | 0.0361 | 100% |
| 08F02 | 0.97025 | 1.13735 | 106% |
| 08F08 | 0.0378 | 0.03775 | 101% |
| 09B07 | 1.706 | 0.5715 | 72% |
| 09C01 | 2.61775 | 1.31865 | 18% |
| 09F04 | 0.04145 | 0.04025 | 103% |
| 09F05 | 0.0412 | 0.04235 | 116% |
| 09F07 | 2.5844 | 1.31555 | 46% |
| 09G07 | 0.04155 | 0.03915 | 123% |
| 10B03 | 0.0439 | 0.0402 | 109% |
| 10B04 | 0.0445 | 0.04275 | 114% |
| 10C07 | 0.04485 | 0.04475 | 118% |
| 10C08 | 3.2374 | 0.12175 | 17% |
| 10D07 | 0.0387 | 0.03835 | 118% |
| 10E05 | 0.04135 | 0.04345 | 112% |
| 10E06 | 2.2714 | 1.5315 | 106% |
| 10F03 | 0.03975 | 0.04215 | 118% |
| 10F04 | 0.04305 | 0.03955 | 118% |
| 10F05 | 0.04405 | 0.04045 | 97% |
| 10F07 | 1.581 | 1.36875 | 101% |
| 10F10 | 0.0412 | 0.0387 | 104% |
| 10G09 | 2.75445 | 1.2858 | 51% |
| 11A01 | 0.0504 | 0.0437 | 111% |
| 11A06 | 0.04655 | 0.0439 | 111% |
| 11A08 | 2.049 | 1.4734 | 107% |
| 11F01 | 2.2419 | 1.62365 | 116% |
| 11H03 | 2.9876 | 1.32625 | 70% |
| 12A02 | 0.35785 | 0.4006 | 76% |
| 12A05 | 2.42665 | 1.40435 | 47% |
| 12B11 | 2.07995 | 1.2203 | 51% |
| 12E08 | 2.0973 | 1.46875 | 32% |
| 12G04 | 2.1039 | 1.37465 | 26% |
| 12H04 | 2.05395 | 1.4835 | 75% |
| 12H06 | 2.0525 | 1.42365 | 109% |
| 13F05 | 2.3518 | 1.4181 | 6% |
| 13G07 | 0.3502 | 0.6019 | 69% |
| 13H02 | 2.2304 | 1.35665 | 22% |
| 14B06 | 0.0383 | 0.0414 | 86% |
| 14C09 | 1.2704 | 1.12745 | 80% |
| 14F11 | 0.0405 | 0.04175 | 80% |
| 14G07 | 2.2582 | 1.4165 | 40% |
| 14H07 | 1.57355 | 1.31875 | 64% |
| 15D01 | 2.41115 | 1.4562 | 37% |
| 15D05 | 0.04585 | 0.0435 | 86% |
| 15E01 | 0.04575 | 0.04655 | 82% |
| 15F06 | 0.9242 | 0.78545 | 111% |
| 15G01 | 0.0506 | 0.04365 | 87% |
| 15G09 | 0.0423 | 0.04545 | 83% |
| 15H04 | 2.221 | 1.4642 | 21% |
| 15H08 | 0.05165 | 0.0468 | 74% |
| 16A04 | 0.0475 | 0.0514 | 67% |
| 16B05 | 0.183 | 0.16385 | 76% |
| 16F01 | 0.64805 | 0.94295 | 65% |
| 16F08 | 2.4565 | 1.32575 | 39% |
| 16G01 | 2.3645 | 0.9678 | 7% |
| 16H04 | 0.0439 | 0.04285 | 82% |
| POS | 2.31 | 1.44 | 10% |
| NEG | 0.034 | 0.03475 | 60% |

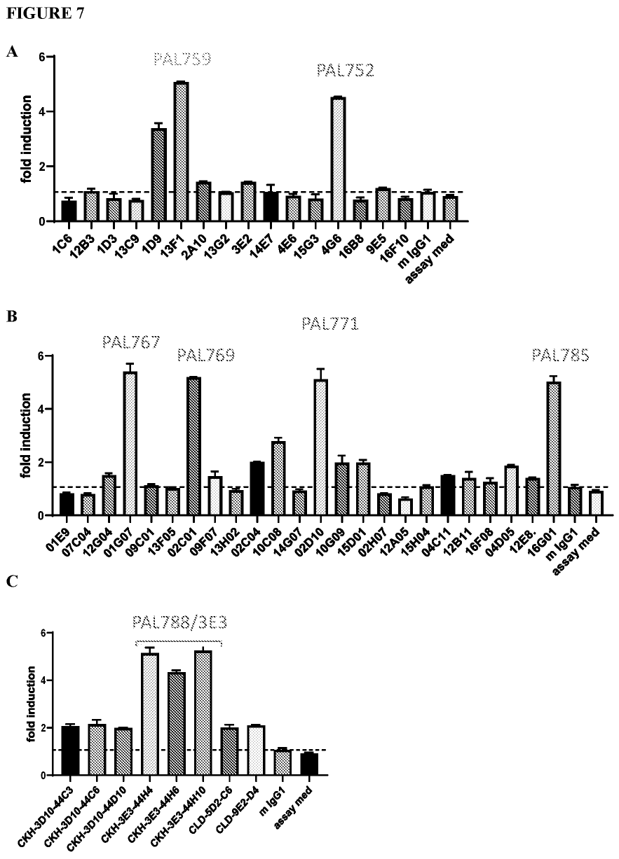

Functional blocking assay. The supernatants from selected PD-L1 antibody-producing hybridomas were tested in co-culture with (i) engineered CHO-K1 cells expressing human PD-L1 and TCR activating protein and (ii) Jurkat T cells expressing human PD-1, TCR and a luciferase reporter driven by an NFAT response element. Absent intervention, PD-L1 interacting with PD-1 inhibits TCR-mediated luminescence. Blockade of the PD-L1/PD-1 interaction, for instance using avelumab or PD-L1 antibody-containing supernatants, results in a luminescent signal. A luciferase substrate was added after 6 hours of incubation and luminescence was measured. Relative light units (RLU) were calculated by subtracting background (substrate and media only) from assay wells. Fold induction was calculated by dividing the RLU of induced cells minus background by the RLU of the no antibody control minus background. As shown in FIG. 7, hybridoma supernatants capable of disrupting the PD-L1/PD-1 interaction, resulting in a dose dependent increase in luminescence, were identified. FIGS. 7A, 7B and 7C represent the testing of three batches antibodies. Avelumab was used as a positive control producing a Fold Induction of 4 to 5 over a range of 0.1 to 10 μg/mL in this assay (data not shown). Fold induction=RLU (induced−background)/RLU (no antibody control−background). EC₅₀s of the indicated PD-L1 antibodies were comparable to the EC₅₀ for avelumab.

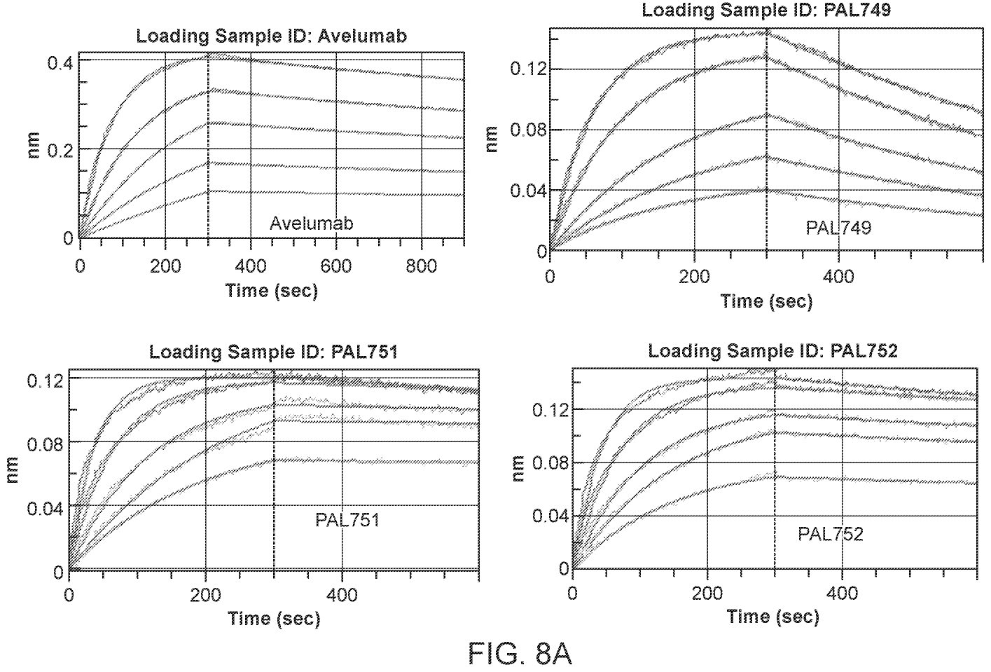

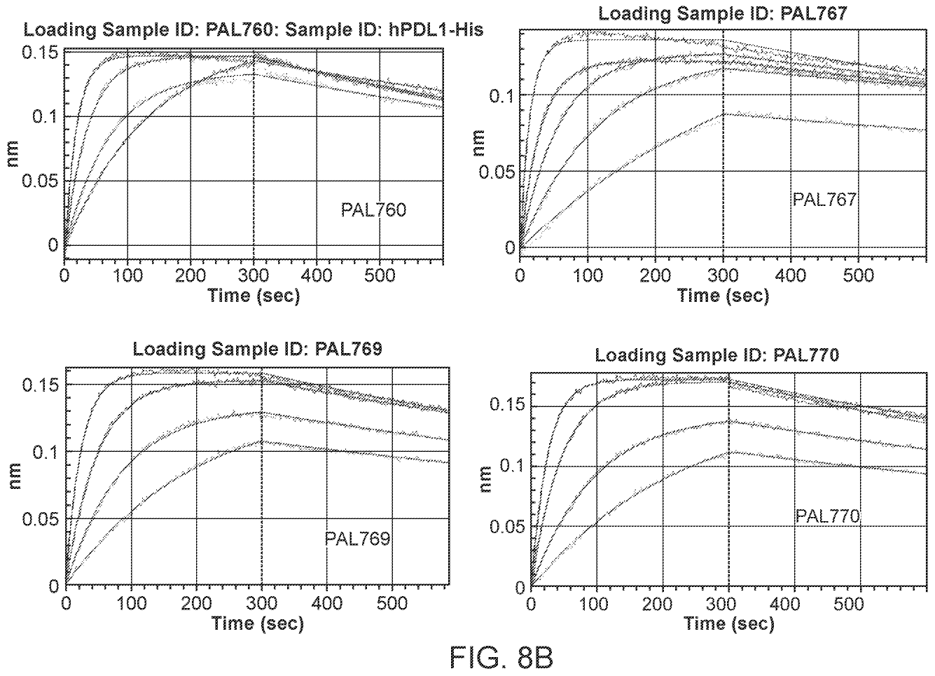

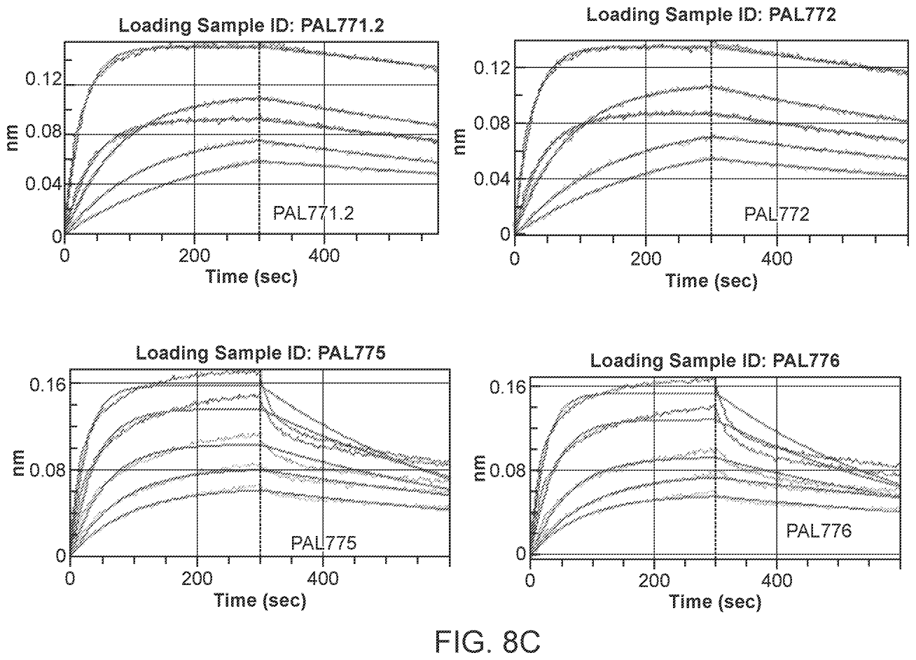

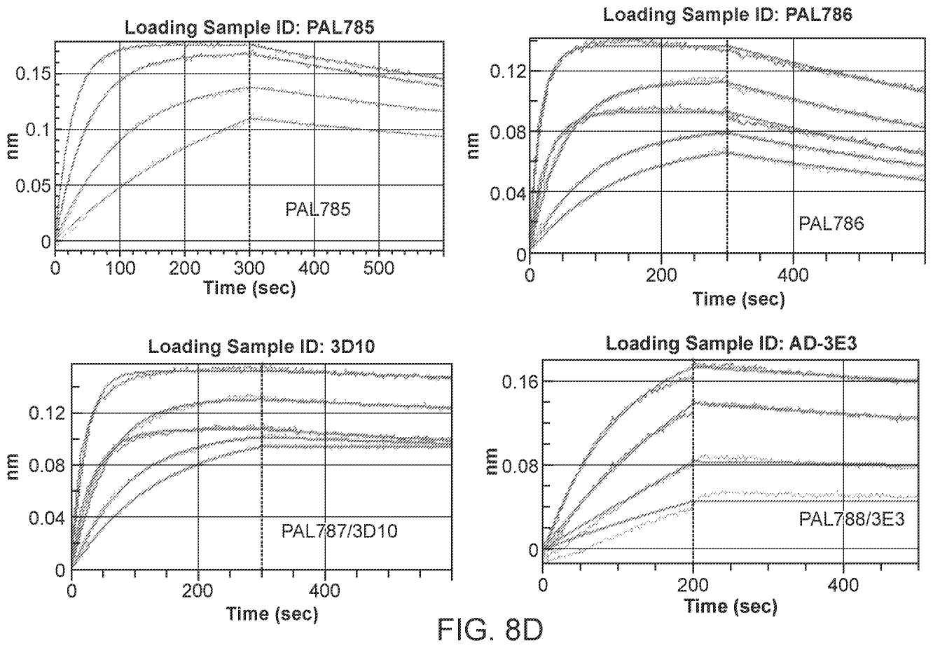

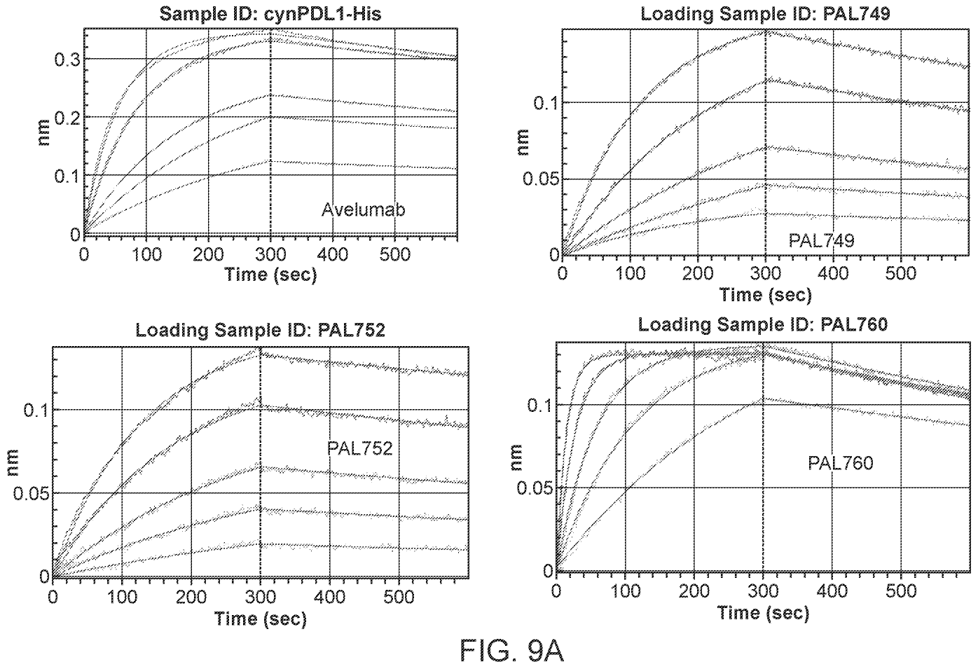

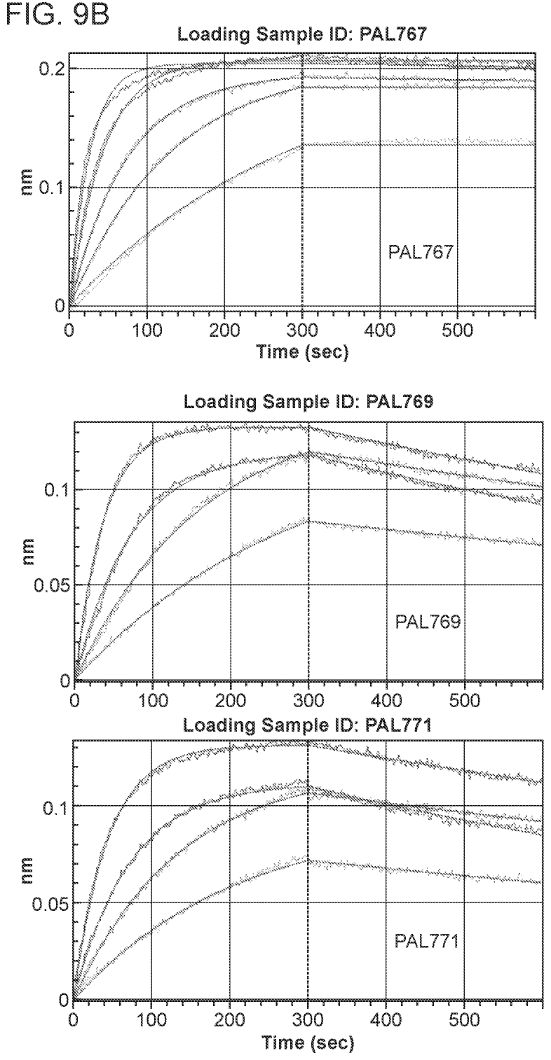

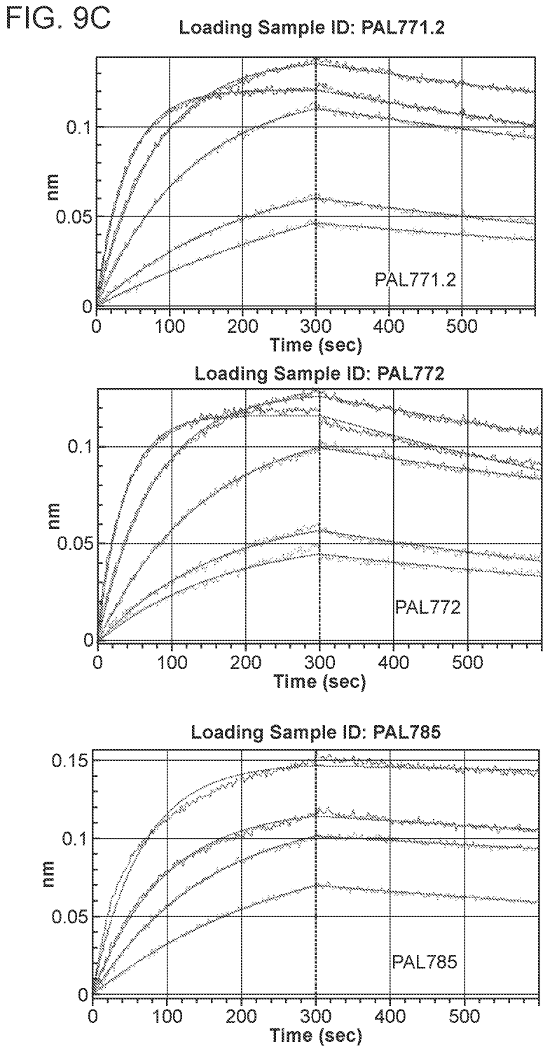

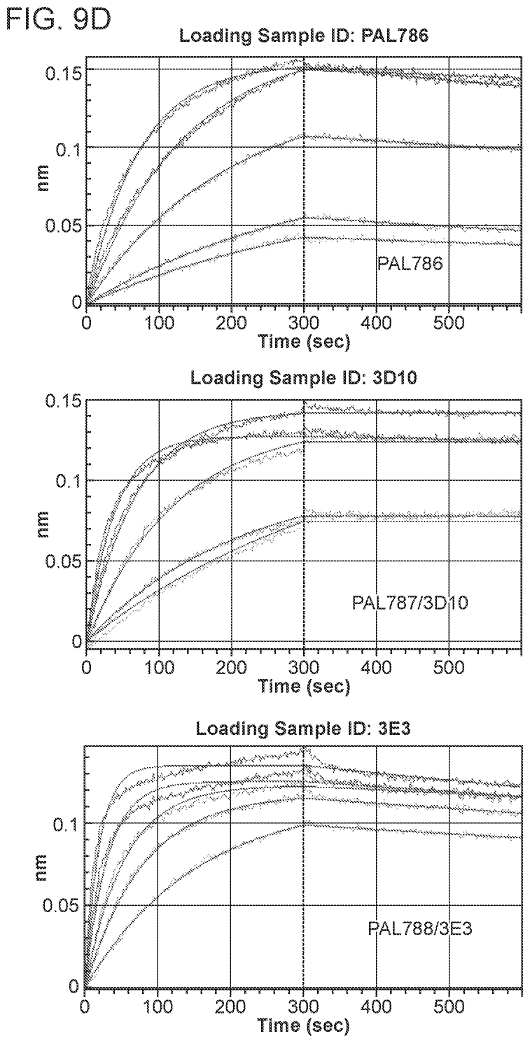

Screening of purified hybridoma antibodies. ForteBio octet binding of purified antibodies to recombinant hPD-L1-his and cPD-L1-his was measured. Mouse IgGs purified from hybridoma supernatant were captured on AMC (Anti-mouse IgG-Capture) Biosensor. hPD-L1-his or cPD-L1-his analytes were titrated from 100 nM in a 2× series dilution. The signal was subtracted with buffer reference and aligned to the baseline. KD, Kon and Koff values were generated using a 1:1 fitting model. The binding kinetics of selected purified hybridomas are shown in FIGS. 8A, 8B, 8C, and 8D for hPD-L1 and FIGS. 9A, 9B, 9C, and 9D for cPD-L1. The X-axes represent assay time in seconds, and the Y-axes represent binding signal on the biosensor. Each line represents the real time signal of antigen association and dissociation at the given antigen concentration in the assay (e.g., the top line represents the signal of the highest antigen concentration in the assay, the second top line represents the second highest concentration in the assay). The vertical dashed line represents the time point that the assay was moved from association step to the dissociation step. All the ForteBio/Octet assays were using standard/conventional settings and the graphs provided in the figures will be understood by one of skill in the art. The calculated KDs are shown in TABLE 11 for hPD-L1 and cPD-L1.

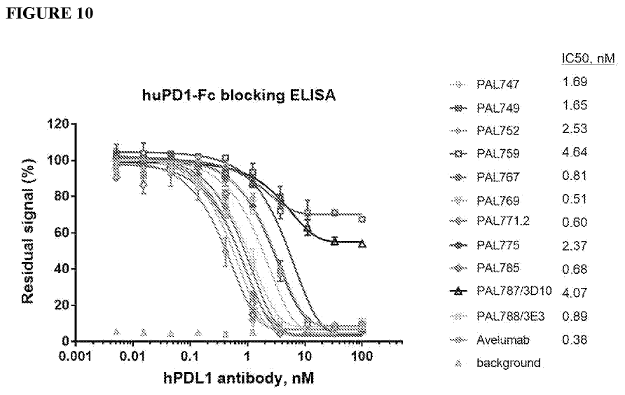

Purified hybridoma antibodies were tested for their ability to block biotinylated hPD-1-Fc from binding to hPD-L1. Antibodies were 3× titrated and mixed with biotinylated hPD-1-Fc at final concentration of 1 μg/mL. The mixture of antibody and biotin-hPD-1-Fc were loaded to hPD-L1-Fc coated ELISA wells for binding. The antibodies that recognize the hPD-1/hPD-L1 epitope bin will compete for the binding and result hPD-1-Fc binding signal reduction. The residual binding of biotin-hPD-1-Fc to hPD-L1-Fc were detected with HRP conjugated Streptavidin. The plate was developed with TMB and Stop buffer, the absorbance at 450 nm was read using SpectraMax plate reader. The A450 absorbance was normalized to the control that without antibody for the percentage values. The curve and IC₅₀ were generated using GraphPad Prism software. As depicted in FIG. 10, antibodies demonstrated either complete (C) or partial (P) blocking of hPD-1 to hPD-L1. The IC₅₀ as determined from the data is shown in FIG. 10 with many in the low single nM range or lower and is also shown in TABLE 11.

TABLE 11
Summary of anti-hPD-L1 hybridoma antibodies

| | | cell | | | ELISA | | |
|---|---|---|---|---|---|---|---|
| | | block | Kinetics KD (M) | | cyno, Fold | IC₅₀, | |
| Hybridoma | PAL ID | C/P | hPD-L1 | cPD-L1 | over Bkg | nM | C/P |
| 1D9.A7 | PAL747 | C | Low signal | Low signal | 1.10 | 1.69* | C |
| 1D9.B6 | PAL748 | | Low signal | | 0.94 | | |
| 3E2.B6 | PAL749 | P | 9.92 × 10⁻⁰⁹ | 7.44 × 10⁻⁰⁹ | 43.49 | 1.65 | P |
| 4G6.A3 | PAL751 | C | 3.85 × 10⁻¹⁰ | | | | |
| 4G6.H4 | PAL752 | | 7.89 × 10⁻¹⁰ | 4.03 × 10⁻⁰⁹ | 18.39 | 2.53 | C |
| 13F1.B7 | PAL759 | C | 1.14 × 10⁻⁰⁹ | | 13.12 | 4.64* | C |
| 13F1.F2 | PAL760 | | 7.11 × 10⁻¹⁰ | 1.09 × 10⁻⁰⁹ | 32.82 | | |
| 1G7.A8 | PAL767 | C | 8.03 × 10⁻¹⁰ | 5.77 × 10⁻¹¹ | 36.91 | 0.81 | C |
| 1G7.C8 | PAL768 | | 5.59 × 10⁻¹⁰ | | 34.91 | | |
| 2C1.C8 | PAL769 | C | 7.58 × 10⁻¹⁰ | 1.3 × 10⁻⁰⁹ | 39.52 | 0.51 | C |
| 2C1.D11 | PAL770 | | 8.82 × 10⁻¹⁰ | | 36.07 | | |
| 2D10.E2.B9 | PAL771 | C | 5.92 × 10⁻¹⁰ | 1.15 × 10⁻⁰⁹ | | | |
| 2D10.E2.H4 | PAL771.2 | | 1.54 × 10⁻⁰⁹ | 2.27 × 10⁻⁰⁹ | 38.14 | 0.6 | C |
| 2D10.G3 | PAL772 | | 1.75 × 10⁻⁰⁹ | 2.64 × 10⁻⁰⁹ | 22.16 | | |
| 10C8.A7 | PAL775 | P | 2 phase | | 1.82 | 2.37 | C |
| 10C8.H5 | PAL776 | | 2 phase | | 1.15 | | |
| 16G1.A5 | PAL785 | C | 1.02 × 10⁻⁰⁹ | 6.42 × 10⁻¹⁰ | 4.98 | 0.68 | C |
| 16G1.D7 | PAL786 | | 1.45 × 10⁻⁰⁹ | 1.73 × 10⁻⁰⁹ | 2.97 | | |
| CKH-3D10 | PAL787(3D10) | P | 2.77 × 10⁻¹⁰ | 2.83 × 10⁻¹⁰ | 38.22 | 4.07 | P |
| CKH-3E3 | PAL788(3E3) | C | 1.88 × 10⁻¹⁰ | 3.92 × 10⁻¹⁰ | 24.81 | 0.89 | C |
| | Avelumab | C | 7.54 × 10⁻¹⁰ | 1.7 × 10⁻⁰⁹ | N/A | 0.38 | C |

### Example 3

This example describes the generation and characterization of chimeric PD-L1 antibodies. VH and VL gene sequences of hybridoma antibodies were isolated and sequenced. VL sequences are shown in TABLE 12, VH sequences in TABLE 13 and Light and Heavy Chain CDRs in TABLE 14. The DNA fragments coding for the V gene of interest were synthesized through conventional vendor. The VH and VL sequences were cloned into human IgG1 constant heavy chain backbone and human constant kappa light chain backbone respectively. The heavy chain and light chain DNA plasmids were transiently co-transfected in HEK293 cells to express the full IgGs.

TABLE 12
Encoded Variable Light sequences

| ID | VL |
|---|---|
| PAL | DIQMTQSSFSFSVSLGDRVTIICKASEDIYNRLAWYQQKPGN |
| 752 | TPRLLISGATSLETGVPSRFSGSGSGKDYTLSITSLQTEDVA |
| | TYYCQQYWSTPWTFGGGTKLEIK (SEQ ID NO: 136) |
| PAL | DILLTQSPAILSVSPGERVSFSCRASQSIGTSIHWYQQRTNG |
| 759 | SPRLLIKYASESISGIPSRFSGSGSGTDFTLSINSVESEDIG |
| | DYYCQQSNNWPFTFGSGTKLEIK (SEQ ID NO: 144) |
| PAL | DIVMTQSPASLAVSLGQKATISCKASKKVTIFGSISVLHWYQ |
| 760 | QKPGQPPKLIYNGAKLESGVSARFSDSGSQNRSPFGNQLNFT |
| | LTIDPVEADDAATYYCLQNKEVPYTFGGGTELEIK (SEQ |
| | ID NO: 152) |
| PAL | DIVMSQSPSSLAVSVGEKVTMSCKSSQSLLYSSNQKNSLAWY |
| 767 | QQKPGQSPKLLIYWASTRESGVPDRFTGSGSGTDFTLTISSV |
| | KAEDLAVYYCQQYYGYPWTFGGGTKLEIK (SEQ ID NO: |
| | 160) |
| PAL | SIVMTQTPKFLLVSAGDRVTITCKASQSVSNDVIWYQQKPGQ |
| 769 | SPKLLIYYASIRFTGVPDRFAGSGYGTDFTFTINTVQAEDLA |
| | VYFCQQDYNSPWTFGGGTKLEIK (SEQ ID NO: 167) |
| PAL | QIVLTQSPAIMSASPGEKVTMTCSASSSVSYMYWYQQKPGSS |
| 771 | PRLLIYDTSNLASGVPLRFSGSGSGTSYSLTLSRMEAEDAAT |
| | YYCQQWSTYPLTFGAGTKLELK (SEQ ID NO: 174) |
| PAL | DIVLTQSPASLAVSLGQRATISCRASESVEFYGTTLMQWYQQ |
| 785 | KPGQPPKLLIYAASNVESGVPARFSGSGSGTDFSLNIHPVEE |
| | GDIGMYFCQQSRKVPYTFGGGTKLEIK (SEQ ID NO: |
| | 182) |
| PAL | DIVMTQSQNFMSTSVGDRVSVTCKASHYVGTFVAWYQQKPGQ |
| 787 | SPKALIFSTSYRHTGVPDRFTGSGSGTDFTLTISNVQSEDLA |
| | DYFCQQYYNSPLTFGAGTKLELK (SEQ ID NO: 190) |
| PAL | NIVLTQSPASLAVSLGQRATISCRASESVDSYGNSFMHWYQQ |
| 788 | KPGQPPKLLIYLASNLQSGVPARFSGSGSRTDFTLTIDPVEA |
| | DDAATYYCQQNNEDPWTFGGGTKLEIK (SEQ ID NO: |
| | 198) |

TABLE 13
Encoded Variable Heavy sequences

| ID | VH |
|---|---|
| PAL | EVQLQESGAELARPGASVKLSCKASGHAFTSDSINWVKQRIG |
| 752 | QGLEWIGEIYPRSGNPYYNEKFKGKATLTADKSSSTAYMELR |
| | SLTSEDSAVYFCATDYYGRYFDVWGTGTTVTVSS (SEQ ID |
| | NO: 132) |
| PAL | EVQLQESGAELVRPGASVKLSCKASGYSFTDYYINWVKQRPG |
| 759 | QGLEWIARIYPGSGNTYYNEKFKGKATLTAEKSSITAYMQLS |
| | SLTSEDSAVYFCARSYYYGSSYLFDYWGQGTTLTVSS (SEQ |
| | ID NO: 140) |
| PAL | EVQLQQSGPELVKPGALVKISCKASGYTFTDYYMNWVKKSHG |
| 760 | RSLEWIGDINPNNGYTNYNQNFKGKATLTVDKSSSTVYMELR |
| | SLTSEDSAVYYCARSAAYYVLDDWGQGTSVTVSS (SEQ ID |
| | NO: 148) |
| PAL | EVQLQESGPSLVKPSQTLSLTCSVTGDSITSGYWNWIRKFPG |
| 767 | NKLEYMGYISYTGSTYYNPSLKRRISITRDTSKNQYYLQLNS |
| | VTTEDTATYYCASQGGWLQAMDYWGQGTSVTVSS (SEQ ID |
| | NO: 156) |
| PAL | EVQLQESGAELVKPGASVTLSCTASGFNIKDTYMHWVKQRPE |
| 769 | QGLEWIGRIDPANDNTKYDPKFQDKATITADTSSDTAYLRLS |
| | SLTSEDTAVYYCAREGYGGSYGEGYWGQGTTLTVSS (SEQ |
| | ID NO: 164) |
| PAL | EVQLQESGAELVKPGASVKLSCTASGFNIKDTYMHWVKQRPE |
| 771 | QGLEWIGRIDPANGNTKYDPKFPGKATITADTSSNTAYLQLS |
| | SLTSEDAAVYYCARPFNYRFYDVYYFDYWGQGTTLTVST |
| | (SEQ ID NO: 170) |
| PAL | EVQLQESGPELVKPGTSVKMSCKASGYTFTSYVMHWVKQRPG |
| 785 | QGLEWIGYINPYNDGSKYNEKFKGKATLTSDTSSSTAYMELS |
| | SLTSEDSAVYYCAKQTLDFWGQGTSVTVST (SEQ ID NO: |
| | 178) |
| PAL | QVTLKESGPGILQPSQTLSLTCSFSGFSLSTYGLGVGWIRQP |
| 787 | SGKGLEWLANIWWNDDKFYDSVLKSRLTISKDTSNNQVFLKI |
| | SSVDTSETATYYCAQTLHYYDGIAWFAYWGQGTLVTVSA |
| | (SEQ ID NO: 186) |
| PAL | QVQLQQPGAELVKPGASVKLSCKASGYTFTSNWMNWVKQRPG |
| 788 | RGLEWIGRIHPSDSETHYHQKFKSKATLTVDKSSSTAYIQLS |
| | SLTSEDSAVYYCAHSSGDYGRDYWGQGTTLTVSS (SEQ ID |
| | NO: 194) |

TABLE 14
Light and Heavy Chain CDRs

| ID | CDR-L1 | CDR-L2 | CDR-L3 | CDR-H1 | CDR-H2 | CDR-H3 |
|---|---|---|---|---|---|---|
| PAL752 | EDIYNR | GAT (SEQ | QQYWSTPWT | GHAFTSDS | IYPRSGNP | DYYGRYFD |
| | (SEQ ID NO: | ID NO: | (SEQ ID NO: | (SEQ ID NO: | (SEQ ID NO: | V (SEQ ID |
| | 133) | 134) | 135) | 129) | 130) | NO: 131) |
| PAL759 | QSIGTS (SEQ | YAS (SEQ | QQSNNWPFT | GYSFTDYY | IYPGSGNT | SYYYGSSYL |
| | ID NO: 141) | ID NO: | (SEQ ID NO: | (SEQ ID NO: | (SEQ ID NO: | FDY (SEQ ID |
| | | 142) | 143) | 137) | 138) | NO: 139) |
| PAL760 | KKVTIFGSIS | NGA (SEQ | LQNKEVPYT | GYTFTDYY | INPNNGYT | SAAYYVLD |
| | V (SEQ ID | ID NO: | (SEQ ID NO: | (SEQ ID NO: | (SEQ ID NO: | D (SEQ ID |
| | NO: 149) | 150) | 151) | 145) | 146) | NO: 147) |
| PAL767 | QSLLYSSNQ | WAS (SEQ | QQYYGYPWT | GDSITSGY | ISYTGST | QGGWLQAM |
| | KNS (SEQ ID | ID NO: | (SEQ ID NO: | (SEQ ID NO: | (SEQ ID NO: | DY (SEQ ID |
| | NO: 157) | 158) | 159) | 153) | 154) | NO: 155) |
| PAL769 | QSVSND | YAS (SEQ | QQDYNSPWT | GFNIKDTY | IDPANDNT | EGYGGSYGE |
| | (SEQ ID | ID NO: | (SEQ ID NO: | (SEQ ID NO: | (SEQ ID NO: | GY (SEQ ID |
| | NO:165) | 142) | 166) | 161) | 162) | NO: 163) |
| PAL771 | SSVSY (SEQ | DTS (SEQ | QQWSTYPLT | GFNIKDTY | IDPANGNT | PFNYRFYDV |
| | ID NO: 171) | ID NO: | (SEQ ID NO: | (SEQ ID NO: | (SEQ ID NO: | YYFDY (SEQ |
| | | 172) | 173) | 161) | 168) | ID NO: 169) |
| PAL785 | ESVEFYGTT | AAS (SEQ | QQSRKVPYT | GYTFTSYV | INPYNDGS | QTLDF (SEQ |
| | L (SEQ ID | ID NO: | (SEQ ID NO: | (SEQ ID NO: | (SEQ ID NO: | ID NO: 177) |
| | NO: 179) | 180) | 181) | 175) | 176) | |
| PAL787 | HYVGTF | STS (SEQ | QQYYNSPLT | GFSLSTYGLG | IWWNDDK | TLHYYDGIA |
| | (SEQ ID NO: | ID NO: | (SEQ ID NO: | (SEQ ID NO: | (SEQ ID NO: | WFAY (SEQ |
| | 187) | 188) | 189) | 183) | 184) | ID NO: 185) |
| PAL788 | ESVDSYGNS | LAS (SEQ | QQNNEDPWT | GYTFTSNW | IHPSDSET | SSGDYGRDY |
| | F (SEQ ID | ID NO: | (SEQ ID NO: | (SEQ ID NO: | (SEQ ID NO: | (SEQ ID NO: |
| | NO: 195) | 196) | 197) | 191) | 192) | 193) |

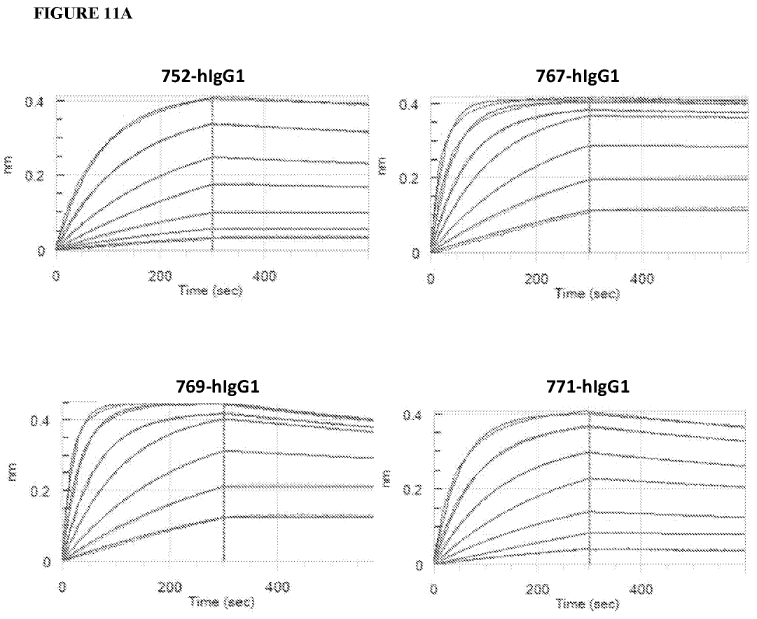

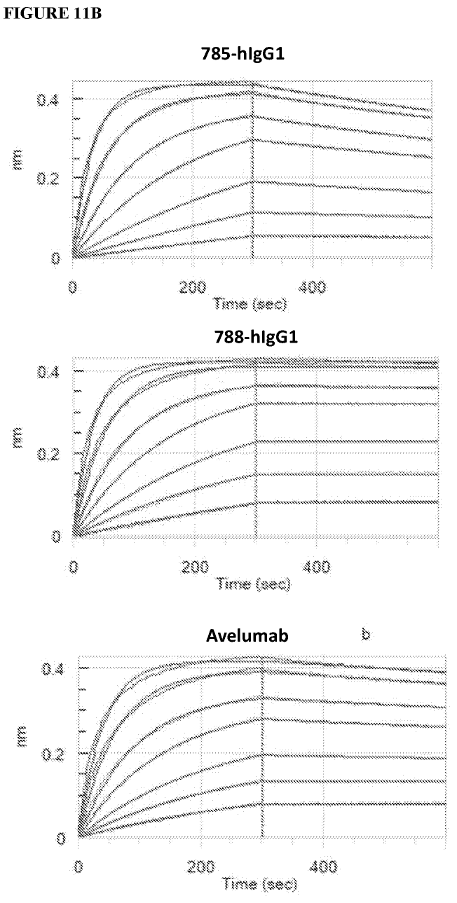

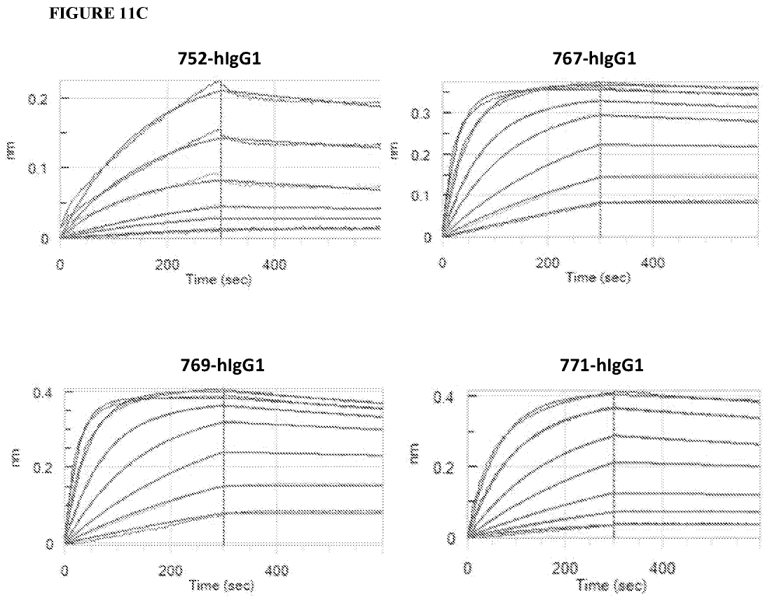

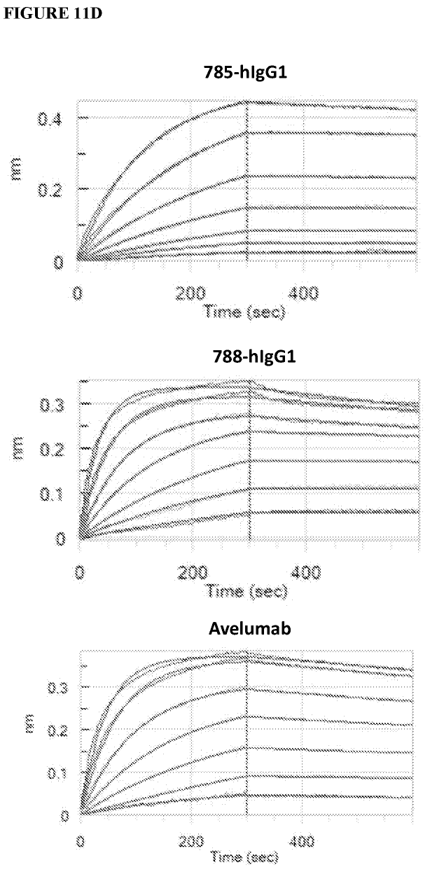

Characterization of chimeric anti-PD-L1 antibodies. ForteBio octet binding of purified chimeric antibodies and recombinant hPD-L1-his and cPD-L1-his was measured. Chimeric human antibodies were captured on AHC (Anti-human IgG-Capture) Biosensor, hPD-L1-his or cPD-L1-his analytes were titrated from 100 nM in a 2× series dilution. The buffer reference was subtracted from the signal and aligned to the baseline. KD, Kon and Koff values were generated using 1:1 fitting model as described above. The binding kinetics of selected chimeric antibodies are shown in FIG. 11A and FIG. 11B for hPD-L1 and FIG. 11C and FIG. 11D for cPD-L1. The calculated KDs are shown in TABLE 15 for hPD-L1 and cPD-L1.

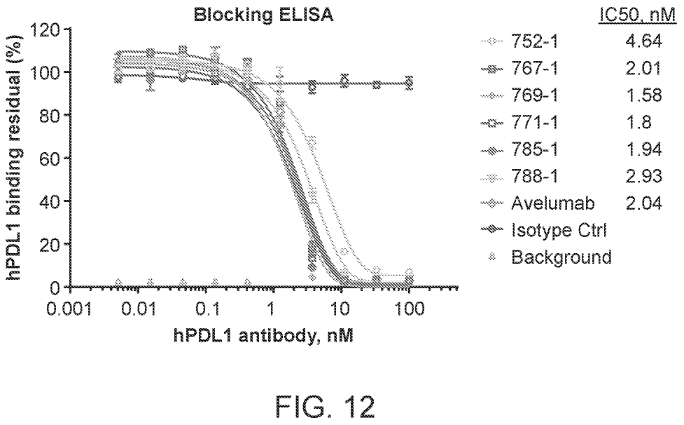

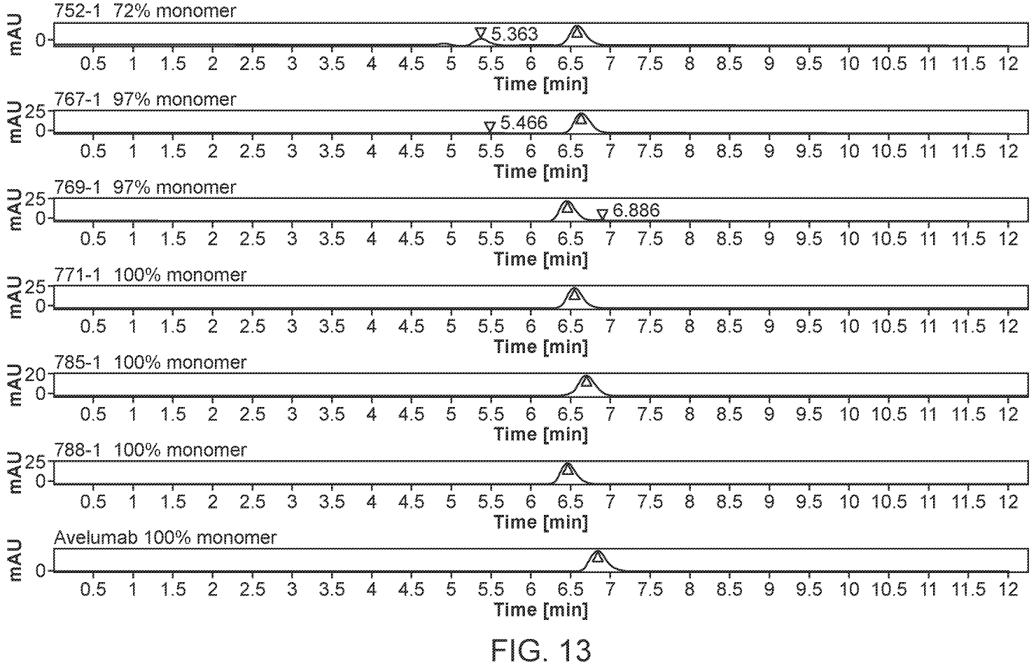

Purified chimeric antibodies were tested for their ability to block biotinylated hPD-1-Fc from binding to hPD-L1 as described above. FIG. 12 depicts the results and the calculated IC₅₀s, all of which are in the single digit nM range. Protein A purified chimeric antibodies were examined by size exclusion chromatography and quantified with a UV-Vis spectrophotometer (NanoDrop; mAU=milli absorbance units). FIG. 13 depicts UV traces of size exclusion chromatographs with the indicated amount of monomeric peaks at the expected retention times.

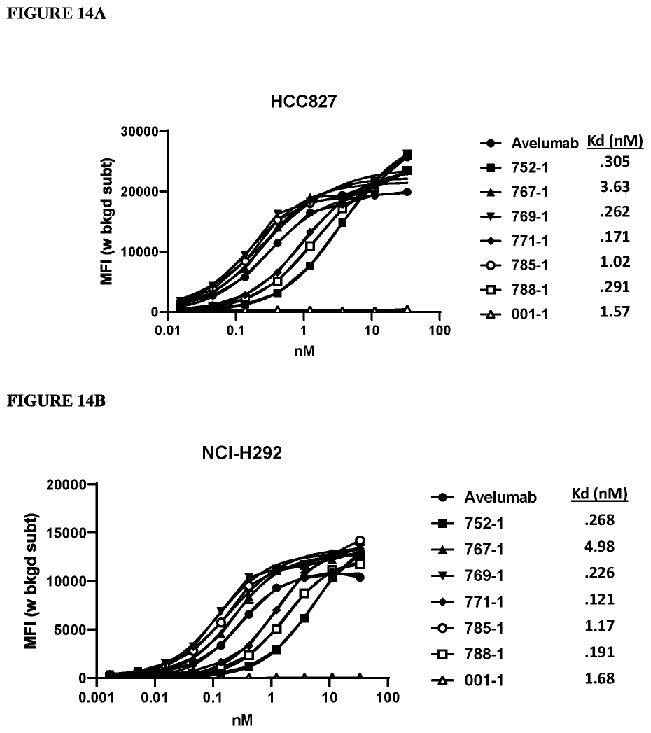

Purified chimeric antibodies were evaluated for their ability to bind PD-L1 expressed on two human cancer cell lines. HCC827 and NCI-292 cells were incubated with titrated antibodies for 30 min at 4° C. Cells were washed and incubated with AF647-labeled goat anti-human IgG(H+L) for 30 min at 4° C. Cells were washed, fixed and analyzed on FACSCelesta. FIG. 14 depicts the binding curves and calculated Kd for selected chimeric antibodies to HCC827 cells (FIG. 14A) and NCI-292 cells (FIG. 14B).

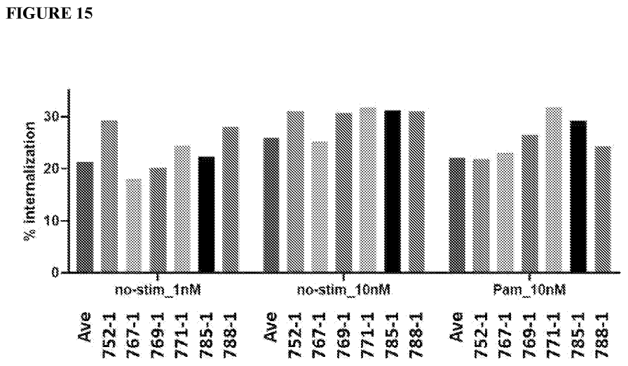

Purified chimeric antibodies were evaluated for their ability to bind and be internalized by human dendritic cells (DC). Monocyte-derived DC (moDC) were either (1) stimulated with Pam3CSK4 at 250 ng/ml before the day of experiment or (2) not stimulated. Cells were blocked for 30 min at room temperature, and incubated with 1 nM or 10 nM antibodies for 30 min on ice. Cells were washed and divided into two equal portions for a 2-hour incubation on either ice or at 37° C. Cells were washed and incubated with goat anti-human IgG(H+L) for 30 min on ice. Cells were washed, fixed, and analyzed on FACSCelesta. Percent internalization was determined as the reduction of bound antibody on cells after 2 hour incubation at 37° C. as compared to 4° C. FIG. 15 depicts the % internalization for the indicated chimeric antibodies under the different conditions. All antibodies had a relatively limited rate of internalization, between 20% and 30%, after 2 hours including with the stimulated cells.

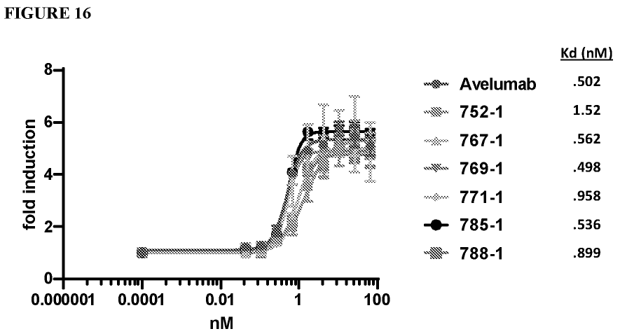

Purified chimeric antibodies were evaluated for their ability to functionally block PD-1 binding to PD-L1 as described above. Purified chimeric PD-L1 antibodies were tested in co-culture with (i) engineered CHO-K1 cells expressing human PD-L1 and TCR activating protein and (ii) Jurkat T cells expressing human PD-1, TCR and a luciferase reporter driven by an NFAT response element. As shown in FIG. 16, the tested chimeric antibodies disrupted the PD-L1/PD-1 interaction, resulting in a dose dependent increase in luminescence. Kds of the PD-L1 antibodies were all single digit nM or sub nM.

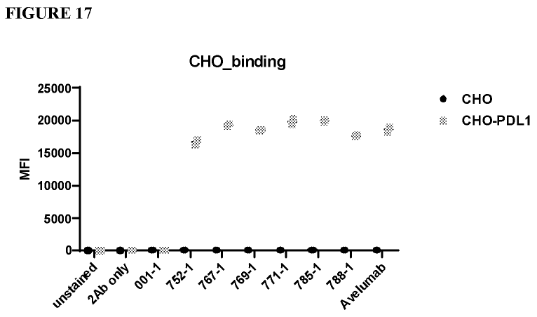

The specificity of the antibodies for cell surface expressed hPD-L1 was confirmed by comparing binding of antibodies to CHO cells expressing hPD-L1 vs wild type CHO cells. CHO cells were incubated with 100 nM antibodies and CHO-PD-L1 cells (from Bioassay) with 10 nM antibodies for 30 min at 4° C. Cells were washed and incubated with goat anti-human IgG(H+L) for 30 in at 4° C. The washed cells were fixed and run on FACSCelesta. FIG. 17 depicts the mean fluorescence intensity (MFI) of the indicated chimeric antibody as compared to isotype and other negative controls and avelumab. Specific staining to CHO-PD-L1 cells was seen for the PD-L1 antibodies with very little non-specific binding to CHO cells, even at high antibody concentrations.

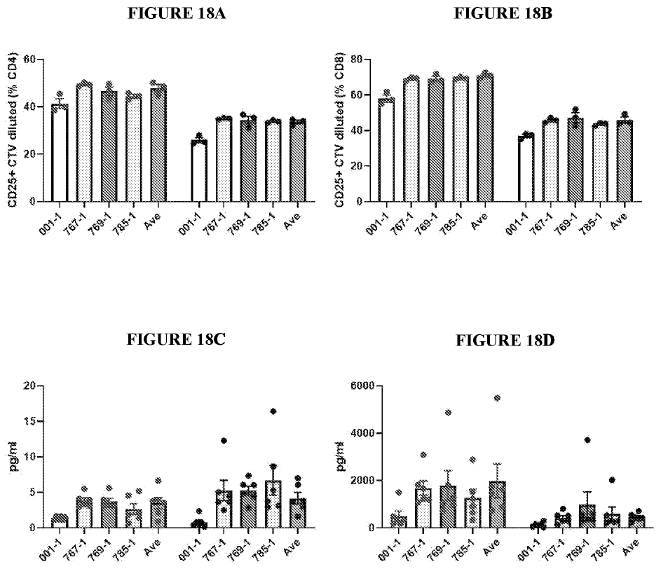

The ability of chimeric PD-L1 antibodies to modulate T cell function was tested. Monocyte-derived Dendritic Cells (moDC) were incubated with CellTrace Violet (CTV)-labeled allogeneic T cells in the presence of antibodies for 5 days. Proliferation was measured by FACSCelesta. Cytokine and cytolytic granules in supernatant were analyzed by multiplex bead-based assay. Each panel of FIGS. 18-20 shows two experiments: the left 5 bars show T cells from donor 1 responding to moDC from donor 2, and the right 5 bars show T cells from donor 2 responding to moDC from donor 1.

FIG. 18 depicts the enhancement of T cell proliferation and cytokine response to allogeneic moDC in the presence of the indicated PD-L1 antibodies compared to isotype control (001-1). FIG. 18 shows CD4 T cell proliferation (FIG. 18A), CD8 T cell proliferation (FIG. 18B), TNFα (FIG. 18C), and IFN-γ levels (FIG. 18D).

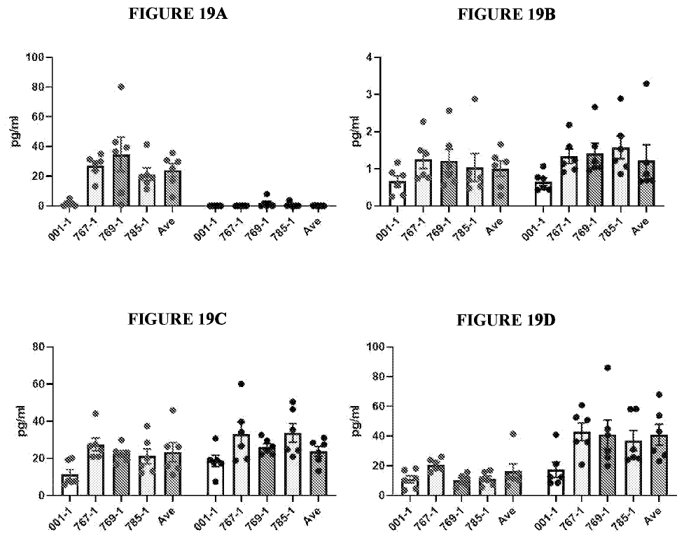

FIG. 19 depicts the enhancement of cytokine response to allogeneic moDC in the presence of indicated PD-L1 antibodies compared to isotype control (001-1). FIG. 19 shows IL-2 (FIG. 19A), IL-4 (FIG. 19B), IL-6 (FIG. 19C) and IL-10 levels (FIG. 19D).

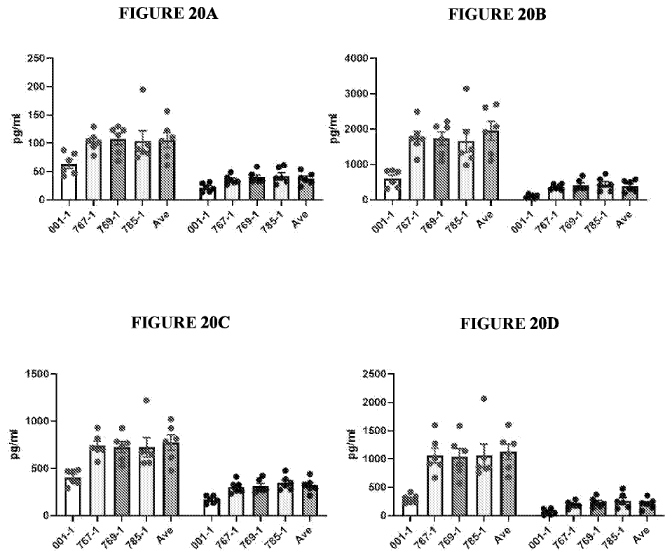

FIG. 20 depicts the enhancement of degranulation in moDC-T cell MLR in the presence of the indicated PD-L1 antibodies compared to isotype control (001-1). FIG. 20 shows soluble Fas Ligand (FIG. 20A), Granzyme A (FIG. 20B), perforin (FIG. 20C) and granulysin (FIG. 20D).

TABLE 15 is a summary of the biochemical and cellular activity of the chimeric PD-L1 antibodies.

TABLE 15
Anti-PD-L1 chimeric IgG characterization summary

| Characterizations | | 752-hIgG1 | 767-hIgG1 | 769-hIgG1 | 771-hIgG1 | 785-hIgG1 | 788-hIgG1 | Avelumab |
|---|---|---|---|---|---|---|---|---|
| Kd to hPD-L1, M | | 1.18 × 10⁻⁰⁹ | 5.22 × 10⁻¹¹ | 6.40 × 10⁻¹⁰ | 1.43 × 10⁻⁰⁹ | 1.64 × 10⁻⁰⁹ | 4.30 × 10⁻¹¹ | 5.53 × 10⁻¹⁰ |
| Kd to cPD-L1, M | | 3.18 × 10⁻⁰⁹ | 2.66 × 10⁻¹⁰ | 5.50 × 10⁻¹⁰ | 1.12 × 10⁻⁰⁹ | 7.24 × 10⁻¹⁰ | 8.67 × 10⁻¹⁰ | 9.94 × 10⁻¹⁰ |
| IC₅₀, nM (ELISA) | | 4.6 | 2 | 1.6 | 1.8 | 1.9 | 2.9 | 2 |
| IC₅₀, nM (Function) | | 1.5 | 0.6 | 0.5 | 1 | 0.5 | 0.9 | 0.5 |
| Monomer % (SEC) | | 72% | 97% | 97% | 100% | 100% | 100% | 100% |
| Bind PD-L1-CHO/CHO | | >100x | >100x | >100x | >100x | >100x | >100x | >100x |
| PD-L2 binding | | No | No | No | No | No | No | No |
| HCC827 Kd, nM | | 3.63 | 0.26 | 0.17 | 1.02 | 0.29 | 1.57 | 0.3 |
| NCI-H292 Kd, nM | | 4.98 | 0.23 | 0.12 | 1.17 | 0.19 | 1.68 | 0.27 |
| DC Int % | NS, 10 nM | 10.19 | 9.15 | 12.19 | 16.95 | 19.01 | 12.48 | 6.04 |
| Donor1 | NS, 1 nM | 14.29 | 9.34 | 4.51 | 17.52 | 16.87 | 13.96 | 7.31 |
| | Pam, 10 nM | 0.94 | 1.13 | 1.34 | 6.85 | 3.44 | −3.44 | −3.47 |
| DC Int % | NS, 1 nM | 29.19 | 18.05 | 20.17 | 24.5 | 22.37 | 28.07 | 21.33 |
| Donor2 | NS, 10 nM | 31.14 | 25.14 | 30.67 | 31.82 | 31.27 | 31.02 | 25.97 |
| | Pam, 10 nM | 21.77 | 22.99 | 26.45 | 31.79 | 29.27 | 24.33 | 21.98 |

### Example 4

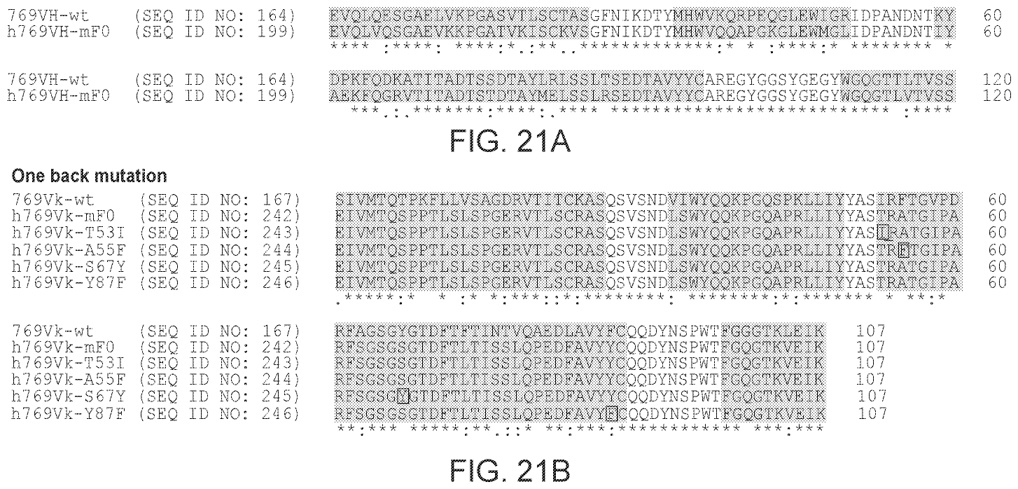

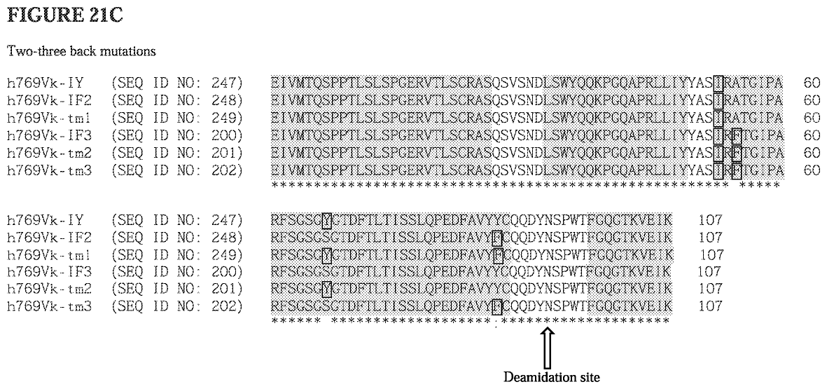

This example describes the PD-L1 antibody humanization. FIG. 21A depicts the PAL769 VH sequence in mouse frameworks (769VH-wt; SEQ ID NO: 164) compared to the VH sequence in human frameworks (h769VH-mF0; SEQ ID NO: 199). CDRs identified by IMGT are shown in red (GFNIKDTY (SEQ ID NO: 161; IDPANDNT (SEQ ID NO: 162; and AREGYGGSYGEGY (amino acids 97-109 of SEQ ID NO: 164). Note that CDRs provided elsewhere in the application may be identified by other definitions (e.g., Kabat) and may vary. FIG. 21B depicts the PAL769 VL sequence in mouse frameworks (769Vk-wt; SEQ ID NO: 167) compared to the VL sequence in human frameworks (h769Vk-mF0; SEQ ID NO: 242). Highlighted amino acids in 769Vk-wt were back mutated and tested for activity (data not shown). A series of single back mutations (h769Vk-T53I (SEQ ID NO: 243); h769Vk-A55F (SEQ ID NO: 244); h769Vk-S67Y (SEQ ID NO: 245); h769Vk-Y87F (SEQ ID NO: 246)) chosen for further study are also shown. FIG. 21C depicts a series of 2 or 3 back mutations as well as a potential deamidation motif on CDR-L3 (h769Vk-IY (SEQ ID NO: 247); h769Vk-IF2 (SEQ ID NO: 248); h769Vk-tm1 (SEQ ID NO: 249); h769Vk-IF3 (SEQ ID NO: 200); h769Vk-tm2 (SEQ ID NO: 201); h769Vk-tm3 (SEQ ID NO: 202)).

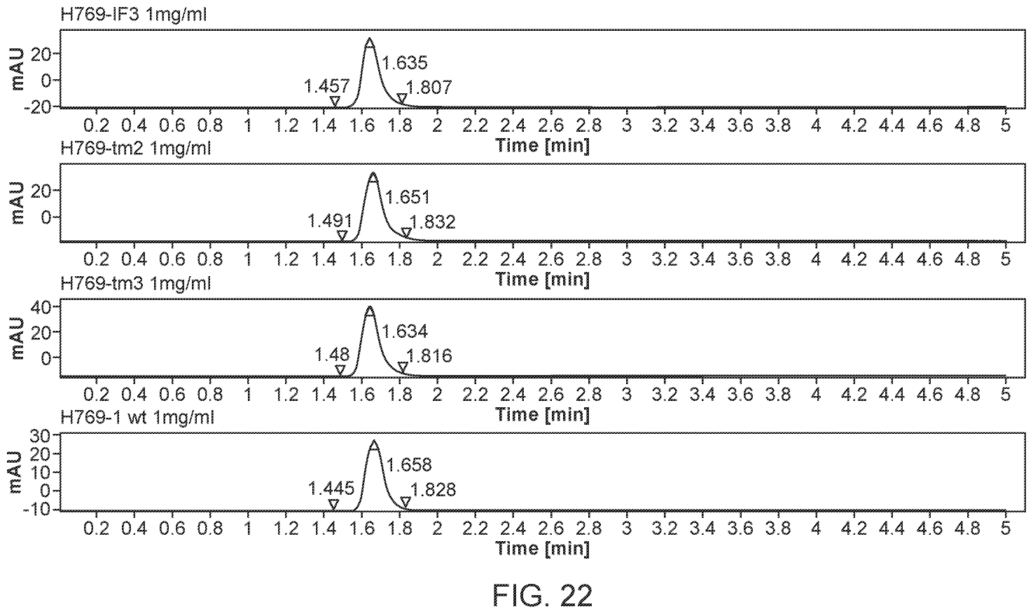

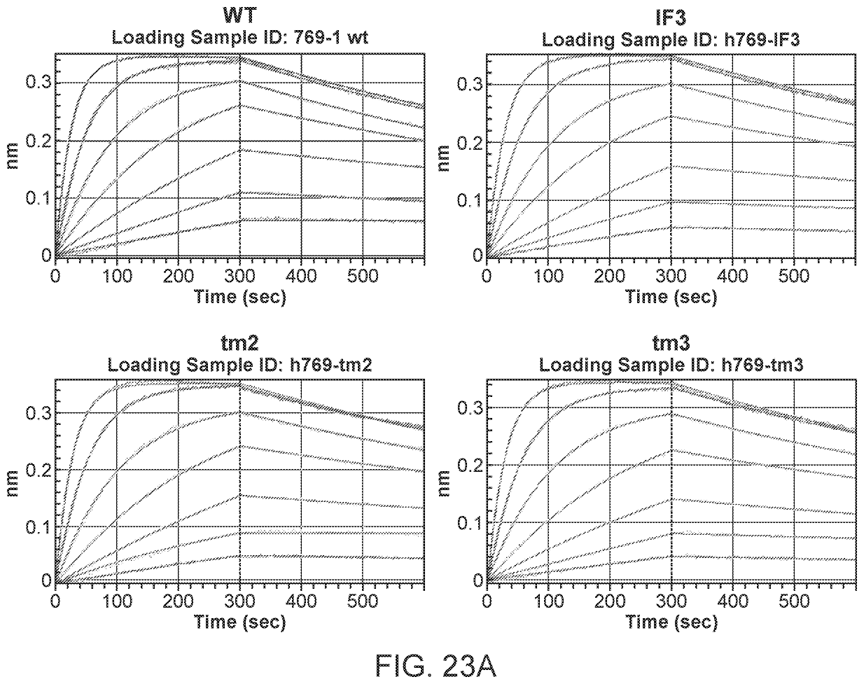

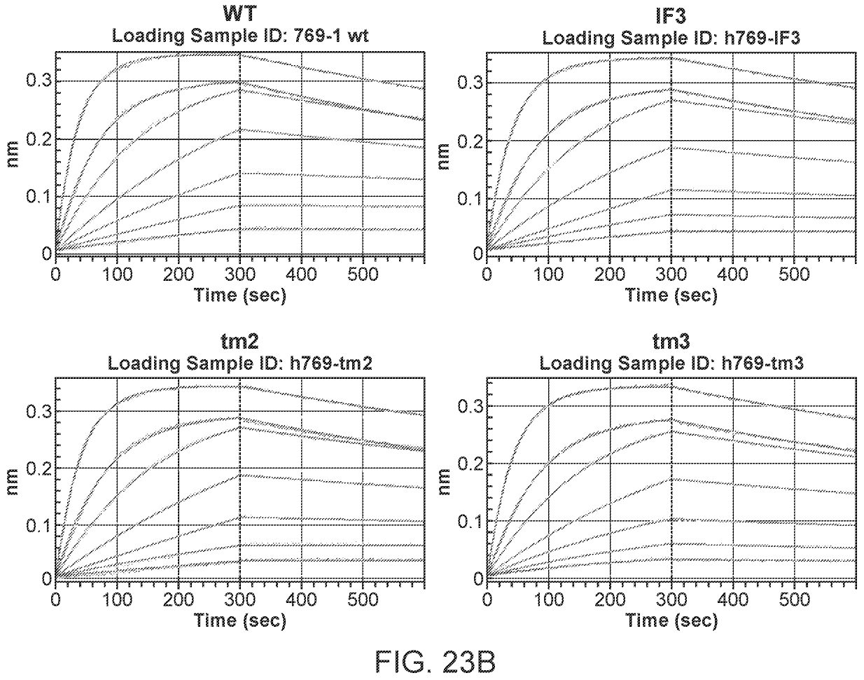

Selected h769 mutations were produced and purified. FIG. 22 depicts UV traces of size exclusion chromatographs of a selected group of humanized PD-L1 antibodies with monomeric peaks at the expected retention times. As described supra, ForteBio octet binding of purified humanized antibodies to recombinant hPD-L1-his and cPD-L1-his was measured with curves for hPD-L1 shown in FIG. 23A and for cPD-L1 shown in FIG. 23B. KD, Kon and Kdis values for human and cyno PD-L1 are shown in TABLE 16.

TABLE 16

| | Human PD-L1 | | | Cyno PD-L1 | | |
|---|---|---|---|---|---|---|
| Clone ID | KD (M) | kon(1/Ms) | kdis(1/s) | KD (M) | kon(1/Ms) | kdis(1/s) |
| 769-wt | 2.59 × 10⁻⁰⁹ | 3.73 × 10⁵ | 9.67 × 10⁻⁰⁴ | 2.92 × 10⁻⁰⁹ | 2.38 × 10⁵ | 6.85 × 10⁻⁰⁴ |
| h769-IF3 | 2.64 × 10⁻⁰⁹ | 3.30 × 10⁵ | 8.71 × 10⁻⁰⁴ | 2.92 × 10⁻⁰⁹ | 2.08 × 10⁵ | 6.02 × 10⁻⁰⁴ |
| h769-tm2 | 2.48 × 10⁻⁰⁹ | 3.31 × 10⁵ | 8.18 × 10⁻⁰⁴ | 3.01 × 10⁻⁰⁹ | 2.04 × 10⁵ | 5.93 × 10⁻⁰⁴ |
| h769-tm3 | 2.95 × 10⁻⁰⁹ | 3.11 × 10⁵ | 9.04 × 10⁻⁰⁴ | 3.11 × 10⁻⁰⁹ | 2.11 × 10⁵ | 6.50 × 10⁻⁰⁴ |

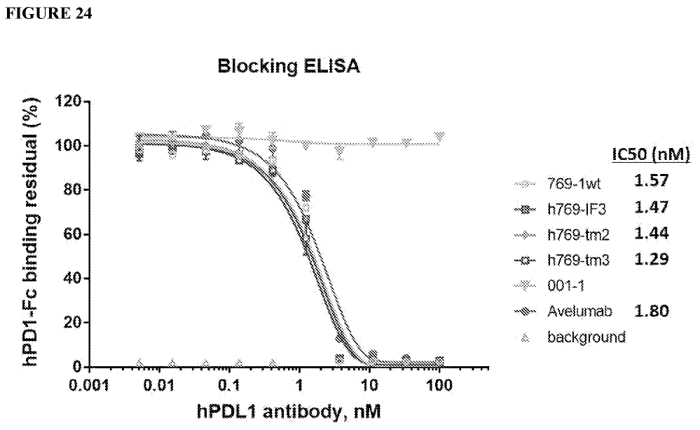

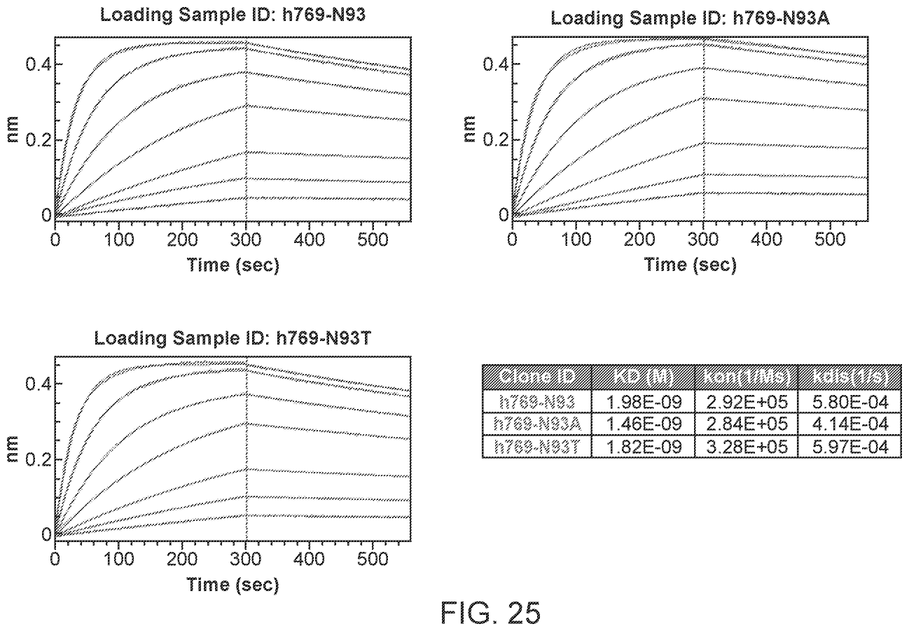

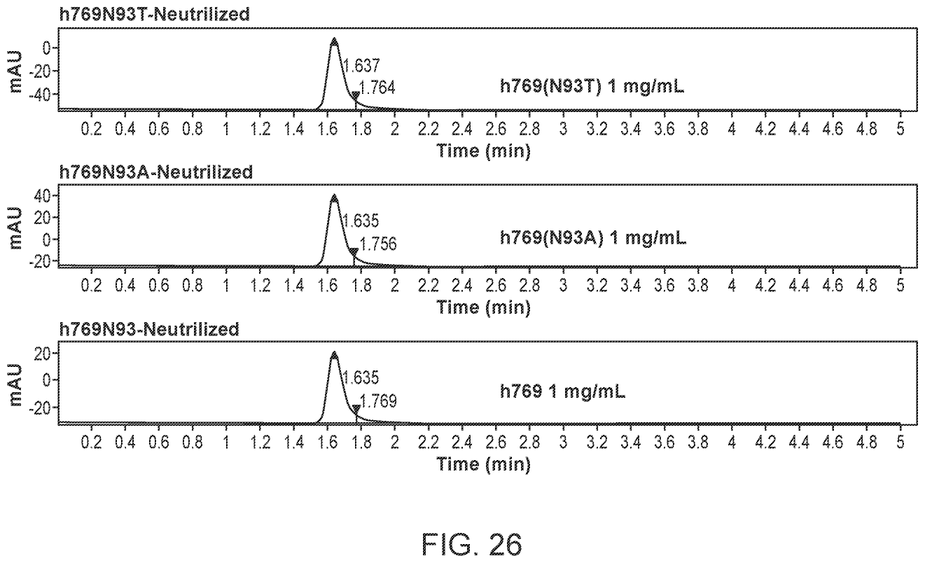

Selected h769 antibodies were tested in the blocking ELISA as described in Example 3 herein. Results are shown in FIG. 24 and calculated IC₅₀s (nM) are indicated. Selected h769 humanized PD-L1 antibodies were characterized following removal of the deamidation motif in CDR-L3. FIG. 25 depicts the ForteBio octet binding of purified humanized antibodies and recombinant hPD-L1-his was measured. KD, Kon and Kdis values for human PD-L1 are shown in TABLE 17. FIG. 26 depicts UV traces of size exclusion chromatographs of monomeric peaks at the expected retention times.

TABLE 17

| Clone ID | KD (M) | kon(1/Ms) | kdis(1/s) |
|---|---|---|---|
| h769-N93 | 1.98 × 10⁻⁰⁹ | 2.92 × 105 | 5.80 × 10⁻⁰⁴ |
| h769-N93A | 1.46 × 10⁻⁰⁹ | 2.84 × 105 | 4.14 × 10⁻⁰⁴ |
| h769-N93T | 1.82 × 10⁻⁰⁹ | 3.28 × 105 | 5.97 × 10⁻⁰⁴ |

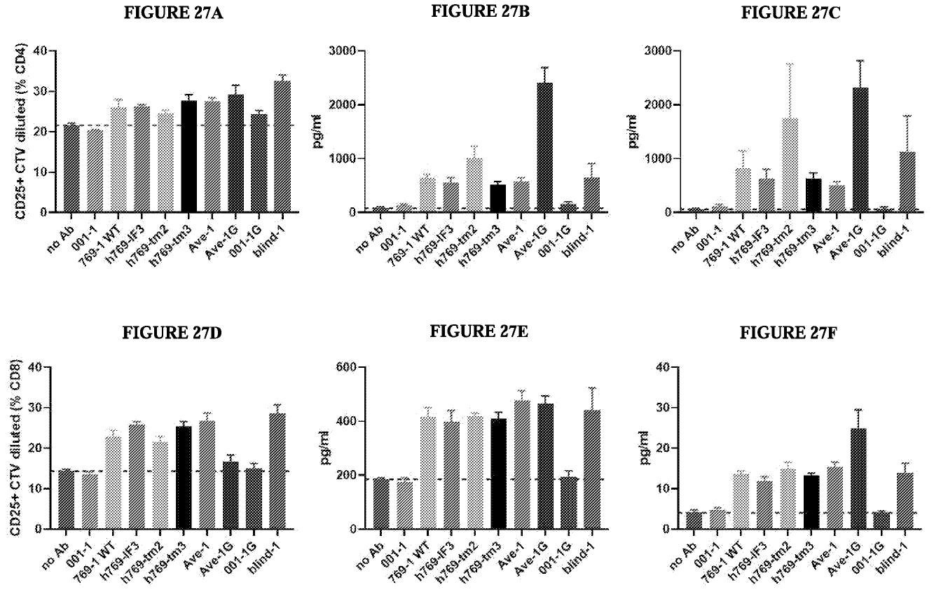

FIG. 27 shows that selected 769-hIgG1 humanized variants enhance T cell response to allogeneic moDC. Avelumab was also tested in both an IgG1 format as well as a IgG1 N297G format. PAL-767-1 was also used as a control (labeled blind in figures). FIG. 27 shows CD4 T cell proliferation (FIG. 27A), Granzyme B (FIG. 27B), and IFN-γ (FIG. 27C) as well as CD8 T cell proliferation (FIG. 27D), Granzyme A (FIG. 27E) and TNFα levels (FIG. 27F).

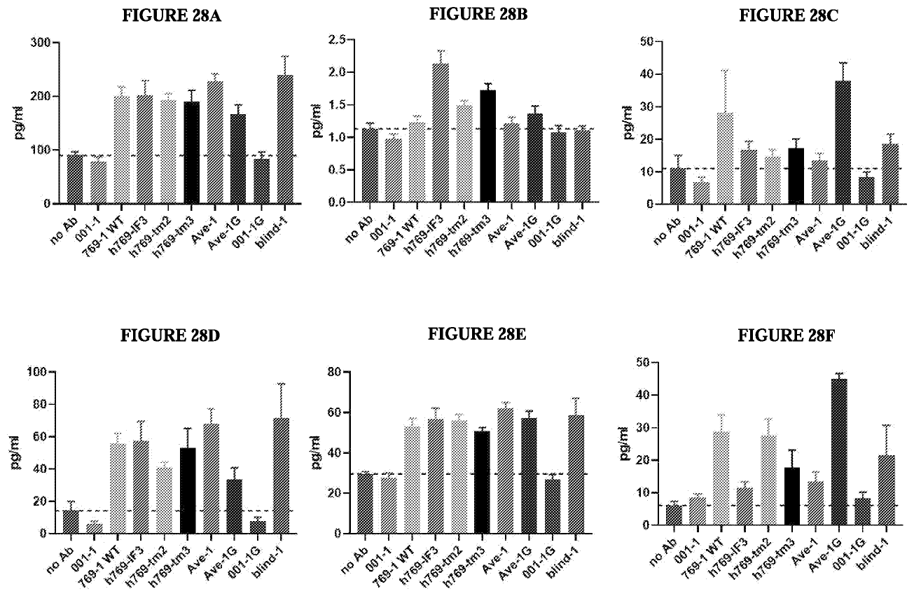

FIG. 28 shows that selected 769-hIgG1 humanized variants enhance T cell response to allogeneic moDC. FIG. 28 shows Perforin (FIG. 28A), soluble Fas (FIG. 28B), IL-6 (FIG. 28C), Granulysin (FIG. 28D), soluble Fas Ligand (FIG. 28E) and IL-10 levels (FIG. 28F).

Selected 769-hIgG1 humanized variants were tested for their ability to enhance PBMC cytokine responses to CMV pp65. PBMCs were incubated with or without CMV pp65 protein stimulation in the presence of antibodies for 4 days. Cytokine and cytolytic granules in supernatant were analyzed by multiplex bead-based assay.

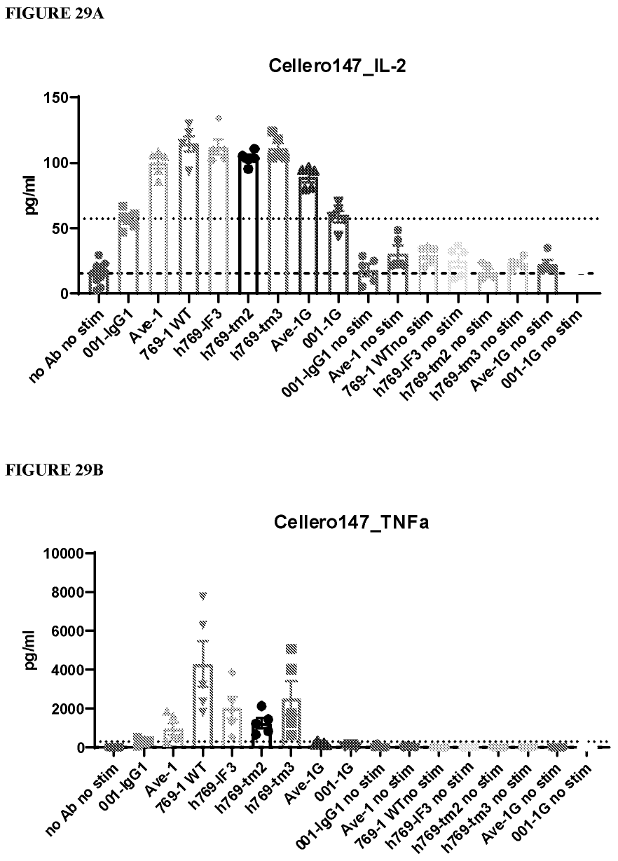

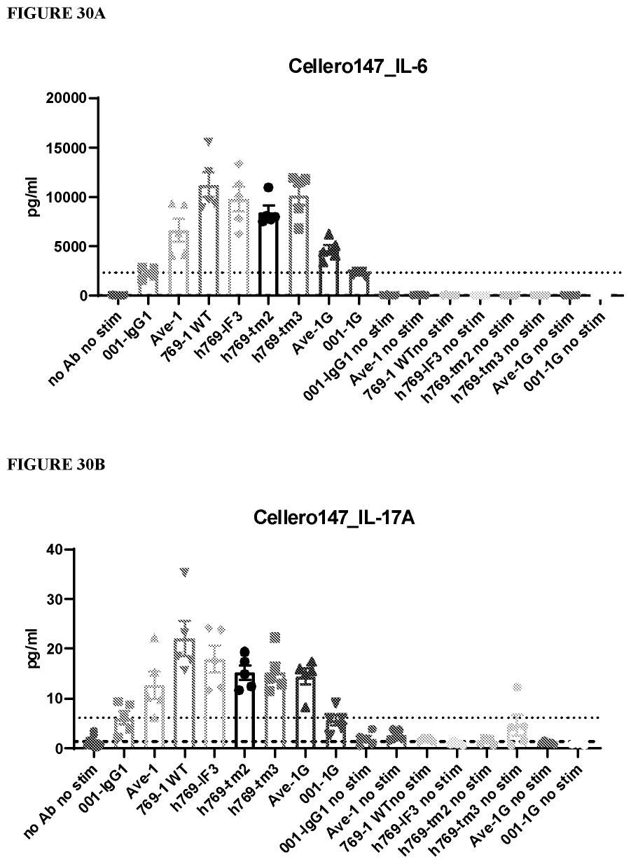

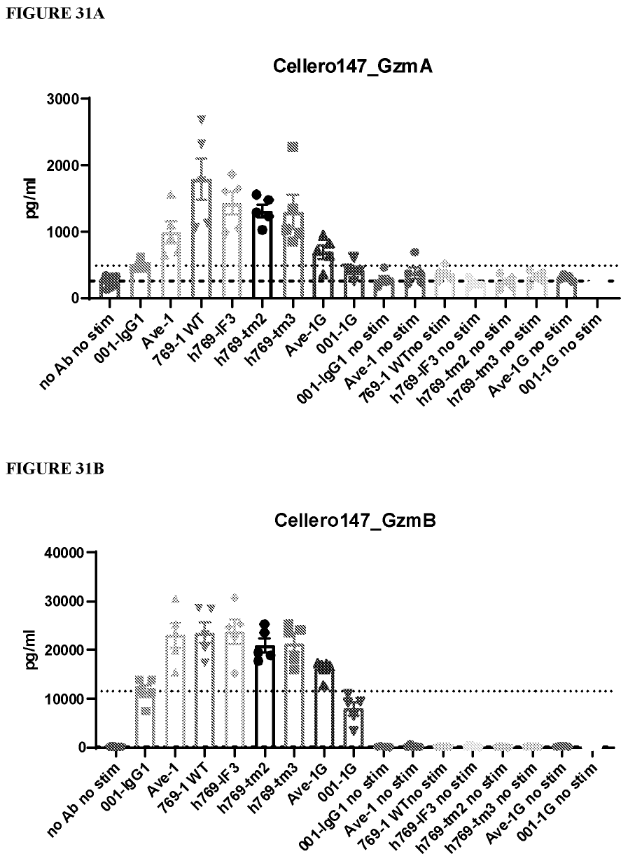

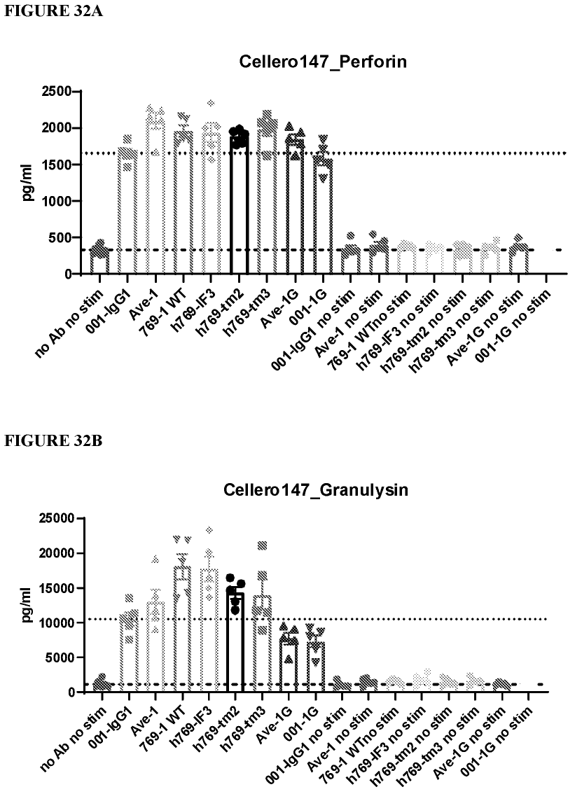

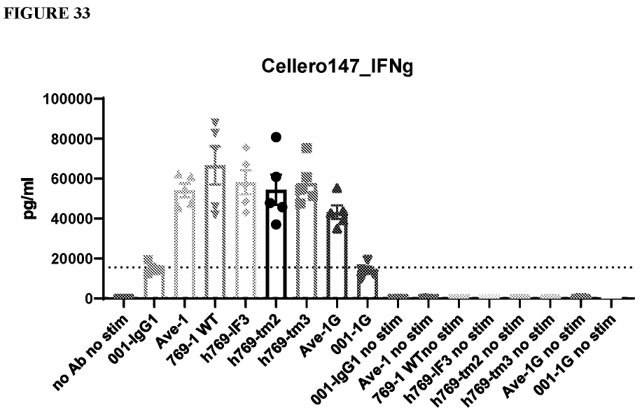

FIGS. 29-33 show that selected 769-hIgG1 humanized variants enhance PBMC cytokine responses to CMV pp65, including by increasing levels of IL2, TNFα, IL-6, IL-17A, Granzyme A, Granulysin, and IFN-γ. These results suggest that the 769-hIgG1 humanized variants may be capable of enhancing an immune response in PBMCs. Specifically, FIG. 29 shows that selected 769-hIgG1 humanized variants enhance PBMC cytokine responses to CMV pp65. Levels of IL-2 (FIG. 29A) and TNFα (FIG. 29B) are shown. FIG. 30 shows that selected 769-hIgG1 humanized variants enhance PBMC cytokine responses to CMV pp65. Levels of IL-6 (FIG. 30A) and IL-17A (FIG. 30B) are shown. FIG. 31 shows that selected 769-hIgG1 humanized variants enhance PBMC cytokine responses to CMV pp65. Levels of Granzyme A (FIG. 31A) and Granzyme B (FIG. 31B) are shown. FIG. 32 shows that selected 769-hIgG1 humanized variants enhance PBMC cytokine responses to CMV pp65. Levels of Perforin (FIG. 32A) and Granulysin (FIG. 32B) are shown. FIG. 33 shows that selected 769-hIgG1 humanized variants enhance PBMC IFN-γ response to CMV pp65.

Next, an epitope binning sandwich assay was developed to determine whether the epitope of h769.T-1A overlapped with PAL-752, PAL-767, PAL-769, PAL-771, PAL-785 or PAL-788. h769.T-1A includes: the variable region h769.T (also referred to as h769-N93T), which includes a heavy chain variable region of SEQ ID NO: 199 and light chain variable region of SEQ ID NO: 204; and a human IgG1 constant region including a N297A mutation. The mouse IgG hybridoma version of each antibody was tested. The assay was performed as follows:
Step 1: 1st antibody of mouse IgGs was captured on AMC biosensor;
Step 2: binding to hPD-L1-his antigen; and
Step 3: binding to h769.T-1A.

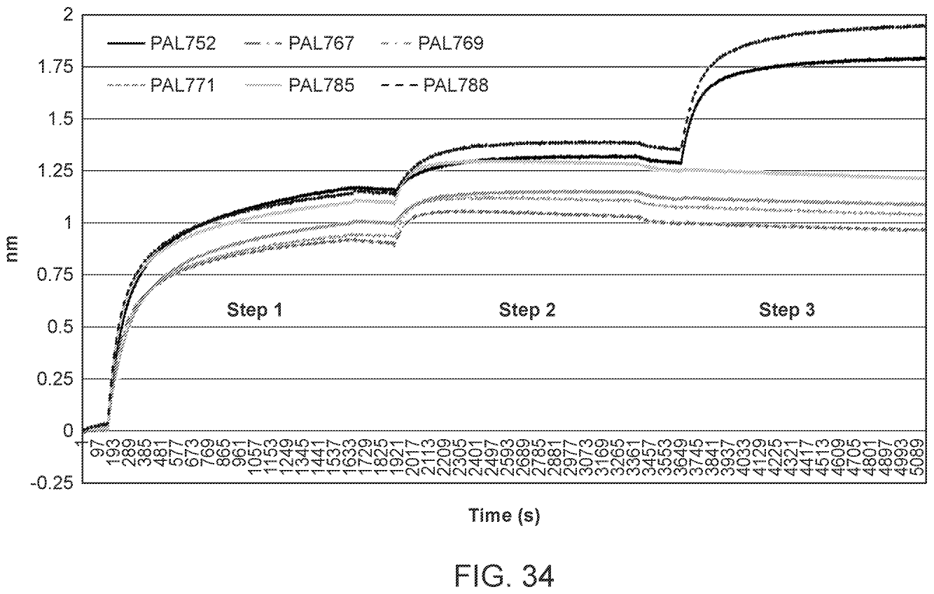

As shown in FIG. 34, hPD-L1-his that was bound to PAL752 (second to top line) and PAL788 (top line) can still bind to h769.T-1A, suggesting that these two antibody epitopes do not overlap with the epitope of h769.T-1A, and that PAL767, PAL769, PAL771 and PAL785 have shared or overlapping epitopes with h769.T-1A.

### Example 5

This example describes the construction of PD-L1 antibody sialidase conjugates.

An exemplary configuration of an anti-PD-L1 antibody ASC is referred to as “Janus,” and contains one antibody arm (with one heavy chain and one light chain), and one sialidase-Fc fusion with a sialidase fused at the N-terminus of one arm of the Fc. Each Fc domain polypeptide in the Janus ASC contains either the “knob” (T366Y) or “hole” (Y407T) mutation for heterodimerization (residue numbers according to EU numbering, Kabat, E. A., et al. (1991) supra) (see, e.g., FIG. 6B).

A Janus PD-L1 antibody sialidase conjugate was constructed using Neu2 with M1D, V6Y, P62G, A93E, I187K, and C332A mutations, the variable region of anti-PD-L1 antibody h769.T (as described in Example 4 herein, and also referred to as h769-N93T), and a human IgG1 Fc domain including an N297A mutation. This Janus PD-L1 antibody sialidase conjugate (referred to as ASC1, and including a first polypeptide chain with amino acid sequence SEQ ID NO: 205, encoded by nucleotide sequence SEQ ID NO: 208, a second polypeptide chain with amino acid sequence SEQ ID NO: 206, encoded by nucleotide sequence SEQ ID NO: 209, and a third polypeptide chain with amino acid sequence SEQ ID NO: 207, encoded by nucleotide sequence SEQ ID NO: 210) was expressed and characterized for purity using SDS-PAGE and enzymatic activity using 4MU-NeuAc as described below.

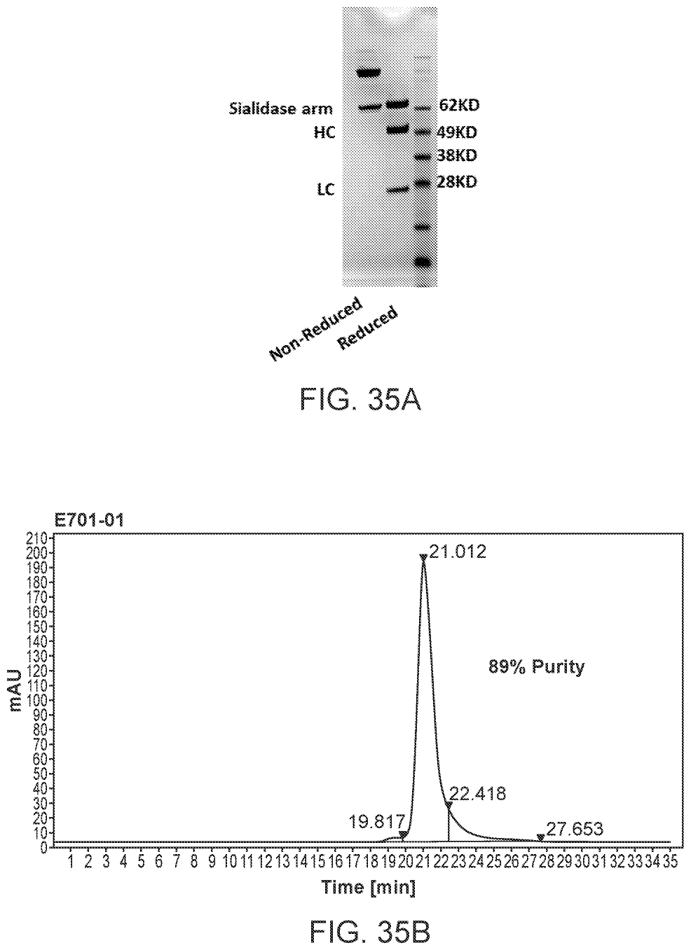

ASC1 was expressed in a 1,000 mL transfection of Expi293 human cells using the pCEP4 mammalian expression vector. The PD-L1 antibody sialidase conjugate was purified using protein A followed by Ceramic Hydroxyapatite chromatography, quantified with a UV-Vis spectrophotometer (NanoDrop), and examined by SDS-PAGE as shown in FIG. 35A. ASC1 expressed well with an 89% purity after purification (FIG. 35B).

The activity of ASC1 was assayed by measuring the release of sialic acid from the fluorogenic substrate 4-methylumbelliferyl-N-acetylneuraminic acid (4MU-NeuAc). Specifically, an enzyme kinetics assay was performed by incubating a fixed concentration of enzyme at 1 nM with fluorogenic substrate 4MU-NeuAc at concentrations ranging from 4000 μM to 7.8 μM. ASC1 was active with a Vmax of 5.5×10⁷, causing the release of sialic acid which generated fluorescence. Assays were conducted at pH 5.6.

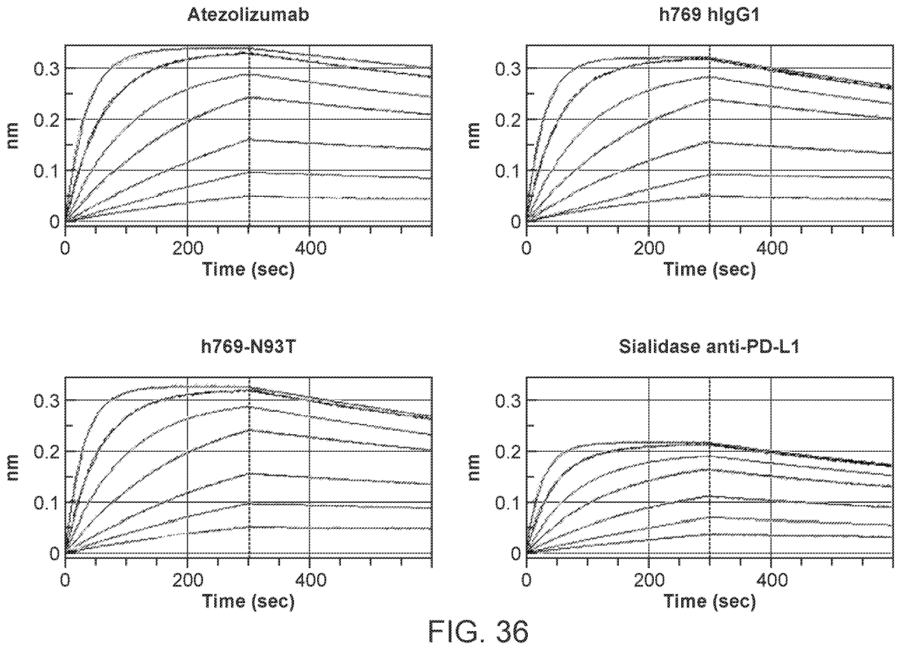

FIG. 36 shows a binding assay (ForteBio octet binding) between atezolizumab, h769 hIgG1, h769-N93T or ASC1 and recombinant human PD-L1. TABLE 18 has the binding kinetics of the four test articles demonstrating very similar KDs in the 1-2 nM range.

TABLE 18

| Clone | KD (M) | kon(1/Ms) | kdis(1/s) |
|---|---|---|---|
| atezolizumab | 1.61 × 10⁻⁰⁹ | 3.02 × 105 | 4.87 × 10⁻⁰⁴ |
| h769 | 1.78 × 10⁻⁰⁹ | 3.53 × 105 | 6.23 × 10⁻⁰⁴ |
| h769.T | 1.76 × 10⁻⁰⁹ | 3.57 × 105 | 6.29 × 10⁻⁰⁴ |
| ASC1 | 1.72 × 10⁻⁰⁹ | 4.56 × 105 | 7.66 × 10⁻⁰⁴ |

A second Janus PD-L1 antibody sialidase conjugate was constructed using Neu2 with M1D, V6Y, P62G, A93E, Q126Y, I187K, A242F, Q270T, and C332A mutations, the variable region of anti-PD-L1 antibody h769.T (as described in Example 4 herein, and also referred to as h769-N93T), and a human IgG1 Fc domain including an N297A mutation. This Janus PD-L1 antibody sialidase conjugate (referred to as ASC3, and including a first polypeptide chain with amino acid sequence SEQ ID NO: 205, encoded by nucleotide sequence SEQ ID NO: 208, a second polypeptide chain with amino acid sequence SEQ ID NO: 213, encoded by nucleotide sequence SEQ ID NO: 215, and a third polypeptide chain with amino acid sequence SEQ ID NO: 214, encoded by nucleotide sequence SEQ ID NO: 216) was expressed and characterized for purity using SDS-PAGE and enzymatic activity using 4MU-NeuAc as described below.

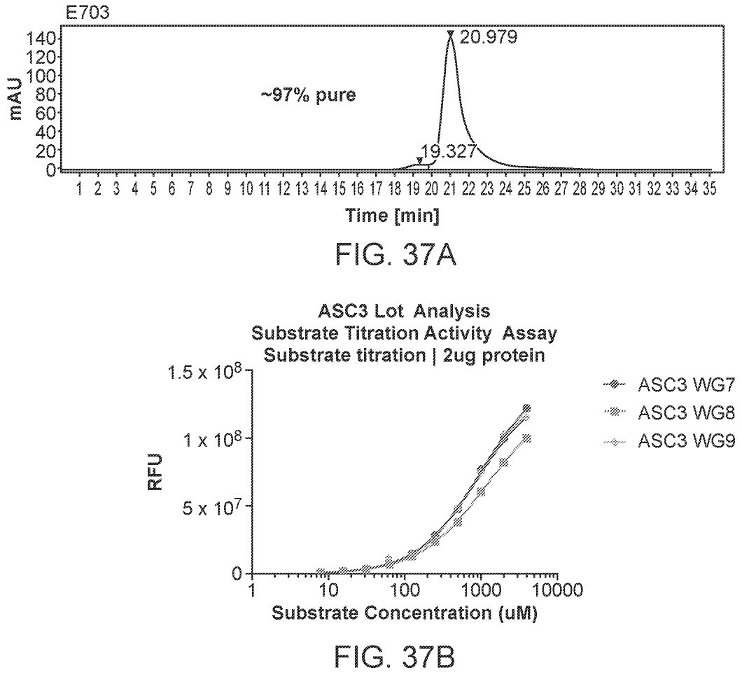

ASC3 was expressed in a 2,000 mL transfection of Expi293 human cells using the pCEP4 mammalian expression vector. The PD-L1 antibody sialidase conjugate was purified using protein A followed by cation exchange and Ceramic Hydroxyapatite chromatography, quantified with a UV-Vis spectrophotometer (NanoDrop), and examined by SDS-PAGE. ASC3 expressed well with a 97% purity by SEC after purification (FIG. 37A).

The activity of ASC3 was assayed by measuring the release of sialic acid from the fluorogenic substrate 4-methylumbelliferyl-N-acetylneuraminic acid (4MU-NeuAc). Specifically, an enzyme kinetics assay was performed by incubating a fixed concentration of enzyme at 1 nM with fluorogenic substrate 4MU-NeuAc at concentrations ranging from 4000 μM to 7.8 μM. Several batches of ASC3 was active with a Vmax of 1.15×10⁸, causing the release of sialic acid which generated fluorescence as shown in FIG. 37B. Assays were conducted at pH 5.6.

A series of additional PD-L1 antibody sialidase conjugates were made. A third was constructed using Neu2 with M1D, V6Y, K9D, I187K, C332A, A93E, V363R, L365R, E218A, and C219N mutations, the variable region of anti-PD-L1 antibody h769.T (as described in Example 4 herein, and also referred to as h769-N93T), and a human IgG1 Fc domain including an N297A mutation. This Janus PD-L1 antibody sialidase conjugate (referred to as ASC4 loss of function (LOF), and including a first polypeptide chain with amino acid sequence SEQ ID NO: 205, encoded by nucleotide sequence SEQ ID NO: 208, a second polypeptide chain with amino acid sequence SEQ ID NO: 213, encoded by nucleotide sequence SEQ ID NO: 215, and a third polypeptide chain with amino acid sequence SEQ ID NO: 217, encoded by nucleotide sequence SEQ ID NO: 218) was expressed and characterized for purity using SDS-PAGE and enzymatic activity using 4MU-NeuAc as described above.

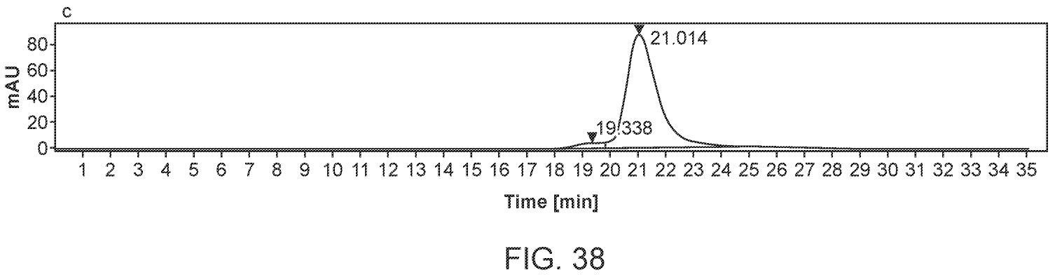

ASC4 LOF was expressed in a 1,000 mL transfection of Expi293 human cells using the pCEP4 mammalian expression vector. The PD-L1 antibody sialidase conjugate was purified using protein A followed by cation exchange and CHT Ceramic Hydroxyapatite chromatography, quantified with a UV-Vis spectrophotometer (NanoDrop), and examined by SDS-PAGE. ASC4 LOF expressed well with a purity of 65% by SEC after purification (FIG. 38). As expected, ASC4 LOF had no detectable activity using 4MU-NeuAc as a substrate.

A fourth PD-L1 antibody sialidase conjugate was constructed using Neu2 with M1D, V6Y, A42R, P62G, A93E, Q126Y, I187K, A242F, Q270T, and C332A mutations, the variable region of anti-PD-L1 antibody h769.T (as described in Example 4 herein, and also referred to as h769-N93T), and a human IgG1 Fc domain including an N297A mutation. This Janus PD-L1 antibody sialidase conjugate (referred to as ASC5, and including a first polypeptide chain with amino acid sequence SEQ ID NO: 205, encoded by nucleotide sequence SEQ ID NO: 208, a second polypeptide chain with amino acid sequence SEQ ID NO: 213, encoded by nucleotide sequence SEQ ID NO: 215, and a third polypeptide chain with amino acid sequence SEQ ID NO: 219, encoded by nucleotide sequence SEQ ID NO: 220) was expressed and characterized for purity using SDS-PAGE and enzymatic activity using 4MU-NeuAc as described above.

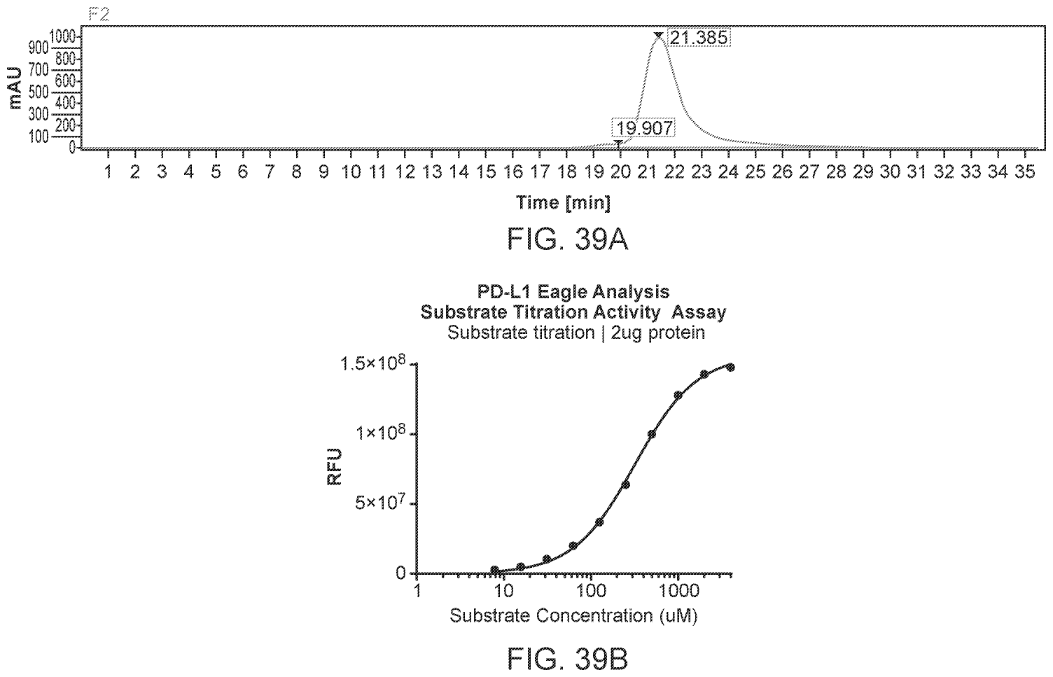

ASC5 was expressed in a 1,000 mL transfection of Expi293 human cells using the pCEP4 mammalian expression vector. The PD-L1 antibody sialidase conjugate was purified using protein A followed by cation exchange and Ceramic Hydroxyapatite chromatography, quantified with a UV-Vis spectrophotometer (NanoDrop), and examined by SDS-PAGE. ASC5 expressed well with a purity of 98% monomeric heterodimer by SEC (FIG. 39A). ASC5 was active with a Vmax of 1.4×10⁸, causing the release of sialic acid which generated fluorescence as shown in FIG. 39B.

A fifth PD-L1 antibody sialidase conjugates was constructed using Neu2 with M1D, V6Y, P62G, A93E, Q126Y, I187K, A242F, Q270T, and C332A mutations, the variable region of anti-PD-L1 antibody h769.T (as described in Example 4 herein, and also referred to as h769-N93T), and a human IgG1 Fc domain including an N297A mutation. This Janus PD-L1 antibody sialidase conjugate (referred to as ASC2, and including a first polypeptide chain with amino acid sequence SEQ ID NO: 205, encoded by nucleotide sequence SEQ ID NO: 208, a second polypeptide chain with amino acid sequence SEQ ID NO: 206, encoded by nucleotide sequence SEQ ID NO: 209, and a third polypeptide chain with amino acid sequence SEQ ID NO: 211, encoded by nucleotide sequence SEQ ID NO: 212) was expressed and characterized for purity using SDS-PAGE and enzymatic activity using 4MU-NeuAc as described above.

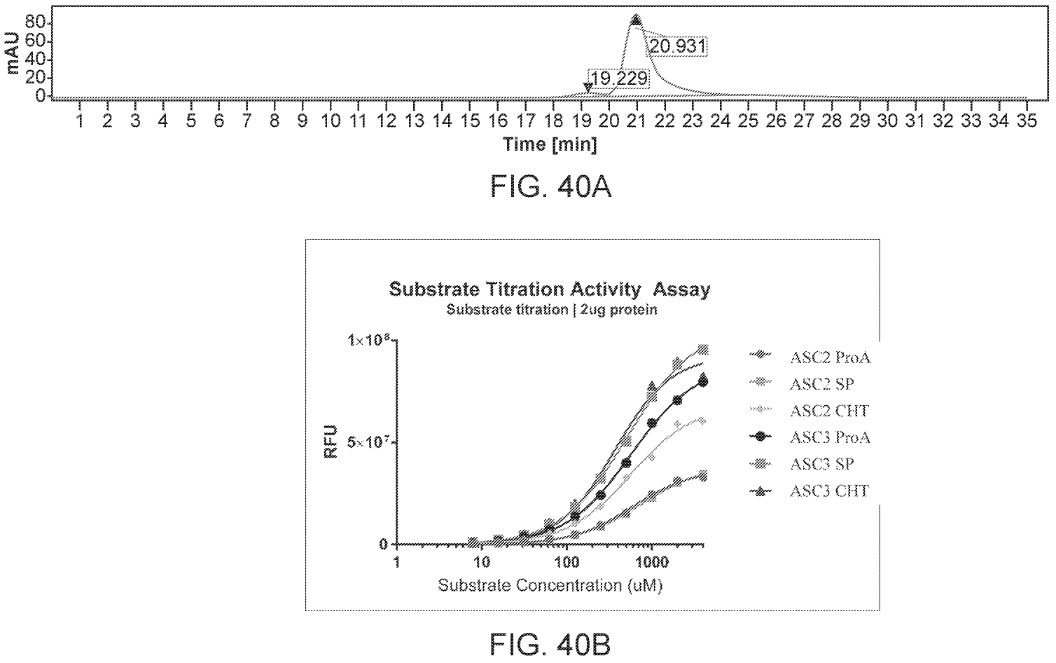

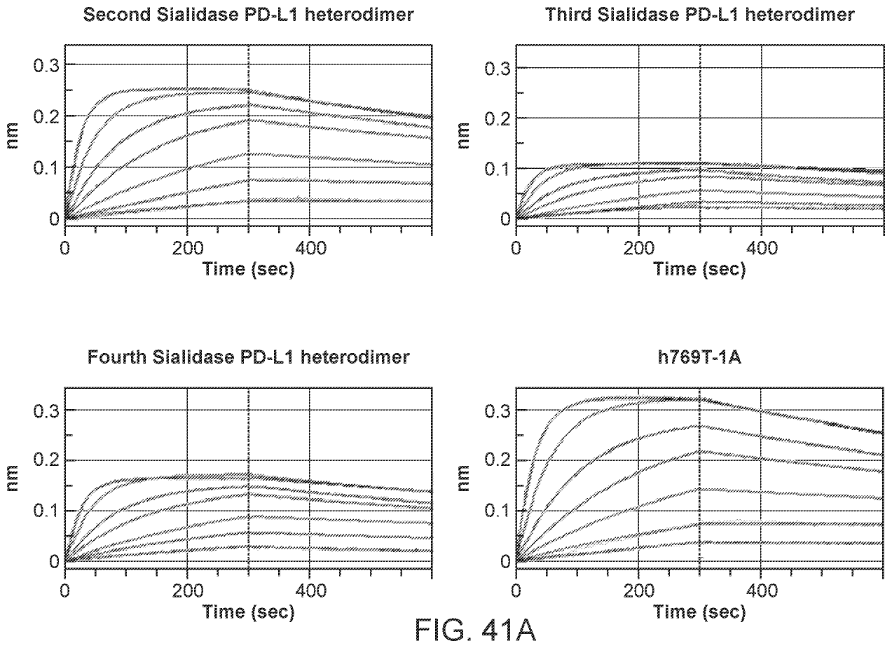

ASC2 was expressed in a 1,000 mL transfection of Expi293 human cells using the pCEP4 mammalian expression vector. The PD-L1 antibody sialidase conjugate was purified using protein A followed by cation exchange and Ceramic Hydroxyapatite chromatography, quantified with a UV-Vis spectrophotometer (NanoDrop), and examined by SDS-PAGE. ASC2 expressed well with a purity of 90% by SEC as shown in FIG. 40A. ASC2 was active with a Vmax of 6.05×10⁷, causing the release of sialic acid which generated fluorescence. FIG. 40B depicts the activity of ASC2 following protein A purification (ProA), after cation exchange (SP) and after Ceramic Hydroxyapatite chromatography (CHT). For comparison, the same results are also shown for ASC3. Both PD-L1 antibody sialidase conjugates demonstrated improved activity as the molecules were purified to homogeneity.

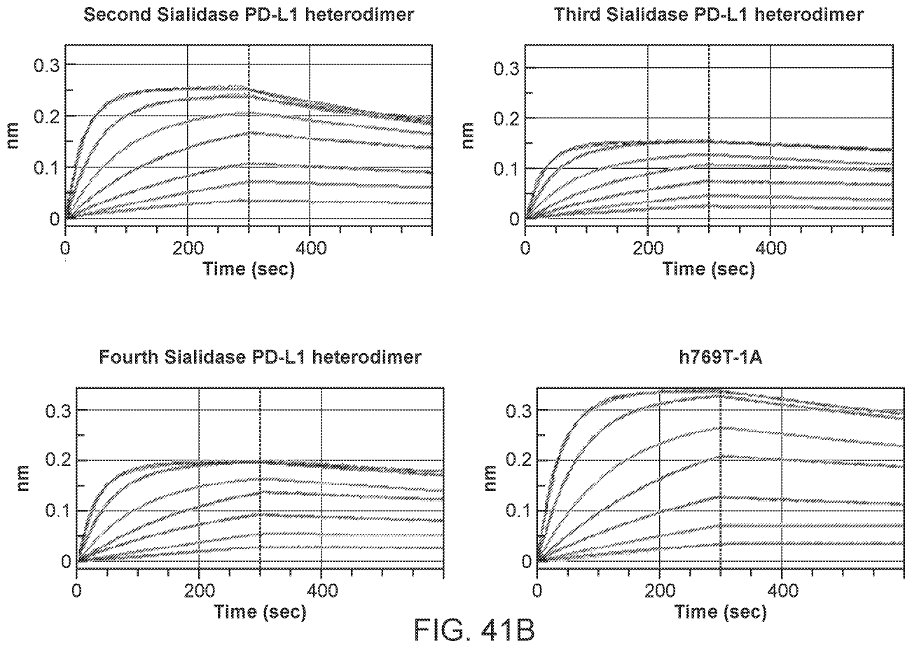

The ability of PD-L1 antibody sialidase conjugates to bind human and cynomolgus PD-L1 was confirmed. FIG. 41 depicts human PD-L1 (FIG. 41A) and cynomolgus PD-L1 (FIG. 41B) binding kinetics to selected of PD-L1 antibody sialidase conjugates as compared to h769.T-1A (as described above in Example 4 herein). KD, Kon and Kdis values for human and cynomolgus PD-L1 of the PD-L1 antibody sialidase conjugates compared to h769.T-1A are shown in TABLE 19.

TABLE 19

| hPD-L1 | KD (M) | kon(1/Ms) | kdis(1/s) | cPD-L1 | KD (M) | kon(1/Ms) | kdis(1/s) |
|---|---|---|---|---|---|---|---|
| ASC3 | 1.73 × 10⁻⁰⁹ | 4.36 × 10⁵ | 7.51 × 10⁻⁰⁴ | ASC3 | 2.26 × 10⁻⁰⁹ | 3.67 × 10⁵ | 8.21 × 10⁻⁰⁴ |
| ASC4 LOF | 1.79 × 10⁻⁰⁹ | 4.49 × 10⁵ | 7.55 × 10⁻⁰⁴ | ASC4 LOF | 1.04 × 10⁻⁰⁹ | 4.23 × 10⁵ | 3.90 × 10⁻⁰⁴ |
| ASC5 | 1.66 × 10⁻⁰⁹ | 4.33 × 10⁵ | 7.21 × 10⁻⁰⁴ | ASC5 | 1.23 × 10⁻⁰⁹ | 3.69 × 10⁵ | 4.38 × 10⁻⁰⁴ |
| h769.T-1A | 2.13 × 10⁻⁰⁹ | 3.46 × 10⁵ | 7.28 × 10⁻⁰⁴ | h769.T-1A | 1.53 × 10⁻⁰⁹ | 2.97 × 10⁵ | 4.43 × 10⁻⁰⁴ |

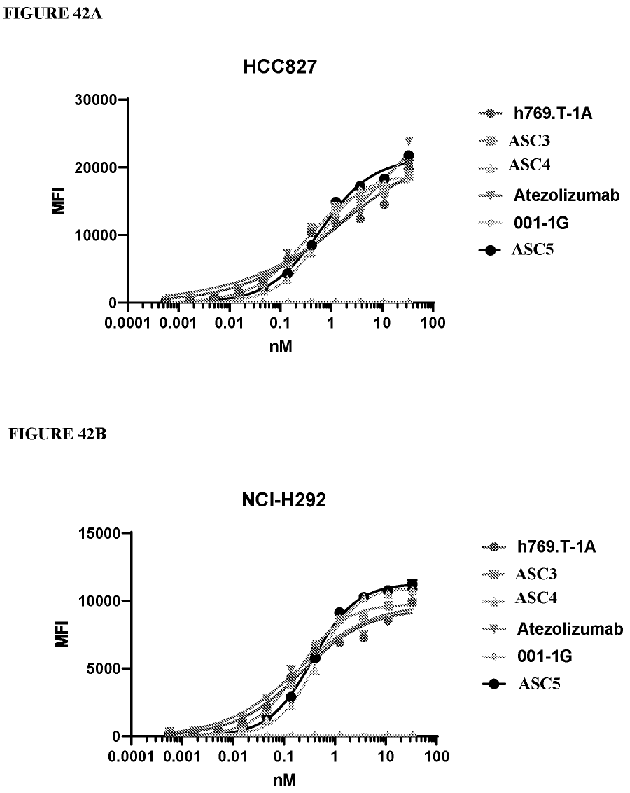

The ability of PD-L1 antibody sialidase conjugates to bind PD-L1 on HCC827 and NCI-H292 cells was examined next. Cells were incubated with antibody (h769.T-1A and atezolizumab) and ASC3, ASC4 LOF, and ASC5 molecules at 4° C. for 30 min. After washing with staining buffer, cells were incubated with AF647 Goat anti-human IgG(H+L) in staining buffer at 4° C. for 30 min. After 2× wash with staining buffer, cells were fixed and run on FACSCelesta. FIG. 42 depicts binding of PD-L1 antibody sialidase conjugates to HCC827 (FIG. 42A) and NCI-H292 (FIG. 42B) lung epithelial cell lines. The apparent Kd (nM) for each antibody is depicted in TABLE 20.

TABLE 20

| | Kd (nM) | Kd (nM) |
|---|---|---|
| | HCC827 | NCI-H292 |
| Atezolizumab | 0.1127* | 0.07495** |
| h769.T-1A | 0.1249** | 0.09732** |
| ASC3 | 0.2330* | 0.1925 |
| ASC4 LOF | 0.5599* | 0.4445 |
| ASC5 | 0.4554* | 0.3633 |

*= Calculated using data points up to 11 nM
**= Calculated using data points up to 3.7 nM

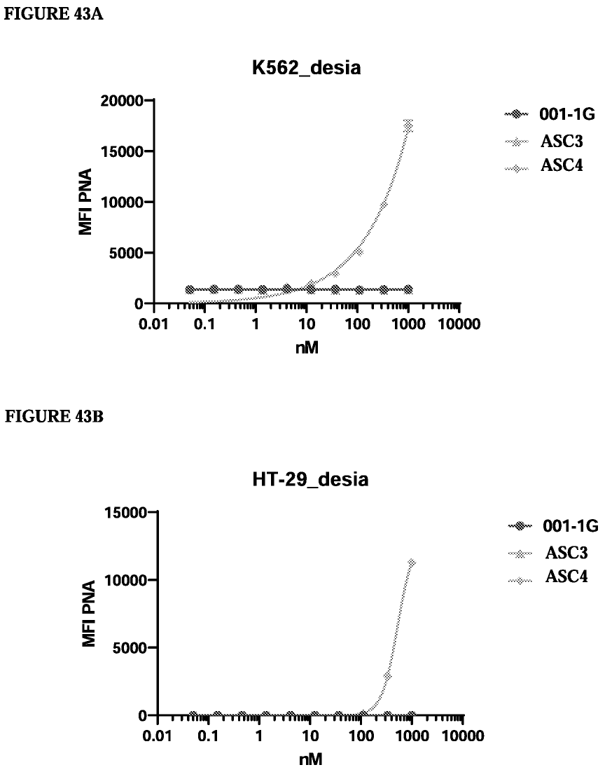

The ability of PD-L1 antibody sialidase conjugates to desialylate K562 and HT-29 cells was examined. Cells were incubated with ASC5 and compared to ASC4 LOF at 37° C. overnight (17 hours). HT-29 cells were lifted in Accutase at 37° C. for 10 min. After wash in staining buffer, cells were incubated in biotin-PNA and live/dead in PBS at 4° C. for 30 min. After washing with PBS, cells were incubated with AF647-Strep in staining buffer at 4° C. for 20 min. Cells were washed twice with staining buffer and run immediately. FIG. 43 depicts desialylation by PD-L1 antibody sialidase conjugates on K562 cells (FIG. 43A) and HT-29 cells (FIG. 43B).

### Example 6

This Example describes the in vivo administration of anti-PD-L1 antibody sialidase conjugates (ASC5) containing human sialidases.

Anti-PD-L1 antibody sialidase conjugates were tested in a transgenic mouse engineered to express human PD-L1 and human PD-1 in which mouse PD-L1 and mouse PD-1 have been disrupted (Biocytogen Inc.). Such double knock-in, knock-out mice were injected with a MC38 murine cancer cell line engineered to express human PD-L1. Mice, 6-8 weeks of age, were inoculated subcutaneously in the right lower flank region with either the parent murine cell line or human PD-L1 expressing tumor cells for tumor development. Mice were randomly allocated to 4 groups of 8 animals each when tumors reached 50-100 mm³, mean ˜75-100 mm³ and treated as shown in TABLE 21.

TABLE 21

| Group | Treatment | Dose | Route | Schedule |
|---|---|---|---|---|
| 1 | 001-1A isotype control | 10 mg/kg | IP | Every other day; |
| | (drug A) | | | 8 doses |
| 2 | ASC5 (drug B) | 10 mg/kg | | |
| 3 | ASC4 LOF (drug C) | 10 mg/kg | | |
| 4 | h769.T-1A (drug D) | 5 mg/kg | | |

Mice were treated via intraperitoneal injection of 10 mg/kg of ASC5 or ASC4 LOF (each as described above in Example 5 herein), 10 mg/kg of isotype control, or 5 mg/kg of h769.T-1A (as described above in Example 4 herein), and tumor volume (mm³) was recorded. Mean tumor volumes for the individual mice for the indicated treatments were determined.

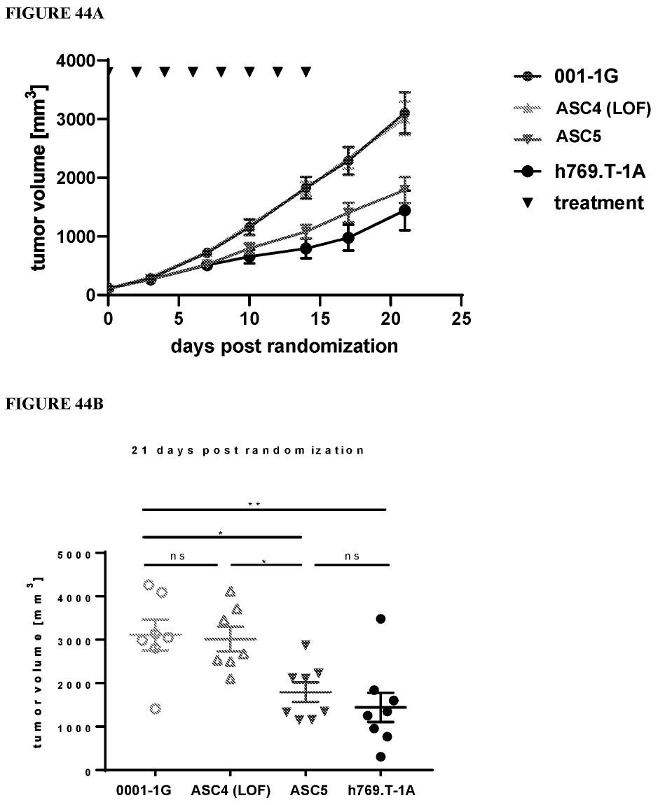

As shown in FIG. 44A, mice treated with ASC5 exhibit statistically meaningful reduced tumor volumes compared to mice treated with the control or ASC4 LOF PD-L1 antibody sialidase conjugates. The reduced tumor volumes following treatment with ASC5 relative to ASC4 LOF demonstrate the importance of the sialidase activity in tumor reduction. Tumor volumes for the individual mice for the indicated treatments are shown in FIG. 44B.

### Example 7

This Example demonstrates the ability of anti-PD-L1 antibody sialidase conjugates (ASCs) to block the PD-1 PD-L1 interaction.

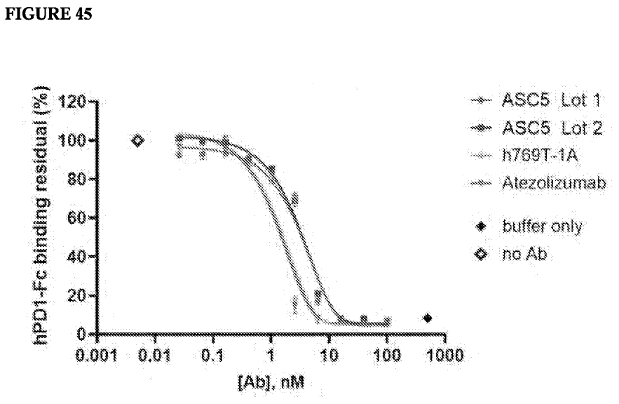

Two lots of ASC5 (as described above in Example 5) were tested for their ability to block a biotinylated human PD-1 Fc fusion (hPD-1-Fc) from binding to human PD-L1 (hPD-L1). ASC5 as well as atezolizumab and h769.T-1A (as described above in Example 4) were 3× titrated and mixed with hPD-1-Fc at a final concentration of 1 μg/mL. The mixture of antibody and hPD-1-Fc was loaded on to hPD-L1 coated ELISA wells for binding. ASCs or antibodies that bind the hPD-1 binding epitope on hPD-L1 will compete for binding and result in a reduction of hPD-1-Fc binding signal. The residual binding of hPD-1-Fc to hPD-L1 was detected with HRP conjugated streptavidin. The plate was developed with TMB and Stop buffer and the absorbance at 450 nm was read using a SpectraMax plate reader. A450 absorbance curves and IC₅₀s were generated using GraphPad Prism software. hPD-1-Fc only (no antibody) and buffer only (no antibody or hPD-1-Fc) were used as controls. As depicted in FIG. 45, ASC5 blocked hPD-1-Fc binding to hPD-L1. IC₅₀s for the two lots of ASC5 were 3.319 nM and 3.134 nM, which was slightly reduced relative to atezolizumab (IC₅₀ of 1.305 nM) or h769.T-1A (IC₅₀ of 1.444 nM). It is contemplated that the difference in IC₅₀ values was due to the difference between the antibody and ASC formats (e.g., ASC5 has only a single PD-L1 binding site while atezolizumab and h769.T-1A each have two PD-L1 binding sites).

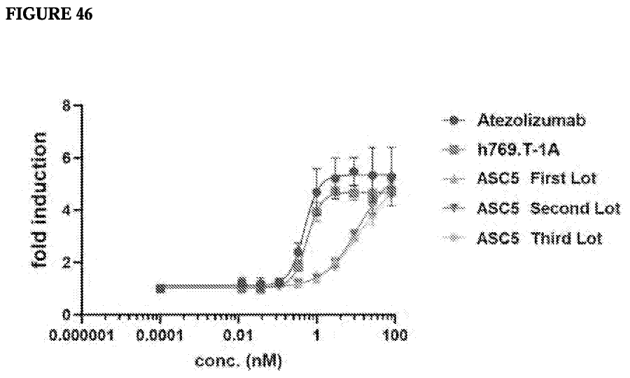

ASC5 was also incubated with (i) engineered CHO-K1 cells expressing human PD-L1 and TCR activating protein and (ii) Jurkat T cells expressing human PD-1, TCR and a luciferase reporter driven by an NFAT response element. Absent intervention, PD-L1 interacting with PD-1 inhibits TCR-mediated luminescence, while blockade of the PD-L1/PD-1 interaction results in a luminescent signal. A luciferase substrate was added after 6 hours of incubation and luminescence was measured. Relative light units (RLU) were calculated by subtracting background (substrate and media only) from assay wells. Fold induction was calculated by dividing the RLU of induced cells minus background by the RLU of a no antibody control minus background (Fold induction=RLU (induced−background)/RLU (no antibody control−background)). Atezolizumab and h769.T-1A were used as positive controls, and each produced a fold induction of 4 to 5 over a range of 0.1 to 10 μg/mL in this assay. As shown in FIG. 46, three different lots of ASC5 caused a dose dependent increase in luminescence, indicating that the ASC is capable of disrupting the PD-L1/PD-1 interaction. EC₅₀s for the three lots of ASC5 were 11.54, 11.59 and 12.71 nM. EC₅₀s for atezolizumab and h769.T-1A were 0.474 nM and 0.5596 nM, respectively. It is contemplated that the difference in EC₅₀ values was due to the difference between the antibody and ASC formats (e.g., ASC5 has only a single PD-L1 binding site while atezolizumab and h769.T-1A each have two PD-L1 binding sites).

### Example 8

This Example demonstrates the ability of anti-PD-L1 antibody sialidase conjugates (ASCs) to remove sialic acid from the surface of human tumor cell lines and primary immune cells.

Following incubation with ASCs, various cell types were stained with α2,3 SiaFind (Lectenz) and PNA lectin. Sialidase activity and removal of α2,3 sialic acid linkages from the cell surface results in decreased staining by α2,3 SiaFind. Sialidase activity and exposure of the underlying galactose sugar upon sialic acid cleavage results in increased staining by PNA lectin.

ASCs were tested on (i) BT-20, HT-29, and SK-BR-3 tumor cell lines, (ii) monocytic-derived dendritic cells (mDCs) generated from two separate healthy donors by treating isolated CD14+ monocytes with 50 ng/ml of both GM-CSF and IL-4, and (iii) PBMCs from two separate healthy donors thawed from frozen stocks. For mDCs and PBMCs only, cells were either stimulated with 300 ng/ml Pam3CSK4 or left unstimulated. For tumor cells, no stimulation was added. Cells were treated overnight (˜15 hours) with prepared 1:3 serial dilutions of ASC5 or ASC4 LOF (each as described above in Example 5), or isotype control with the highest concentration starting at 2,000 μg/ml. An 18-point curve was generated for each cell with each condition and each ASC or isotype concentration for each cell condition was completed in duplicate. After overnight treatment, tumor cells only were treated with Accutase for 15 minutes at 37° C. to loosen cells off of the plate. All cells were washed with PBS and stained with Zombie Aqua cell viability kit at 1:1000 dilution in PBS on ice for 15 minutes to identify live cell populations. Subsequent cell washes using cell staining buffer (Biolegend) were completed between each blocking and staining step including after the cell viability stain. Additionally, staining and resuspension steps were also completed with cell staining buffer. Primary immune cells only were treated with Fc Receptor blocking agent FcX (Biolegend) at 1:20 dilution on ice for 15 minutes. All cells were stained with a mixture of PNA-AF647 (15 μg/ml) and SureLight488-α2,3 SiaFind (30 μg/ml; Lectenz) on ice for 30 minutes. Tumor cells were resuspended and immediately read on BD FACS Celesta via BD Diva Software.

Monocytic DCs were stained with BV421-CD11c and PE-DC-Sign while PBMCs were stained with PE-CD8, PercpCy5.5-CD56, BV421-CD14, BV650-CD19, and BV785-CD3 (Biolegend) on ice for 30 minutes at a 1:40 dilution for all staining antibodies (Biolegend). Primary immune cells were resuspended and immediately read on BD FACS Celesta via BD Diva Software. FloJo software was used to gate out non-debris, single, and live cells. Additionally, mDCs were gated as CD11c+/DC-Sign+ while PBMC populations were separated as CD56hi and CD56int NK cells, CD14hi and CD14int monocytic cells, and CD3/CD8 T Cells. The gMFI of alpha 2,3 SiaFind (Lectenz) and PNA for each population was put into GraphPad Prism software to generate IC₅₀ (TABLE 22) and EC₅₀ (TABLE 23) values, respectively.

ASC5 desialylated both tumor cells and primary human cells as measured by a reduction in α2,3 SiaFind staining, with IC₅₀s between 10 and 100 μg/mL. Following Pam3K stimulation of the primary human cell populations, a clear reduction in IC₅₀s was observed in mDCs from two different donors by 3 orders of magnitude as well as reduced IC₅₀s in CD14hi and CD14int monocytes (TABLE 22). Pam3K stimulation was also shown to increase PD-L1 expression in these cell types which would correlate with the reduced IC₅₀s. Increased PD-L1 expression leads to increased desialylation efficiency of ASC5.

Likewise, ASC5 desialylated both tumor cells and primary human cells as measured by an increase in PNA staining, with EC₅₀s between −100 and 1,000 μg/mL. Following Pam3K stimulation of the primary human cell populations, a clear reduction in EC₅₀s was observed in mDCs from two different donors (TABLE 23). Pam3K stimulation was also shown to increase PD-L1 expression in these cell types which appears to correlate with the reduced EC₅₀s. Increased PD-L1 expression appears to lead to increased desialylation efficiency of ASC5.

TABLE 22
IC₅₀ Measured by α2,3 SiaFind-SL488 Staining

| Cell Category | | IC₅₀ | |
|---|---|---|---|
| (Donor if Applicable) | Cell | No Stim | Pam3K Stim |
| Tumor Cells (NA) | BT-20 | 90.26 | NA |
| | HT-29 | 68.84 | NA |
| | SK-BR-3 | 52.53 | NA |
| Differentiated Cells (Donor 1) | mDCs | 22.31 | 0.02999 |
| Differentiated Cells (Donor 2) | mDCs | 17.81 | 0.02217 |
| PBMCs (Donor 3) | CD14hi Monocytes | 19.35 | 3.468 |
| | CD14int Monocytes | 14.82 | 1.223 |
| | CD56hi NK Cells | 60.11 | 76.14 |
| | CD56int NK Cells | 31.08 | 22.81 |
| | CD8 T Cells | 51.09 | 63.62 |
| PBMCs (Donor 4) | CD14hi Monocytes | 8.998 | 3.633 |
| | CD14int Monocytes | 6.472 | 3.385 |
| | CD56hi NK Cells | 10.92 | 5.329 |
| | CD56int NK Cells | 13.24 | 10.17 |
| | CD8 T Cells | 9.435 | 13.86 |

TABLE 23
EC₅₀ Measured by PNA-AF647 Staining

| Cell Category | | EC₅₀ | |
|---|---|---|---|
| (Donor if Applicable) | Cell | No Stim | Pam3K Stim |
| Tumor Cells (NA) | BT-20 | 1769 | NA |
| | HT-29 | 1102 | NA |
| | SK-BR-3 | 1549 | NA |
| Differentiated Cells (Donor 1) | mDCs | 2353 | 511.1 |
| Differentiated Cells (Donor 2) | mDCs | 79557 | 5847 |
| PBMCs (Donor 3) | CD14hi Monocytes | 86.15 | 91.12 |
| | CD14int Monocytes | 1435 | 3612 |
| | CD56hi NK Cells | 248.3 | 542.8 |
| | CD56int NK Cells | 257.8 | 252.9 |
| | CD8 T Cells | 617 | 628.2 |
| PBMCs (Donor 4) | CD14hi Monocytes | 168.3 | 162 |
| | CD14int Monocytes | 1212 | 2272 |
| | CD56hi NK Cells | 225.9 | 229 |
| | CD56int NK Cells | 243 | 603.6 |
| | CD8 T Cells | 917.2 | 905.3 |

### Example 9

This Example demonstrates the impact of anti-PD-L1 antibody sialidase conjugates (ASCs) on cytokine release in a human dendritic cell and T cell coculture experiment.

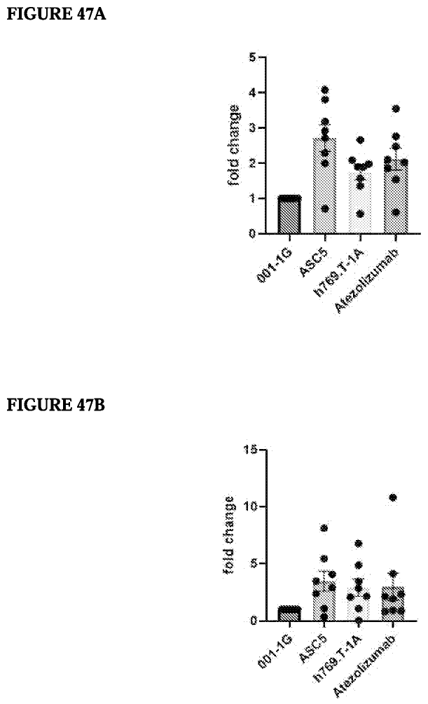

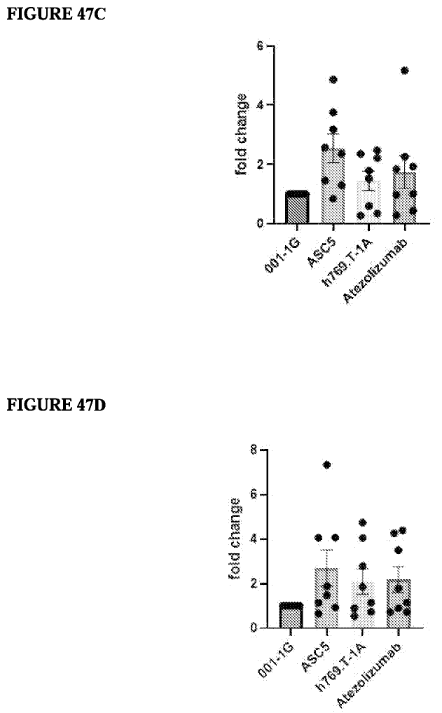

CD14+ monocyte-derived dendritic cells were generated by a 6-day culture in GM-CSF and IL-4 (50 ng/ml each) and co-incubated with allogeneic T cells at 1:2 DC:T ratio in the presence of test articles for 3 days. Supernatants were collected for cytokine analysis by LEGENDplex 13-plex panel. Each data point represents a separate DC-T donor pair (for each test condition two independent experiments were conducted that each included four replicates). ASC5 (as described above in Example 5) was used at 700 nM (100 mg/mL), h769.T-1A (as described above in Example 4) and atezolizumab were used at 70 nM (10 mg/mL), and isotype control was used at 100 mg/mL. FIG. 47A depicts the fold change in IL-2 following treatment with ASC5, h769.T-1A, and atezolizumab compared to isotype control. FIG. 47B, FIG. 47C, and FIG. 47D show similar data for IFN-γ, IL-8 and MCP1, respectively. All four cytokines increased following ASC5 treatment by more than 2 fold, and the increase was at least as much as following treatment with h769.T-1A or atezolizumab.

### Example 10

This Example describes the in vivo administration of anti-PD-L1 antibody sialidase conjugates (ASCs) containing human sialidases.

ASC5 (as described above in Example 5) was tested in a transgenic C57BL6 mouse engineered to express human PD-L1 and human PD-1 in which mouse PD-L1 and mouse PD-1 have been disrupted (Biocytogen Inc.). Mice were injected with a MC38 murine cancer cell line engineered to express human PD-L1. Mice, 6-9 weeks of age, were inoculated subcutaneously in the right lower flank region with tumor cells for tumor development. Mice were randomly allocated to groups of 8 animals each when tumors reached 90-136 mm³, mean˜109 mm³

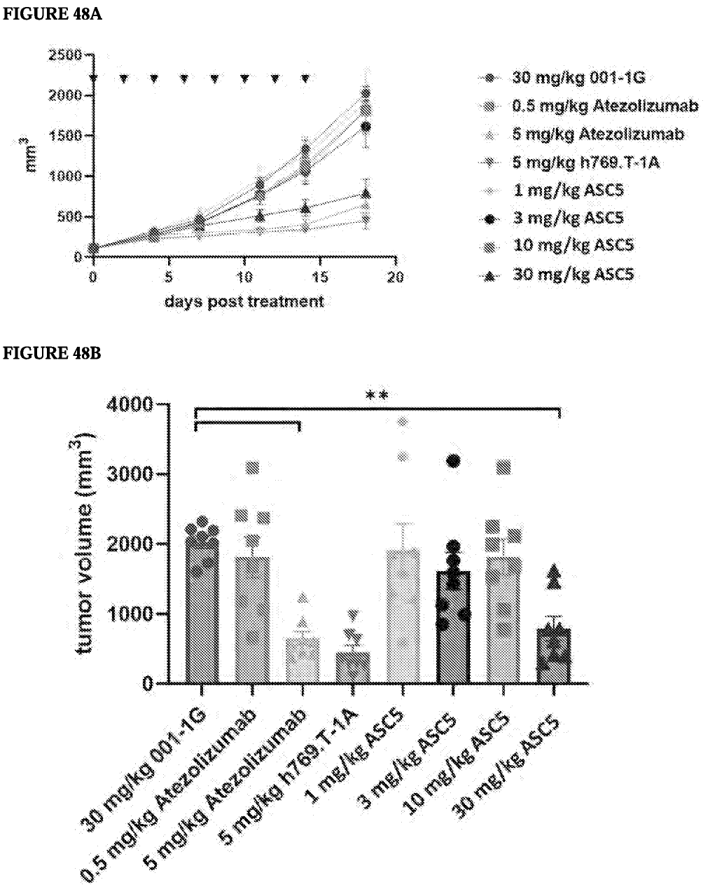

Mice were treated via intraperitoneal injection of ASC5 at either 1, 3, 10, or 30 mg/kg, atezolizumab at 0.5 or 5 mg/kg, h769.T-1A (as described above in Example 4) at 5 mg/kg, or isotype control at 30 mg/kg, and tumor volume (mm³) was recorded. FIG. 48A shows tumor growth through Day 18. FIG. 48B is an analysis of the Day 18 data, demonstrating a significant reduction in tumor growth upon administration of ASC5 at 30 mg/kg, comparable to the response of atezolizumab and h769.T-1A at 5 mg/kg. TABLE 24 depicts tumor growth inhibition (TGI_{TV}) calculated at day 18 for each treatment. TGI_{TV}={1−(TV_{test group}−TV_{test group at day 0})/(TV_{control group}−TV_{control group at day 0})}×100%.

TABLE 24

| Treatment | TGI-_{TV} |
|---|---|
| 0.5 mg/kg atezolizumab | 11.1% |
| 5 mg/kg atezolizumab | 71.8% |
| 5 mg/kg h769. T-1A | 82.1% |
| 1 mg/kg ASC5 | 5.7% |
| 3 mg/kg ASC5 | 21.4% |
| 10 mg/kg ASC5 | 11.1% |
| 30 mg/kg ASC5 | 64.4% |

A CT26 mouse tumor line engineered to express human PD-L1 was grown as a syngeneic subcutaneous tumor in a transgenic BALB/c mouse engineered to express human PD-L1 and human PD-1 and in which mouse PD-L1 and mouse PD-1 have been disrupted (Gempharmatech Inc.). Mice, 8-9 weeks of age, were inoculated subcutaneously in the right lower flank region with tumor cells for tumor development. Mice were randomly allocated to three groups of six animals each when tumors reached 90-120 mm³, with a group mean of 104.06-104.36 mm³.

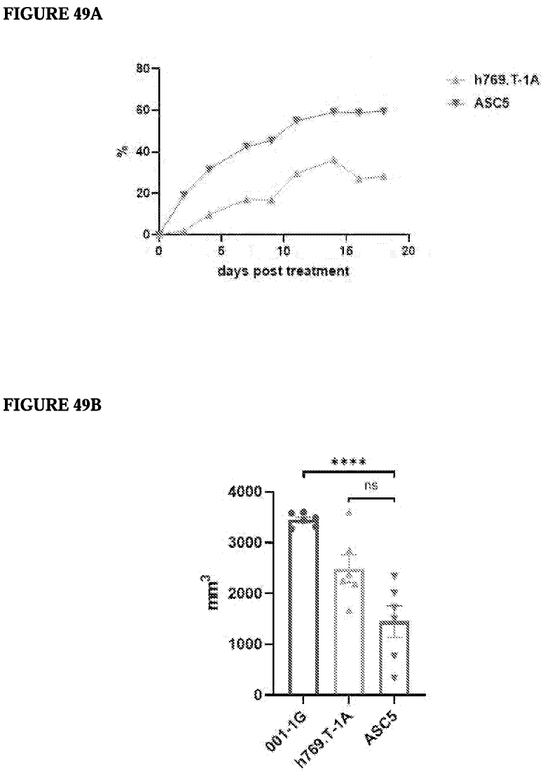

Mice were treated via intraperitoneal injection of ASC5 (as described above in Example 5; 10 mg/kg), h769.T-1A (as described above in Example 4; 5 mg/kg), or isotype control (10 mg/kg), and tumor volume (mm³) was recorded. FIG. 49A shows percent tumor growth inhibition (TGI) through Day 18. FIG. 49B is an analysis of the Day 18 data, demonstrating significant reduction in tumor growth upon administration of ASC5, which was greater than the reduction for h769.T-1A.

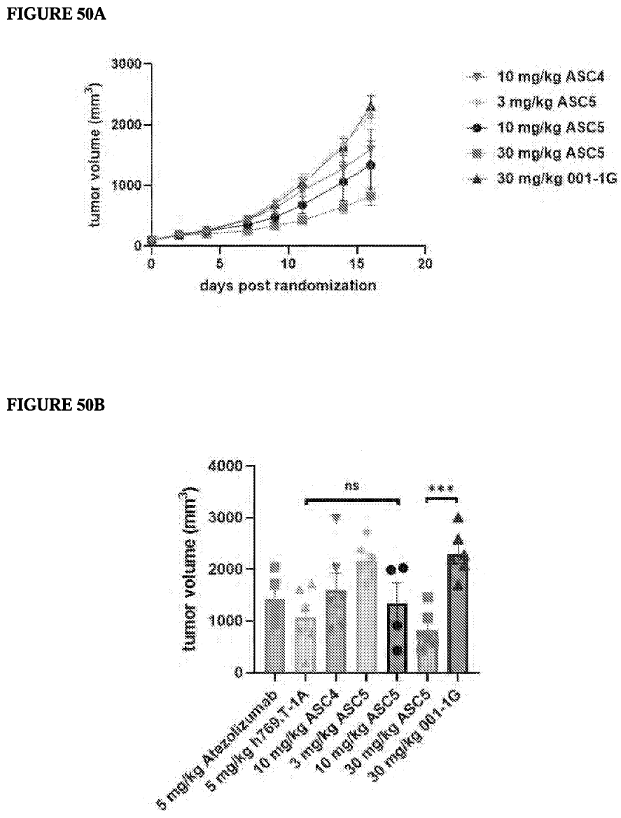

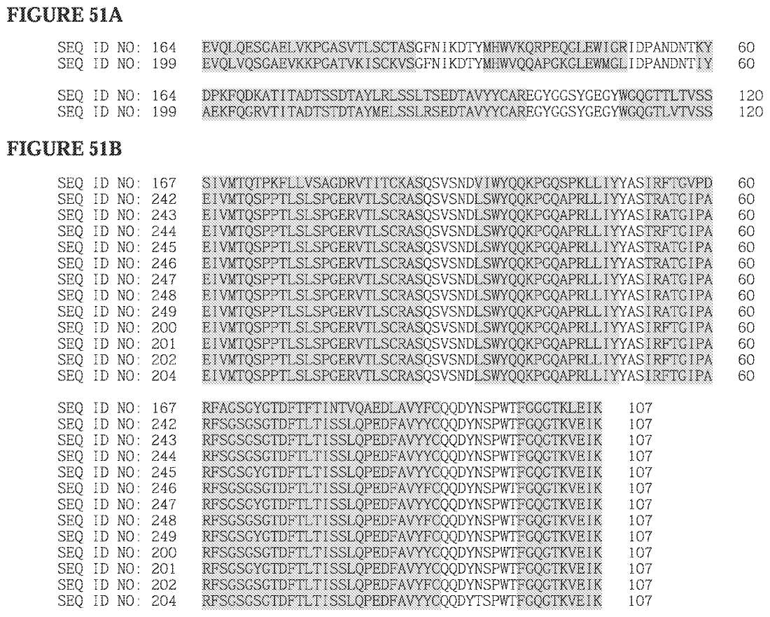

A dose response experiment with ASC5 was carried out in the CT26 mouse model. Mice were treated via intraperitoneal injection at 3, 10 and 30 mg/kg of ASC5, 10 mg/kg ASC4 (LOF), 5 mg/kg atezolizumab, and 30 mg/kg isotype control. 6 mice per group (7-9 weeks of age at inoculation) were randomized when tumors reached 76-125 mm 3 (group mean 102-103 mm³). The humane endpoint was at 3,000 mm³ tumor volume. FIG. 50A shows tumor growth inhibition (TGI) through Day 16. FIG. 50B is an analysis of the Day 16 data demonstrating significant dose dependent reduction in tumor growth upon administration of ASC5.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. An isolated antibody that binds human PD-L1 comprising:
(i) an immunoglobulin heavy chain variable region comprising a CDR_{H1} comprising the amino acid sequence of SEQ ID NO: 161, a CDR_{H2} comprising the amino acid sequence of SEQ ID NO: 162, and a CDR_{H3} comprising the amino acid sequence of SEQ ID NO: 163 (PAL769-h769-VH); and/or an immunoglobulin light chain variable region comprising a CDR_{L1} comprising the amino acid sequence of SEQ ID NO: 165, a CDR_{L2} comprising the amino acid sequence of SEQ ID NO: 142, and a CDR_{L3} comprising the amino acid sequence of SEQ ID NO: 166 (PAL769-VL, h769-IF3-VL, h769-tm2-VL, h769-tm3-VL);
(ii) an immunoglobulin heavy chain variable region comprising a CDR_{H1} comprising the amino acid sequence of SEQ ID NO: 161, a CDR_{H2} comprising the amino acid sequence of SEQ ID NO: 162, and a CDR_{H3} comprising the amino acid sequence of SEQ ID NO: 163 (PAL769-h769-VH); and/or an immunoglobulin light chain variable region comprising a CDR_{L1} comprising the amino acid sequence of SEQ ID NO: 165, a CDRL2 comprising the amino acid sequence of SEQ ID NO: 142, and a CDRL3 comprising the amino acid sequence of SEQ ID NO: 203 (h769.T-VL);
(iii) an immunoglobulin heavy chain variable region comprising a CDR_{H1} comprising the amino acid sequence of SEQ ID NO: 129, a CDR_{H2} comprising the amino acid sequence of SEQ ID NO: 130, and a CDR_{H3} comprising the amino acid sequence of SEQ ID NO: 131 (PAL752-VH); and/or an immunoglobulin light chain variable region comprising a CDR_{L1} comprising the amino acid sequence of SEQ ID NO: 133, a CDR_{L2} comprising the amino acid sequence of SEQ ID NO: 134, and a CDR_{L3} comprising the amino acid sequence of SEQ ID NO: 135 (PAL752-VL);
(iv) an immunoglobulin heavy chain variable region comprising a CDR_{H1} comprising the amino acid sequence of SEQ ID NO: 137, a CDR_{H2} comprising the amino acid sequence of SEQ ID NO: 138, and/or a CDR_{H3} comprising the amino acid sequence of SEQ ID NO: 139 (PAL759-VH); and an immunoglobulin light chain variable region comprising a CDR_{L1} comprising the amino acid sequence of SEQ ID NO: 141, a CDR_{L2} comprising the amino acid sequence of SEQ ID NO: 142, and a CDR_{L3} comprising the amino acid sequence of SEQ ID NO: 143 (PAL759-VL);
(v) an immunoglobulin heavy chain variable region comprising a CDR_{H1} comprising the amino acid sequence of SEQ ID NO: 145, a CDR_{H2} comprising the amino acid sequence of SEQ ID NO: 146, and a CDR_{H3} comprising the amino acid sequence of SEQ ID NO: 147 (PAL760-VH); and/or an immunoglobulin light chain variable region comprising a CDR_{L1} comprising the amino acid sequence of SEQ ID NO: 149, a CDR_{L2} comprising the amino acid sequence of SEQ ID NO: 150, and a CDR_{L3} comprising the amino acid sequence of SEQ ID NO: 151 (PAL760-VL);
(vi) an immunoglobulin heavy chain variable region comprising a CDR_{H1} comprising the amino acid sequence of SEQ ID NO: 153, a CDR_{H2} comprising the amino acid sequence of SEQ ID NO: 154, and a CDR_{H3} comprising the amino acid sequence of SEQ ID NO: 155 (PAL767-VH); and/or an immunoglobulin light chain variable region comprising a CDR_{L1} comprising the amino acid sequence of SEQ ID NO: 157, a CDR_{L2} comprising the amino acid sequence of SEQ ID NO: 158, and a CDR_{L3} comprising the amino acid sequence of SEQ ID NO: 159 (PAL767-VL);
(vii) an immunoglobulin heavy chain variable region comprising a CDR_{H1} comprising the amino acid sequence of SEQ ID NO: 161, a CDR_{H2} comprising the amino acid sequence of SEQ ID NO: 168, and a CDR_{H3} comprising the amino acid sequence of SEQ ID NO: 169 (PAL771-VH); and/or an immunoglobulin light chain variable region comprising a CDR_{L1} comprising the amino acid sequence of SEQ ID NO: 171, a CDR_{L2} comprising the amino acid sequence of SEQ ID NO: 172, and a CDR_{L3} comprising the amino acid sequence of SEQ ID NO: 173 (PAL771-VL);
(viii) an immunoglobulin heavy chain variable region comprising a CDR_{H1} comprising the amino acid sequence of SEQ ID NO: 175, a CDR_{H2} comprising the amino acid sequence of SEQ ID NO: 176, and a CDR_{H3} comprising the amino acid sequence of SEQ ID NO: 177 (PAL785-VH); and/or an immunoglobulin light chain variable region comprising a CDR_{L1} comprising the amino acid sequence of SEQ ID NO: 179, a CDR_{L2} comprising the amino acid sequence of SEQ ID NO: 180, and a CDR_{L3} comprising the amino acid sequence of SEQ ID NO: 181 (PAL785-VL);
(ix) an immunoglobulin heavy chain variable region comprising a CDR_{H1} comprising the amino acid sequence of SEQ ID NO: 183, a CDR_{H2} comprising the amino acid sequence of SEQ ID NO: 184, and a CDR_{H3} comprising the amino acid sequence of SEQ ID NO: 185 (PAL787-VH); and/or an immunoglobulin light chain variable region comprising a CDR_{L1} comprising the amino acid sequence of SEQ ID NO: 187, a CDR_{L2} comprising the amino acid sequence of SEQ ID NO: 188, and a CDR_{L3} comprising the amino acid sequence of SEQ ID NO: 189 (PAL787-VL); or
(x) an immunoglobulin heavy chain variable region comprising a CDR_{H1} comprising the amino acid sequence of SEQ ID NO: 191, a CDR_{H2} comprising the amino acid sequence of SEQ ID NO: 192, and a CDR_{H3} comprising the amino acid sequence of SEQ ID NO: 193 (PAL788-VH); and/or an immunoglobulin light chain variable region comprising a CDR_{L1} comprising the amino acid sequence of SEQ ID NO: 195, a CDR_{L2} comprising the amino acid sequence of SEQ ID NO: 196, and a CDR_{L3} comprising the amino acid sequence of SEQ ID NO: 197 (PAL788-VL).

2. The isolated antibody of claim 1, wherein the CDRs are interposed between human or humanized immunoglobulin framework regions.

3. An isolated antibody that binds human PD-L1 comprising:
(i) an immunoglobulin heavy chain variable region comprising the amino acid sequence of SEQ ID NO: 164 (PAL769-VH), and an immunoglobulin light chain variable region comprising the amino acid sequence of SEQ ID NO: 167 (PAL769-VL);
(ii) an immunoglobulin heavy chain variable region comprising the amino acid sequence of SEQ ID NO: 199 (h769 VH), and an immunoglobulin light chain variable region comprising the amino acid sequence of SEQ ID NO: 200 (h769-IF3-VL);
(iii) an immunoglobulin heavy chain variable region comprising the amino acid sequence of SEQ ID NO: 199 (h769-VH), and an immunoglobulin light chain variable region comprising the amino acid sequence of SEQ ID NO: 201 (h769-tm2-VL);
(iv) an immunoglobulin heavy chain variable region comprising the amino acid sequence of SEQ ID NO: 199 (h769 VH), and an immunoglobulin light chain variable region comprising the amino acid sequence of SEQ ID NO: 202 (h769-tm3-VL);
(v) an immunoglobulin heavy chain variable region comprising the amino acid sequence of SEQ ID NO: 199 (h769-VH), and an immunoglobulin light chain variable region comprising the amino acid sequence of SEQ ID NO: 204 (h769.T-VL);
(vi) an immunoglobulin heavy chain variable region comprising the amino acid sequence of SEQ ID NO: 132 (PAL752-VH), and an immunoglobulin light chain variable region comprising the amino acid sequence of SEQ ID NO: 136 (PAL752-VL);
(vii) an immunoglobulin heavy chain variable region comprising the amino acid sequence of SEQ ID NO: 140 (PAL759-VH), and an immunoglobulin light chain variable region comprising the amino acid sequence of SEQ ID NO: 144 (PAL759-VL);
(viii) an immunoglobulin heavy chain variable region comprising the amino acid sequence of SEQ ID NO: 148 (PAL760-VH), and an immunoglobulin light chain variable region comprising the amino acid sequence of SEQ ID NO: 152 (PAL760-VL);
(ix) an immunoglobulin heavy chain variable region comprising the amino acid sequence of SEQ ID NO: 156 (PAL767-VH), and an immunoglobulin light chain variable region comprising the amino acid sequence of SEQ ID NO: 160 (PAL767-VL);
(x) an immunoglobulin heavy chain variable region comprising the amino acid sequence of SEQ ID NO: 170 (PAL771-VH), and an immunoglobulin light chain variable region comprising the amino acid sequence of SEQ ID NO: 174 (PAL771-VL);
(xi) an immunoglobulin heavy chain variable region comprising the amino acid sequence of SEQ ID NO: 178 (PAL785-VH), and an immunoglobulin light chain variable region comprising the amino acid sequence of SEQ ID NO: 182 (PAL785-VL);
(xii) an immunoglobulin heavy chain variable region comprising the amino acid sequence of SEQ ID NO: 186 (PAL787-VH), and an immunoglobulin light chain variable region comprising the amino acid sequence of SEQ ID NO: 190 (PAL787-VL); or
(xiii) an immunoglobulin heavy chain variable region comprising the amino acid sequence of SEQ ID NO: 194 (PAL788-VH), and an immunoglobulin light chain variable region comprising the amino acid sequence of SEQ ID NO: 198 (PAL788-VL).

4. The isolated antibody of any one of claims 1-3, further comprising a heavy chain and/or light chain constant region.

5. The isolated antibody of claim 4, wherein the heavy chain constant region is selected from an IgG1, IgG2, IgG3, and IgG4 heavy chain constant region.

6. The isolated antibody of any one of claims 1-5, wherein the antibody binds to human PD-L1 with a K_{D} of 5 nM or lower, 1 nM or lower, 0.75 nM or lower, 0.5 nM or lower, 0.1 nM, 0.075 nM, or 0.05 nM or lower, as measured by surface plasmon resonance or bio-layer interferometry.

7. The isolated antibody of any one of claims 1-6, wherein the antibody also binds to Macaca fascicularis (cynomolgus) PD-L1.

8. An isolated antibody that competes with the antibody of any one of claims 1-7 for binding to human PD-L1.

9. An isolated antibody that binds to the same epitope on human PD-L1 as the antibody of any one of claims 1-8.

10. An isolated nucleic acid comprising a nucleotide sequence encoding the immunoglobulin heavy chain variable region of any one of claims 1-7 and/or a nucleotide sequence encoding the immunoglobulin light chain variable region of any one of claims 1-7.

11. An expression vector comprising: (i) a nucleic acid comprising a nucleotide sequence encoding the immunoglobulin heavy chain variable region of any one of claims 1-7; and/or (ii) a nucleic acid comprising a nucleotide sequence encoding the immunoglobulin light chain variable region of any one of claims 1-7.

12. A host cell comprising the expression vector of claim 11.

13. A fusion protein comprising:
(a) a sialidase enzyme; and
(b) an anti-PD-L1 immunoglobulin antigen-binding domain derived from the anti-PD-L1 antibody of any one of claims 1-9.

14. The fusion protein of claim 13, wherein the sialidase is a human sialidase.

15. The fusion protein of claim 13 or 14, wherein the sialidase is a recombinant mutant human sialidase.

16. The fusion protein of claim 15, wherein the sialidase comprises:
(a) a substitution or deletion of a methionine residue at a position corresponding to position 1 of wild-type human Neu2 (M1);
(b) a substitution of a valine residue at a position corresponding to position 6 of wild-type human Neu2 (V6);
(c) a substitution of a lysine residue at a position corresponding to position 9 of wild-type human Neu2 (K9);
(d) a substitution of an alanine residue at a position corresponding to position 42 of wild-type human Neu2 (A42);
(e) a substitution of a proline residue at a position corresponding to position 62 of wild-type human Neu2 (P62);
(f) a substitution of an alanine residue at a position corresponding to position 93 of wild-type human Neu2 (A93);
(g) a substitution of a glutamine residue at a position corresponding to position 126 of wild-type human Neu2 (Q126);
(h) a substitution of an isoleucine residue at a position corresponding to position 187 of wild-type human Neu2 (I187);
a substitution of an alanine residue at a position corresponding to position 242 of wild-type human Neu2 (A242);
a substitution of a glutamine residue at a position corresponding to position 270 of wild-type human Neu2 (Q270);
(k) a substitution of a serine residue at a position corresponding to position 301 of wild-type human Neu2 (S301);
(l) a substitution of a tryptophan residue at a position corresponding to position 302 of wild-type human Neu2 (W302);
(m) a substitution of a cysteine residue at a position corresponding to position 332 of wild-type human Neu2 (C332);
(n) a substitution of a valine residue at a position corresponding to position 363 of wild-type human Neu2 (V363); or
(o) a substitution of a leucine residue at a position corresponding to position 365 of wild-type human Neu2 (L365);
or a combination of any of the foregoing substitutions.

17. The fusion protein of claim 16, wherein, in the sialidase:
(a) the methionine residue at a position corresponding to position 1 of wild-type human Neu2 is deleted (ΔM1), is substituted by alanine (M1A), or is substituted by aspartic acid (M1D);
(b) the valine residue at a position corresponding to position 6 of wild-type human Neu2 is substituted by tyrosine (V6Y);
(c) the alanine residue at a position corresponding to position 42 of wild-type human Neu2 is substituted by arginine (A42R)
(d) the lysine residue at a position corresponding to position 9 of wild-type human Neu2 is substituted by aspartic acid (K9D);
(e) the proline residue at a position corresponding to position 62 of wild-type human Neu2 is substituted by asparagine (P62N), aspartic acid (P62D), histidine (P62H), glutamic acid (P62E), glycine (P62G), serine (P62S), or threonine (P62T);
(f) the alanine residue at a position corresponding to position 93 of wild-type human Neu2 is substituted by glutamic acid (A93E) or lysine (A93K);
(g) the glutamine residue at a position corresponding to position 126 of wild-type human Neu2 is substituted by leucine (Q126L), glutamic acid (Q126E), phenylalanine (Q126F), histidine (Q126H), isoleucine (Q126I), or tyrosine (Q126Y);
(h) the isoleucine residue at a position corresponding to position 187 of wild-type human Neu2 is substituted by lysine (I187K);
(i) the alanine residue at a position corresponding to position 242 of wild-type human Neu2 is substituted by cysteine (A242C), phenylalanine (A242F), glycine (A242G), histidine (A242H), isoleucine (A242I), lysine (A242K), leucine (A242L), methionine (A242M), asparagine (A242N), glutamine (A242Q), arginine (A242R), serine (A242S), valine (A242V), tryptophan (A242W), or tyrosine (A242Y);
(j) the glutamine residue at a position corresponding to position 270 of wild-type human Neu2 is substituted by alanine (Q270A), histidine (Q270H), phenylalanine (Q270F), proline (Q270P), serine (Q270S), or threonine (Q270T);
(k) the serine residue at a position corresponding to position 301 of wild-type human Neu2 is substituted by alanine (S301A), aspartic acid (S301D), glutamic acid (S301E), phenylalanine (S301F), histidine (S301H), lysine (S301K), leucine (S301L), methionine (S301M), asparagine (S301N), proline (S301P), glutamine (S301Q), arginine (S301R), threonine (S301T), valine (S301V), tryptophan (S301W), or tyrosine (S301Y);
(l) the tryptophan residue at a position corresponding to position 302 of wild-type human Neu2 is substituted by alanine (W302A), aspartic acid (W302D), phenylalanine (W302F), glycine (W302G), histidine (W302H), isoleucine (W3021), lysine (W302K), leucine (W302L), methionine (W302M), asparagine (W302N), proline (W302P), glutamine (W302Q), arginine (W302R), serine (W302S), threonine (W302T), valine (W302V), or tyrosine (W302Y);
(m) the cysteine residue at a position corresponding to position 332 of wild-type human Neu2 is substituted by alanine (C332A);
(n) the valine residue at a position corresponding to position 363 of wild-type human Neu2 is substituted by arginine (V363R); or
(o) the leucine residue at a position corresponding to position 365 of wild-type human Neu2 is substituted by glutamine (L365Q), histidine (L365H), isoleucine (L365I), lysine (L365K) or serine (L365S);
or the sialidase comprises a combination of any of the foregoing substitutions.

18. The fusion protein of claim 17, wherein the sialidase comprises a substitution selected from ΔM1, M1A, M1D, V6Y, K9D, A42R, P62G, P62N, P62S, P62T, A93E, Q126Y, I187K, A242F, A242W, A242Y, Q270A, Q270T, S301A, S301R, W302K, W302R, C332A, V363R, and L365I, or a combination of any of the foregoing substitutions.

19. The fusion protein of claim 18, wherein the sialidase comprises:
(a) the M1D, V6Y, P62G, A93E, I187K, and C332A substitutions;
(b) the M1D, V6Y, K9D, A93E, I187K, C332A, V363R, and L365I substitutions;
(c) the M1D, V6Y, P62N, I187K, and C332A substitutions;
(d) the M1D, V6Y, I187K, Q270A, S301R, W302K, and C332A substitutions;
(e) the M1D, V6Y, P62S, I187K, Q270A, S301R, W302K, and C332A substitutions;
(f) the M1D, V6Y, P62T, I187K, Q270A, S301R, W302K, and C332A substitutions;
(g) the M1D, V6Y, P62N, I187K, Q270A, S301R, W302K, and C332A substitutions;
(h) the M1D, V6Y, P62G, A93E, I187K, S301A, W302R, and C332A substitutions;
(i) the M1D, V6Y, P62G, A93E, Q126Y, I187K, Q270T, and C332A substitutions;
(j) the M1D, V6Y, P62G, A93E, Q126Y, I187K, and C332A substitutions;
(k) the M1D, V6Y, P62G, A93E, Q126Y, I187K, A242F, Q270T, and C332A substitutions; or
(l) the M1D, V6Y, A42R, P62G, A93E, Q126Y, I187K, A242F, Q270T, and C332A mutations.

20. The fusion protein of any one of claims 13-19, wherein the sialidase is selected from Neu1, Neu2, Neu3, and Neu4.

21. The fusion protein of claim 20, wherein the sialidase is Neu2.

22. The fusion protein of any one of claims 13-21, wherein the sialidase has a different substrate specificity than the corresponding wild-type sialidase.

23. The fusion protein of claim 22, wherein the sialidase can cleave α2,3, α2,6, and/or α2,8 linkages.

24. The fusion protein of claim 23, wherein the sialidase can cleave α2,3 and α2,8 linkages.

25. The fusion protein of any one of claims 13-24, wherein the sialidase comprises any one of SEQ ID NOs: 48-62, 94, 97, 100, 126, or 234.

26. The fusion protein of any one of claims 13-25, wherein the sialidase comprises a mutation set forth in any one of Tables 1-9.

27. The fusion protein of any one of claims 13-26, wherein the fusion protein further comprises an immunoglobulin Fc domain.

28. The fusion protein of claim 27, wherein the immunoglobulin Fc domain is derived from a human IgG1, IgG2, IgG3, IgG4, IgA1, IgA2, IgD, IgE, or IgM Fc domain.

29. The fusion protein of claim 28, wherein the immunoglobulin Fc domain is derived from a human IgG1, IgG2, IgG3, or IgG4 Fc domain.

30. The fusion protein of claim 29, wherein the immunoglobulin Fc domain is derived from a human IgG1 Fc domain.

31. The fusion protein of any one of claims 13-30, wherein the anti-PD-L1 immunoglobulin antigen-binding domain is associated with a second anti-PD-L1 immunoglobulin antigen-binding domain derived from the anti-PD-L1 antibody of any one of claims 1-9 to produce an anti-PD-L1 antigen-binding site.

32. The fusion protein of any one of claims 13-31, wherein the sialidase and the immunoglobulin Fc domain and/or the anti-PD-L1 immunoglobulin antigen-binding domain are linked by a peptide bond or an amino acid linker.

33. The fusion protein of any one of claims 13-32, wherein the fusion protein comprises any one of SEQ ID NOs: 205-207, 211, 213, 214, and 219.

34. An antibody conjugate comprising the fusion protein of any one of claims 13-33.

35. The antibody conjugate of claim 34, wherein the antibody conjugate comprises a single sialidase.

36. The antibody conjugate of claim 34, wherein the antibody conjugate comprises two sialidases.

37. The antibody conjugate of claim 36, wherein the two sialidases are identical.

38. The antibody conjugate of any one of claims 34-37, wherein the antibody conjugate comprises a single anti-PD-L1 antigen-binding site.

39. The antibody conjugate of any one of claims 34-37, wherein the antibody conjugate comprises two anti-PD-L1 antigen-binding sites.

40. The antibody conjugate of claim 39, wherein the two anti-PD-L1 antigen-binding sites are identical.

41. The antibody conjugate of any one of claims 34-40, wherein the antibody conjugate has a molecular weight from about 135 kDa to about 165 kDa.

42. The antibody conjugate of any one of claims 34-40, wherein the antibody conjugate has a molecular weight from about 215 kDa to about 245 kDa.

43. The antibody conjugate of any one of claims 34-42, wherein the antibody conjugate comprises:
(a) a first polypeptide comprising an immunoglobulin light chain;
(b) a second polypeptide comprising an immunoglobulin heavy chain; and
(c) a third polypeptide comprising an immunoglobulin Fc domain and a sialidase;
wherein the first and second polypeptides are covalently linked together and the second and third polypeptides are linked together, and wherein the first polypeptide and the second polypeptide together define an anti-PD-L1 antigen-binding site.

44. The antibody conjugate of claim 43, wherein the third polypeptide comprises the sialidase and the immunoglobulin Fc domain in an N- to C-terminal orientation.

45. The antibody conjugate of claim 43 or 44, wherein the first polypeptide comprises SEQ ID NO: 205.

46. The antibody conjugate of any one of claims 43-45, wherein the second polypeptide comprises SEQ ID NOs: 206 or 213.

47. The antibody conjugate of any one of claims 43-46, wherein the third polypeptide comprises SEQ ID NOs: 207, 211, 214, or 219.

48. The antibody conjugate of any one of claims 34-42, wherein the fusion protein comprises:
(a) a first polypeptide comprising a first immunoglobulin light chain;
(b) a second polypeptide comprising a first immunoglobulin heavy chain and a first sialidase;
(c) a third polypeptide comprising a second immunoglobulin heavy chain and a second sialidase; and
(d) a fourth polypeptide comprising a second immunoglobulin light chain;
wherein the first and second polypeptides are covalently linked together, the third and fourth polypeptides are covalently linked together, and the second and third polypeptides are covalently linked together, and wherein the first polypeptide and the second polypeptide together define a first anti-PD-L1 antigen-binding site, and the third polypeptide and the fourth polypeptide together define a second anti-PD-L1 antigen-binding site.

49. The antibody conjugate of claim 48, wherein the second and third polypeptides comprise the first and second immunoglobulin heavy chain and the first and second sialidase, respectively, in an N- to C-terminal orientation.

50. The antibody conjugate of any one of claims 34-42, wherein the fusion protein comprises:
(a) a first polypeptide comprising a first sialidase, a first immunoglobulin Fc domain, and a first single chain variable fragment (scFv); and
(b) a second polypeptide comprising a second sialidase, a second immunoglobulin Fc domain, and a second single chain variable fragment (scFv);
wherein the first and second polypeptides are covalently linked together, and wherein the first scFv defines a first anti-PD-L1 antigen-binding site, and the second scFv defines a second anti-PD-L1 antigen-binding site.

51. The antibody conjugate of claim 50, wherein the first polypeptide comprises the first sialidase, the first immunoglobulin Fc domain, and the first scFv in an N- to C-terminal orientation, and the second polypeptide comprises the second sialidase, the second immunoglobulin Fc domain, and the second scFv in an N- to C-terminal orientation.

52. The antibody conjugate of any one of claims 34-42, wherein the antibody conjugate comprises:
(a) a first polypeptide comprising an immunoglobulin light chain;
(b) a second polypeptide comprising an immunoglobulin heavy chain and a single chain variable fragment (scFv); and
(c) a third polypeptide comprising an immunoglobulin Fc domain and a sialidase;
wherein the first and second polypeptides are covalently linked together and the second and third polypeptides are covalently linked together, and wherein the immunoglobulin light chain and immunoglobulin heavy chain together define a first anti-PD-L1 antigen-binding site and the scFv defines a second anti-PD-L1 antigen-binding site.

53. The antibody conjugate of claim 52, wherein the second polypeptide comprises the immunoglobulin heavy chain and the scFv in an N- to C-terminal orientation, and the third polypeptide comprises the sialidase and the immunoglobulin Fc domain in an N- to C-terminal orientation.

54. An isolated nucleic acid comprising a nucleotide sequence encoding the fusion protein of any one of claims 13-33, or at least a portion of the antibody conjugate of any one of claims 34-53.

55. An expression vector comprising the nucleic acid of claim 54.

56. A host cell comprising the expression vector of claim 55.

57. A pharmaceutical composition comprising the antibody of any one of claims 1-9, the fusion protein of any one of claims 13-33 or the antibody conjugate of any one of claims 34-53.

58. A method of treating cancer in a subject in need thereof, the method comprising administering to the subject an effective amount of the antibody of any one of claims 1-9, the fusion protein of any one of claims 13-33, the antibody conjugate of any one of claims 34-53, or the pharmaceutical composition of claim 57.

59. The method of claim 58, wherein the cancer is selected from NSCLC, melanoma, bladder, breast, cervical, esophageal, gastric, kidney, lung, ovary, metastatic Merkel cell carcinoma (MCC), metastatic urothelial carcinoma (UC), and pancreatic cancer.
